(19) United States
(12) Patent Application Publication
(10) Pub. No.: US 2013/0345218 A1
(43) Pub. Date: Dec. 26, 2013

(54) INHIBITORS OF INFLUENZA VIRUSES REPLICATION

(71) Applicant: Vertex Pharmaceuticals Incorporated, Cambridge, MA (US)

(72) Inventors: Paul S. Charifson, Framingham, MA (US); Michael P. Clark, Concord, MA (US); Upul K. Bandarage, Lexington, MA (US); Hongbo Deng, Southborough, MA (US); Ioana Davies, Watertown, MA (US); John P. Duffy, Northborough, MA (US); Luc J. Farmer, Montreal (CA); Huai Gao, Arlington, MA (US); Wenxin Gu, Concord, MA (US); Joseph M. Kennedy, Charlestown, MA (US); Mark W. Ledeboer, Acton, MA (US); Brian Ledford, Norton, MA (US); Francois Maltais, Tewksbury, MA (US); Emanuele Perola, Brookline, MA (US); Marion W. Wannamaker, Bolton, MA (US); Tiansheng Wang, Concord, MA (US); Francesco G. Salituro, Marlborough, MA (US)

(21) Appl. No.: 13/918,115

(22) Filed: Jun. 14, 2013

Related U.S. Application Data
(63) Continuation of application No. PCT/US2011/065389, filed on Dec. 16, 2011.
(60) Provisional application No. 61/527,277, filed on Aug. 25, 2011.
(60) Provisional application No. 61/423,933, filed on Dec. 16, 2010.

(51) Int. Cl. C07D 471/04

(52) U.S. Cl. CPC C07D 471/04
USPC 514/234.5; 544/331; 514/275; 544/127; 546/113; 514/300; 544/182; 514/242; 544/212; 514/245

(57) ABSTRACT

Methods of inhibiting the replication of influenza viruses in a biological sample or patient, of reducing the amount of influenza viruses in a biological sample or patient, and of treating influenza in a patient, comprises administering to said biological sample or patient an effective amount of a compound represented by Structural Formula (I):

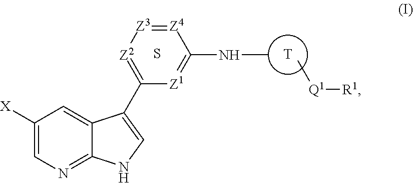

or a pharmaceutically acceptable salt thereof, wherein the values of Structural Formula (I) are as described herein. A compound is represented by Structural Formula (I) or a pharmaceutically acceptable salt thereof, wherein the values of Structural Formula (I) are as described herein. A pharmaceutical composition comprises an effective amount of such a compound or pharmaceutically acceptable salt thereof, and a pharmaceutically acceptable carrier, adjuvant or vehicle.

### CROSS REFERENCE TO RELATED APPLICATIONS

This application is a continuation of PCT Application Number PCT/US2011/065389, filed Dec. 16, 2011, which claims priority to U.S. Provisional Application No. 61/527,277, filed Aug. 25, 2011 and U.S. Provisional Application No. 61/423,933, filed Dec. 16, 2010, the entire contents of which are incorporated herein by reference.

### BACKGROUND OF THE INVENTION

Influenza spreads around the world in seasonal epidemics, resulting in the deaths of hundreds of thousands annually—millions in pandemic years. For example, three influenza pandemics occurred in the 20th century and killed tens of millions of people, with each of these pandemics being caused by the appearance of a new strain of the virus in humans. Often, these new strains result from the spread of an existing influenza virus to humans from other animal species.

Influenza is primarily transmitted from person to person via large virus-laden droplets that are generated when infected persons cough or sneeze; these large droplets can then settle on the mucosal surfaces of the upper respiratory tracts of susceptible individuals who are near (e.g. within about 6 feet) infected persons. Transmission might also occur through direct contact or indirect contact with respiratory secretions, such as touching surfaces contaminated with influenza virus and then touching the eyes, nose or mouth. Adults might be able to spread influenza to others from 1 day before getting symptoms to approximately 5 days after symptoms start. Young children and persons with weakened immune systems might be infectious for 10 or more days after onset of symptoms.

Influenza viruses are RNA viruses of the family Orthomyxoviridae, which comprises five genera: Influenza virus A, Influenza virus B, Influenza virus C, Isavirus and Thogoto virus.

The Influenza virus A genus has one species, influenza A virus. Wild aquatic birds are the natural hosts for a large variety of influenza A. Occasionally, viruses are transmitted to other species and may then cause devastating outbreaks in domestic poultry or give rise to human influenza pandemics. The type A viruses are the most virulent human pathogens among the three influenza types and cause the most severe disease. The influenza A virus can be subdivided into different serotypes based on the antibody response to these viruses. The serotypes that have been confirmed in humans, ordered by the number of known human pandemic deaths, are: H1N1 (which caused Spanish influenza in 1918), H2N2 (which caused Asian Influenza in 1957), H3N2 (which caused Hong Kong Flu in 1968), H5N1 (a pandemic threat in the 2007-08 influenza season), H7N7 (which has unusual zoonotic potential), H1N2 (endemic in humans and pigs), H9N2, H7N2, H7N3 and H10N7.

The Influenza virus B genus has one species, influenza B virus. Influenza B almost exclusively infects humans and is less common than influenza A. The only other animal known to be susceptible to influenza B infection is the seal. This type of influenza mutates at a rate 2-3 times slower than type A and consequently is less genetically diverse, with only one influenza B serotype. As a result of this lack of antigenic diversity, a degree of immunity to influenza B is usually acquired at an early age. However, influenza B mutates enough that lasting immunity is not possible. This reduced rate of antigenic change, combined with its limited host range (inhibiting cross species antigenic shift), ensures that pandemics of influenza B do not occur.

The Influenza virus C genus has one species, influenza C virus, which infects humans and pigs and can cause severe illness and local epidemics. However, influenza C is less common than the other types and usually seems to cause mild disease in children.

Influenza A, B and C viruses are very similar in structure. The virus particle is 80-120 nanometers in diameter and usually roughly spherical, although filamentous forms can occur. Unusually for a virus, its genome is not a single piece of nucleic acid; instead, it contains seven or eight pieces of segmented negative-sense RNA. The Influenza A genome encodes 11 proteins: hemagglutinin (HA), neuraminidase (NA), nucleoprotein (NP), M1, M2, NS1, NS2(NEP), PA, PB1, PB1-F2 and PB2.

HA and NA are large glycoproteins on the outside of the viral particles. HA is a lectin that mediates binding of the virus to target cells and entry of the viral genome into the target cell, while NA is involved in the release of progeny virus from infected cells, by cleaving sugars that bind the mature viral particles. Thus, these proteins have been targets for antiviral drugs. Furthermore, they are antigens to which antibodies can be raised. Influenza A viruses are classified into subtypes based on antibody responses to HA and NA, forming the basis of the H and N distinctions (vide supra) in, for example, H5N1.

Influenza produces direct costs due to lost productivity and associated medical treatment, as well as indirect costs of preventative measures. In the United States, influenza is responsible for a total cost of over $10 billion per year, while it has been estimated that a future pandemic could cause hundreds of billions of dollars in direct and indirect costs. Preventative costs are also high. Governments worldwide have spent billions of U.S. dollars preparing and planning for a potential H5N1 avian influenza pandemic, with costs associated with purchasing drugs and vaccines as well as developing disaster drills and strategies for improved border controls.

Current treatment options for influenza include vaccination, and chemotherapy or chemoprophylaxis with anti-viral medications. Vaccination against influenza with an influenza vaccine is often recommended for high-risk groups, such as children and the elderly, or in people that have asthma, diabetes, or heart disease. However, it is possible to get vaccinated and still get influenza. The vaccine is reformulated each season for a few specific influenza strains but cannot possibly include all the strains actively infecting people in the world for that season. It takes about six months for the manufacturers to formulate and produce the millions of doses required to deal with the seasonal epidemics; occasionally, a new or overlooked strain becomes prominent during that time and infects people although they have been vaccinated (as by the H3N2 Fujian flu in the 2003-2004 influenza season). It is also possible to get infected just before vaccination and get sick with the very strain that the vaccine is supposed to prevent, as the vaccine takes about two weeks to become effective.

Further, the effectiveness of these influenza vaccines is variable. Due to the high mutation rate of the virus, a particular influenza vaccine usually confers protection for no more than a few years. A vaccine formulated for one year may be ineffective in the following year, since the influenza virus changes rapidly over time, and different strains become dominant.

Also, because of the absence of RNA proofreading enzymes, the RNA-dependent RNA polymerase of influenza vRNA makes a single nucleotide insertion error roughly every 10 thousand nucleotides, which is the approximate length of the influenza vRNA. Hence, nearly every newly-manufactured influenza virus is a mutantantigenic drift. The separation of the genome into eight separate segments of vRNA allows mixing or reassortment of vRNAs if more than one viral line has infected a single cell. The resulting rapid change in viral genetics produces antigenic shifts and allows the virus to infect new host species and quickly overcome protective immunity.

Antiviral drugs can also be used to treat influenza, with neuraminidase inhibitors being particularly effective, but viruses can develop resistance to the standard antiviral drugs.

Thus, there is still a need for drugs for treating influenza infections, such as for drugs with expanded treatment window, and/or reduced sensitivity to viral titer.

### SUMMARY OF THE INVENTION

The present invention generally relates to methods of treating influenza, to methods of inhibiting the replication of influenza viruses, to methods of reducing the amount of influenza viruses, to compounds and compositions that can be employed for such methods.

In one embodiment, the present invention is directed to a compound represented by Structural Formula (I):

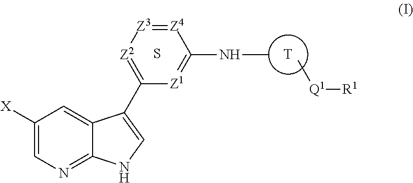

or a pharmaceutically acceptable salt thereof, wherein:

X is —Cl, —Br, —F, —CN, —O(C₁₋₄ alkyl), or C₁-C₆ aliphatic optionally substituted with one or more instances of J¹;

Z¹, Z², Z³, and Z⁴ are each and independently CR² or N, provided that up to three N are selected for Z¹, Z², Z³, and Z⁴, and provided that when Z³ and Z⁴ are both CR², then Z¹ and Z² are both not N at the same time;

Ring S is a 6-membered aromatic ring;

Ring T is a C₃-C₁₀ carbocycle optionally further substituted with one or more instances of J^{T};

Q¹ is —C(O)—, —CO₂—, —OC(O)—, —O(CR^{t}R^{s})ₖ—C(O)O—, —C(O)NR′—, —C(O)N(R′)—O—, —C(O)NRC(O)O—, —NRC(O)—, —NRC(O)NR′—, —NRCO₂—, —OC(O)NR′—, —OSO₂NR′—, —S(O)—, —SO₂—, —SO₂NR′—, —NRSO₂—, —NRSO₂NR′—, —P(O)(OR)O—, —OP(O)(OR^{a})O—, —P(O)₂O—, —CO₂SO₂—, —B(O)₂—, or —(CR^{t}R^{s})ₚ—Y¹—;

Y¹ is —C(O)—, —CO₂—, —OC(O)—, —O(CR^{t}R^{s})ₖ—C(O)O—, —C(O)NR′—, —C(O)N(R′)—O—, —C(O)NRC(O)O—, —NRC(O)—, —NRC(O)NR′—, —NRCO₂—, —OC(O)NR′—, —OSO₂NR′—, —S(O)—, —SO₂—, —SO₂NR′—, —NRSO₂—, —NRSO₂NR′—, —P(O)(OR)O—, —OP(O)(OR^{a})O—, —P(O)₂O—, —B(O)₂—, or —CO₂SO₂—;

R¹ is: i) —H; ii) a C₁-C₆ aliphatic group optionally substituted with one or more instances of J^{A}; iii) a C₃-C₁₀ carbocyclic group or 4-10 membered heterocyclic group, each optionally and independently substituted with one or more instances of J^{B}; or iv) a 6-10 membered aryl group or 5-10 membered heteroaryl group, each optionally and independently substituted with one or more instances of J^{C}; or

optionally R¹, together with R′ and the nitrogen to which they are attached, form a 4-8 membered heterocyclic group optionally substituted with one or more instances of J²; or

optionally —-Q-R¹ is a 4-10 membered, non-aromatic, spiro ring optionally substituted with one or more instances of J⁴; and

R² is —H, halogen, —CN, —NO₂, —C(O)NH₂, —C(O)NH(CH₃), —C(O)N(CH₃)₂, or C₁-C₆ aliphatic optionally substituted with one or more instances of J¹;

J^{A}, J^{B}, and J^{T} are each and independently oxo or J^{C};

J^{C} are each and independently selected from the group consisting of halogen, cyano, M, R^{a}, or R^{a}-M;

M is independently selected from the group consisting of —OR^{b}, —SR^{b}, —S(O)R^{a}, —SO₂R^{a}, —NR^{b}R^{c}, —C(O)R^{a}, —C(═NR)R^{c}, —C(═NR)NR^{b}R^{c}, —NRC(═NR)NR^{b}R^{c}, —C(O)OR^{b}, —OC(O)R^{b}, —NRC(O)R^{b}, —C(O)NR^{b}R^{c}, —NRC(O)NR^{b}R^{c}, —NRC(O)OR^{b}, —OCONR^{b}R^{c}, —C(O)NRCO₂R^{b}, —NRC(O)NRC(O)OR^{b}, —C(O)NR(OR^{b}), —OSO₂NR^{b}R^{c}, —SO₂NR^{c}R^{b}, —NRSO₂R^{b}, —NRSO₂NR^{c}R^{b}, —P(O)(OR^{b})₂, —OP(O)(OR^{b})₂, —P(O)₂OR^{b} and —CO₂SO₂R^{b}; or

optionally, two J^{T}, two J^{A}, two J^{B}, and two J^{C}, respectively, together with the atom(s) to which they are attached, independently form a 4-10-membered ring that is optionally substituted with one or more instances of J⁴; and

R^{a} is independently:

i) a C₁-C₆ aliphatic group optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), C₃-C₈ carbocyclic group optionally substituted with one or more instances of J², 4-8 membered heterocyclic group optionally substituted with one or more instances of J², 5-10 membered heteroaryl group optionally substituted with one or more instances of J³, and 6-10 membered aryl group optionally substituted with one or more instances of J³;
ii) a C₃-C₈ carbocyclic group, or 4-8 membered heterocyclic group, each of which is optionally and independently substituted with one or more instances of J²; or
iii) a 5-10 membered heteroaryl group, or 6-10 membered aryl group, each of which is optionally and independently substituted with one or more instances of J³; and

R^{b} and R^{c} are each independently R^{a} or H; or optionally, R^{b} and R^{c}, together with the nitrogen atom(s) to which they are attached, each independently form a 4-8 membered heterocyclic group optionally substituted with one or more instances of J²;

R^{t} and R^{s} are each independently H, halogen, or C₁-C₆ alkyl optionally substituted with one or more instances of J¹, or optionally, R^{t} and R^{s}, together with the carbon atom to which they are attached, form a cyclopropane ring optionally substituted with one or more instances of methyl;

R and R′ are each independently H or C₁-C₆ alkyl optionally and independently substituted with one or more instances of J¹, or optionally R and R′, together with the nitrogen to which they are attached, form a 4-8 membered heterocyclic group optionally substituted with one or more instances of J²;

each J¹ is independently selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), and phenyl;

each J² is independently selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂ (C₁-C₄ alkyl), C₁-C₄ alkyl, C₁-C₄ haloalkyl, and —O(C₁-C₄ alkyl);

each of J³ and J⁴ is independently selected from the group consisting of halogen, cyano, hydroxy, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl);

p is 1, 2, 3 or 4; and

k is 1, 2, 3 or 4; and

provided that Q¹-R¹ is not at the same carbon atom to which —NH group that is attached to Ring S is attached.

In some embodiments, p is 1 or 2, and k is 1 or 2.

In another embodiment, the present invention is directed to a pharmaceutical composition comprising a compound disclosed herein (e.g., a compound represented by Structural Formula (I) or a pharmaceutically acceptable salt thereof) and a pharmaceutically acceptable carrier, adjuvant or vehicle.

In yet another embodiment, the present invention is directed to a method of inhibiting the replication of influenza viruses in a biological sample or patient, comprising the step of administering to said biological sample or patient an effective amount of a compound disclosed herein (e.g., a compound represented by Structural Formula (I) or a pharmaceutically acceptable salt thereof).

In yet another embodiment, the present invention is directed to a method of reducing the amount of influenza viruses in a biological sample or in a patient, comprising administering to said biological sample or patient an effective amount of a compound disclosed herein (e.g., a compound represented by Structural Formula (I) or a pharmaceutically acceptable salt thereof).

In yet another embodiment, the present invention is directed to a method of method of treating influenza in a patient, comprising administering to said patient an effective amount of a compound disclosed herein (e.g., a compound represented by Structural Formula (I) or a pharmaceutically acceptable salt thereof).

The present invention also provides use of the compounds described herein for inhibiting the replication of influenza viruses in a biological sample or patient, for reducing the amount of influenza viruses in a biological sample or patient, or for treating influenza in a patient.

Also provided herein is use of the compounds described herein for the manufacture of a medicament for treating influenza in a patient, for reducing the amount of influenza viruses in a biological sample or in a patient, or for inhibiting the replication of influenza viruses in a biological sample or patient.

Also provided here in are the compounds represented by Structural Formula (XX):

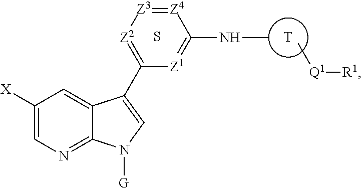

or a pharmaceutically acceptable salt thereof. Without being bound to a particular theory, the compounds of Structural Formula (XX) can be used for synthesizing the compound of Formula (I). The variables of Structural Formula (XX) are each and independently as defined herein; and G is tosyl (Ts) (i.e., CH₃C₆H₄SO₂—) or trityl (Tr) (i.e., C(Ph)₃ where Ph is phenyl).

The invention also provides methods of preparing a compound represented by Structural Formula (I) or a pharmaceutically acceptable salt thereof. In one embodiment, the method comprises the steps of: i) reacting compound A:

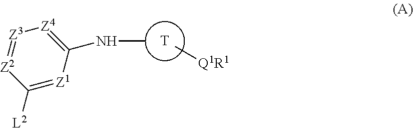

with compound (B):

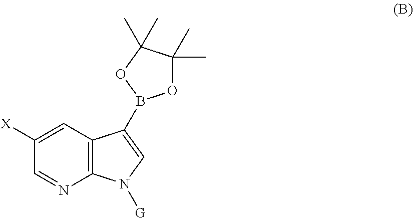

to form a compound represented by Structural Formula (XX):

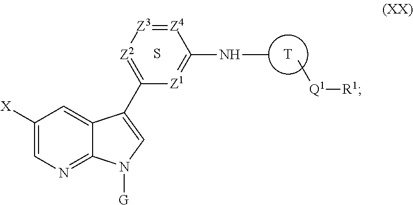

and
ii) deprotecting the G group of the compound of Structural Formula (XX) under suitable conditions to form the compound of Structural Formula (I), wherein: the variables of Structural Formulae (I) and (XX), and compounds (A) and (B) are each independently as defined in herein; L² is a halogen; and G is tosyl or trityl. In another embodiment, the method comprises the steps of: i) reacting compound (K) or (L):

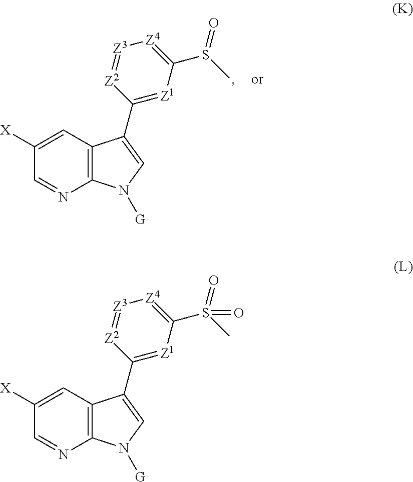

with compound (D):

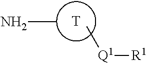

under suitable conditions to form a compound represented by Structural Formula (XX); and
ii) deprotecting the G group of the compound of Structural Formula (XX) under suitable conditions to form the compound of Structural Formula (I), wherein: the variables of Structural Formulae (I) and (XX), and compounds (K), (L), and (D) are each and independently as defined herein; and G is tosyl or trityl. In yet another embodiment, the method comprises the steps of: i) reacting Compound (G) with Compound (D):

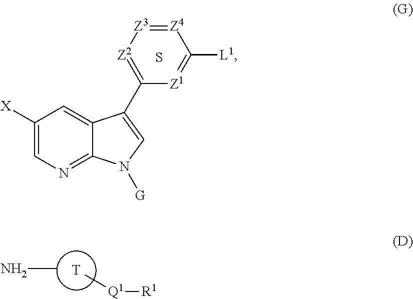

under suitable conditions to form a compound represented by Structural Formula (XX); and ii) deprotecting the G group of the compound of Structural Formula (XX) under suitable conditions to form the compound of Structural Formula (I), wherein: the variables of Structural Formulae (I) and (XX), and Compounds (A) and (B) are each and independently as defined herein; L¹ is a halogen; and G is tosyl or trityl.

### DETAILED DESCRIPTION OF THE INVENTION

The compounds of the invention are as described in the claims. In some embodiments, the compounds of the invention are represented by any one of Structural Formula (I) or pharmaceutically acceptable salts thereof, wherein the variables are each and independently as described herein. In some embodiments, the compounds of the invention are represented by any chemical formulae depicted in Table 1, or pharmaceutically acceptable salts thereof. In some embodiments, the compounds of the invention are represented by any chemical formulae depicted in Table 2, or pharmaceutically acceptable salts thereof. In some embodiments, the compounds of the invention are presented by Structural Formula (I) or a pharmaceutically acceptable salt thereof, wherein the variables are each and independently as depicted in the chemical formulae in Table 1. In some embodiments, the compounds of the invention are presented by Structural Formula (I) or a pharmaceutically acceptable salt thereof, wherein the variables are each and independently as depicted in the chemical formulae in Table 2.

In one embodiment, the compounds of the invention are represented by Structural Formula (I) or pharmaceutically acceptable salts thereof, wherein the first set of values of the variables of Structural Formula (I) is as follows:

X is —Cl, —Br, —F, —CN, —O(C₁₋₄ alkyl), or C₁-C₆ aliphatic optionally substituted with one or more instances of J¹. Typically, X is —F, —Cl, —CN, —O(C₁₋₄ alkyl), C₁₋₄ alkyl, -or C₁₋₄ haloalkyl. Typically, X is —F, —Cl, —CN, C₁₋₄ alkyl, -or C₁₋₄ haloalkyl. Typically, X is —F, —Cl, —CN, C₁₋₄ alkyl, or C₁₋₄ haloalkyl. More typically, X is —F, —Cl, —CF₃, or —CH₃. More typically, X is —F, —Cl, or —CF₃. Even more typically, X is —F or —Cl.

Z¹, Z², Z³, and Z⁴ are each and independently CR² or N, provided that up to three N are selected for Z¹, Z², Z³, and Z⁴, and provided that when Z³ and Z⁴ are both CR², then Z¹ and Z² are both not N at the same time. In one aspect, at least one of Z¹-Z⁴ is N.

Ring S is a 6-membered aromatic ring. Typical examples of Ring S include:

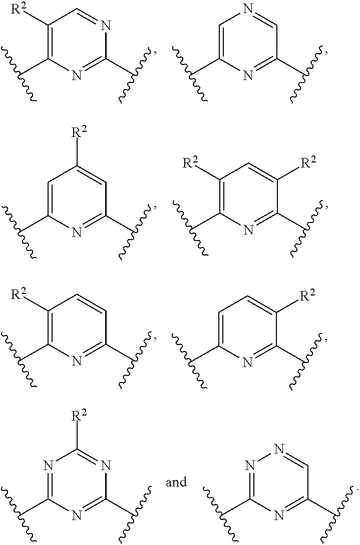

More typical examples of Ring S include:

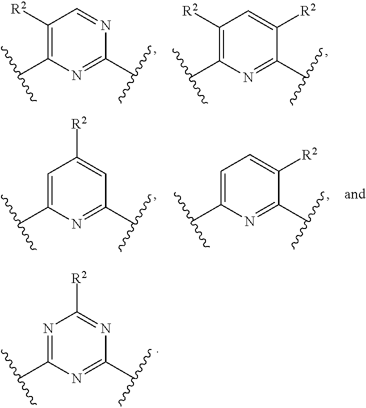

Specific examples of Ring S include:

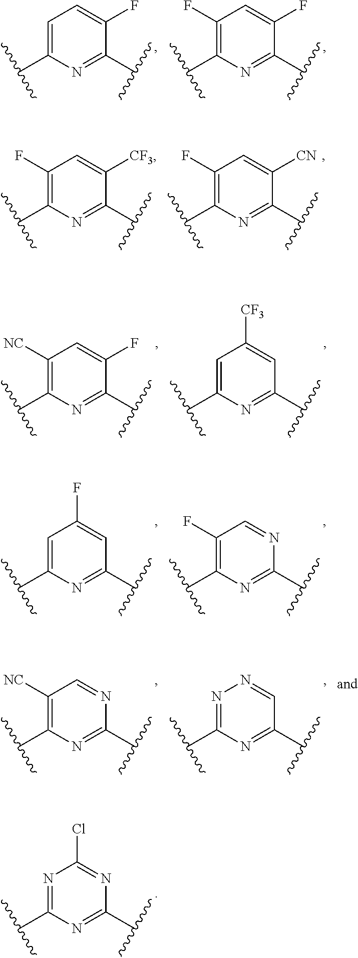

Ring T is a C₃-C₁₀ carbocycle optionally further substituted with one or more instances of J^{T}. In one aspect, Ring T is an optionally substituted, bridged, C₅-C₁₀ carbocyclic group. In another aspect, Ring T is an optionally substituted, monocyclic, C₅-C₈ carbocyclic group. A specific example of Ring T is:

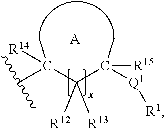

wherein x is 0, 1 or 2. Typical examples of Ring T include:

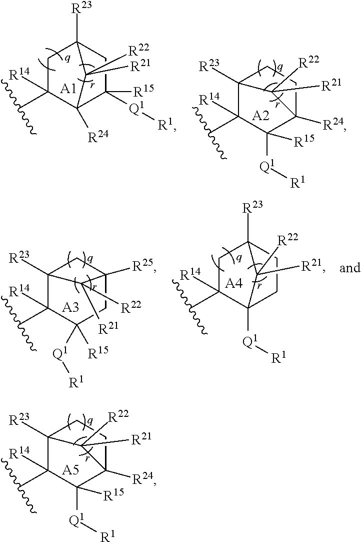

wherein q is 0, 1 or 2; and r is 1 or 2.
Additional typical examples of Ring T include:

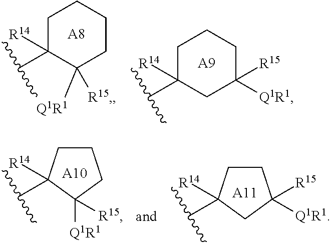

Additional typical examples of Ring T include:

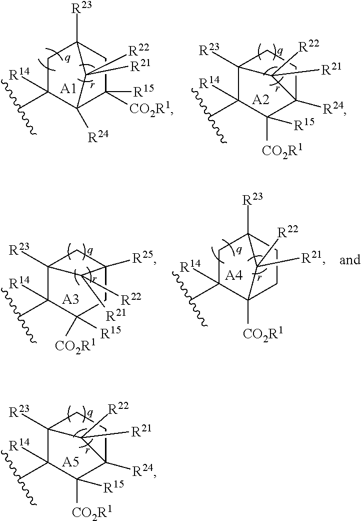

wherein q is 0, 1 or 2; and r is 1 or 2.

Ring A (including Rings A1-A5) is a 5-10 membered carbocyclic group optionally further substituted with one or more instances of J^{T}; or optionally Ring A and R¹⁵, Ring A and R¹⁴, or Ring A and R¹³ independently and optionally form a 5-10 membered, bridged carbocyclic ring optionally further substituted with one or more instances of J^{T}. In one aspect, Ring A is optionally and independently further substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl); or Ring A and R¹⁵, Ring A and R¹⁴, or Ring A and R¹³ independently and optionally form a bridged carbocyclic group optionally and independently substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl). In another aspect, Ring A and R¹⁵, Ring A and R¹⁴, or Ring A and R¹³ independently form an optionally substituted, bridged carbocyclic group.

Each of Rings A1-A5 is independently a 5-10 membered, bridged carbocycle optionally further substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl). Typically, each of Rings A1-A5 is independently and optionally further substituted with one or more substituents selected from the group consisting of halogen, cyano, hydroxy, C₁-C₄ alkyl, C₁-C₄ haloalkyl, and —O(C₁-C₄ alkyl).

Each of Rings A8-A11 is independently and optionally substituted with one or more substitutents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl).

Q¹ is —C(O)—, —CO₂—, —OC(O)—, —O(CR^{t}R^{s})ₖ—C(O)O—, —C(O)NR′—, —C(O)N(R′)—O—, —C(O)NRC(O)O—, —NRC(O)—, —NRC(O)NR′—, —NRCO₂—, —OC(O)NR′—, —OSO₂NR′—, —S(O)—, —SO₂—, —SO₂NR′—, —NRSO₂—, —NRSO₂NR′—, —P(O)(OR)O—, —OP(O)(OR^{a})O—, —P(O)₂O—, —CO₂SO₂—, or —(CR^{t}R^{s})ₚ—Y¹—. Typically, Q¹ is —C(O)—, —CO₂—, —OC(O)—, —O(CR^{t}R^{s})ₖ—C(O)O—, —C(O)NR′—, —C(O)N(R′)—O—, —C(O)NRC(O)O—, —NRC(O)—, —NRC(O)NR′—, —NRCO₂—, —OC(O)NR′—, —OSO₂NR′—, —S(O)—, —SO₂—, —SO₂NR′—, —NRSO₂—, —NRSO₂NR′—, —B(O)₂—, or —(CR^{t}R^{s})ₚ—Y¹—. More typically, Q¹ is —CO₂—, —O(CR^{t}R^{s})ₖ—C(O)O—, —P(O)(OR)O—, —OP(O)(OR^{a})O—, —P(O)₂O—, —CO₂SO₂—, —B(O)₂—, or —(CR^{t}R^{s})ₚ—Y¹—. More typically, Q¹ is —CO₂—, —O(CR^{t}R^{s})ₖ—C(O)O—, —P(O)(OR)O—, —OP(O)(OR^{a})O—, —P(O)₂O—, —CO₂SO₂—, or —(CR^{t}R^{s})ₚ—Y¹—. More typically, Q¹ is —C(O)O—, —NRC(O)—, —C(O)NR—, —NRC(O)NR′—, or —(CR^{t}R^{s})_{1,2}—Y¹—. Q¹ is —C(O)—, —C(O)O—, —NRC(O)—, —C(O)NR—, —NRC(O)NR′—, or —(CH₂)_{1,2}—Y—. Even more typically, Q¹ is independently —C(O)O—, —NRC(O)—, —C(O)NR—, —NRC(O)NR′—, or —(CH₂)_{1,2}—Y—. Even more typically, Q¹ is —C(O)O—, —NRC(O)—, —C(O)NR—, or —NRC(O)NR′—. Specific examples of Q¹ include —C(O)O—, —NHC(O)—, or —C(O)NH—.

Y¹ is —C(O)—, —CO₂—, —OC(O)—, —O(CR^{t}R^{s})ₖ—C(O)O—, —C(O)NR′—, —C(O)N(R′)—O—, —C(O)NRC(O)O—, —NRC(O)—, —NRC(O)NR′—, —NRCO₂—, —OC(O)NR′—, —OSO₂NR′—, —S(O)—, —SO₂—, —SO₂NR′—, —NRSO₂—, —NRSO₂NR′—, —P(O)(OR)O—, —OP(O)(OR^{a})O—, —P(O)₂O—, —B(O)₂—, or —CO₂SO₂—. Typically, Y¹ is —C(O)—, —CO₂—, —OC(O)—, —O(CR^{t}R^{s})ₖ—C(O)O—, —C(O)NR′—, —C(O)N(R′)—O—, —C(O)NRC(O)O—, —NRC(O)—, —NRC(O)NR′—, —NRCO₂—, —OC(O)NR′—, —OSO₂NR′—, —S(O)—, —SO₂—, —SO₂NR′—, —NRSO₂—, —B(O)₂—, or —NRSO₂NR′—. More typically, Y¹ is —C(O)—, —CO₂—, —OC(O)—, —O(CR^{t}R^{s})ₖ—C(O)O—, —C(O)NR′—, —C(O)N(R′)—O—, —C(O)NRC(O)O—, —NRC(O)—, —NRC(O)NR′—, —NRCO₂—, —OC(O)NR′—, —OSO₂NR′—, —S(O)—, —SO₂—, —SO₂NR′—, —NRSO₂—, or —NRSO₂NR′—. More typically, Y¹ is —CO₂—, —O(CR^{t}R^{s})ₖ—C(O)O—, —P(O)(OR)O—, —OP(O)(OR^{a})O—, —P(O)₂O—, or —CO₂SO₂—. More typically, Y¹ is —C(O)—, —C(O)O—, —NRC(O)—, —C(O)NR—, or —NRC(O)NR′—. More typically, Y¹ is —C(O)O—, —NRC(O)—, —C(O)NR—, or —NRC(O)NR—. Specific examples of Y¹ include —C(O)O—, —NHC(O)—, —C(O)NH—, or —NHC(O)NH—.

R¹ is: i) —H; ii) a C₁-C₆ aliphatic group optionally substituted with one or more instances of J^{A}; iii) a C₃-C₁₀ carbocyclic group or 4-10 membered heterocyclic group, each optionally and independently substituted with one or more instances of J^{B}; or iv) a 6-10 membered aryl group or 5-10 membered heteroaryl group, each optionally and independently substituted with one or more instances of J^{C}; or

optionally R¹, together with R′ and the nitrogen to which they are attached, form a 4-8 membered heterocyclic group optionally substituted with one or more instances of J²; or

optionally -Q¹-R¹ forms, together with Ring T, a 4-10 membered, non-aromatic, spiro ring optionally substituted with one or more instances of J⁴; and

provided that Q¹-R¹ is not at the same carbon atom to which —NH group that is attached to Ring S is attached.

In one aspect, R¹ is independently i) —H; ii) a C₁-C₆-aliphatic group optionally substituted with one or more instances of J^{A}; iii) a C₃-C₈ carbocyclic group or 4-8 membered heterocyclic group, each of which is optionally and independently substituted with one or more instances of J^{B}; iv) a phenyl group or 5-6 membered heteroaryl group, each of which is optionally and independently substituted with one or more instances of J^{C}; optionally R¹, together with R′ and the nitrogen to which they are attached, form an optionally substituted, 4-8 membered heterocyclic group; or optionally -Q¹-R¹ forms, together with Ring T, an optionally substituted, 4-10 membered, non-aromatic, spiro ring.

In another aspect, R¹ is independently i) —H; ii) a C₁-C₆-aliphatic group optionally substituted with one or more instances of J^{A}; iii) a C₃-C₈ carbocyclic group or 4-8 membered heterocyclic group, each of which is optionally and independently substituted with one or more instances of J^{B}; iv) a phenyl group or 5-6 membered heteroaryl group, each of which is optionally and independently substituted with one or more instances of J^{C}; or optionally R¹, together with R′ and the nitrogen to which they are attached, form an optionally substituted, 4-8 membered heterocyclic group.

In yet another aspect, R¹ is independently: i) —H; ii) a C₁-C₆ aliphatic group optionally substituted with one or more substituents independently selected from the group consisting of halogen, cyano, hydroxy, oxo, —O(C₁-C₄ alkyl), —NH₂, —NH(C₁-C₄ alkyl), —N(C₁-C₄ alkyl)₂, —C(O)(C₁-C₄ alkyl), —OC(O)(C₁-C₄ alkyl), —C(O)O(C₁-C₄ alkyl), —CO₂H, C₃-C₈ carbocyclic group, 4-8 membered heterocyclic group, phenyl, and 5-6 membered heteroaryl; iii) a C₃-C₇ carbocyclic group; iv) a 4-7 membered heterocyclic group; v) a phenyl group; or vi) a 5-6 membered heteroaryl group; or optionally R¹, together with R′ and the nitrogen to which they are attached, form an optionally substituted, 4-8 membered heterocyclic group; and

each of said carbocyclic, phenyl, heterocyclic, and heteroaryl groups represented by R¹ and for the substituents of the C₁-C₆-aliphatic group represented by R¹, and said heterocyclic group formed with R¹ and R′ is independently and optionally substituted with one or more substituents independently selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl).

In yet another aspect, R¹ is independently —H or an optionally substituted C₁-C₆ aliphatic group, such as —H or optionally substituted C₁₋₆ alkyl.

In yet another aspect, R¹ is independently a 4-7 membered heterocyclic group, a phenyl group, or a 5-6 membered heteroaryl group, wherein each of said heterocyclic, phenyl and heteroaryl groups is independently and optionally substituted with one or more substituents independently selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl); or optionally R¹ and R′, together with the nitrogen atom to which they are attached, form an optionally substituted, 4-8 membered heterocyclic group.

R² is —H, halogen, —CN, —NO₂, —C(O)NH₂, —C(O)NH(CH₃), —C(O)N(CH₃)₂, or C₁-C₆ aliphatic optionally substituted with one or more instances of J¹. Typically, R² is —H, halogen, —CN, —NO₂, —C(O)NH₂, —C(O)NH(CH₃), —C(O)N(CH₃)₂, C₁-C₆ aliphatic (e.g., C₁-C₆ alkyl), or C₁-C₆ haloalkyl. More typically, R² is —H, halogen, —CN, —NO₂, —C(O)NH₂, —C(O)NH(CH₃), —C(O)N(CH₃)₂, —CH₃, or —CF₃. More typically, R² is halogen, —CN, —NO₂, —C(O)NH₂, —C(O)NH(CH₃), —C(O)N(CH₃)₂, —CH₃, or —CF₃. More typically, R² is halogen, —CN, or —CF₃. More typically, R² is —F, —Cl, —CN, —CH₃, or —CF₃. More typically, R² is —F, —Cl, —CN, or —CF₃. More typically, R² is —F, —CN, or —CF₃.

Each of R¹², R¹³, and R¹⁴ is independently —H, halogen, cyano, hydroxy, C₁-C₆ alkyl, —O(C₁-C₆ alkyl), —NH₂, —NH(C₁-C₆ alkyl), —N(C₁-C₆ alkyl)₂, —OCO(C₁-C₆ alkyl), —CO(C₁-C₆ alkyl), —CO₂H, or —CO₂(C₁-C₆ alkyl), wherein each said C₁-C₆ alkyl is optionally and independently substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), and —O(C₁-C₄ alkyl). Typically, R¹², R¹³, and R¹⁴ are each and independently —H, halogen, cyano, hydroxy, —O(C₁-C₆ alkyl), or optionally substituted C₁-C₆ alkyl. More typically, R¹², R¹³, and R¹⁴ are each and independently —H, halogen, hydroxy, C₁-C₆ alkyl, C₁-C₆ haloalkyl, or —O(C₁-C₆ alkyl).

Each R¹⁵ is independently —H, halogen, cyano, hydroxy, or C₁-C₆ alkyl optionally and independently substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), and —O(C₁-C₄ alkyl). Typically, R¹⁵ is —H or optionally substituted C₁-C₆ alkyl. More typically, R¹⁵ are each independently —H, C₁-C₆ alkyl, or C₁-C₆ haloalkyl.

In one aspect, R¹², R¹³, and R¹⁴ are each and independently —H, halogen, cyano, hydroxy, —O(C₁-C₆ alkyl), or optionally substituted C₁-C₆ alkyl; and R¹⁵ is —H or optionally substituted C₁-C₆ alkyl.

In another aspect, R¹² and R¹³ are each independently —H, halogen, hydroxy, C₁-C₆ alkyl, C₁-C₆ haloalkyl, or —O(C₁-C₆ alkyl); and R¹⁴ and R¹⁵ are each independently —H, C₁-C₆ alkyl, or C₁-C₆ haloalkyl.

R²¹, R²², R²³, R²⁴, and R²⁵ are each independently —H, halogen, —OH, C₁-C₆ alkoxy, or C₁-C₆ alkyl optionally substituted with one or more substituents independently selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl). Typically, R²¹, R²², R²³, R²⁴, and R²⁵ are each independently —H, halogen, hydroxy, C₁-C₆ alkoxy, C₁-C₆ alkyl, or C₁-C₆ haloalkyl.

J^{A}, J^{B}, and J^{T} are each and independently oxo or J^{C}; and J^{C} are each and independently selected from the group consisting of halogen, cyano, M, R^{a}, or R^{a}-M. Optionally, two J^{T}, two J^{A}, two J^{B}, and two J^{C}, respectively, together with the atom(s) to which they are attached, independently form a 4-10-membered ring (e.g., 5-7-membered or 5-6-membered) that is optionally substituted with one or more instances of J⁴.

M is independently selected from the group consisting of —OR^{b}, —SR^{b}, —S(O)R^{a}, —SO₂R^{a}, —NR^{b}R^{c}, —C(O)R^{a}, —C(═NR)R^{c}, —C(═NR)NR^{b}R^{c}, —NRC(═NR)NR^{b}R^{c}, —C(O)OR^{b}, —OC(O)R^{b}, —NRC(O)R^{b}, —C(O)NR^{b}R^{c}, —NRC(O)NR^{b}R^{c}, —NRC(O)OR^{b}, —OCONR^{b}R^{c}, —C(O)NRCO₂R^{b}, —NRC(O)NRC(O)OR^{b}, —C(O)NR(OR^{b}), —OSO₂NR^{b}R^{c}, —SO₂NR^{c}R^{b}, —NRSO₂R^{b}, —NRSO₂NR^{c}R^{b}, —P(O)(OR^{b})₂, —OP(O)(OR^{b})₂, —P(O)₂OR^{b} and —CO₂SO₂R^{b}.

Typically, J^{C} is selected from the group consisting of halogen, cyano, —OR^{b}, —SR^{b}, —S(O)R^{a}, —SO₂R^{a}, —NHR^{c}, —C(O)R^{b}, —C(O)OR^{b}, —OC(O)R^{b}, —NHC(O)R^{b}, —C(O)NHR^{c}, —NHC(O)NHR^{c}, —NHC(O)OR^{b}, —OCONHR^{c}, —NHC(O)NHC(O)OR^{b}, —N(CH₃)R^{c}, —N(CH₃)C(O)R^{b}, —C(O)N(CH₃)R^{c}, —N(CH₃)C(O)NHR^{b}, —N(CH₃)C(O)OR^{b}, —OCON(CH₃)R^{c}, —C(O)NHCO₂R^{b}, —C(O)N(CH₃)CO₂R^{b}, —N(CH₃)C(O)NHC(O)OR^{b}, —NHSO₂R^{b}, —SO₂NHR^{b}, —SO₂N(CH₃)R^{b}, and —N(CH₃)SO₂R^{b}; or two J^{C}, respectively, together with the atom(s) to which they are attached, independently form an optionally substituted, 4-10-membered, non-aromatic ring.

In one aspect, J^{A}, J^{B}, J^{C}, and J^{T} are each independently selected from the group consisting of halogen, cyano, R^{a}, —OR^{b}, —NHR^{c}, —C(O)R^{b}, —C(O)OR^{b}, —OC(O)R^{b}, —NHC(O)R^{b}, —C(O)NHR^{c}, —NHC(O)NHR^{c}, —NHC(O)OR^{b}, —OCONHR^{c}, —N(CH₃)R^{c}, —N(CH₃)C(O)R^{b}, —C(O)N(CH₃)R^{c}, —N(CH₃)C(O)NHR^{c}, —N(CH₃)C(O)OR^{b}, —NHSO₂R^{b}, —SO₂NHR^{b}, —SO₂N(CH₃)R^{b}, and —N(CH₃)SO₂R^{b}; or

optionally, two J^{T}, two J^{A}, two J^{B}, and two J^{C}, respectively, together with the atom(s) to which they are attached, independently form a 4-10-membered ring that is optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), and —O(C₁-C₄ alkyl).

Typically, J^{A} is halogen, cyano, hydroxy, oxo, —O(C₁-C₄ alkyl), —NH₂, —NH(C₁-C₄ alkyl), —N(C₁-C₄ alkyl)₂, —C(O)(C₁-C₄ alkyl), —OC(O)(C₁-C₄ alkyl), —C(O)O(C₁-C₄ alkyl), —CO₂H, C₃-C₈ carbocyclic group, 4-8 membered heterocyclic group, phenyl, or 5-6 membered heteroaryl, wherein each of said carbocyclic, phenyl, heterocyclic, and heteroaryl groups is independently and optionally substituted with one or more substituents independently selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl). Optionally, two J^{A}, together with the atom(s) to which they are attached, form an optionally substituted, 4-10-membered (or 5-7 membered, or 5-6 membered) ring.

Typically, J^{B} and J^{C} are each and independently halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, or —O(C₁-C₄ alkyl). Optionally, two J^{B} and two J^{C}, together with the atom(s) to which they are attached, independently form an optionally substituted, 4-10-membered (or 5-7 membered, or 5-6 membered) ring.

Typically, J^{T} is halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, or —O(C₁-C₄ alkyl). More typically, J^{T} is halogen, cyano, hydroxy, C₁-C₄ alkyl, C₁-C₄ haloalkyl, and —O(C₁-C₄ alkyl). Optionally, two J^{T}, together with the atom(s) to which they are attached, form an optionally substituted, 4-10-membered (or 5-7 membered, or 5-6 membered) ring.

Typically, the ring formed with two J^{T}, two J^{A}, two J^{B}, and two J^{C} independently is an optionally substituted non-aromatic ring, such as carbocycle or heterocycle. More typically, the ring is an optionally substituted carbocycle.

R^{a} is independently:

i) a C₁-C₆ aliphatic group optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), C₃-C₈ carbocyclic group optionally substituted with one or more instances of J², 4-8 membered heterocyclic group optionally substituted with one or more instances of J², 5-10 membered heteroaryl group optionally substituted with one or more instances of J³, and 6-10 membered aryl group optionally substituted with one or more instances of J³;
ii) a C₃-C₈ carbocyclic group, or 4-8 membered heterocyclic group, each of which is optionally and independently substituted with one or more instances of J²; or
iii) a 5-10 membered heteroaryl group, or 6-10 membered aryl group, each of which is optionally and independently substituted with one or more instances of J³; and

R^{b} and R^{c} are each independently R^{a} or —H; or optionally, R^{b} and R^{c}, together with the nitrogen atom(s) to which they are attached, each independently form a 4-8 membered heterocyclic group optionally substituted with one or more instances of J².

In one aspect, R^{a} is independently: i) a C₁-C₆ alkyl group optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), optionally substituted C₃-C₈ carbocyclic group, optionally substituted 4-8 membered heterocyclic group, optionally substituted 5-6 membered heteroaryl, and optionally substituted phenyl group; ii) an optionally substituted C₃-C₈ carbocyclic group; iii) optionally substituted 4-8 membered heterocyclic group; iv) an optionally substituted 5-6 membered heteroaryl group; v) or optionally substituted phenyl group;

R^{b} and R^{c} are each independently R^{a} or —H; or optionally, R^{b} and R^{c}, together with the nitrogen atom(s) to which they are attached, each independently form an optionally substituted, 4-8 membered heterocyclic group.

In another aspect, R^{a} is independently: i) a C₁-C₆ alkyl group optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), C₃-C₈ carbocycle, 4-8 membered heterocycle, 5-6 membered heteroaryl, and phenyl; ii) a C₃-C₈ carbocyclic group or 4-8 membered heterocyclic group, each of which is independently and optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl); or iii) a 5-6 membered heteroaryl group or phenyl group, each of which is independently and optionally substituted with one or more substituents selected from the group consisting of halogen, cyano, hydroxy, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl); and

R^{b} and R^{c} are each independently R^{a} or —H; or optionally, R^{b} and R^{c}, together with the nitrogen atom(s) to which they are attached, each independently form a 4-8 membered heterocyclic group optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl).

R^{t} and R^{s} are each independently —H, halogen, or C₁-C₆ alkyl optionally substituted with one or more instances of J¹, or optionally, R^{t} and R^{s}, together with the carbon atom to which they are attached, form a cyclopropane ring optionally substituted with one or more instances of methyl. Typically, R^{t} and R^{s} are each independently —H, halogen, C₁-C₆ alkyl, or C₁-C₆ haloalkyl. More typically, R^{t} and R^{s} are each independently —H or C₁-C₆ alkyl.

R and R′ are each independently —H or C₁-C₆ alkyl optionally and independently substituted with one or more instances of J¹, or optionally R and R′, together with the nitrogen to which they are attached, form a 4-8 membered heterocyclic group optionally substituted with one or more instances of J². Typically, R and R′ are each and independently —H or C₁₋₄ alkyl; or optionally R¹, together with R′ and the nitrogen to which they are attached, form an optionally substituted, 4-8 membered heterocyclic group. More typically, R and R′ are each and independently —H or —CH₃; or optionally R¹, together with R′ and the nitrogen to which they are attached, form an optionally substituted, 4-8 membered heterocyclic group.

Each J¹ is independently selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), and phenyl;

Each J² is independently selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl);

Each of J³ and J⁴ is independently selected from the group consisting of halogen, cyano, hydroxy, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl).

Each p is independently 1, 2, 3 or 4, and each k is independently 1, 2, 3 or 4. Typically, each of p and k independently is 1 or 2.

The second set of values of the variables of Structural Formula (I) is as follows:

At least one of Z¹-Z⁴ is N; and if Z¹ and Z⁴ are both N and Z² and Z³ are each independently CR², or if Z¹ is N and Z², Z³ and Z⁴ are each and independently CR², then at least one of R² is other than —H. Typically, non-H values of R² include —F, —Cl, —CN, —CH₃, or —CF₃. More typical non-H values of R² include —F, —Cl, —CN, or —CF₃. More typical non-H values of R² include —F, —CN, or —CF₃.

The remaining variables of Structural Formula (I) are each and independently as described above in the first set of values of the variables of Structural Formula (I).

The third set of values of the variables of Structural Formula (I) is as follows:

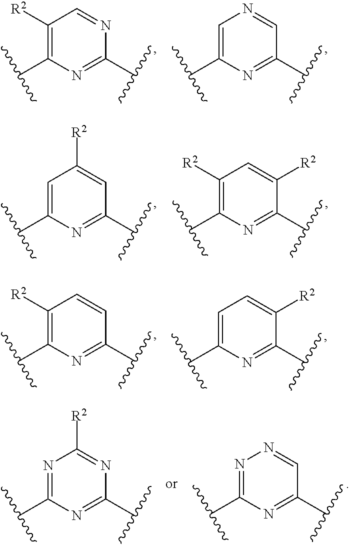

The remaining variables of Structural Formula (I) are each and independently as described above in the first set of values of the variables of Structural Formula (I).

The fourth set of values of the variables of Structural Formula (I) is as follows:

Values of Ring S are as described above in the third set of values of the variables of Structural Formula (I); wherein R² is —F, —Cl, —CN, C₁-C₄ aliphatic, or C₁-C₄ alkyl. More typically, R² is —F, —Cl, —CN, —CH₃, or —CF₃.

The remaining variables of Structural Formula (I) are each and independently as described above in the first set of values of the variables of Structural Formula (I).

The fifth set of values of the variables of Structural Formula (I) is as follows:

Values of Z¹-Z⁴ and R² are each and independently as described above in the second set of values of the variables of Structural Formula (I).

X is —Cl, —Br, —F, —CN, —CH₃, or CF₃.

The remaining variables of Structural Formula (I) are each and independently as described above in the first set of values of the variables of Structural Formula (I).

The sixth set of values of the variables of Structural Formula (I) is as follows:

Values of Z¹-Z⁴ and R² are each and independently as described above in the first or second set of values of the variables of Structural Formula (I).

Values of Ring S are as described above in the third set of values of the variables of Structural Formula (I).

R² is —F, —Cl, —CN, or —CF₃.

X is —Cl, —Br, —F, —CN, —CH₃, or CF₃.

The remaining variables of Structural Formula (I) are each and independently as described above in the first set of values of the variables of Structural Formula (I).

In the seventh set of values of the variables of Structural Formula (I), Q¹R¹ is other than —C(O)NH₂; and values of Z¹-Z⁴, R², and Ring S are each and independently as described above in any one of the first through sixth sets of values of the variables of Structural Formula (I).

The remaining variables of Structural Formula (I) are each and independently as described above in the first set of values of the variables of Structural Formula (I).

The eighth set of values of the variables of Structural Formula (I) is as follows:

Values of Z¹-Z⁴, R², Ring S, and X are each and independently as described above in any one of the first through seventh sets of values of the variables of Structural Formula (I).

Ring T is an optionally substituted, bridged, C₅-C₁₀ carbocyclic group.

The remaining variables of Structural Formula (I) are each and independently as described above in the first set of values of the variables of Structural Formula (I).

The ninth set of values of the variables of Structural Formula (I) is as follows:

Values of Z¹-Z⁴, R², Ring S, and X are each and independently as described above in any one of the first through eighth sets of values of the variables of Structural Formula (I).

Ring T is an optionally substituted, monocyclic, C₅-C₈ carbocyclic group.

The remaining variables of Structural Formula (I) are each and independently as described above in the first set of values of the variables of Structural Formula (I).

The tenth set of values of the variables of Structural Formula (I) is as follows:

Values of Z¹-Z⁴, R², Ring S, Ring T, and X are each and independently as described above in any one of the first through ninth sets of values of the variables of Structural Formula (I).

R¹ is independently i) —H; ii) a C₁-C₆-aliphatic group optionally substituted with one or more instances of J^{A}; iii) a C₃-C₈ carbocyclic group or 4-8 membered heterocyclic group, each of which is optionally and independently substituted with one or more instances of J^{B}; iv) a phenyl group or 5-6 membered heteroaryl group, each of which is optionally and independently substituted with one or more instances of J^{C}; or optionally R¹, together with R′ and the nitrogen to which they are attached, form an optionally substituted, 4-8 membered heterocyclic group; or optionally -Q¹-R¹ forms, together with Ring T, an optionally substituted, 4-10 membered, non-aromatic, spiro ring.

J^{A}, J^{B}, and J^{T} are each independently oxo or J^{C}.

J^{C} is selected from the group consisting of halogen, cyano, R^{a}, —OR^{b}, —SR^{b}, —S(O)R^{a}, —SO₂R^{a}, —NHR^{c}, —C(O)R^{b}, —C(O)OR^{b}, —OC(O)R^{b}, —NHC(O)R^{b}, —C(O)NHR^{c}, —NHC(O)NHR^{c}, —NHC(O)OR^{b}, —OCONHR^{c}, —NHC(O)NHC(O)OR^{b}, —N(CH₃)R^{c}, —N(CH₃)C(O)R^{b}, —C(O)N(CH₃)R^{c}, —N(CH₃)C(O)NHR^{c}, —N(CH₃)C(O)OR^{b}, —OCON(CH₃)R^{c}, —C(O)NHCO₂R^{b}, —C(O)N(CH₃)CO₂R^{b}, —N(CH₃)C(O)NHC(O)OR^{b}, —NHSO₂R^{b}, —SO₂NHR^{b}, —SO₂N(CH₃)R^{b}, and —N(CH₃)SO₂R^{b}.

Optionally, two J^{T}, two J^{A}, two J^{B}, and two J^{C}, respectively, together with the atom(s) to which they are attached, independently form an optionally substituted, 4-10-membered, non-aromatic ring.

The remaining variables of Structural Formula (I) are each and independently as described above in the first set of values of the variables of Structural Formula (I).

The eleventh set of values of the variables of Structural Formula (I) is as follows:

Values of Z¹-Z⁴, R², Ring S, Ring T, X, R¹, J^{A}, J^{B}, J^{C}, and J^{T} are each and independently as described above in any one of the first through tenth sets of values of the variables of Structural Formula (I).

R^{a} is independently: i) a C₁-C₆ alkyl group optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), optionally substituted C₃-C₈ carbocyclic group, optionally substituted 4-8 membered heterocyclic group, optionally substituted 5-6 membered heteroaryl, and optionally substituted phenyl group; ii) an optionally substituted C₃-C₈ carbocyclic group; iii) optionally substituted 4-8 membered heterocyclic group; iv) an optionally substituted 5-6 membered heteroaryl group; v) or optionally substituted phenyl group.

R^{b} and R^{c} are each independently R^{a} or —H; or optionally, R^{b} and R^{c}, together with the nitrogen atom(s) to which they are attached, each independently form an optionally substituted, 4-8 membered heterocyclic group.

R and R′ are each and independently —H or C₁₋₄ alkyl, or optionally R and R′, together with the nitrogen to which they are attached, form an optionally substituted 4-8 membered heterocyclic group, or optionally R′, together with R¹ and the nitrogen to which they are attached, form an optionally substituted 4-8 membered heterocyclic group.

The remaining variables of Structural Formula (I) are each and independently as described above in the first set of values of the variables of Structural Formula (I).

The twelfth set of values of the variables of Structural Formula (I) is as follows:

Values of Z¹-Z⁴, R², Ring S, Ring T, X, R¹, J^{A}, J^{B}, J^{C}, J^{T}, R^{a}, R^{b}, R^{c}, R, and R′ are each and independently as described above in any one of the first through eleventh sets of values of the variables of Structural Formula (I).

Q¹ is —C(O)—, —CO₂—, —OC(O)—, —O(CR^{t}R^{s})ₖ—C(O)O—, —C(O)NR′—, —C(O)N(R′)—O—, —C(O)NRC(O)O—, —NRC(O)—, —NRC(O)NR′—, —NRCO₂—, —OC(O)NR′—, —OSO₂NR′—, —S(O)—, —SO₂—, —SO₂NR′—, —NRSO₂—, —NRSO₂NR′—, —B(O)₂—, or —(CR^{t}R^{s})ₚ—Y¹—.

Y¹ is —C(O)—, —CO₂—, —OC(O)—, —O(CR^{t}R^{s})ₖ—C(O)O—, —C(O)NR′—, —C(O)N(R′)—O—, —C(O)NRC(O)O—, —NRC(O)—, —NRC(O)NR′—, —NRCO₂—, —OC(O)NR′—, —OSO₂NR′—, —S(O)—, —SO₂—, —SO₂NR′—, —NRSO₂—, —B(O)₂—, or —NRSO₂NR′—.

The remaining variables of Structural Formula (I) are each and independently as described above in the first set of values of the variables of Structural Formula (I).

The thirteenth set of values of the variables of Structural Formula (I) is as follows:

Values of Z¹-Z⁴, R², Ring S, Ring T, X, R¹, J^{A}, J^{B}, J^{C}, J^{T}, R^{a}, R^{b}, R^{c}, R, and R′ are each and independently as described above in any one of the first through eleventh sets of values of the variables of Structural Formula (I).

Q¹ is —CO₂—, —O(CR^{t}R^{s})ₖ—C(O)O—, —P(O)(OR)O—, —OP(O)(OR^{a})O—, —P(O)₂O—, —CO₂SO₂—, or —(CR^{t}R^{s})ₚ—Y¹—; and

Y¹ is —CO₂—, —O(CR^{t}R^{s})ₖ—C(O)O—, —P(O)(OR)O—, —OP(O)(OR^{a})O—, —P(O)₂O—, or —CO₂SO₂—.

The remaining variables of Structural Formula (I) are each and independently as described above in the first set of values of the variables of Structural Formula (I).

The fourteenth set of values of the variables of Structural Formula (I) is as follows:

Values of Z¹-Z⁴, R², Ring T, X, R¹, J^{A}, J^{B}, J^{C}, J^{T}, R^{a}, R^{b}, R^{c}, R, R′, Q¹, and Y¹ are each and independently as described above in any one of the first through thirteenth sets of values of the variables of Structural Formula (I).

Ring S is

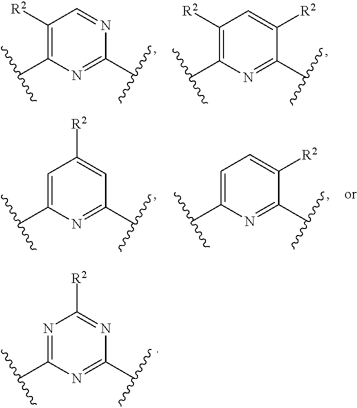

The remaining variables of Structural Formula (I) are each and independently as described above in the first set of values of the variables of Structural Formula (I).

The fifteenth set of values of the variables of Structural Formula (I) is as follows:

Values of Z¹-Z⁴, R², Ring T, X, R¹, J^{A}, J^{B}, J^{C}, J^{T}, R^{a}, R^{b}, R^{c}, R, R′, Q¹, and Y¹ are each and independently as described above in any one of the first through thirteenth sets of values of the variables of Structural Formula (I).

Ring S is selected from:

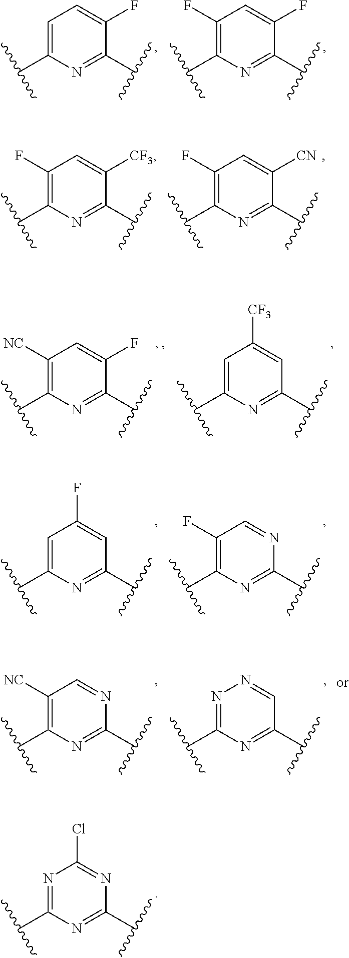

The remaining variables of Structural Formula (I) are each and independently as described above in the first set of values of the variables of Structural Formula (I).

The sixteenth set of values of the variables of Structural Formula (I) is as follows:

Values of Z¹-Z⁴, R², Ring S, X, R¹, J^{A}, J^{B}, J^{C}, J^{T}, R^{a}, R^{b}, R^{c}, R, R′, Q¹, and Y¹ are each and independently as described above in any one of the first through fifteenth sets of values of the variables of Structural Formula (I).

Ring T is:

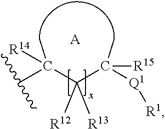

and wherein:

Ring A is a 5-10 membered carbocyclic group optionally further substituted with one or more instances of J^{T}; or optionally Ring A and R¹⁵, Ring A and R¹⁴, or Ring A and R¹³ independently and optionally form a 5-10 membered, bridged carbocyclic ring optionally further substituted with one or more instances of J^{T};

each of R¹², R¹³, and R¹⁴ is independently —H, halogen, cyano, hydroxy, C₁-C₆ alkyl, —O(C₁-C₆ alkyl), —NH₂, —NH(C₁-C₆ alkyl), —N(C₁-C₆ alkyl)₂, —OCO(C₁-C₆ alkyl), —CO(C₁-C₆ alkyl), —CO₂H, or —CO₂(C₁-C₆ alkyl), wherein each said C₁-C₆ alkyl is optionally and independently substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), and —O(C₁-C₄ alkyl);

each R¹⁵ is independently —H, halogen, cyano, hydroxy, or C₁-C₆ alkyl optionally and independently substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), and —O(C₁-C₄ alkyl); and

x is 0, 1 or 2.

The remaining variables of Structural Formula (I) are each and independently as described above in the first set of values of the variables of Structural Formula (I).

The seventeenth set of values of the variables of Structural Formula (I) is as follows:

Values of Z¹-Z⁴, R², Ring S, Ring T, X, R¹, R¹², R¹³, R¹⁴, R¹⁵, R^{a}, R^{b}, R^{c}, R, R′, Q¹, Y¹, and x are each and independently as described above in any one of the first through sixteenth sets of values of the variables of Structural Formula (I).

J^{A}, J^{B}, J^{C}, and J^{T} are each independently selected from the group consisting of halogen, cyano, R^{a}, —OR^{b}, —NHR^{c}, —C(O)R^{b}, —C(O)OR^{b}, —OC(O)R^{b}, —NHC(O)R^{b}, —C(O)NHR^{c}, —NHC(O)NHR^{c}, —NHC(O)OR^{b}, —OCONHR^{c}, —N(CH₃)R^{c}, —N(CH₃)C(O)R^{b}, —C(O)N(CH₃)R^{c}, —N(CH₃)C(O)NHR^{c}, —N(CH₃)C(O)OR^{b}, —NHSO₂R^{b}, —SO₂NHR^{b}, —SO₂N(CH₃)R^{b}, and —N(CH₃)SO₂R^{b}; or

optionally, two J^{T}, two J^{A}, two J^{B}, and two J^{C}, respectively, together with the atom(s) to which they are attached, independently form a 4-10-membered ring that is optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), and —O(C₁-C₄ alkyl).

The remaining variables of Structural Formula (I) are each and independently as described above in the first set of values of the variables of Structural Formula (I).

The eighteenth set of values of the variables of Structural Formula (I) is as follows:

Values of Z¹-Z⁴, R², Ring S, Ring T, X, R¹, R¹², R¹³, R¹⁴, R¹⁵, J^{A}, J^{B}, J^{C}, J^{T}, R, R′, Q¹, Y¹, and x are each and independently as described above in any one of the first through seventeenth sets of values of the variables of Structural Formula (I).

R^{a} is independently: i) a C₁-C₆ alkyl group optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), C₃-C₈ carbocycle, 4-8 membered heterocycle, 5-6 membered heteroaryl, and phenyl; ii) a C₃-C₈ carbocyclic group or 4-8 membered heterocyclic group, each of which is independently and optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl); or iii) a 5-6 membered heteroaryl group or phenyl group, each of which is independently and optionally substituted with one or more substituents selected from the group consisting of halogen, cyano, hydroxy, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl).

R^{b} and R^{c} are each independently R^{a} or —H; or optionally, R^{b} and R^{c}, together with the nitrogen atom(s) to which they are attached, each independently form a 4-8 membered heterocyclic group optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl).

The remaining variables of Structural Formula (I) are each and independently as described above in the first set of values of the variables of Structural Formula (I).

The nineteenth set of values of the variables of Structural Formula (I) is as follows:

Values of Z¹-Z⁴, R², Ring S, Ring T, X, R¹, R¹², R¹³, R¹⁴, R¹⁵, J^{A}, J^{B}, J^{C}, J^{T}, R^{a}, R^{b}, R^{c}, R, and R′ are each and independently as described above in any one of the first through eighteenth sets of values of the variables of Structural Formula (I).

Q¹ is —C(O)O—, —NRC(O)—, —C(O)NR—, —NRC(O)NR′—, or —(CR^{t}R^{s})_{1,2}—Y—.

Y¹ is —C(O)O—, —NRC(O)—, —C(O)NR—, or —NRC(O)NR′—.

The remaining variables of Structural Formula (I) are each and independently as described above in the first set of values of the variables of Structural Formula (I).

The twentieth set of values of the variables of Structural Formula (I) is as follows:

Values of Z¹-Z⁴, R², Ring T, X, R¹, R¹², R¹³, R¹⁴, R¹⁵, J^{A}, J^{B}, J^{C}, J^{T}, R^{a}, R^{b}, R^{c}, R, Q¹, and Y¹ are each and independently as described above in any one of the first through nineteenth sets of values of the variables of Structural Formula (I).

Ring S is selected from:

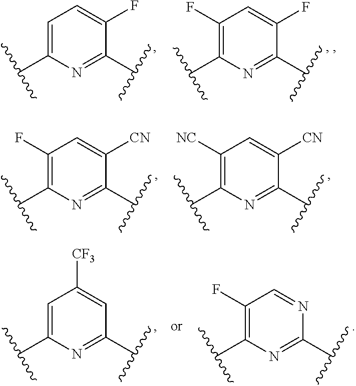 [SMILES: *c1ccc(F)c(*)n1 ; *c1nc(*)c(F)cc1F ; *c1nc(*)c(C#N)cc1F ; *c1nc(*)c(C#N)cc1C#N ; *c1cc(C(F)(F)F)cc(*)n1 ; *c1ncc(F)c(*)n1]

The remaining variables of Structural Formula (I) are each and independently as described above in the first set of values of the variables of Structural Formula (I).

The twenty first set of values of the variables of Structural Formula (I) is as follows:

Values of Z¹-Z⁴, R², Ring S, Ring T, X, R¹, J^{A}, J^{B}, J^{C}, J^{T}, R, R′, Q¹, and Y¹ are each and independently as described above in any one of the first through twentieth sets of values of the variables of Structural Formula (I).

R¹², R¹³, and R¹⁴ are each and independently —H, halogen, cyano, hydroxy, —O(C₁-C₆ alkyl), or optionally substituted C₁-C₆ alkyl.

R¹⁵ is —H or optionally substituted C₁-C₆ alkyl.

R^{t} and R^{s} are each independently —H, halogen, C₁-C₆ alkyl, or C₁-C₆ haloalkyl.

The remaining variables of Structural Formula (I) are each and independently as described above in the first set of values of the variables of Structural Formula (I).

The twenty second set of values of the variables of Structural Formula (I) is as follows:

Values of Z¹-Z⁴, R², Ring S, Ring T, X, R¹, J^{A}, J^{B}, J^{C}, J^{T}, R, R′, Q¹, and Y¹ are each and independently as described above in any one of the second through twenty first sets of values of the variables of Structural Formula (I).

R¹² and R¹³ are each independently —H, halogen, hydroxy, C₁-C₆ alkyl, C₁-C₆ haloalkyl, or —O(C₁-C₆ alkyl).

R¹⁴ and R¹⁵ are each independently —H, C₁-C₆ alkyl, or C₁-C₆ haloalkyl.

R^{t} and R^{s} are each independently —H or C₁-C₆ alkyl.

The remaining variables of Structural Formula (I) are each and independently as described above in the first set of values of the variables of Structural Formula (I).

The twenty third set of values of the variables of Structural Formula (I) is as follows:

Values of Z¹-Z⁴, R², Ring S, Ring T, X, J^{A}, J^{B}, J^{C}, T, R, R′, Q¹, Y¹, R¹², R¹³, R¹⁴, R¹⁵, R^{s} and R^{t} are each and independently as described above in any one of the first through twenty second sets of values of the variables of Structural Formula (I).

R¹ is independently: i) —H; ii) a C₁-C₆ aliphatic group optionally substituted with one or more substituents independently selected from the group consisting of halogen, cyano, hydroxy, oxo, —O(C₁-C₄ alkyl), —NH₂, —NH(C₁-C₄ alkyl), —N(C₁-C₄ alkyl)₂, —C(O)(C₁-C₄ alkyl), —OC(O)(C₁-C₄ alkyl), —C(O)O(C₁-C₄ alkyl), —CO₂H, C₃-C₈ carbocyclic group, 4-8 membered heterocyclic group, phenyl, and 5-6 membered heteroaryl; iii) a C₃-C₇ carbocyclic group; iv) a 4-7 membered heterocyclic group; v) a phenyl group; or vi) a 5-6 membered heteroaryl group;

optionally R¹, together with R′ and the nitrogen to which they are attached, form an optionally substituted, 4-8 membered heterocyclic group; and

each of said carbocyclic, phenyl, heterocyclic, and heteroaryl groups represented by R¹ and for the substituents of the C₁-C₆-aliphatic group represented by R¹, and said heterocyclic group formed with R¹ and R′ is independently and optionally substituted with one or more substituents independently selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl).

The remaining variables of Structural Formula (I) are each and independently as described above in the first set of values of the variables of Structural Formula (I).

The twenty fourth set of values of the variables of Structural Formula (I) is as follows:

Values of Z¹-Z⁴, R¹, R², Ring S, X, J^{A}, J^{B}, J^{C}, J^{T}, R, R′, Q¹, Y¹, R¹², R¹³, R¹⁴, R¹⁵, R^{s} and R^{t} are each and independently as described above in any one of the first through twenty third sets of values of the variables of Structural Formula (I).

Ring T is:

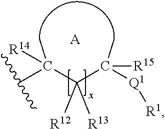

and wherein:

Ring A is a 5-10 membered carbocyclic group optionally further substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl); or Ring A and R¹⁵, Ring A and R¹⁴, or Ring A and R¹³ independently and optionally form a bridged carbocyclic group optionally and independently substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl);

each of R¹², R¹³, and R¹⁴ is independently —H, halogen, cyano, hydroxy, C₁-C₆ alkyl, —O(C₁-C₆ alkyl), —NH₂, —NH(C₁-C₆ alkyl), —N(C₁-C₆ alkyl)₂, —OCO(C₁-C₆ alkyl), —CO(C₁-C₆ alkyl), —CO₂H, or —CO₂(C₁-C₆ alkyl), wherein each said C₁-C₆ alkyl is optionally and independently substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), and —O(C₁-C₄ alkyl);

each R¹⁵ is independently —H, halogen, cyano, hydroxy, or C₁-C₆ alkyl optionally and independently substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), and —O(C₁-C₄ alkyl); and

x is 0, 1 or 2.

The remaining variables of Structural Formula (I) are each and independently as described above in the first set of values of the variables of Structural Formula (I).

The twenty fifth set of values of the variables of Structural Formula (I) is as follows:

Values of Z¹-Z⁴, R¹, R², Ring S, X, J^{A}, J^{B}, J^{C}, J^{T}, R, R′, Q¹, Y¹, R¹², R¹³, R¹⁴, R¹⁵, R^{s} and R^{t} are each and independently as described above in any one of the first through twenty fourth sets of values of the variables of Structural Formula (I).

Ring T is:

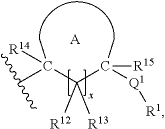

and wherein Ring A and R¹⁵, Ring A and R¹⁴, or Ring A and R¹³ independently form an optionally substituted, bridged carbocyclic group.

The remaining variables of Structural Formula (I) are each and independently as described above in the first set of values of the variables of Structural Formula (I).

The twenty sixth set of values of the variables of Structural Formula (I) is as follows:

Values of Z¹-Z⁴, R¹, R², Ring S, X, J^{A}, J^{B}, J^{C}, J^{T}, R, R′, Q¹, Y¹, R⁴, R¹⁵, R^{s} and R^{t} are each and independently as described above in any one of the first through twenty fourth sets of values of the variables of Structural Formula (I).

Ring T is:

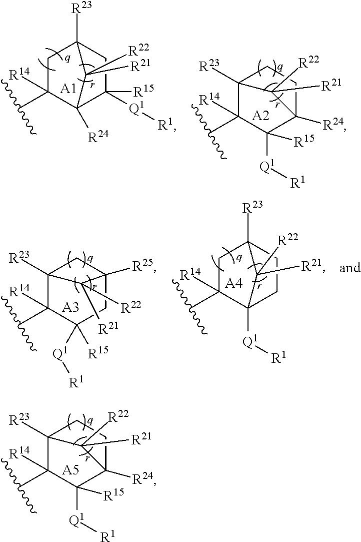

wherein:

each of Rings A1-A5 is independently a 5-10 membered, bridged carbocycle optionally further substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl);

R¹⁴ is —H, halogen, cyano, hydroxy, C₁-C₆ alkyl, —O(C₁-C₆ alkyl), —NH₂, —NH(C₁-C₆ alkyl), —N(C₁-C₆ alkyl)₂, —OCO(C₁-C₆ alkyl), —CO(C₁-C₆ alkyl), —CO₂H, or —CO₂(C₁-C₆ alkyl), wherein each said C₁-C₆ alkyl is optionally and independently substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), and —O(C₁-C₄ alkyl);

each R¹⁵ is independently —H, halogen, cyano, hydroxy, or C₁-C₆ alkyl optionally and independently substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), and —O(C₁-C₄ alkyl); and

R²¹, R²², R²³, R²⁴, and R²⁵ are each independently —H, halogen, —OH, C₁-C₆ alkoxy, or C₁-C₆ alkyl optionally substituted with one or more substituents independently selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl);

q is 0, 1 or 2; and

r is 1 or 2.

The remaining variables of Structural Formula (I) are each and independently as described above in the first set of values of the variables of Structural Formula (I).

The twenty seventh set of values of the variables of Structural Formula (I) is as follows:

Values of Z¹-Z⁴, R¹, R², Ring S, Ring T, X, J^{A}, J^{B}, J^{C}, J^{T}, R, R′, Q¹, Y¹, R¹², R¹³, R^{s} and R^{t} are each and independently as described above in the twenty sixth set of values of the variables of Structural Formula (I).

R¹⁴ and each R¹⁵ are each independently —H, C₁-C₆ alkyl, or C₁-C₆ haloalkyl.

R²¹, R²², R²³, R²⁴, and R²⁵ are each independently —H, halogen, hydroxy, C₁-C₆ alkoxy, C₁-C₆ alkyl, or C₁-C₆ haloalkyl.

The remaining variables of Structural Formula (I) are each and independently as described above in the first set of values of the variables of Structural Formula (I).

The twenty eighth set of values of the variables of Structural Formula (I) is as follows:

Values of Z¹-Z⁴, R¹, R², Ring S, Ring T, X, J^{A}, J^{B}, J^{C}, J^{T}, R, R′, R¹², R¹³, R¹⁴, R¹⁵, R^{s}, R^{t}, R²¹, R²², R²³, R²⁴, and R²⁵ are each and independently as described above in the twenty sixth or twenty seventh set of values of the variables of Structural Formula (I).

Q¹ is independently —C(O)O—, —NRC(O)—, —C(O)NR—, —NRC(O)NR′—, or —(CH₂)_{1,2}—Y—,

Y¹ is independently —C(O)O—, —NRC(O)—, —C(O)NR—, or —NRC(O)NR′—. R¹⁴ and each R¹⁵ are each independently —H, C₁-C₆ alkyl, or C₁-C₆ haloalkyl.

The remaining variables of Structural Formula (I) are each and independently as described above in the first set of values of the variables of Structural Formula (I).

The twenty ninth set of values of the variables of Structural Formula (I) is as follows:

Values of Z¹-Z⁴, R¹, R², Ring S, Ring T, X, J^{A}, J^{B}, J^{C}, J^{T}, R, R′, R¹², R¹³, R¹⁴, R¹⁵, R^{s}, R^{t}, R²¹, R²², R²³, R²⁴, and R²⁵ are each and independently as described above in the twenty sixth or twenty seventh set of values of the variables of Structural Formula (I).

Q¹ is independently —C(O)O—, —NRC(O)—, or —C(O)NR—.

The remaining variables of Structural Formula (I) are each and independently as described above in the first set of values of the variables of Structural Formula (I).

The thirtieth set of values of the variables of Structural Formula (I) is as follows:

Values of Z¹-Z⁴, R¹, R², Ring S, Ring T, X, J^{A}, J^{B}, J^{C}, J^{T}, R, R′, R¹², R¹³, R¹⁴, R¹⁵, R^{s}, R^{t}, R²¹, R²², R²³, R²⁴, and R²⁵ are each and independently as described above in the twenty sixth or twenty seventh set of values of the variables of Structural Formula (I).

Q¹ is independently —C(O)O—, —NHC(O)—, or —C(O)NH—.

The remaining variables of Structural Formula (I) are each and independently as described above in the first set of values of the variables of Structural Formula (I).

The thirty first set of values of the variables of Structural Formula (I) is as follows:

Values of Z¹-Z⁴, R², Ring S, Ring T, X, J^{A}, B, C, T, Q¹, Y¹, R¹², R¹³, R¹⁴, R¹⁵, R^{s}, R^{t}, R²¹, R²², R²³, R²⁴, and R²⁵ are each and independently as described above in any one of the twenty sixth through thirtieth sets of values of the variables of Structural Formula (I).

R¹ is independently —H or an optionally substituted C₁-C₆ aliphatic group; and

R and R′ are each and independently —H or —CH₃; or

optionally R¹, together with R′ and the nitrogen to which they are attached, form an optionally substituted, 4-8 membered heterocyclic group.

The remaining variables of Structural Formula (I) are each and independently as described above in the first set of values of the variables of Structural Formula (I).

The thirty second set of values of the variables of Structural Formula (I) is as follows:

Values of Z¹-Z⁴, R¹, R², Ring S, X, J^{A}, J^{B}, J^{C}, J^{T}, Q¹, Y¹, R, R′, R¹², R¹³, R¹⁴, R¹⁵, R^{s}, R^{t}, R²¹, R²², R²³, R²⁴, and R²⁵ are each and independently as described above in any one of the twenty sixth through thirty first sets of values of the variables of Structural Formula (I).

Ring T is:

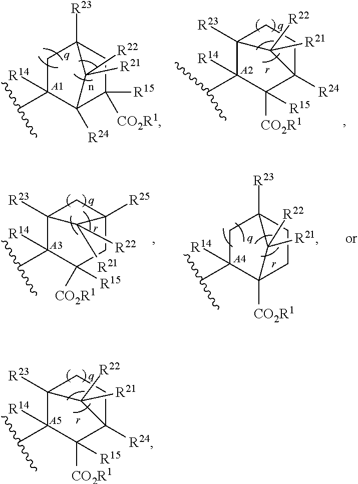

wherein each of Rings A1-A5 is independently and optionally further substituted with one or more substituents selected from the group consisting of halogen, cyano, hydroxy, C₁-C₄ alkyl, C₁-C₄ haloalkyl, and —O(C₁-C₄ alkyl).

The remaining variables of Structural Formula (I) are each and independently as described above in the first set of values of the variables of Structural Formula (I).

The thirty third set of values of the variables of Structural Formula (I) is as follows:

Values of Z¹-Z⁴, R¹, R², Ring S, Ring T, X, J^{A}, J^{B}, J^{C}, J^{T}, Q¹, Y¹, R, R′, R¹², R¹³, R^{s}, and R^{t}, are each and independently as described above in any one of the twenty sixth through thirty second sets of values of the variables of Structural Formula (I).

R¹⁴ and each R¹⁵ are each independently —H or C₁₋₆ alkyl.

R²¹, R²², R²³, R²⁴, and R²⁵ are each independently —H or C₁₋₆ alkyl.

The remaining variables of Structural Formula (I) are each and independently as described above in the first set of values of the variables of Structural Formula (I).

The thirty fourth set of values of the variables of Structural Formula (I) is as follows:

Values of Z¹-Z⁴, R², Ring S, Ring T, X, J^{A}, J^{B}, J^{C}, J^{T}, Q¹, Y¹, R, R′, R¹², R¹³, R^{s}, R^{t}, are each and independently as described above in any one of the twenty sixth through thirty second sets of values of the variables of Structural Formula (I).

R¹ is H or optionally substituted C₁₋₆ alkyl.

R¹⁴, R¹⁵, R²¹, R²², R²³, R²⁴, and R²⁵ are each independently —H.

The remaining variables of Structural Formula (I) are each and independently as described above in the first set of values of the variables of Structural Formula (I).

The thirty fifth set of values of the variables of Structural Formula (I) is as follows:

Values of Z¹-Z⁴, R¹, R², Ring S, Ring T, X, J^{A}, J^{B}, J^{C}, J^{T}, Q¹, Y¹, R, R′, R¹², R¹³, R^{s}, R^{t}, R²¹, R²², R²³, R²⁴, and R²⁵ are each and independently as described above in any one of the twenty sixth through thirty fourth sets of values of the variables of Structural Formula (I).

q is 1.

The remaining variables of Structural Formula (I) are each and independently as described above in the first set of values of the variables of Structural Formula (I).

The thirty sixth set of values of the variables of Structural Formula (I) is as follows:

Values of Z¹-Z⁴, R¹, R², Ring S, X, J^{A}, J^{B}, J^{C}, J^{T}, Q¹, Y¹, R, R′, R^{s}, and R^{t} are each and independently as described above in any one of the second through twenty fifth sets of values of the variables of Structural Formula (I).

Ring T is selected from:

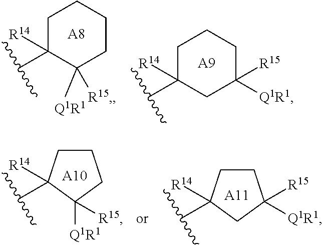

wherein:

R¹⁴ and each R¹⁵ are each independently —H, C₁-C₆ alkyl, or C₁-C₆ haloalkyl; and

each of Rings A8-A11 is independently and optionally substituted with one or more substitutents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl).

The remaining variables of Structural Formula (I) are each and independently as described above in the first set of values of the variables of Structural Formula

The thirty seventh set of values of the variables of Structural Formula (I) is as follows:

Values of Z¹-Z⁴, R¹, R², Ring S, Ring T, X, J^{A}, J^{B}, J^{C}, J^{T}, R, R′, R^{s}, R^{t}, R¹⁴, and R¹⁵ are each and independently as described above in the thirty sixth set of values of the variables of Structural Formula (I).

Q¹ is independently —C(O)—, —C(O)O—, —NRC(O)—, —C(O)NR—, —NRC(O)NR′—, or —(CH₂)_{1,2}—Y—; and

Y¹ is independently —C(O)—, —C(O)O—, —NRC(O)—, —C(O)NR—, or —NRC(O)NR′—.

The remaining variables of Structural Formula (I) are each and independently as described above in the first set of values of the variables of Structural Formula

The thirty eighth set of values of the variables of Structural Formula (I) is as follows:

Values of Z¹-Z⁴, R¹, R², Ring S, Ring T, X, J^{A}, J^{B}, J^{C}, J^{T}, Q¹, Y¹, R, R′, R^{s}, and R^{t} are each and independently as described above in the thirty sixth or thirty seventh set of values of the variables of Structural Formula (I).

R¹⁴ and each R¹⁵ are each independently —H or C₁₋₆ alkyl.

Each of Rings A8-A11 is independently and optionally substituted with one or more substitutents selected from the group consisting of halogen, cyano, hydroxy, C₁-C₄ alkyl, C₁-C₄ haloalkyl, and —O(C₁-C₄ alkyl).

The remaining variables of Structural Formula (I) are each and independently as described above in the first set of values of the variables of Structural Formula

The thirty ninth set of values of the variables of Structural Formula (I) is as follows:

Values of Z¹-Z⁴, R¹, R², Ring S, Ring T, X, J^{A}, J^{B}, J^{C}, J^{T}, R, R′, R^{s}, R^{t}, R¹⁴, and R¹⁵ are each and independently as described above in the thirty sixth set of values of the variables of Structural Formula (I).

Q¹ is independently —NRC(O)—, —C(O)NR—, or —NRC(O)NR′—.

The remaining variables of Structural Formula (I) are each and independently as described above in the first set of values of the variables of Structural Formula

The fortieth set of values of the variables of Structural Formula (I) is as follows:

Values of Z¹-Z⁴, R¹, R², Ring S, Ring T, X, J^{A}, J^{B}, J^{C}, J^{T}, Q¹, Y¹, R, R′, R^{s}, R^{t}, R¹⁴, and R¹⁵ are each and independently as described above in any one of the thirty sixth through thirty ninth sets of values of the variables of Structural Formula (I).

R and R′ are each and independently —H or —CH₃.

The remaining variables of Structural Formula (I) are each and independently as described above in the first set of values of the variables of Structural Formula

The forty first set of values of the variables of Structural Formula (I) is as follows:

Values of Z¹-Z⁴, R¹, R², Ring S, Ring T, X, J^{A}, J^{B}, J^{C}, J^{T}, Q¹, Y¹, R, R′, R^{s}, R^{t}, R¹⁴, and R¹⁵ are each and independently as described above in the thirty sixth through thirty ninth sets of values of the variables of Structural Formula (I).

R and R′ are each and independently —H or —CH₃.

R¹ is independently a 4-7 membered heterocyclic group, a phenyl group, or a 5-6 membered heteroaryl group, wherein each of said heterocyclic, phenyl and heteroaryl groups is independently and optionally substituted with one or more substituents independently selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl); or

optionally R¹ and R′, together with the nitrogen atom to which they are attached, form an optionally substituted, 4-8 membered heterocyclic group.

The remaining variables of Structural Formula (I) are each and independently as described above in the first set of values of the variables of Structural Formula

In the forty second set of values of the variables of Structural Formula (I), X is —F, —Cl, —CH₃, or —CF₃, and the remaining variables are each and independently as described above in any one of the sets of values of the variables of Structural Formula (I).

In the forty third set of values of the variables of Structural Formula (I), p is 1 or 2, k is 1 or 2, and the remaining variables are each and independently as described above in any one of the sets of values of the variables of Structural Formula (I).

In the forty fourth set of values of the variables of Structural Formula (I), X is —F, or —Cl, and the remaining variables are each and independently as described above in any one of the sets of values of the variables of Structural Formula (I).

Specific examples of the compounds represented by Structural Formula (I) include:

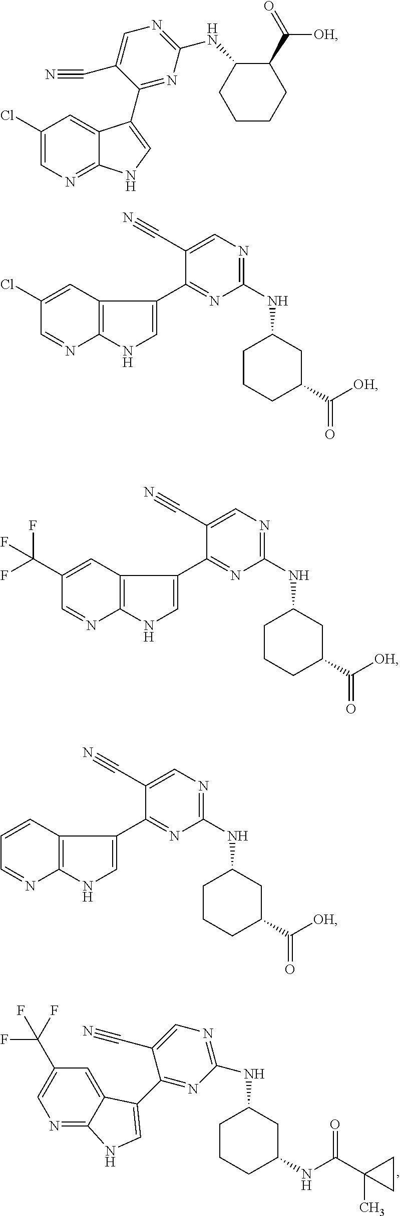

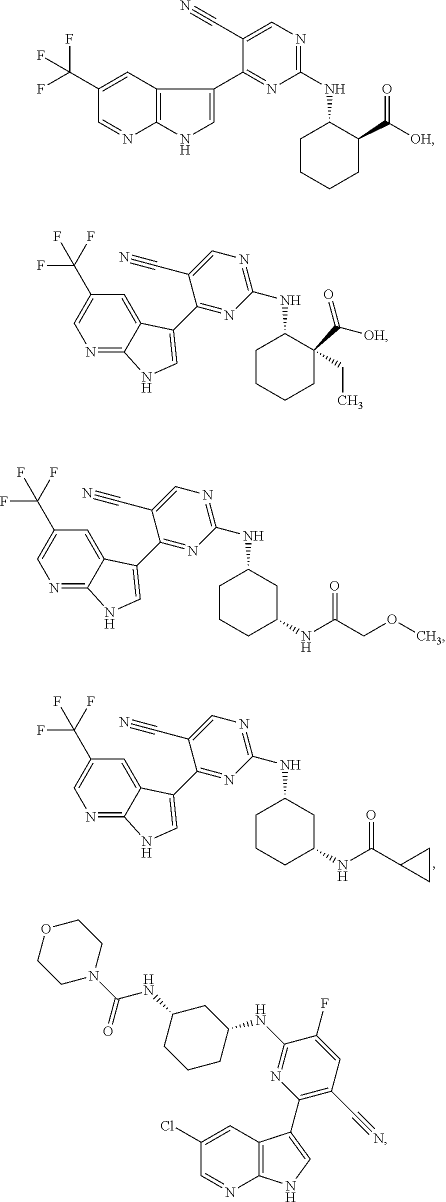 [SMILES: N#Cc1cnc(N[C@H]2CCCC[C@@H]2C(=O)O)nc1-c1c[nH]c2ncc(C(F)(F)F)cc12 ; CC[C@]1(C(=O)O)CCCC[C@@H]1Nc1ncc(C#N)c(-c2c[nH]c3ncc(C(F)(F)F)cc23)n1 ; COCC(=O)N[C@@H]1CCC[C@H](Nc2ncc(C#N)c(-c3c[nH]c4ncc(C(F)(F)F)cc34)n2)C1 ; N#Cc1cnc(N[C@H]2CCC[C@@H](NC(=O)C3CC3)C2)nc1-c1c[nH]c2ncc(C(F)(F)F)cc12 ; N#Cc1cc(F)c(N[C@@H]2CCC[C@H](NC(=O)N3CCOCC3)C2)nc1-c1c[nH]c2ncc(Cl)cc12]

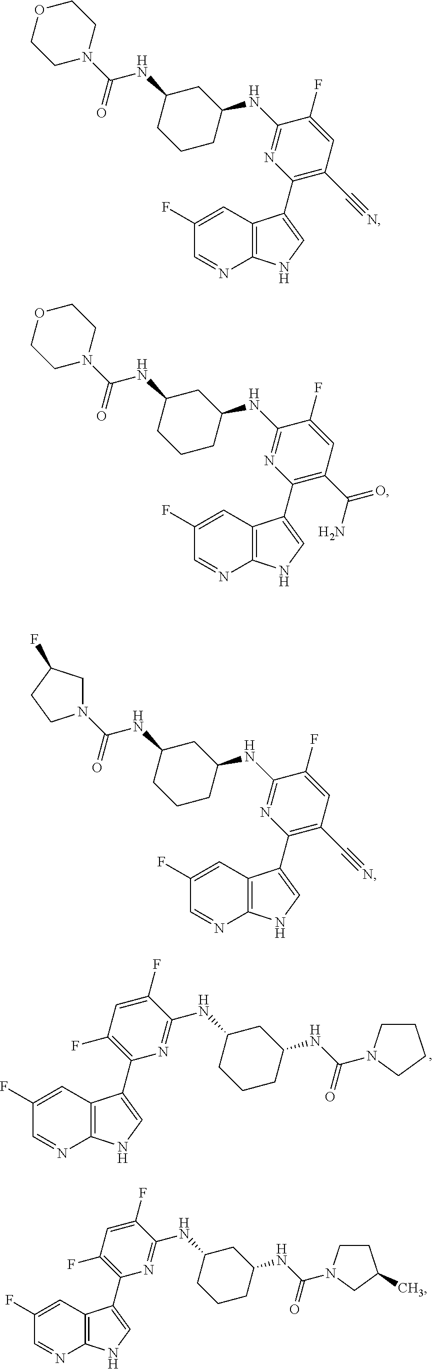 [SMILES: N#Cc1cc(F)c(N[C@H]2CCC[C@@H](NC(=O)N3CCOCC3)C2)nc1-c1c[nH]c2ncc(F)cc12 ; NC(=O)c1cc(F)c(N[C@H]2CCC[C@@H](NC(=O)N3CCOCC3)C2)nc1-c1c[nH]c2ncc(F)cc12 ; N#Cc1cc(F)c(N[C@H]2CCC[C@@H](NC(=O)N3CC[C@@H](F)C3)C2)nc1-c1c[nH]c2ncc(F)cc12 ; O=C(N[C@@H]1CCC[C@H](Nc2nc(-c3c[nH]c4ncc(F)cc34)c(F)cc2F)C1)N1CCCC1 ; C[C@@H]1CCN(C(=O)N[C@@H]2CCC[C@H](Nc3nc(-c4c[nH]c5ncc(F)cc45)c(F)cc3F)C2)C1]

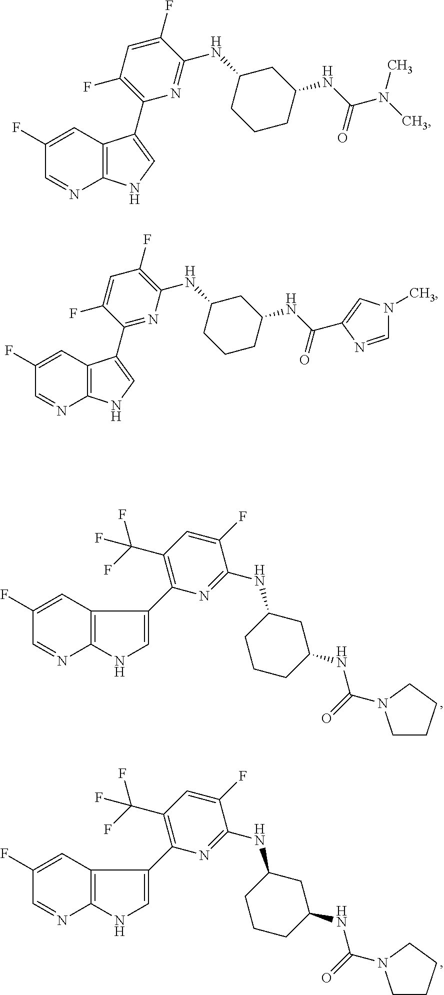 [SMILES: CN(C)C(=O)N[C@@H]1CCC[C@H](Nc2nc(-c3c[nH]c4ncc(F)cc34)c(F)cc2F)C1 ; Cn1cnc(C(=O)N[C@@H]2CCC[C@H](Nc3nc(-c4c[nH]c5ncc(F)cc45)c(F)cc3F)C2)c1 ; O=C(N[C@@H]1CCC[C@H](Nc2nc(-c3c[nH]c4ncc(F)cc34)c(C(F)(F)F)cc2F)C1)N1CCCC1 ; O=C(N[C@H]1CCC[C@@H](Nc2nc(-c3c[nH]c4ncc(F)cc34)c(C(F)(F)F)cc2F)C1)N1CCCC1]

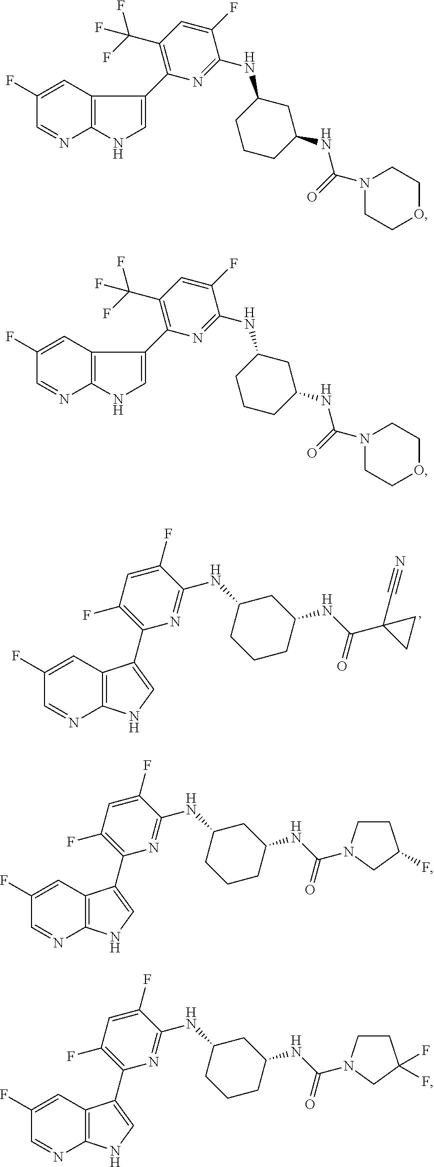 [SMILES: O=C(N[C@H]1CCC[C@@H](Nc2nc(-c3c[nH]c4ncc(F)cc34)c(C(F)(F)F)cc2F)C1)N1CCOCC1 ; O=C(N[C@@H]1CCC[C@H](Nc2nc(-c3c[nH]c4ncc(F)cc34)c(C(F)(F)F)cc2F)C1)N1CCOCC1 ; N#CC1(C(=O)N[C@@H]2CCC[C@H](Nc3nc(-c4c[nH]c5ncc(F)cc45)c(F)cc3F)C2)CC1 ; O=C(N[C@@H]1CCC[C@H](Nc2nc(-c3c[nH]c4ncc(F)cc34)c(F)cc2F)C1)N1CC[C@H](F)C1 ; O=C(N[C@@H]1CCC[C@H](Nc2nc(-c3c[nH]c4ncc(F)cc34)c(F)cc2F)C1)N1CCC(F)(F)C1]

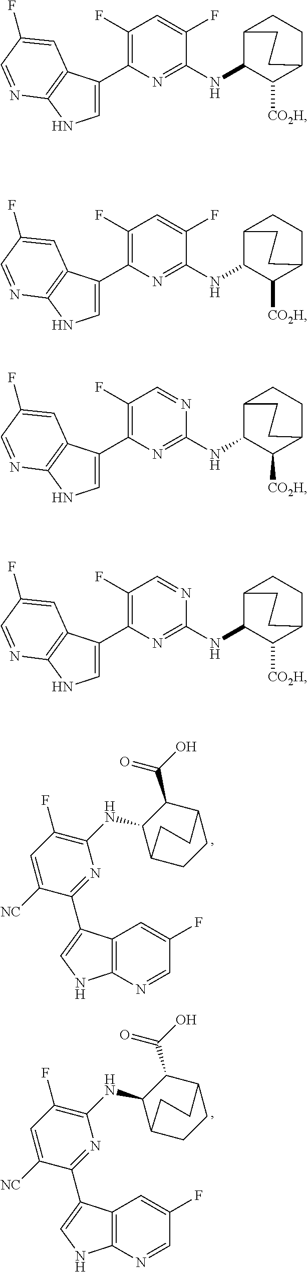

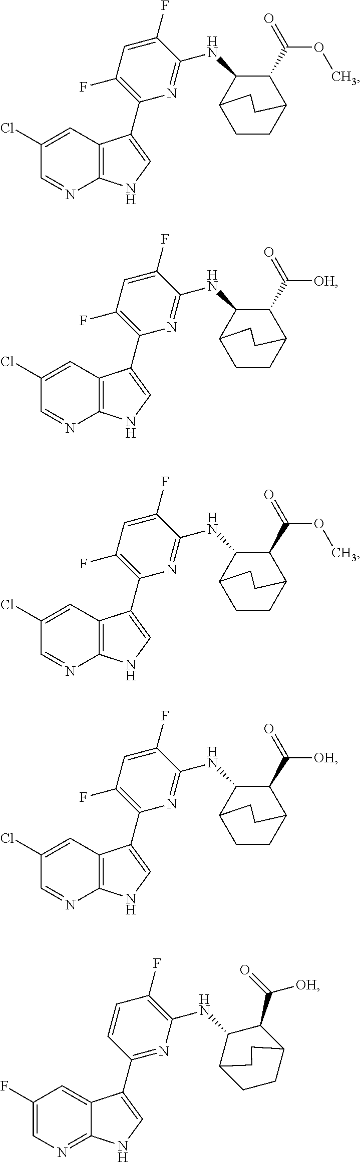 [SMILES: COC(=O)[C@@H]1C2CCC(CC2)[C@H]1Nc1nc(-c2c[nH]c3ncc(Cl)cc23)c(F)cc1F ; O=C(O)[C@@H]1C2CCC(CC2)[C@H]1Nc1nc(-c2c[nH]c3ncc(Cl)cc23)c(F)cc1F ; COC(=O)[C@H]1C2CCC(CC2)[C@@H]1Nc1nc(-c2c[nH]c3ncc(Cl)cc23)c(F)cc1F ; O=C(O)[C@H]1C2CCC(CC2)[C@@H]1Nc1nc(-c2c[nH]c3ncc(Cl)cc23)c(F)cc1F ; O=C(O)[C@H]1C2CCC(CC2)[C@@H]1Nc1nc(-c2c[nH]c3ncc(F)cc23)ccc1F]

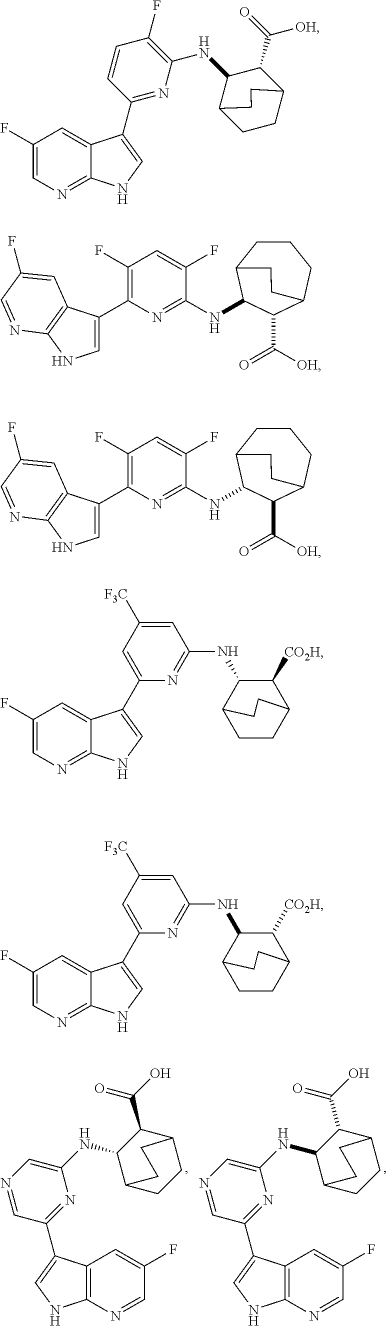

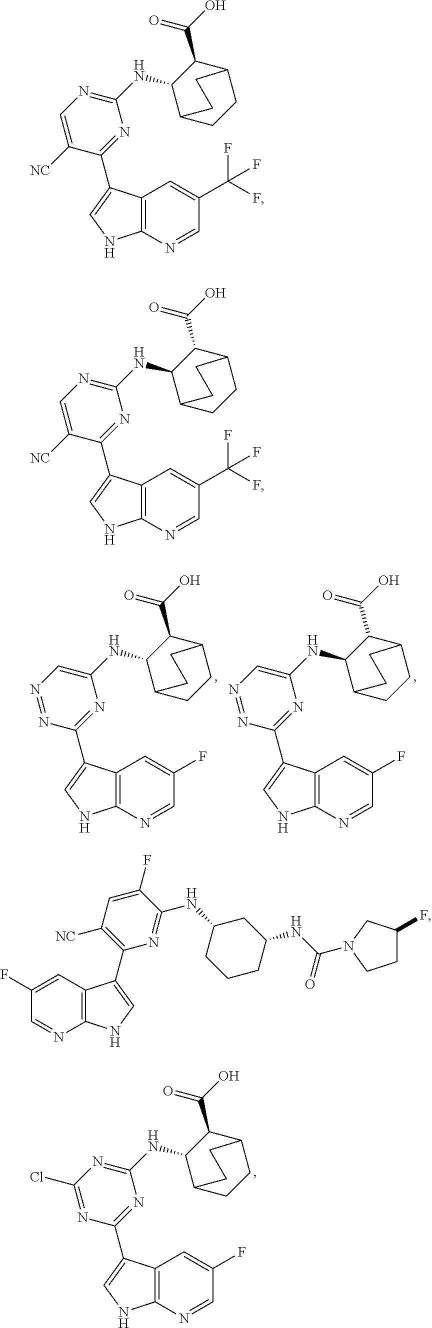

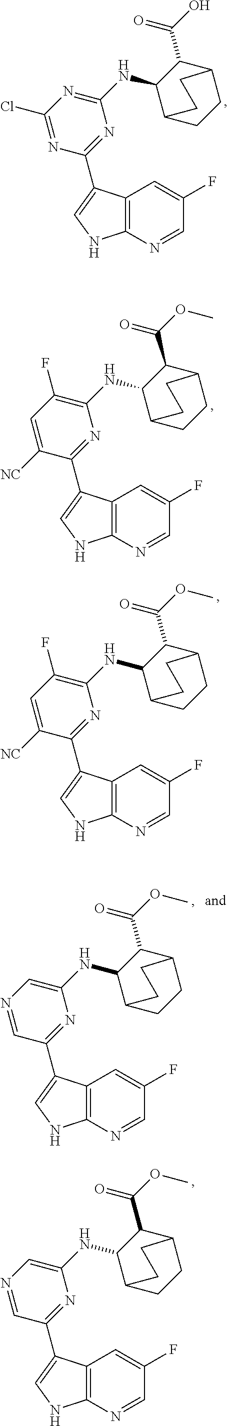

and pharmaceutically acceptable salts thereof.

Additional specific examples include:

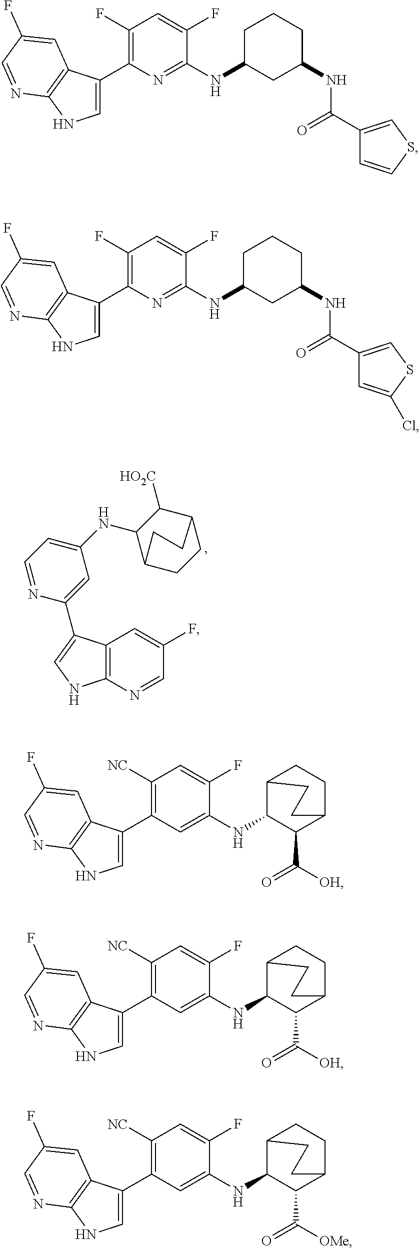

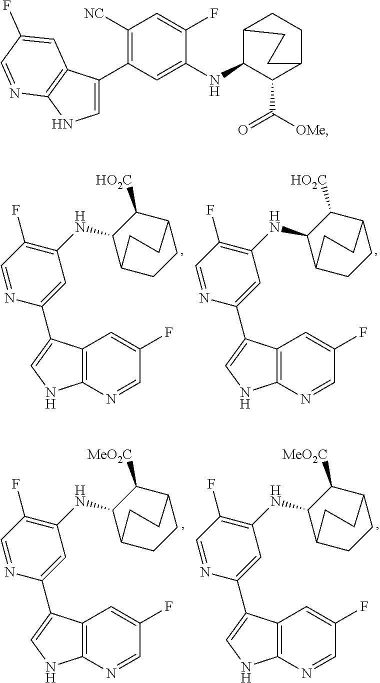

and pharmaceutically acceptable salts thereof.

In some embodiments, the compounds of the invention are selected from any one of the compounds depicted in Tables 1 and 2, or pharmaceutically acceptable salts thereof.

As used herein, a reference to compound(s) of the invention, for example the compound(s) of Structural Formula (I), or compounds(s) of claim 1, will include pharmaceutically acceptable salts thereof.

The compounds of the invention described herein can be prepared by any suitable method known in the art. For example, they can be prepared in accordance with procedures described in WO 2005/095400, WO 2007/084557, WO 2010/011768, WO 2010/011756, WO 2010/011772, WO 2009/073300, and PCT/US2010/038988 filed on Jun. 17, 2010. For example, the compounds shown in Tables 1 and 2 and the specific compounds depicted above can be prepared by any suitable method known in the art, for example, WO 2005/095400, WO 2007/084557, WO 2010/011768, WO 2010/011756, WP 2010/011772, WO 2009/073300, and PCT/US2010/038988, and by the exemplary syntheses described below under Exemplification.

The present invention provides methods of preparing a compound represented by Structural Formula (I). In one embodiment, the compounds of the invention can be prepared as depicted in General Schemes 1-4. Any suitable condition(s) known in the art can be employed in the invention for each step depicted in the schemes.

In a specific embodiment, as shown in General Scheme 1, the methods comprise the step of reacting Compound (A) with Compound (B) under suitable conditions to form a compound of Structural Formula (XX), wherein L² is a halogen (F, Cl, Br, or I), G is tosyl or trityl, and the remaining variables of Compounds (A), (B) and Structural Formula (XX) are each and independently as described herein. Typically, G is tosyl. Typical examples for L² are F, Cl or Br. More typical examples for L² are Cl or Br. The methods further comprise the step of deprotecting the G group under suitable conditions to form the compounds of Structural Formula (I). Any suitable condition(s) known in the art can be employed in the invention for each step depicted in the schemes. For example, any suitable condition described in WO 2005/095400 and WO 2007/084557 for the coupling of a dioxaboraolan with a chloro-pyrimidine can be employed for the reaction between Compounds (A) and (B). Specifically, the reaction between compounds (A) and (B) can be performed in the presence of Pd(PPh₃)₄ or Pd₂(dba)₃ (dba is dibenzylidene acetone). For example, the de-tosylation step can be performed under a basic condition, for example, in the presence of LiOH or NaOH, or under acidic conditions, for example, in the presence of HCl (e.g., in acetonitrile at 70° C.). For example, the de-tritylation step can be performed under an acidic condition (e.g., trifluoroacetic acid (TFA)) in the presence of, for example, Et₃SiH (Et is ethyl). Specific exemplary conditions are described in the Exemplification below

Optionally, the method further comprises the step of preparing Compound (A) by reacting Compound (E) with Compound (D), wherein each of L¹ and L² independently is a halogen (F, Cl, Br, or I), G is tosyl or trityl, and the remaining variables of Compounds (A), (B) and Structural Formula (XX) are each and independently as described herein. Typical examples for L¹ and L² are each and independently F, Cl or Br. More typical examples for L¹ and L² are each and independently Cl or Br. Any suitable conditions know in the art can be employed in this step, and Compounds (E) and (D) can be prepared by any suitable method known in the art. Specific exemplary conditions are described in the Exemplification below.

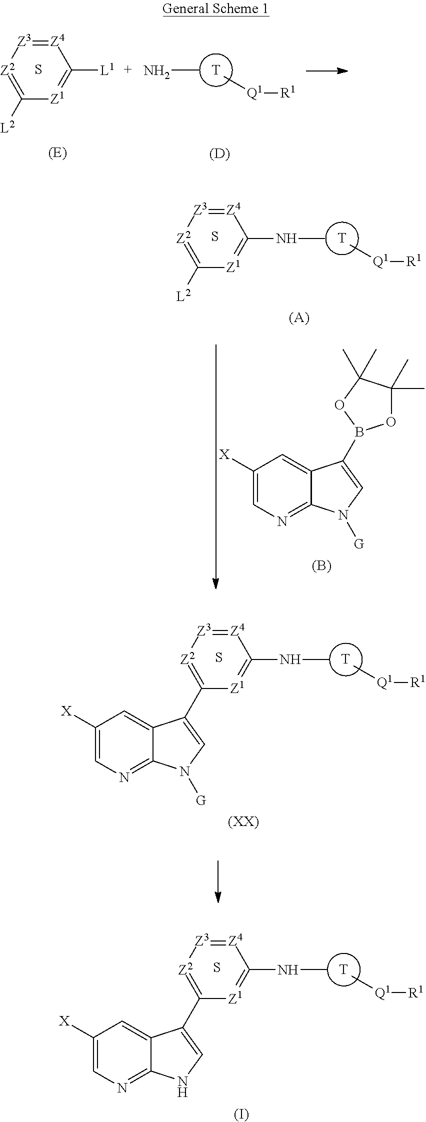

In another specific embodiment, as shown in General Scheme 2, the methods comprise the step of reacting Compound (G) with Compound (D) under suitable conditions to form a compound of Structural Formula (XX), wherein L¹ is a halogen (F, Cl, Br, or I), G is tosyl or trityl, and the remaining variables of Compounds (G), (D) and Structural Formula (XX) are each and independently as described herein. Typically, G is tosyl. Typically, L¹ is F, Cl, or Br. More typically, L¹ is Cl or Br. The methods further comprise the step of deprotecting the G group under suitable conditions to form the compounds of Structural Formula (I). Any suitable condition(s) known in the art can be employed in the invention for each step depicted in the schemes. For example, any suitable amination condition known in the art can be employed in the invention for the reaction of Compounds (G) and (D), and any suitable condition for deprotecting a Ts group can be employed in the invention for the deprotection step. For example, the amination step can be performed in the presence of a base, such as NEt₃ or N(ⁱPr)₂Et. For example, the de-tosylation step can be performed under a basic condition, for example, in the presence of LiOH or NaOH, or under acidic conditions, for example, in the presence of HCl (e.g., in acetonitrile at 70° C.). For example, the de-tritylation step can be performed under an acidic condition (e.g., trifluoroacetic acid (TFA)) in the presence of, for example, Et₃SiH (Et is ethyl). Additional specific exemplary conditions are described in the Exemplification below

Optionally, the method further comprises the step of preparing Compound (G) by reacting Compound (F) with Compound (B), wherein each of L¹ and L² independently is a halogen (F, Cl, Br, or I), G is tosyl or trityl, and the remaining variables of Compounds (A), (B) and Structural Formula (XX) are each and independently as described herein. Typical examples for L¹ and L² are each and independently F, Cl or Br. More typical examples for L¹ and L² are each and independently Cl or Br. Any suitable conditions know in the art can be employed in this step. For example, any suitable condition described in WO 2005/095400 and WO 2007/084557 for the coupling of a dioxaboralan with a chloro-pyrimidine can be employed for the reaction between Compounds (F) and (B). Specifically, the reaction between compounds (F) and (B) can be performed in the presence of Pd(PPh₃)₄ or Pd₂(dba)₃ (dba is dibenzylidene acetone). Specific exemplary conditions are described in the Exemplification below.

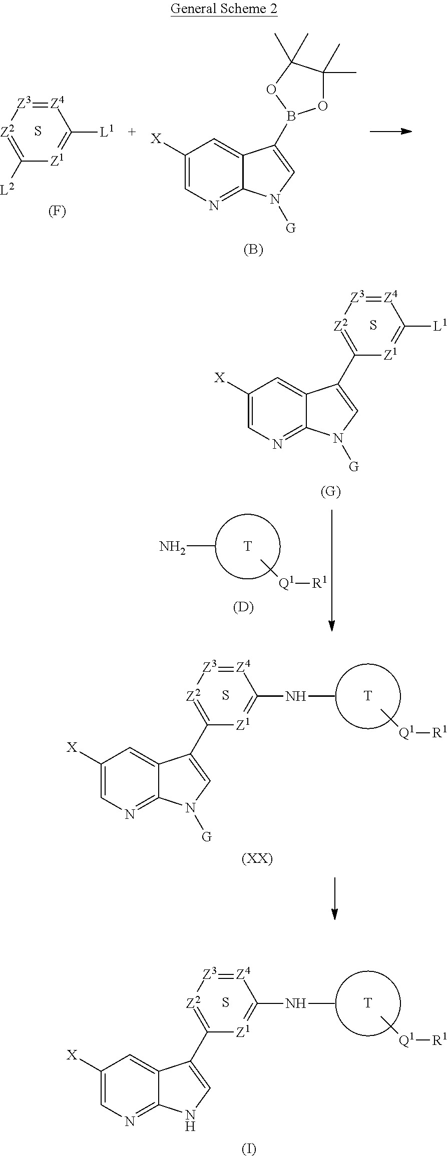

In yet another specific embodiment, as shown in General Scheme 3, the methods comprise the step of reacting Compound (K) with Compound (D) under suitable conditions to form a compound of Structural Formula (XX), wherein G is tosyl or trityl, and the remaining variables of Compounds (K), (D) and Structural Formula (XX) are each and independently as described herein. Typically G is tosyl. The methods further comprise the step of deprotecting the G group under suitable conditions to form the compounds of Structural Formula (I). Any suitable condition(s) known in the art can be employed in the invention for each step depicted in the schemes. For example, any suitable reaction condition known in the art, for example, in WO 2005/095400 and WO 2007/084557 for the coupling of an amine with a sulfinyl group can be employed for the reaction of Compounds (K) with Compound (D). For example, Compounds (D) and (K) can be reacted in the presence of a base, such as NEt₃ or N(ⁱPr)₂(Et). For example, the de-tosylation step can be performed under a basic condition, for example, in the presence of LiOH or NaOH, (e.g., in acetonitrile at 70° C.). For example, the de-tritylation step can be performed under an acidic condition (e.g., trifluoroacetic acid (TFA)) in the presence of, for example, Et₃SiH (Et is ethyl). Additional specific exemplary conditions are described in the Exemplification below

Optionally, the method further comprises the step of preparing Compound (K) by oxidizing Compound (J), for example, by treatment with meta-chloroperbenzoic acid.

Optionally, the method further comprises the step of preparing Compound (J) by reacting Compound (H) with Compound (B). Any suitable conditions know in the art can be employed in this step. For example, any suitable condition described in WO 2005/095400 and WO 2007/084557 for the coupling of a dioxaboraolan with a chloro-pyrimidine can be employed for the reaction between Compounds (H) and (B). Specifically, the reaction between compounds (H) and (B) can be performed in the presence of Pd(PPh₃)₄ or Pd₂(dba)₃ (dba is dibenzylidene acetone) Specific exemplary conditions are described in the Exemplification below.

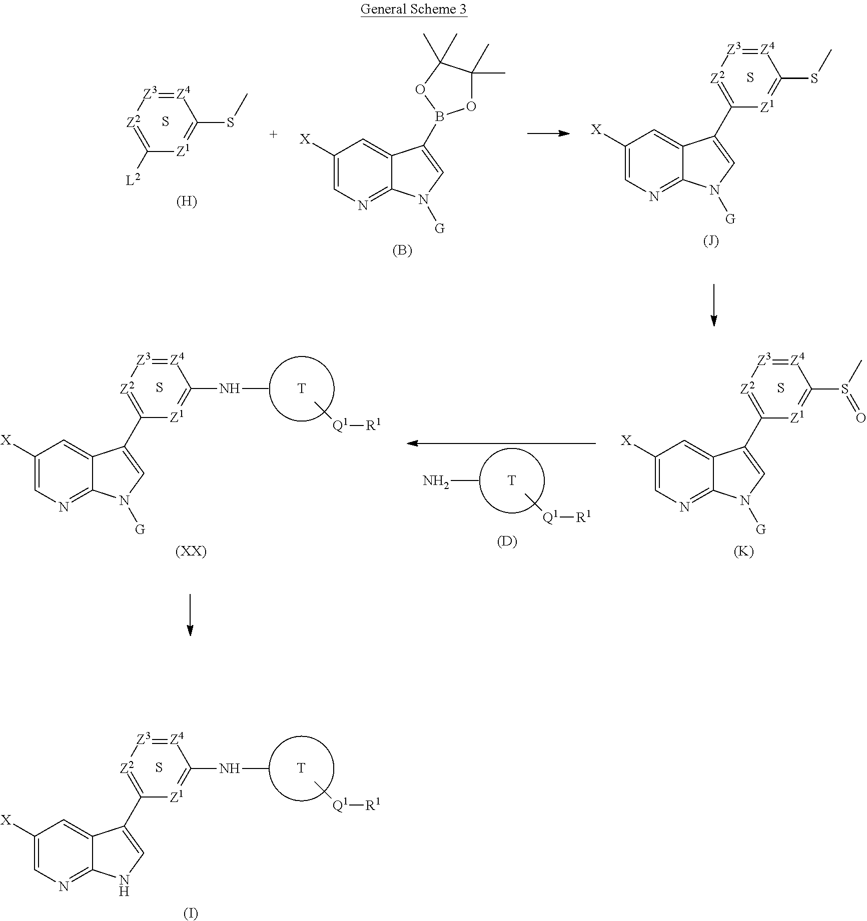

In yet another specific embodiment, as shown in General Scheme 4, the methods comprise the step of reacting Compound (L) with Compound (D) under suitable conditions to form a compound of Structural Formula (XX), wherein L² is a halogen (F, Cl, Br, or I), G is tosyl or trityl, and the remaining variables of Compounds (L), (D) and Structural Formula (XX) are each and independently as described herein. Typically, G is tosyl. Typical examples of L² are F, Cl or Br. More typical examples of L² are Cl or Br. The methods further comprise the step of deprotecting the G group under suitable conditions to form the compounds of Structural Formula (I). Any suitable condition(s) known in the art can be employed in the invention for each step depicted in the schemes. For example, any suitable reaction condition known in the art, for example, in WO 2005/095400 and WO 2007/084557 for the coupling of an amine with a sulfonyl group can be employed for the reaction of Compounds (L) with Compound (D). For example, Compounds (D) and (L) can be reacted in the presence of a base, such as NEt₃ or N(ⁱPr)₂(Et). For example, the de-tosylation step can be performed under a basic condition, for example, in the presence of LiOH or NaOH, or under acidic conditions, for example, in the presence of HCl (e.g., in acetonitrile at 70° C.). For example, the de-tritylation step can be performed under an acidic condition (e.g., trifluoroacetic acid (TFA)) in the presence of, for example, Et₃SiH (Et is ethyl). Additional specific exemplary conditions are described in the Exemplification below

Optionally, the method further comprises the step of preparing Compound (L) by oxidizing Compound (J), for example, by treatment with meta-chloroperbenzoic acid.

Optionally, the method further comprises the step of preparing Compound (J) by reacting Compound (H) with Compound (B). Reaction conditions are as described above for General Scheme 3.

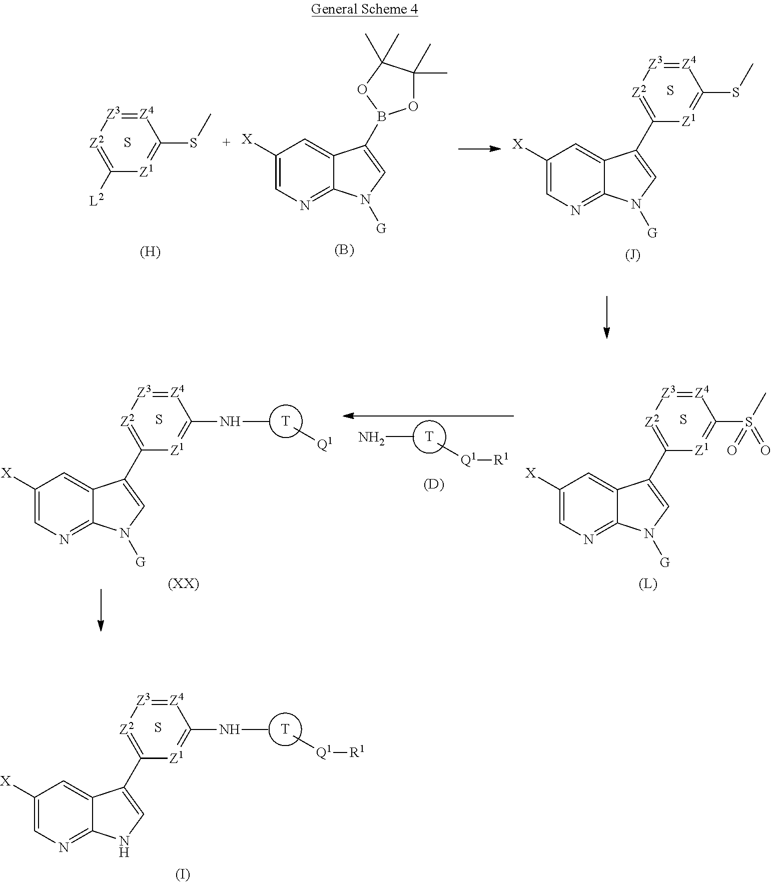

Compounds (A)-(L) can be prepared by any suitable method known in the art. Specific exemplary synthetic methods of these compounds are described below in the Exemplification. In one embodiment, Compounds (A), (G), (J), (K), and (L) can be prepared as described in General Schemes 1-4.

In some embodiments, the present invention is directed to a compound represented by Structural Formula (XX), wherein the variables of Structural Formula (XX) are each and independently as described herein, and G is trityl or tosyl. Typically, G is tosyl. The compounds represented by Structural formula (XX) can be prepared as described above. In one embodiment, the compounds of the invention can be prepared as depicted in General Schemes 1-4.

In some embodiments, the present invention is directed to a compound represented by Structural Formula (XX), wherein the variables of Structural Formula (XX) are each and independently as described herein, and G is tosyl or trityl. Specific examples of the compounds of Structural Formula (XX) include:

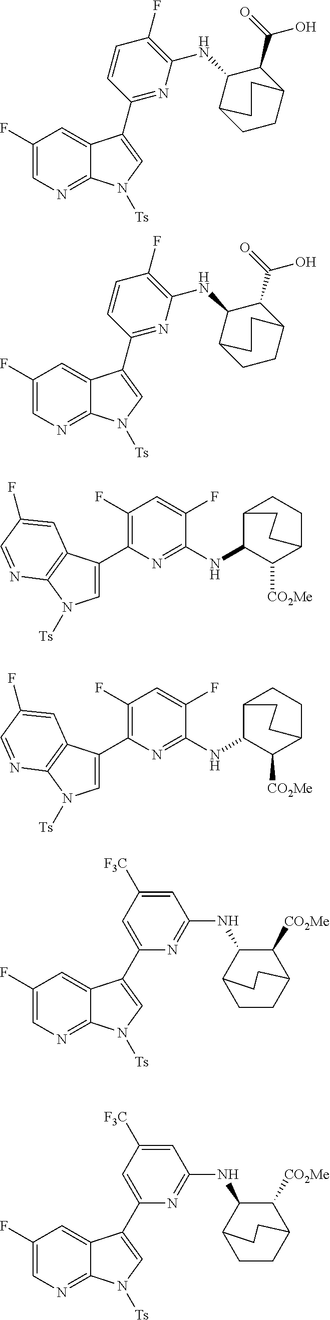

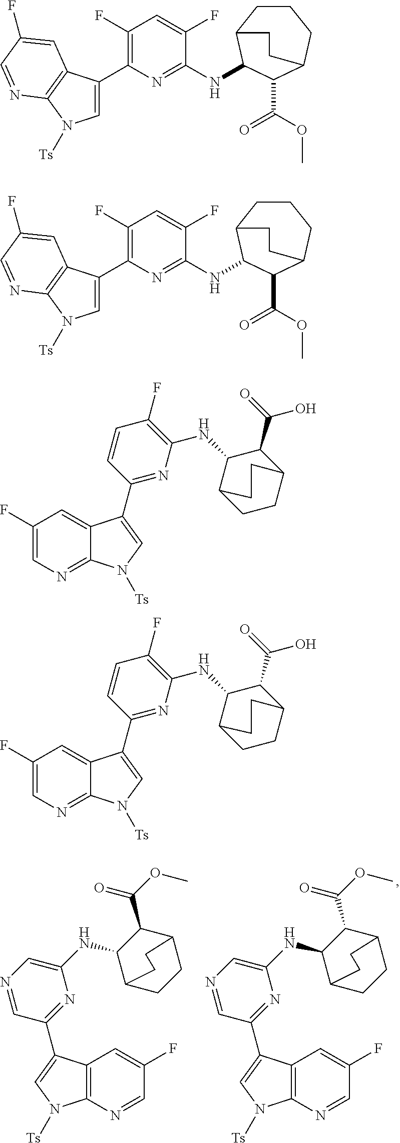

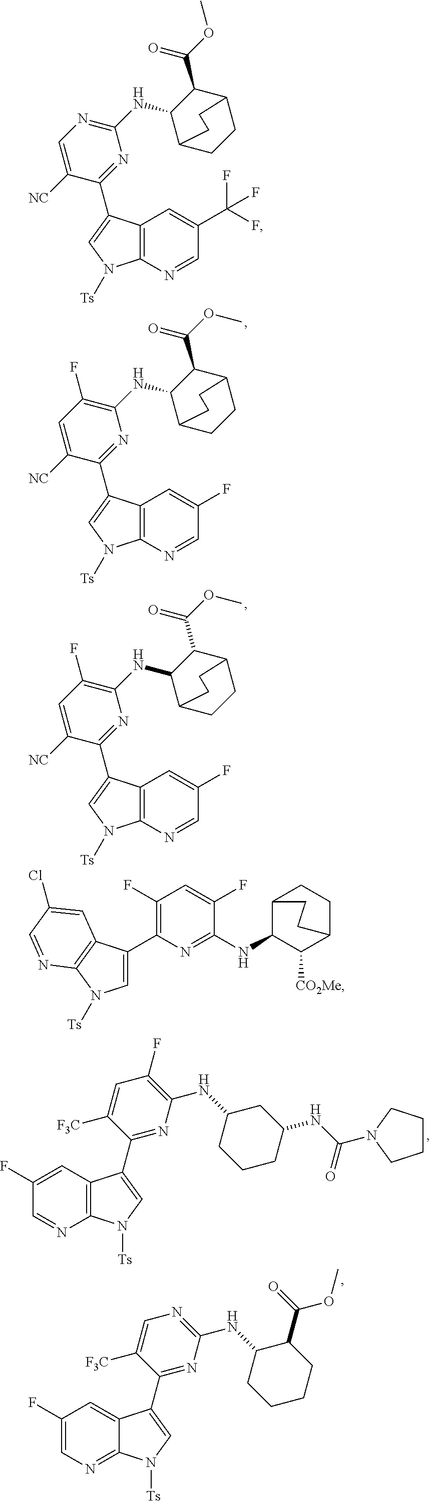

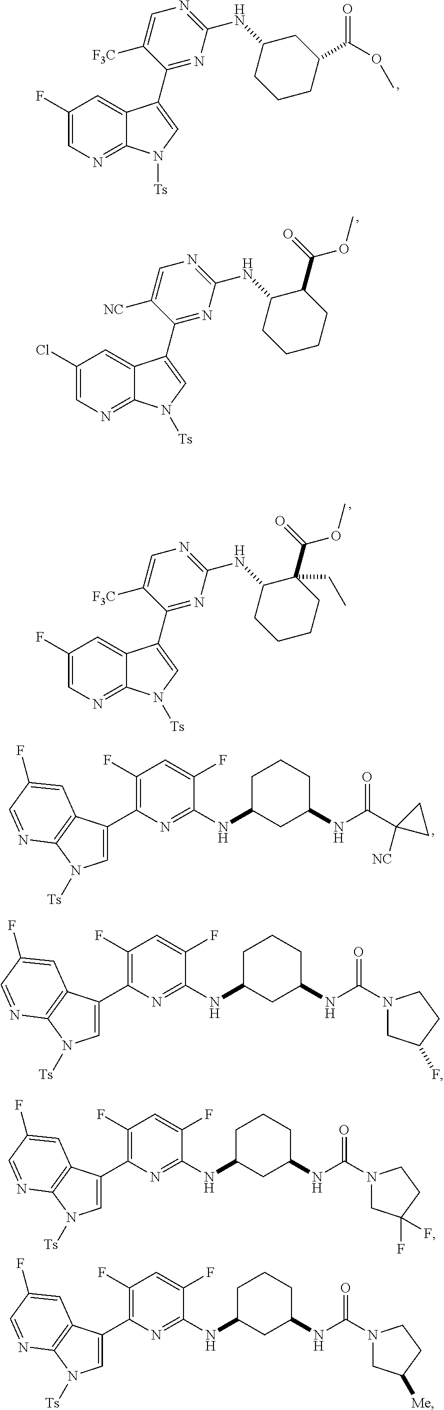

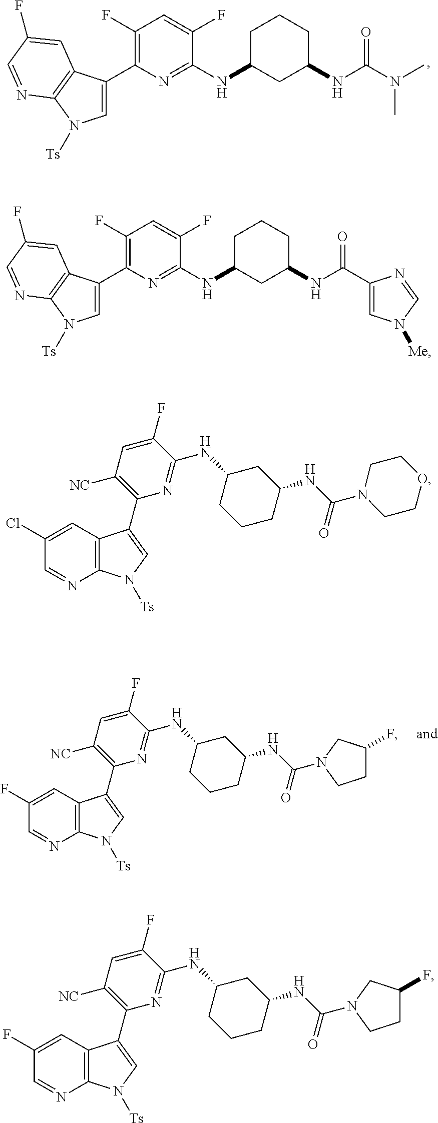

and pharmaceutically acceptable salts thereof, wherein Ts is tosyl. Additional examples include:

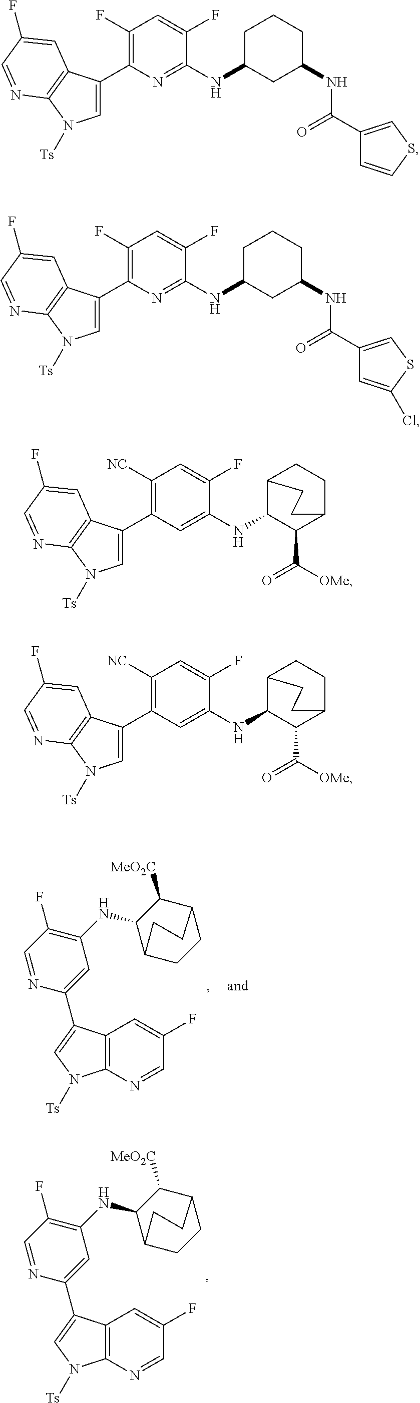

and pharmaceutically acceptable salts thereof, wherein Ts is tosyl.

### Definitions and General Terminology

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausolito: 1999, and “March's Advanced Organic Chemistry”, 5th Ed., Ed.: Smith, M. B. and March, J., John Wiley & Sons, New York: 2001, the entire contents of which are hereby incorporated by reference.

As described herein, compounds of the invention may optionally be substituted with one or more substituents, such as illustrated generally below, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. When the term “optionally substituted” precedes a list, said term refers to all of the subsequent substitutable groups in that list. If a substituent radical or structure is not identified or defined as “optionally substituted”, the substituent radical or structure is unsubstituted. For example, if X is optionally substituted C₁₋-C₃alkyl or phenyl; X may be either optionally substituted C₁-C₃ alkyl or optionally substituted phenyl. Likewise, if the term “optionally substituted” follows a list, said term also refers to all of the substitutable groups in the prior list unless otherwise indicated. For example: if X is C₁-C₃alkyl or phenyl wherein X is optionally and independently substituted by J^{X}, then both C₁-C₃alkyl and phenyl may be optionally substituted by J^{X}.

The phrase “up to”, as used herein, refers to zero or any integer number that is equal or less than the number following the phrase. For example, “up to 3” means any one of 0, 1, 2, and 3. As described herein, a specified number range of atoms includes any integer therein. For example, a group having from 1-4 atoms could have 1, 2, 3, or 4 atoms.

Selection of substituents and combinations of substituents envisioned by this invention are those that result in the formation of stable or chemically feasible compounds. The term “stable”, as used herein, refers to compounds that are not substantially altered when subjected to conditions to allow for their production, detection, and, specifically, their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week. Only those choices and combinations of substituents that result in a stable structure are contemplated. Such choices and combinations will be apparent to those of ordinary skill in the art and may be determined without undue experimentation.

The term “aliphatic” or “aliphatic group”, as used herein, means a straight-chain (i.e., unbranched), or branched, hydrocarbon chain that is completely saturated or that contains one or more units of unsaturation but is non-aromatic. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms, and in yet other embodiments, aliphatic groups contain 1-4 aliphatic carbon atoms. Aliphatic groups may be linear or branched, substituted or unsubstituted alkyl, alkenyl, or alkynyl groups. Specific examples include, but are not limited to, methyl, ethyl, isopropyl, n-propyl, sec-butyl, vinyl, n-butenyl, ethynyl, and tert-butyl and acetylene.

The term “alkyl” as used herein means a saturated straight or branched chain hydrocarbon. The term “alkenyl” as used herein means a straight or branched chain hydrocarbon comprising one or more double bonds. The term “alkynyl” as used herein means a straight or branched chain hydrocarbon comprising one or more triple bonds. Each of the “alkyl”, “alkenyl” or “alkynyl” as used herein can be optionally substituted as set forth below. In some embodiments, the “alkyl” is C₁-C₆ alkyl or C₁-C₄ alkyl. In some embodiments, the “alkenyl” is C₂-C₆ alkenyl or C₂-C₄ alkenyl. In some embodiments, the “alkynyl” is C₂-C₆ alkynyl or C₂-C₄ alkynyl.

The term “cycloaliphatic” (or “carbocycle” or “carbocyclyl” or “carbocyclic”) refers to a non-aromatic carbon only containing ring system which can be saturated or contains one or more units of unsaturation, having three to fourteen ring carbon atoms. In some embodiments, the number of carbon atoms is 3 to 10. In other embodiments, the number of carbon atoms is 4 to 7. In yet other embodiments, the number of carbon atoms is 5 or 6. The term includes monocyclic, bicyclic or polycyclic, fused, spiro or bridged carbocyclic ring systems. The term also includes polycyclic ring systems in which the carbocyclic ring can be “fused” to one or more non-aromatic carbocyclic or heterocyclic rings or one or more aromatic rings or combination thereof, wherein the radical or point of attachment is on the carbocyclic ring. “Fused” bicyclic ring systems comprise two rings which share two adjoining ring atoms. Bridged bicyclic group comprise two rings which share three or four adjacent ring atoms. Spiro bicyclic ring systems share one ring atom. Examples of cycloaliphatic groups include, but are not limited to, cycloalkyl and cycloalkenyl groups. Specific examples include, but are not limited to, cyclohexyl, cyclopropenyl, and cyclobutyl.

The term “heterocycle” (or “heterocyclyl”, or “heterocyclic” or “non-aromatic heterocycle”) as used herein refers to a non-aromatic ring system which can be saturated or contain one or more units of unsaturation, having three to fourteen ring atoms in which one or more ring carbons is replaced by a heteroatom such as, N, S, or O and each ring in the system contains 3 to 7 members. In some embodiments, non-aromatic heterocyclic rings comprise up to three heteroatoms selected from N, S and O within the ring. In other embodiments, non-aromatic heterocyclic rings comprise up to two heteroatoms selected from N, S and O within the ring system. In yet other embodiments, non-aromatic heterocyclic rings comprise up to two heteroatoms selected from N and O within the ring system. The term includes monocyclic, bicyclic or polycyclic fused, spiro or bridged heterocyclic ring systems. The term also includes polycyclic ring systems in which the heterocyclic ring can be fused to one or more non-aromatic carbocyclic or heterocyclic rings or one or more aromatic rings or combination thereof, wherein the radical or point of attachment is on the heterocyclic ring. Examples of heterocycles include, but are not limited to, piperidinyl, piperizinyl, pyrrolidinyl, pyrazolidinyl, imidazolidinyl, azepanyl, diazepanyl, triazepanyl, azocanyl, diazocanyl, triazocanyl, oxazolidinyl, isoxazolidinyl, thiazolidinyl, isothiazolidinyl, oxazocanyl, oxazepanyl, thiazepanyl, thiazocanyl, benzimidazolonyl, tetrahydrofuranyl, tetrahydrofuranyl, tetrahydrothiophenyl, tetrahydrothiophenyl, morpholino, including, for example, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolanyl, benzodithianyl, 3-(1-alkyl)-benzimidazol-2-onyl, and 1,3-dihydro-imidazol-2-onyl.

The term “aryl” (or “aryl ring” or “aryl group”) used alone or as part of a larger moiety as in “aralkyl”, “aralkoxy”, “aryloxyalkyl”, or “heteroaryl” refers to carbocyclic aromatic ring systems. The term “aryl” may be used interchangeably with the terms “aryl ring” or “aryl group”.

“Carbocyclic aromatic ring” groups have only carbon ring atoms (typically six to fourteen) and include monocyclic aromatic rings such as phenyl and fused polycyclic aromatic ring systems in which two or more carbocyclic aromatic rings are fused to one another. Examples include 1-naphthyl, 2-naphthyl, 1-anthracyl and 2-anthracyl. Also included within the scope of the term “carbocyclic aromatic ring” or “carbocyclic aromatic”, as it is used herein, is a group in which an aromatic ring is “fused” to one or more non-aromatic rings (carbocyclic or heterocyclic), such as in an indanyl, phthalimidyl, naphthimidyl, phenanthridinyl, or tetrahydronaphthyl, where the radical or point of attachment is on the aromatic ring.

The terms “heteroaryl”, “heteroaromatic”, “heteroaryl ring”, “heteroaryl group”, “aromatic heterocycle” or “heteroaromatic group”, used alone or as part of a larger moiety as in “heteroaralkyl” or “heteroarylalkoxy”, refer to heteroaromatic ring groups having five to fourteen members, including monocyclic heteroaromatic rings and polycyclic aromatic rings in which a monocyclic aromatic ring is fused to one or more other aromatic ring. Heteroaryl groups have one or more ring heteroatoms. Also included within the scope of the term “heteroaryl”, as it is used herein, is a group in which an aromatic ring is “fused” to one or more non-aromatic rings (carbocyclic or heterocyclic), where the radical or point of attachment is on the aromatic ring. Bicyclic 6,5 heteroaromatic ring, as used herein, for example, is a six membered heteroaromatic ring fused to a second five membered ring, wherein the radical or point of attachment is on the six membered ring. Examples of heteroaryl groups include pyridyl, pyrazinyl, pyrimidinyl, pyridazinyl, imidazolyl, pyrrolyl, pyrazolyl, triazolyl, tetrazolyl, oxazolyl, isoxazolyl, oxadiazolyl, thiazolyl, isothiazolyl or thiadiazolyl including, for example, 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 3-pyrazolyl, 4-pyrazolyl, 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 3-pyridazinyl, 2-thiazolyl, 4-thiazolyl, 5-thiazolyl, 2-triazolyl, 5-triazolyl, tetrazolyl, 2-thienyl, 3-thienyl, carbazolyl, benzimidazolyl, benzothienyl, benzofuranyl, indolyl, benzotriazolyl, benzothiazolyl, benzoxazolyl, benzimidazolyl, isoquinolinyl, indolyl, isoindolyl, acridinyl, benzisoxazolyl, isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, “cyclo”, “cyclic”, “cyclic group” or “cyclic moiety”, include mono-, bi-, and tri-cyclic ring systems including cycloaliphatic, heterocycloaliphatic, carbocyclic aryl, or heteroaryl, each of which has been previously defined.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic carbocyclic aryls, and bicyclic heteroaryls.

As used herein, a “bridged bicyclic ring system” refers to a bicyclic heterocycloalipahtic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, carbocyclic aryl, heteroaryl, alkoxy, cycloalkyloxy, heterocycloalkyloxy, (carbocyclic aryl)oxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, (carbocyclic aryl)carbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, “bridge” refers to a bond or an atom or an unbranched chain of atoms connecting two different parts of a molecule. The two atoms that are connected through the bridge (usually but not always, two tertiary carbon atoms) are detonated as “bridgeheads”.

As used herein, the term “spiro” refers to ring systems having one atom (usually a quaternary carbon) as the only common atom between two rings.

The term “ring atom” is an atom such as C, N, O or S that is in the ring of an aromatic group, cycloalkyl group or non-aromatic heterocyclic ring.

A “substitutable ring atom” in an aromatic group is a ring carbon or nitrogen atom bonded to a hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to a moiety other than hydrogen.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

As used herein an optionally substituted aralkyl can be substituted on both the alkyl and the aryl portion. Unless otherwise indicated as used herein optionally substituted aralkyl is optionally substituted on the aryl portion.

In some embodiments, an aliphatic or heteroaliphatic group, or a non-aromatic heterocyclic ring may contain one or more substituents. Suitable substituents on the saturated carbon of an aliphatic or heteroaliphatic group, or of a heterocyclic ring are selected from those listed above. Other suitable substitutents include those listed as suitable for the unsaturated carbon of a carbocyclic aryl or heteroaryl group and additionally include the following: ═O, ═S, ═NNHR*, ═NN(R*)₂, ═NNHC(O)R*, ═NNHCO₂(alkyl), ═NNHSO₂(alkyl), or ═NR*, wherein each R* is independently selected from hydrogen or an optionally substituted C₁₋₆ aliphatic. Optional substituents on the aliphatic group of R* are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄ aliphatic groups of R* is unsubstituted.

In some embodiments, optional substituents on the nitrogen of a heterocyclic ring include those used above. Other suitable substituents include —R⁺, —N(R⁺)₂, —C(O)R⁺, —CO₂R⁺, —C(O)C(O)R⁺, —C(O)CH₂C(O)R⁺, —SO₂R⁺, —SO₂N(R⁺)₂, —C(═S)N(R⁺)₂, —C(═NH)—N(R⁺)₂, or —NR⁺SO₂R⁺; wherein R⁺ is hydrogen, an optionally substituted C₁₋₆ aliphatic, optionally substituted phenyl, optionally substituted —O(Ph), optionally substituted —CH₂(Ph), optionally substituted —(CH₂)₁₋₂(Ph); optionally substituted —CH═CH(Ph); or an unsubstituted 5-6 membered heteroaryl or heterocyclic ring having one to four heteroatoms independently selected from oxygen, nitrogen, or sulfur, or, two independent occurrences of R⁺, on the same substituent or different substituents, taken together with the atom(s) to which each R⁺ group is bound, form a 5-8-membered heterocyclyl, carbocyclic aryl, or heteroaryl ring or a 3-8-membered cycloalkyl ring, wherein said heteroaryl or heterocyclyl ring has 1-3 heteroatoms independently selected from nitrogen, oxygen, or sulfur. Optional substituents on the aliphatic group or the phenyl ring of R⁺ are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R⁺ is unsubstituted.

In some embodiments, an aryl (including aralkyl, aralkoxy, aryloxyalkyl and the like) or heteroaryl (including heteroaralkyl and heteroarylalkoxy and the like) group may contain one or more substituents. Suitable substituents on the unsaturated carbon atom of a carbocyclic aryl or heteroaryl group are selected from those listed above. Other suitable substituents include: halogen; —R^{∘}; —OR^{∘}; —SR^{∘}; 1,2-methylenedioxy; 1,2-ethylenedioxy; phenyl (Ph) optionally substituted with R^{∘}; —O(Ph) optionally substituted with R^{∘}; —(CH₂)₁₋₂(Ph), optionally substituted with R^{∘}; —CH═CH(Ph), optionally substituted with R^{∘}; —NO₂; —CN; —N(R^{∘})₂; —NR^{∘}C(O)R^{∘}; —NR^{∘}C(S)R^{∘}; —NR^{∘}C(O)N(R^{∘})₂; —NR^{∘}C(S)N(R^{∘})₂; —NR^{∘}CO₂R^{∘}; —NR^{∘}NR^{∘}C(O)R^{∘}; —NR^{∘}NR^{∘}C(O)N(R^{∘})₂; —NR^{∘}NR^{∘}CO₂R^{∘}; —C(O)C(O)R^{∘}; —C(O)CH₂C(O)R^{∘}; —CO₂R^{∘}; —C(O)R^{∘}; —C(S)R^{∘}; —C(O)N(R^{∘})₂; —C(S)N(R^{∘})₂; —OC(O)N(R^{∘})₂; —OC(O)R^{∘}; —C(O)N(OR^{∘})R^{∘}; —C(NOR^{∘}) R^{∘}; —S(O)₂R^{∘}; —S(O)₃R^{∘}; —SO₂N(R^{∘})₂; —S(O)R^{∘}; —NR^{∘}SO₂N(R^{∘})₂; —NR^{∘}SO₂R^{∘}; —N(OR^{∘})R^{∘}; —C(═NH)—N(R^{∘})₂; or —(CH₂)₀₋₂NHC(O)R^{∘}; wherein each independent occurrence of R^{∘} is selected from hydrogen, optionally substituted C₁₋₆ aliphatic, an unsubstituted 5-6 membered heteroaryl or heterocyclic ring, phenyl, —O(Ph), or —CH₂(Ph), or, two independent occurrences of R^{∘}, on the same substituent or different substituents, taken together with the atom(s) to which each R^{∘} group is bound, form a 5-8-membered heterocyclyl, carbocyclic aryl, or heteroaryl ring or a 3-8-membered cycloalkyl ring, wherein said heteroaryl or heterocyclyl ring has 1-3 heteroatoms independently selected from nitrogen, oxygen, or sulfur. Optional substituents on the aliphatic group of R^{∘} are selected from NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄ aliphatic), O(haloC₁₋₄ aliphatic), or haloC₁₋₄aliphatic, CHO, N(CO)(C₁₋₄ aliphatic), C(O)N(C₁₋₄ aliphatic), wherein each of the foregoing C₁₋₄aliphatic groups of R^{∘} is unsubstituted.

Non-aromatic nitrogen containing heterocyclic rings that are substituted on a ring nitrogen and attached to the remainder of the molecule at a ring carbon atom are said to be N substituted. For example, an N alkyl piperidinyl group is attached to the remainder of the molecule at the two, three or four position of the piperidinyl ring and substituted at the ring nitrogen with an alkyl group. Non-aromatic nitrogen containing heterocyclic rings such as pyrazinyl that are substituted on a ring nitrogen and attached to the remainder of the molecule at a second ring nitrogen atom are said to be N′ substituted-N-heterocycles. For example, an N′ acyl N-pyrazinyl group is attached to the remainder of the molecule at one ring nitrogen atom and substituted at the second ring nitrogen atom with an acyl group.

The term “unsaturated”, as used herein, means that a moiety has one or more units of unsaturation.

As detailed above, in some embodiments, two independent occurrences of R^{∘} (or R⁺, or any other variable similarly defined herein), may be taken together with the atom(s) to which each variable is bound to form a 5-8-membered heterocyclyl, carbocyclic aryl, or heteroaryl ring or a 3-8-membered cycloalkyl ring. Exemplary rings that are formed when two independent occurrences of R^{∘} (or R⁺, or any other variable similarly defined herein) are taken together with the atom(s) to which each variable is bound include, but are not limited to the following: a) two independent occurrences of R^{∘} (or R⁺, or any other variable similarly defined herein) that are bound to the same atom and are taken together with that atom to form a ring, for example, N(R^{∘})₂, where both occurrences of R^{∘} are taken together with the nitrogen atom to form a piperidin-1-yl, piperazin-1-yl, or morpholin-4-yl group; and b) two independent occurrences of R^{∘} (or R⁺, or any other variable similarly defined herein) that are bound to different atoms and are taken together with both of those atoms to form a ring, for example where a phenyl group is substituted with two occurrences of OR^{∘}

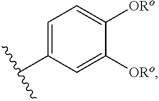

these two occurrences of R^{∘} are taken together with the oxygen atoms to which they are bound to form a fused 6-membered oxygen containing ring:

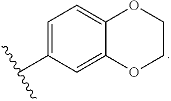 [SMILES: *c1ccc2c(c1)OCCO2]

It will be appreciated that a variety of other rings can be formed when two independent occurrences of R^{∘} (or R⁺, or any other variable similarly defined herein) are taken together with the atom(s) to which each variable is bound and that the examples detailed above are not intended to be limiting.

The term “hydroxyl” or “hydroxy” or “alcohol moiety” refers to —OH.

As used herein, an “alkoxycarbonyl,” which is encompassed by the term carboxy, used alone or in connection with another group refers to a group such as (alkyl-O)—C(O)—.

As used herein, a “carbonyl” refers to —C(O)—.

As used herein, an “oxo” refers to ═O.

As used herein, the term “alkoxy”, or “alkylthio”, as used herein, refers to an alkyl group, as previously defined, attached to the molecule through an oxygen (“alkoxy” e.g., —O-alkyl) or sulfur (“alkylthio” e.g., —S-alkyl) atom.

As used herein, the terms “halogen”, “halo”, and “hal” mean F, Cl, Br, or I.

As used herein, the term “cyano” or “nitrile” refer to —CN or —CEN.

The terms “alkoxyalkyl”, “alkoxyalkenyl”, “alkoxyaliphatic”, and “alkoxyalkoxy” mean alkyl, alkenyl, aliphatic or alkoxy, as the case may be, substituted with one or more alkoxy groups.

The terms “haloalkyl”, “haloalkenyl”, “haloaliphatic”, and “haloalkoxy” mean alkyl, alkenyl, aliphatic or alkoxy, as the case may be, substituted with one or more halogen atoms. This term includes perfluorinated alkyl groups, such as —CF₃ and —CF₂CF₃.

The terms “cyanoalkyl”, “cyanoalkenyl”, “cyanoaliphatic”, and “cyanoalkoxy” mean alkyl, alkenyl, aliphatic or alkoxy, as the case may be, substituted with one or more cyano groups. In some embodiments, the cyanoalkyl is (NC)-alkyl-.

The terms “aminoalkyl”, “aminoalkenyl”, “aminoaliphatic”, and “aminoalkoxy” mean alkyl, alkenyl, aliphatic or alkoxy, as the case may be, substituted with one or more amino groups, wherein the amino group is as defined above. In some embodiments, the aminoaliphatic is a C1-C6 aliphatic group substituted with one or more —NH₂ groups. In some embodiments, the aminoalkyl refers to the structure (R^{X}R^{Y})N-alkyl-, wherein each of R^{X} and R^{Y} independently is as defined above. In some specific embodiments, the aminoalkyl is C1-C6 alkyl substituted with one or more —NH₂ groups. In some specific embodiments, the aminoalkenyl is C1-C6 alkenyl substituted with one or more —NH₂ groups. In some embodiments, the aminoalkoxy is —O(C1-C6 alkyl) wherein the alkyl group is substituted with one or more —NH₂ groups.

The terms “hydroxyalkyl”, “hydroxyaliphatic”, and “hydroxyalkoxy” mean alkyl, aliphatic or alkoxy, as the case may be, substituted with one or more —OH groups.

The terms “alkoxyalkyl”, “alkoxyaliphatic”, and “alkoxyalkoxy” mean alkyl, aliphatic or alkoxy, as the case may be, substituted with one or more alkoxy groups. For example, an “alkoxyalkyl” refers to an alkyl group such as (alkyl-O)-alkyl-, wherein alkyl is as defined above.

The term “carboxyalkyl” means alkyl substituted with one or more carboxy groups, wherein alkyl and carboxy are as defined above.

The term “protecting group” and “protective group” as used herein, are interchangeable and refer to an agent used to temporarily block one or more desired functional groups in a compound with multiple reactive sites. In certain embodiments, a protecting group has one or more, or specifically all, of the following characteristics: a) is added selectively to a functional group in good yield to give a protected substrate that is b) stable to reactions occurring at one or more of the other reactive sites; and c) is selectively removable in good yield by reagents that do not attack the regenerated, deprotected functional group. As would be understood by one skilled in the art, in some cases, the reagents do not attack other reactive groups in the compound. In other cases, the reagents may also react with other reactive groups in the compound. Examples of protecting groups are detailed in Greene, T. W., Wuts, P. G in “Protective Groups in Organic Synthesis”, Third Edition, John Wiley & Sons, New York: 1999 (and other editions of the book), the entire contents of which are hereby incorporated by reference. The term “nitrogen protecting group”, as used herein, refers to an agent used to temporarily block one or more desired nitrogen reactive sites in a multifunctional compound. Preferred nitrogen protecting groups also possess the characteristics exemplified for a protecting group above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

As used herein, the term “displaceable moiety” or “leaving group” refers to a group that is associated with an aliphatic or aromatic group as defined herein and is subject to being displaced by nucleophilic attack by a nucleophile.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this invention, unless only one of the isomers is drawn specifically. As would be understood to one skilled in the art, a substituent can freely rotate around any rotatable bonds. For example, a substituent drawn as

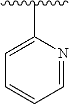 [SMILES: *c1ccccn1]

also represent

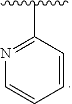 [SMILES: *c1ccccn1]

Therefore, single stereochemical isomers as well as enantiomeric, diastereomeric, cis/trans, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or 14C-enriched carbon are within the scope of this invention. Such compounds are useful, for example, as analytical tools or probes in biological assays. Such compounds, especially deuterium analogs, can also be therapeutically useful.

The terms “a bond” and “absent” are used interchangeably to indicate that a group is absent.

The compounds of the invention are defined herein by their chemical structures and/or chemical names. Where a compound is referred to by both a chemical structure and a chemical name, and the chemical structure and chemical name conflict, the chemical structure is determinative of the compound's identity.

### Pharmaceutically Acceptable Salts, Solvates, Chlatrates, Prodrugs and Other Derivatives

The compounds described herein can exist in free form, or, where appropriate, as salts. Those salts that are pharmaceutically acceptable are of particular interest since they are useful in administering the compounds described below for medical purposes. Salts that are not pharmaceutically acceptable are useful in manufacturing processes, for isolation and purification purposes, and in some instances, for use in separating stereoisomeric forms of the compounds of the invention or intermediates thereof.

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxy group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄alkyl)₄ salts. This invention also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminium. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminium hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, dietanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, while not in themselves pharmaceutically acceptable, may be employed in the preparation of salts useful as intermediates in obtaining the compounds described herein and their pharmaceutically acceptable acid or base addition salts.

It should be understood that this invention includes mixtures/combinations of different pharmaceutically acceptable salts and also mixtures/combinations of compounds in free form and pharmaceutically acceptable salts.

In addition to the compounds described herein, pharmaceutically acceptable solvates (e.g., hydrates) and clathrates of these compounds may also be employed in compositions to treat or prevent the herein identified disorders.

As used herein, the term “pharmaceutically acceptable solvate,” is a solvate formed from the association of one or more pharmaceutically acceptable solvent molecules to one of the compounds described herein. The term solvate includes hydrates (e.g., hemihydrate, monohydrate, dihydrate, trihydrate, tetrahydrate, and the like).

As used herein, the term “hydrate” means a compound described herein or a salt thereof that further includes a stoichiometric or non-stoichiometric amount of water bound by non-covalent intermolecular forces.

As used herein, he term “clathrate” means a compound described herein or a salt thereof in the form of a crystal lattice that contains spaces (e.g., channels) that have a guest molecule (e.g., a solvent or water) trapped within.

In addition to the compounds described herein, pharmaceutically acceptable derivatives or prodrugs of these compounds may also be employed in compositions to treat or prevent the herein identified disorders.

A “pharmaceutically acceptable derivative or prodrug” includes any pharmaceutically acceptable ester, salt of an ester or other derivative or salt thereof of a compound described herein which, upon administration to a recipient, is capable of providing, either directly or indirectly, a compound described herein or an inhibitorily active metabolite or residue thereof. Particularly favoured derivatives or prodrugs are those that increase the bioavailability of the compounds when such compounds are administered to a patient (e.g., by allowing an orally administered compound to be more readily absorbed into the blood) or which enhance delivery of the parent compound to a biological compartment (e.g., the brain or lymphatic system) relative to the parent species.

As used herein and unless otherwise indicated, the term “prodrug” means a derivative of a compound that can hydrolyze, oxidize, or otherwise react under biological conditions (in vitro or in vivo) to provide a compound described herein. Prodrugs may become active upon such reaction under biological conditions, or they may have activity in their unreacted forms. Examples of prodrugs contemplated in this invention include, but are not limited to, analogs or derivatives of compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds described herein that comprise —NO, —NO₂, —ONO, or —ONO₂ moieties. Prodrugs can typically be prepared using well-known methods, such as those described by BURGER'S MEDICINAL CHEMISTRY AND DRUG DISCOVERY (1995) 172-178, 949-982 (Manfred E. Wolff ed., 5th ed).

A “pharmaceutically acceptable derivative” is an adduct or derivative which, upon administration to a patient in need, is capable of providing, directly or indirectly, a compound as otherwise described herein, or a metabolite or residue thereof. Examples of pharmaceutically acceptable derivatives include, but are not limited to, esters and salts of such esters.

Pharmaceutically acceptable prodrugs of the compounds described herein include, without limitation, esters, amino acid esters, phosphate esters, metal salts and sulfonate esters.

### Uses of Disclosed Compounds

One aspect of the present invention is generally related to the use of the compounds described herein or pharmaceutically acceptable salts, or pharmaceutically acceptable compositions comprising such a compound or a pharmaceutically acceptable salt thereof, for inhibiting the replication of influenza viruses in a biological sample or in a patient, for reducing the amount of influenza viruses (reducing viral titer) in a biological sample or in a patient, and for treating influenza in a patient.

In one embodiment, the present invention is generally related to the use of compounds represented by Structural Formula I or pharmaceutically acceptable salts thereof for any of the uses specified above:

In yet another embodiment, the present invention is directed to the use of any compound selected from the compounds depicted in Tables 1 and 2, or a pharmaceutically acceptable salt thereof, for any of the uses described above.

In some embodiments, the compounds are represented by any one of Structural Formula I and the variables are each independently as depicted in the compounds of Tables 1 and 2.

In yet another embodiment, the compounds described herein or pharmaceutically acceptable salts thereof can be used to reduce viral titre in a biological sample (e.g. an infected cell culture) or in humans (e.g. lung viral titre in a patient).

The terms “influenza virus mediated condition”, “influenza infection”, or “Influenza”, as used herein, are used interchangeable to mean the disease caused by an infection with an influenza virus.

Influenza is an infectious disease that affects birds and mammals caused by influenza viruses. Influenza viruses are RNA viruses of the family Orthomyxoviridae, which comprises five genera: Influenzavirus A, Influenzavirus B, Influenzavirus C, Isavirus and Thogotovirus. Influenzavirus A genus has one species, influenza A virus which can be subdivided into different serotypes based on the antibody response to these viruses: H1N1, H2N2, H3N2, H5N1, H7N7, H1N2, H9N2, H7N2, H7N3 and H10N7. Influenzavirus B genus has one species, influenza B virus. Influenza B almost exclusively infects humans and is less common than influenza A. Influenzavirus C genus has one species, Influenzavirus C virus, which infects humans and pigs and can cause severe illness and local epidemics. However, Influenzavirus C is less common than the other types and usually seems to cause mild disease in children.

In some embodiments of the invention, influenza or influenza viruses are associated with Influenzavirus A or B. In some embodiments of the invention, influenza or influenza viruses are associated with Influenzavirus A. In some specific embodiments of the invention, Influenzavirus A is H1N1, H2N2, H3N2 or H5N1.

In humans, common symptoms of influenza are chills, fever, pharyngitis, muscle pains, severe headache, coughing, weakness, and general discomfort. In more serious cases, influenza causes pneumonia, which can be fatal, particularly in young children and the elderly. Although it is often confused with the common cold, influenza is a much more severe disease and is caused by a different type of virus. Influenza can produce nausea and vomiting, especially in children, but these symptoms are more characteristic of the unrelated gastroenteritis, which is sometimes called “stomach flu” or “24-hour flu”.

Symptoms of influenza can start quite suddenly one to two days after infection. Usually the first symptoms are chills or a chilly sensation, but fever is also common early in the infection, with body temperatures ranging from 38-39° C. (approximately 100-103° F.). Many people are so ill that they are confined to bed for several days, with aches and pains throughout their bodies, which are worse in their backs and legs. Symptoms of influenza may include: body aches, especially joints and throat, extreme coldness and fever, fatigue, Headache, irritated watering eyes, reddened eyes, skin (especially face), mouth, throat and nose, abdominal pain (in children with influenza B). Symptoms of influenza are non-specific, overlapping with many pathogens (“influenza-like illness). Usually, laboratory data is needed in order to confirm the diagnosis.

The terms, “disease”, “disorder”, and “condition” may be used interchangeably here to refer to an influenza virus mediated medical or pathological condition.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a “human”.

The term “biological sample”, as used herein, includes, without limitation, cell cultures or extracts thereof; biopsied material obtained from a mammal or extracts thereof; blood, saliva, urine, feces, semen, tears, or other body fluids or extracts thereof.

As used herein, “multiplicity of infection” or “MOI” is the ratio of infectious agents (e.g. phage or virus) to infection targets (e.g. cell). For example, when referring to a group of cells inoculated with infectious virus particles, the multiplicity of infection or MOI is the ratio defined by the number of infectious virus particles deposited in a well divided by the number of target cells present in that well.

As used herein the term “inhibition of the replication of influenza viruses” includes both the reduction in the amount of virus replication (e.g. the reduction by at least 10%) and the complete arrest of virus replication (i.e., 100% reduction in the amount of virus replication). In some embodiments, the replication of influenza viruses are inhibited by at least 50%, at least 65%, at least 75%, at least 85%, at least 90%, or at least 95%.

Influenza virus replication can be measured by any suitable method known in the art. For example, influenza viral titre in a biological sample (e.g. an infected cell culture) or in humans (e.g. lung viral titre in a patient) can be measured. More specifically, for cell based assays, in each case cells are cultured in vitro, virus is added to the culture in the presence or absence of a test agent, and after a suitable length of time a virus-dependent endpoint is evaluated. For typical assays, the Madin-Darby canine kidney cells (MDCK) and the standard tissue culture adapted influenza strain, A/Puerto Rico/8/34 can be used. A first type of cell assay that can be used in the invention depends on death of the infected target cells, a process called cytopathic effect (CPE), where virus infection causes exhaustion of the cell resources and eventual lysis of the cell. In the first type of cell assay, a low fraction of cells in the wells of a microtiter plate are infected (typically 1/10 to 1/1000), the virus is allowed to go through several rounds of replication over 48-72 hours, then the amount of cell death is measured using a decrease in cellular ATP content compared to uninfected controls. A second type of cell assay that can be employed in the invention depends on the multiplication of virus-specific RNA molecules in the infected cells, with RNA levels being directly measured using the branched-chain DNA hybridization method (bDNA). In the second type of cell assay, a low number of cells are initially infected in wells of a microtiter plate, the virus is allowed to replicate in the infected cells and spread to additional rounds of cells, then the cells are lysed and viral RNA content is measured. This assay is stopped early, usually after 18-36 hours, while all the target cells are still viable. Viral RNA is quantitated by hybridization to specific oligonucleotide probes fixed to wells of an assay plate, then amplification of the signal by hybridization with additional probes linked to a reporter enzyme.

As used herein a “viral titer (or titre)” is a measure of virus concentration. Titer testing can employ serial dilution to obtain approximate quantitative information from an analytical procedure that inherently only evaluates as positive or negative. The titer corresponds to the highest dilution factor that still yields a positive reading; for example, positive readings in the first 8 serial twofold dilutions translate into a titer of 1:256. A specific example is viral titer. To determine the titer, several dilutions will be prepared, such as 10⁻¹, 10⁻², 10⁻³, . . . , 10⁻⁸. The lowest concentration of virus that still infects cells is the viral titer.

As used herein, the terms “treat”, “treatment” and “treating” refer to both therapeutic and prophylactic treatments. For example, therapeutic treatments includes the reduction or amelioration of the progression, severity and/or duration of influenza viruses mediated conditions, or the amelioration of one or more symptoms (specifically, one or more discernible symptoms) of influenza viruses mediated conditions, resulting from the administration of one or more therapies (e.g., one or more therapeutic agents such as a compound or composition of the invention). In specific embodiments, the therapeutic treatment includes the amelioration of at least one measurable physical parameter of an influenza virus mediated condition. In other embodiments the therapeutic treatment includes the inhibition of the progression of an influenza virus mediated condition, either physically by, e.g., stabilization of a discernible symptom, physiologically by, e.g., stabilization of a physical parameter, or both. In other embodiments the therapeutic treatment includes the reduction or stabilization of influenza viruses mediated infections. Antiviral drugs can be used in the community setting to treat people who already have influenza to reduce the severity of symptoms and reduce the number of days that they are sick.

The term “chemotherapy” refers to the use of medications, e.g. small molecule drugs (rather than “vaccines”) for treating a disorder or disease.

The terms “prophylaxis” or “prophylactic use” and “prophylactic treatment” as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a person with the disease. The term “chemoprophylaxis” refers to the use of medications, e.g. small molecule drugs (rather than “vaccines”) for the prevention of a disorder or disease.

As used herein, prophylactic use includes the use in situations in which an outbreak has been detected, to prevent contagion or spread of the infection in places where a lot of people that are at high risk of serious influenza complications live in close contact with each other (e.g. in a hospital ward, daycare center, prison, nursing home, etc). It also includes the use among populations who require protection from the influenza but who either do not get protection after vaccination (e.g. due to weak immunse system), or when the vaccine is unavailable to them, or when they cannot get the vaccine because of side effects. It also includes use during the two weeks following vaccination, since during that time the vaccine is still ineffective. Prophylactic use may also include treating a person who is not ill with the influenza or not considered at high risk for complications, in order to reduce the chances of getting infected with the influenza and passing it on to a high-risk person in close contact with him (for instance, healthcare workers, nursing home workers, etc).

According to the US CDC, an influenza “outbreak” is defined as a sudden increase of acute febrile respiratory illness (AFRI) occurring within a 48 to 72 hour period, in a group of people who are in close proximity to each other (e.g. in the same area of an assisted living facility, in the same household, etc) over the normal background rate or when any subject in the population being analyzed tests positive for influenza. One case of confirmed influenza by any testing method is considered an outbreak.

A “cluster” is defined as a group of three or more cases of AFRI occurring within a 48 to 72 hour period, in a group of people who are in close proximity to each other (e.g. in the same area of an assisted living facility, in the same household, etc).

As used herein, the “index case”, “primary case” or “patient zero” is the initial patient in the population sample of an epidemiological investigation. When used in general to refer to such patients in epidemiological investigations, the term is not capitalized. When the term is used to refer to a specific person in place of that person's name within a report on a specific investigation, the term is capitalized as Patient Zero. Often scientists search for the index case to determine how the disease spread and what reservoir holds the disease in between outbreaks. Note that the index case is the first patient that indicates the existence of an outbreak. Earlier cases may be found and are labeled primary, secondary, tertiary, etc.

In one embodiment, the methods of the invention are a preventative or “pre-emptive” measure to a patient, specifically a human, having a predisposition to complications resulting from infection by an influenza virus. The term “pre-emptive” as used herein as for example in pre-emptive use, “pre-emptively”, etc, is the prophylactic use in situations in which an “index case” or an “outbreak” has been confirmed, in order to prevent the spread of infection in the rest of the community or population group.

In another embodiment, the methods of the invention are applied as a “pre-emptive” measure to members of a community or population group, specifically humans, in order to prevent the spread of infection.

As used herein, an “effective amount” refers to an amount sufficient to elicit the desired biological response. In the present invention the desired biological response is to inhibit the replication of influenza virus, to reduce the amount of influenza viruses or to reduce or ameliorate the severity, duration, progression, or onset of a influenza virus infection, prevent the advancement of an influenza viruses infection, prevent the recurrence, development, onset or progression of a symptom associated with an influenza virus infection, or enhance or improve the prophylactic or therapeutic effect(s) of another therapy used against influenza infections. The precise amount of compound administered to a subject will depend on the mode of administration, the type and severity of the infection and on the characteristics of the subject, such as general health, age, sex, body weight and tolerance to drugs. The skilled artisan will be able to determine appropriate dosages depending on these and other factors. When co-administered with other anti viral agents, e.g., when co-administered with an anti-influenza medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between approximately 0.01 to 100 mg/kg body weight/day for therapeutic or prophylactic treatment.

Generally, dosage regimens can be selected in accordance with a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the renal and hepatic function of the subject; and the particular compound or salt thereof employed, the duration of the treatment; drugs used in combination or coincidental with the specific compound employed, and like factors well known in the medical arts. The skilled artisan can readily determine and prescribe the effective amount of the compounds described herein required to treat, to prevent, inhibit (fully or partially) or arrest the progress of the disease.

Dosages of the compounds described herein can range from between about 0.01 to about 100 mg/kg body weight/day, about 0.01 to about 50 mg/kg body weight/day, about 0.1 to about 50 mg/kg body weight/day, or about 1 to about 25 mg/kg body weight/day. It is understood that the total amount per day can be administered in a single dose or can be administered in multiple dosing, such as twice a day (e.g., every 12 hours), tree times a day (e.g., every 8 hours), or four times a day (e.g., every 6 hours).

For therapeutic treatment, the compounds described herein can be administered to a patient within, for example, 48 hours (or within 40 hours, or less than 2 days, or less than 1.5 days, or within 24 hours) of onset of symptoms (e.g., nasal congestion, sore throat, cough, aches, fatigue, headaches, and chills/sweats). The therapeutic treatment can last for any suitable duration, for example, for 5 days, 7 days, 10 days, 14 days, etc. For prophylactic treatment during a community outbreak, the compounds described herein can be administered to a patient within, for example, 2 days of onset of symptoms in the index case, and can be continued for any suitable duration, for example, for 7 days, 10 days, 14 days, 20 days, 28 days, 35 days, 42 days, etc.

Various types of administration methods can be employed in the invention, and are described in detail below under the section entitled “Administration Methods.”

### Combination Therapy

An effective amount can be achieved in the method or pharmaceutical composition of the invention employing a compound of the invention (including a pharmaceutically acceptable salt or solvate (e.g., hydrate)) alone or in combination with an additional suitable therapeutic agent, for example, an antiviral agent or a vaccine. When “combination therapy” is employed, an effective amount can be achieved using a first amount of a compound of the invention and a second amount of an additional suitable therapeutic agent (e.g. an antiviral agent or vaccine).

In another embodiment of this invention, a compound of the invention and the additional therapeutic agent, are each administered in an effective amount (i.e., each in an amount which would be therapeutically effective if administered alone). In another embodiment, a compound of the invention and the additional therapeutic agent, are each administered in an amount which alone does not provide a therapeutic effect (a sub-therapeutic dose). In yet another embodiment, a compound of the invention can be administered in an effective amount, while the additional therapeutic agent is administered in a sub-therapeutic dose. In still another embodiment, a compound of the invention can be administered in a sub-therapeutic dose, while the additional therapeutic agent, for example, a suitable cancer-therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Coadministration encompasses administration of the first and second amounts of the compounds of the coadministration in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order.

In one embodiment, the present invention is directed to methods of combination therapy for inhibiting Flu viruses replication in biological samples or patients, or for treating or preventing Influenza virus infections in patients using the compounds or pharmaceutical compositions of the invention. Accordingly, pharmaceutical compositions of the invention also include those comprising an inhibitor of Flu virus replication of this invention in combination with an anti-viral compound exhibiting anti-Influenza virus activity.

Methods of use of the compounds and compositions of the invention also include combination of chemotherapy with a compound or composition of the invention, or with a combination of a compound or composition of this invention with another anti-viral agent and vaccination with a Flu vaccine.

When co-administration involves the separate administration of the first amount of a compound of the invention and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound of the invention and the second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a compound of the invention) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-cancer agent) to a subject.

It is understood that the method of co-administration of a first amount of a compound of the invention and a second amount of an additional therapeutic agent can result in an enhanced or synergistic therapeutic effect, wherein the combined effect is greater than the additive effect that would result from separate administration of the first amount of a compound of the invention and the second amount of an additional therapeutic agent.

As used herein, the term “synergistic” refers to a combination of a compound of the invention and another therapy (e.g., a prophylactic or therapeutic agent), which is more effective than the additive effects of the therapies. A synergistic effect of a combination of therapies (e.g., a combination of prophylactic or therapeutic agents) can permit the use of lower dosages of one or more of the therapies and/or less frequent administration of said therapies to a subject. The ability to utilize lower dosages of a therapy (e.g., a prophylactic or therapeutic agent) and/or to administer said therapy less frequently can reduce the toxicity associated with the administration of said therapy to a subject without reducing the efficacy of said therapy in the prevention, management or treatment of a disorder. In addition, a synergistic effect can result in improved efficacy of agents in the prevention, management or treatment of a disorder. Finally, a synergistic effect of a combination of therapies (e.g., a combination of prophylactic or therapeutic agents) may avoid or reduce adverse or unwanted side effects associated with the use of either therapy alone.

When the combination therapy using the compounds of the present invention is in combination with a Flu vaccine, both therapeutic agents can be administered so that the period of time between each administration can be longer (e.g. days, weeks or months).

The presence of a synergistic effect can be determined using suitable methods for assessing drug interaction. Suitable methods include, for example, the Sigmoid-Emax equation (Holford, N. H. G. and Scheiner, L. B., Clin. Pharmacokinet. 6: 429-453 (1981)), the equation of Loewe additivity (Loewe, S, and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied with experimental data to generate a corresponding graph to aid in assessing the effects of the drug combination. The corresponding graphs associated with the equations referred to above are the concentration-effect curve, isobologram curve and combination index curve, respectively.

Specific examples that can be co-administered with a compound described herein include neuraminidase inhibitors, such as oseltamivir (Tamiflu®) and Zanamivir (Rlenza®), viral ion channel (M2 protein) blockers, such as amantadine (Symmetrel®) and rimantadine (Flumadine®), and antiviral drugs described in WO 2003/015798, including T-705 under development by Toyama Chemical of Japan. (See alsoRuruta et al., Antiviral Reasearch, 82: 95-102 (2009), “T-705 (flavipiravir) and related compounds: Novel broad-spectrum inhibitors of RNA viral infections.”) In some embodiments, the compounds described herein can be co-administered with a traditional influenza vaccine.

### Pharmaceutical Compositions

The compounds described herein can be formulated into pharmaceutical compositions that further comprise a pharmaceutically acceptable carrier, diluent, adjuvant or vehicle. In one embodiment, the present invention relates to a pharmaceutical composition comprising a compound of the invention described above, and a pharmaceutically acceptable carrier, diluent, adjuvant or vehicle. In one embodiment, the present invention is a pharmaceutical composition comprising an effective amount of a compound of the present invention or a pharmaceutically acceptable salt thereof and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

An “effective amount” includes a “therapeutically effective amount” and a “prophylactically effective amount”. The term “therapeutically effective amount” refers to an amount effective in treating and/or ameliorating an influenza virus infection in a patient infected with influenza. The term “prophylactically effective amount” refers to an amount effective in preventing and/or substantially lessening the chances or the size of influenza virus infection outbreak. Specific examples of effective amounts are described above in the section entitled Uses of Disclosed Compounds.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, its use is contemplated to be within the scope of this invention. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Administration Methods

The compounds and pharmaceutically acceptable compositions described above can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compositions described herein may be administered orally, parenterally, by inhalation spray, topically, rectally, nasally, buccally, vaginally or via an implanted reservoir. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Specifically, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions described herein may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions described herein may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.

The pharmaceutical compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, specifically, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds for use in the methods of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

### EXEMPLIFICATION

### Preparation of Compounds

The compounds disclosed herein can be prepared by any suitable method known in the art, for example, WO 2005/095400, WO 2007/084557, WO 2010/011768, WO 2010/011756, WP 2010/011772, WO 2009/073300, and PCT/US2010/038988 filed on Jun. 17, 2010. For example, the compounds shown in Tables 1 and 2 can be prepared by any suitable method known in the art, for example, WO 2005/095400, WO 2007/084557, WO 2010/011768, WO 2010/011756, WP 2010/011772, WO 2009/073300, and PCT/US2010/038988, and by the exemplary syntheses described below. Generally, the compounds of the invention can be prepared as shown in those syntheses optionally with any desired appropriate modification.

### Methodology for Synthesis and Characterization of Compounds

Syntheses of certain exemplary compounds of the invention are described below. NMR and Mass Spectroscopy data of certain specific compounds are summarized in Tables 1 and 2. As used herein the term RT (min) refers to the LCMS retention time, in minutes, associated with the compound.

### Preparation Intermediate Compound 4

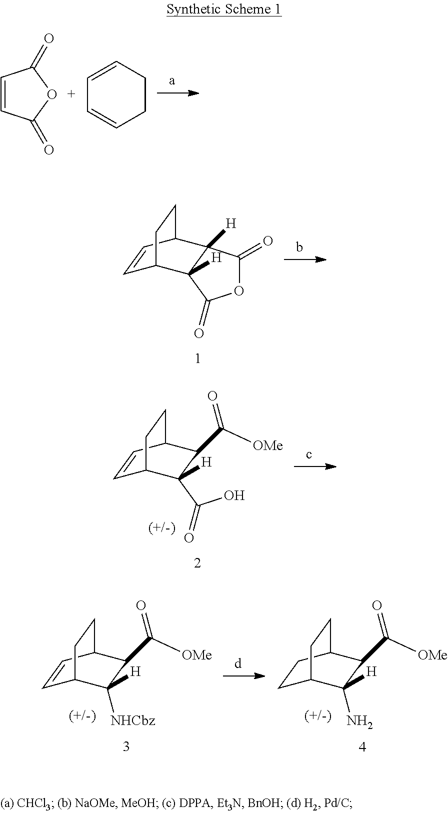

### Formation of endo-tetrahydro-4,7-ethanoisobenzofuran-1,3-dione (1)

To a cold (0° C.) solution of maleic anhydride (210.0 g, 2142.0 mmol) in CHCl₃ (2.3 L) was added cyclohexa-1,3-diene (224.5 mL, 2356.0 mmol) slowly over 50 minutes. The reaction was warmed to room temperature and stirred overnight in the dark. After removing the solvent under reduced pressure, 2.1 L of MeOH was added to the mixture and the mixture was heated to 50° C. for 10 min and then cooled down to 0° C. The resulting precipitate was filtered and dried in an oven at 45° C. overnight to afford 283 g of a white solid. The resulting endo (meso) Diels-Alder cycloaddition product was used without further purification.

### Formation of (+/−)-trans-3-(methoxycarbonyl)bicyclo[2.2.2]oct-5-ene-2-carboxylic acid (2)

A solution of endo-(+/−)-tetrahydro-4,7-ethanoisobenzofuran-1,3-dione, 1, (74.5 g, 418.1 mmol) was stirred in NaOMe (764.9 mL of 25% w/w solution in MeOH, 3345.0 mmol). The reaction mixture was stirred at room temperature for 4 days yielding a white suspension. The reaction mixture was concentrated in vacuo to remove approximately 300 mL of MeOH. In another flask, HCl (315.9 mL of 36.5% w/w, 3763.0 mmol) in 300 mL of water was cooled to 0° C. Added reaction mixture into this HCl solution slowly, white solid precipitated. The remaining methanol was removed under reduced pressure. The mixture was cooled to 0° C. and stirred for 30 minutes. The precipitate was filtered, washed with water 3 times, giving an off-white solid. The remaining water was removed under reduced pressure to afford 82 g of a white solid.

### Formation of (+/−)-trans-methyl 3-(((benzyloxy)carbonyl)amino)bicyclo[2.2.2]oct-5-ene-2-carboxylate (3)

Dissolved (+/−)-trans-3-methoxycarbonylbicyclo[2.2.2]oct-5-ene-2-carboxylic acid, 2, (100.0 g, 475.7 mmol) in toluene (1.0 L). Added diphenylphosphoryl azide (112.8 mL, 523.3 mmol) and triethylamine (72.9 mL, 523.3 mmol). Heated reaction mixture to 90° C. for 2 hours. Added benzyl alcohol (49.2 mL, 475.7 mmol) and heated to 90° C. over 3 days. The mixture was cooled to room temperature and diluted with EtOAc (500 mL) and aqueous sat. NaHCO₃ solution. The organic phase was washed with brine, dried (MgSO₄), filtered and concentrated in vacuo. The resulting crude material was purified by silica gel chromatography (1500 g silica ISCO column) with dichloromethane to afford 115 g oil. ¹H NMR show it contains BnOH (about 0.05 equiv). Product was used without further purification: ¹H NMR (300 MHz, CDCl₃) δ 7.40-7.24 (m, 5H), 6.41 (t, J^{=7.4} Hz, 1H), 6.21-6.04 (m, 1H), 5.15-4.94 (m, 2H), 4.63-4.45 (m, 1H), 4.30-4.18 (m, 1H), 3.70 (s, 2H), 3.49 (s, 1H), 2.81 (br s, 1H), 2.68 (br s, 1H), 2.08 (s, 1H), 1.76-1.56 (m, 1H), 1.52-1.35 (m, 1H), 1.33-1.14 (m, 1H), 1.12-0.87 (m, 1H).

### Formation of (+/−)-trans-methyl 3-aminobicyclo[2.2.2]octane-2-carboxylate (4)

A solution of racemic trans-methyl 3-(((benzyloxy)carbonyl)amino)-bicyclo[2.2.2]oct-5-ene-2-carboxylate (115.0 g, 364.7 mmol) in THF (253 mL) and MeOH (253 mL) was placed under 40 psi of hydrogen overnight. Some exotherm was observed. ¹H NMR shows the reaction is complete and there is BnOH present. Filtered reaction mixture through celite, and washed with MeOH. Concentrated filtrate in vacuo to afford 69 g oil: ¹H NMR (400 MHz, CDCl₃) δ 3.63 (d, J^{=5.6} Hz, 3H), 3.30 (d, J^{=6.7} Hz, 1H), 2.11 (d, J^{=6.6} Hz, 1H), 1.91 (t, J=7.3 Hz, 1H), 1.80-1.64 (m, 1H), 1.63-1.38 (m, 6H), 1.36-1.23 (m, 2H).

### Preparation of Compound I-35

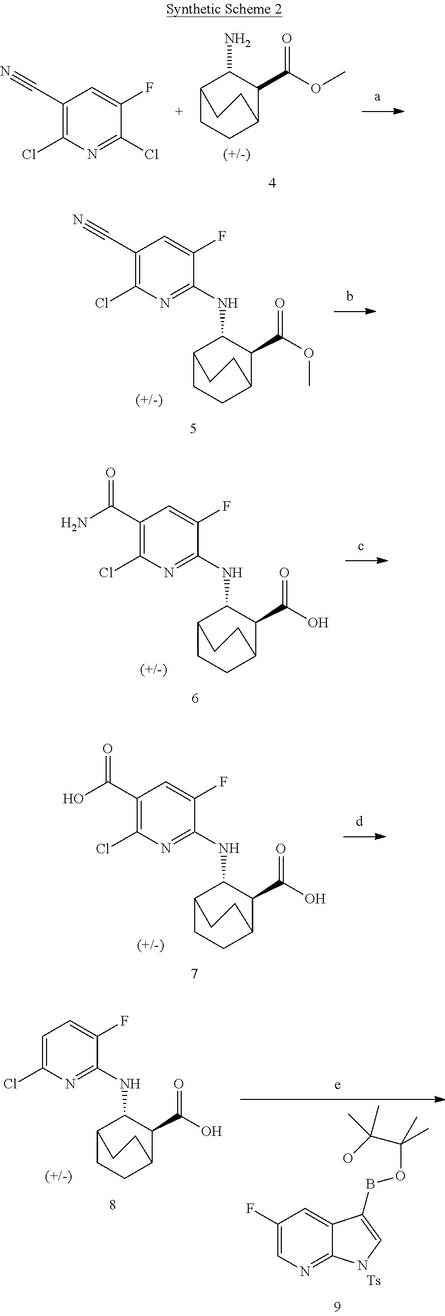

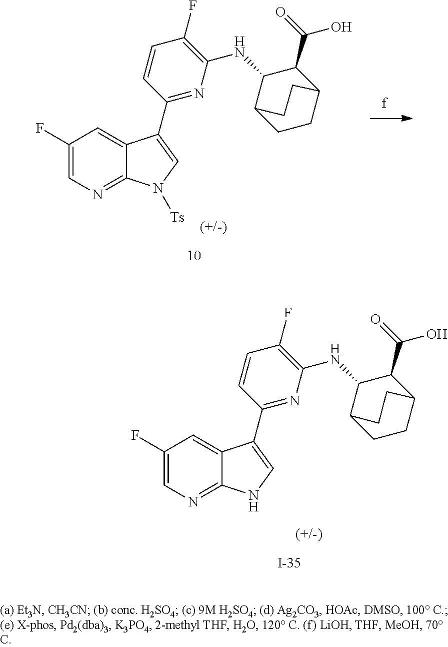

### Formation of (+/−) methyl 3-(6-chloro-5-cyano-3-fluoropyridin-2-ylamino)-bicyclo[2.2.2]octane-2-carboxylate (5)

A solution of (+/−)-methyl-3-aminobicyclo[2.2.2]octane-2-carboxylate, 4, (2.00 g, 10.91 mmol), 2,6-dichloro-5 fluoro-pyridine-3-carbonitrile (2.29 g, 12.00 mmol) and Et₃N (3.35 mL, 24.00 mmol) in acetonitrile (25 mL) was refluxed for 4 h. LC/MS indicated the desired product only. The reaction mixture was diluted into EtOAc and brine. The organic phase was dried over MgSO₄, filtered and the solvent was removed under reduced pressure. The resulting crude residue was purified by silica gel chromatography (20% EtOAc/hexanes) to afford 3.15 g of desired product: ¹H NMR (400 MHz, CDCl₃) δ 7.32-7.28 (m, 1H), 5.32 (s, 1H), 4.48 (s, 1H), 3.77 (s, 3H), 2.39 (d, J=5.6 Hz, 1H), 2.03-1.97 (m, 1H), 1.88 (d, J=2.2 Hz, 1H), 1.81 (d, J=13.5 Hz, 1H), 1.74-1.62 (m, 5H), 1.47 (d, J=13.2 Hz, 1H); LCMS Gradient 10-90%, 0.1% formic acid, 5 min, C18/ACN, Retention Time=3.60 minutes, (M+H) 338.35.

### Formation of (+/−) 3-(5-carbamoyl-6-chloro-3-fluoropyridin-2-ylamino)-bicyclo[2.2.2]octane-2-carboxylic acid (6):

To H₂SO₄ (35 mL of 18 M solution, 630.0 mmol) was added methyl 3-[(6-chloro-5-cyano-3-fluoro-2-pyridyl)amino]bicyclo[2.2.2]octane-2-carboxylate, 5, (3.15 g, 9.33 mmol). The reaction mixture was heated at 80° C. for 1 h. LC/MS indicated starting ester is consumed. LCMS Gradient 10-90%, 0.1% formic acid, 5 min, C18/ACN, Retention Time=2.39 minutes (M+H) 342.28. The crude product was used without further purification.

### Formation of (+/−) 6-(3-carboxybicyclo[2.2.2]octan-2-ylamino)-2-chloro-5-fluoropyridine-3-carboxylic acid (7)

A solution of (+/−)-3-(5-carbamoyl-6-chloro-3-fluoropyridin-2-ylamino)bicycle[2.2.2]octane-2-carboxylic acid, 6, in concentrated H₂SO₄ (35 mL of 18 M solution) was cooled to room temperature and was transferred to a flask with 35 mL H₂O slowly. The reaction mixture was then heated and stirred at 100° C. for 5 hours. The reaction mixture was cooled to room temperature and to it was added ice to total 250 mL volume. The resulting precipitate was filtered. The filtration cake was dissolved in CH₂Cl₂ and purified by silica gel chromatography (40% EtOAc/hexanes) to afford 2.0 g product. Overall yield for two steps 62%: ¹H NMR (400 MHz, DMSO-d6) δ 7.76 (d, J=11.2 Hz, 1H), 7.69 (d, J=6.9 Hz, 1H), 4.42 (t, J=6.8 Hz, 1H), 2.78 (d, J=6.8 Hz, 1H), 1.95 (s, 1H), 1.74 (s, 1H), 1.69 (d, J=8.5 Hz, 2H), 1.62-1.36 (m, 5H), 1.32 (t, J=10.4 Hz, 1H); LCMS Gradient 10-90%, 0.1% formic acid, 5 min, C18/ACN, Retention Time=2.84 minutes (M+H) 343.07.

### Formation of (+/−)-3-(6-chloro-3-fluoropyridin-2-ylamino)bicyclo[2.2.2]octane-2-carboxylic acid (8)

A solution of 6-[(2-carboxy-3-bicyclo[2.2.2]octanyl)amino]-2-chloro-5-fluoro-pyridine-3-carboxylic acid, 7, (2.00 g, 5.84 mmol), Ag₂CO₃ (0.16 g, 0.58 mmol) and acetic acid (0.02 mL, 0.29 mmol) in DMSO (20 mL) was heated and stirred at 120° C. for 5 h. The reaction mixture was diluted with EtOAc and aqueous saturated NH₄Cl solution. The organic phase was dried (MgSO₄), filtered and the solvent was removed under reduced pressure. The product was purified by silica gel chromatography (20% EtOAc/hexanes) to afford 1.34 g of desired product: ¹H NMR (400 MHz, CDCl₃) δ 7.19 (dd, J=10.0, 8.2 Hz, 1H), 6.59 (dd, J=8.1, 2.9 Hz, 1H), 5.22 (s, 1H), 4.03 (d, J=4.3 Hz, 1H), 2.50 (s, 1H), 2.17 (s, 1H), 2.04 (dd, J=17.6, 7.1 Hz, 1H), 1.87 (s, 1H), 1.82-1.64 (m, 4H), 1.63-1.50 (m, 6H), 1.44 (dd, J=19.8, 11.4 Hz, 1H); LCMS Gradient 10-90%, 0.1% formic acid, 5 min, C18/ACN, Retention Time=3.22 minutes (M+H) 299.07.

### Formation of (+/−)-3-(3-fluoro-6-(5-fluoro-1-tosyl-1H-pyrrolo[2,3-b]pyridin-3-yl)pyridin-2-ylamino)bicyclo[2.2.2]octane-2-carboxylic acid (10)

Degassed with nitrogen a solution of 5-fluoro-1-(p-tolylsulfonyl)-3-(4,4,5,5-tetramethyl-1,3,2-dioxaborolan-2-yl)pyrrolo[2,3-b]pyridine, 9, (0.42 g, 1.00 mmol), 3-[(6-chloro-3-fluoro-2-pyridyl)amino]bicyclo[2.2.2]octane-2-carboxylic acid, 8, (0.25 g, 0.84 mmol) and K₃PO₄ (0.53 g, 2.51 mmol) in 2-methyl THF and H₂O for 40 min. To this solution was added X-Phos (0.05 g, 0.100 mmol) and 1,5-diphenylpenta-1,4-dien-3-one; palladium (0.02 g, 0.02 mmol). The reaction mixture was heated and stirred at 120° C. in a pressure tube for 1 h. The aqueous phase was removed and the organic phase was filtered through a pad of celite and solvent was removed under reduced pressure. The product was purified by silica gel chromatography (40% EtOAc/hexanes) to afford 300 mg of the desired product: ¹H NMR (400 MHz, CDCl₃) δ 8.32 (s, 1H), 8.24 (s, 1H), 8.18 (d, J=8.0 Hz, 2H), 7.35 (d, J=8.1 Hz, 2H), 7.29 (d, J=3.4 Hz, 1H), 6.86 (dd, J=7.8, 2.8 Hz, 1H), 5.23 (s, 1H), 4.34 (s, 1H), 2.50 (s, 1H), 2.38 (s, 3H), 2.19 (s, 1H), 1.91 (d, J=53.8 Hz, 4H), 1.75 (s, 2H), 1.69-1.45 (m, 4H); LCMS Gradient 10-90%, 0.1% formic acid, 5 min, C18/ACN, Retention Time=3.92 minutes (M+H) 553.26.

### Formation of (+/−) 3-(3-fluoro-6-(5-fluoro-1H-pyrrolo[2,3-b]pyridin-3-yl)pyridin-2-ylamino)bicyclo[2.2.2]octane-2-carboxylic acid (I-35)

A solution of 3-[[3-fluoro-6-[5-fluoro-1-(p-tolylsulfonyl)pyrrolo[2,3-b]pyridin-3-yl]-2-pyridyl]amino]bicyclo[2.2.2]octane-2-carboxylic acid, 10, (0.30 g, 0.54 mmol) and lithium hydroxide hydrate (0.09 g, 2.17 mmol) in THF (20 mL) and H₂O (5 mL) was stirred at 70° C. for 1.5 h until LC/MS indicated the reaction was complete. To the reaction mixture was added HCl (0.27 mL of 6 M solution, 1.63 mmol) and aqueous saturated NH₄Cl solution. The product was extracted with EtOAc and the organic phase was dried over MgSO₄, filtered and the solvent was removed under reduced pressure. The crude residue was purified by silica gel chromatography (4% MeOH/CH₂Cl₂): ¹H NMR (400 MHz, CDCl₃) δ 10.75 (s, 1H), 8.12 (dd, J=9.2, 2.4 Hz, 1H), 7.76 (d, J=11.5 Hz, 2H), 7.21 (dd, J=10.7, 8.1 Hz, 1H), 6.74 (dd, J=8.0, 2.8 Hz, 1H), 5.06 (d, J=6.8 Hz, 1H), 4.70 (s, 1H), 2.44 (d, J=4.2 Hz, 1H), 2.02 (d, J=17.8 Hz, 2H), 1.96-1.82 (m, 3H), 1.80-1.61 (m, 4H), 1.48 (t, J=11.4 Hz, 1H); LCMS Gradient 10-90%, 0.1% formic acid, 5 min, C18/ACN, Retention Time=3.14 minutes (M+H) 399.18.

### Preparation of Compound I-28

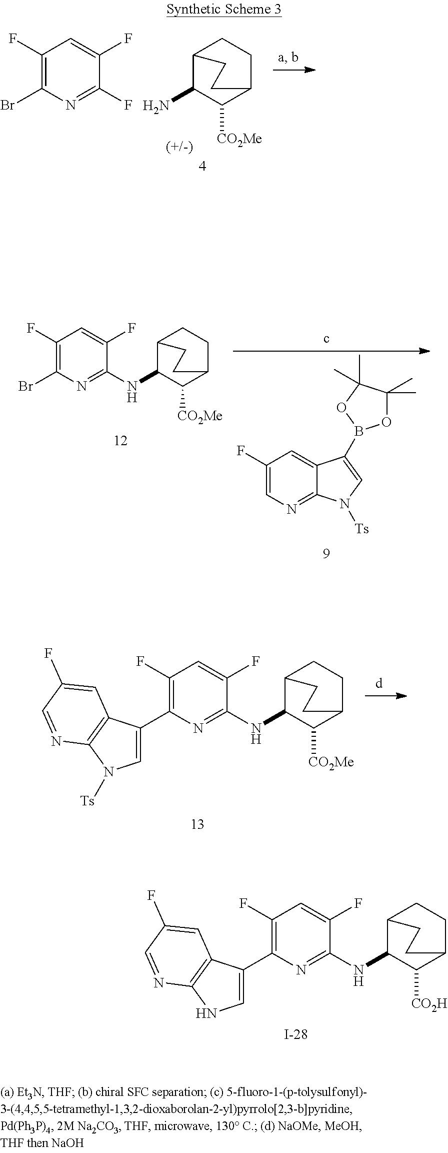

### Formation of (2S,3S)-methyl 3-((6-bromo-3,5-difluoropyridin-2-yl)amino)-bicyclo[2.2.2]octane-2-carboxylate (12)

To a solution of (+/−)-trans-methyl-3-aminobicyclo[2.2.2]octane-2-carboxylate, 4, (12.50 g, 68.21 mmol) and 2-bromo-3,5,6-trifluoro-pyridine (11.70 g, 55.20 mmol) in THF (78 mL) was added triethylamine (18.47 mL, 132.5 mmol). The reaction mixture was heated at 100° C. for 24 h followed by heating at 85° C. for 2 additional days. A white precipitate was observed. The solvent was evaporated and the crude product was purified by silica gel chromatography (0% to 33% EtOAc/Hexanes gradient) to afford 13.7 g of the desired product as a racemic mixture. The racemic mixture underwent SFC separation to provide 5.38 G of desired (2S,3S) enantiomer: LCMS Gradient 10-90%, 0.1% formic acid, 5 min, C18/ACN, RT=3.87 minutes (M+H) 375.38.

### Formation of (2S,3S)-methyl 3-((3,5-difluoro-6-(5-fluoro-1-tosyl-1H-pyrrolo[2,3-b]pyridin-3-yl)pyridin-2-yl)amino)bicyclo[2.2.2]octane-2-carboxylate (13)

To a microwave vial was added methyl (2S,3S)-3-[(6-bromo-3,5-difluoro-2-pyridyl)amino]bicyclo[2.2.2]octane-2-carboxylate, 12, (1.55 mg, 4.13 mmol) and 5-fluoro-1-(p-tolylsulfonyl)-3-(4,4,5,5-tetramethyl-1,3,2-dioxaborolan-2-yl)pyrrolo[2,3-b]pyridine, 9, (1.89 g, 4.54 mmol), THF (43.06 mL) and Na₂CO₃ (6.19 mL of 2 M, 12.39 mmol). The solution was degassed with N₂ for 15 minutes. Catalytic Pd(PPh₃)₄ was added to the reaction mixture which was then heated in a microwave for 30 minutes at 130° C. The reaction was split into two layers, and the upper layer was separated, shaken and a white precipitate was formed quickly. The mixture was diluted with ACN/H₂O and stirred for 30 mins, filtered and the cake was washed again with acetonitrile to provide the desired product as a white solid: LCMS Gradient 10-90%, 0.1% formic acid, 5 min, C18/ACN, RT=3.91 minutes (M+H) 447.56.

### Formation of (2S,3S)-3-((3,5-difluoro-6-(5-fluoro-1H-pyrrolo[2,3-b]pyridin-3-yl)pyridin-2-yl)amino)bicyclo[2.2.2]octane-2-carboxylic acid (I-28)

A solution of methyl (2S,3S)-3-[[3,5-difluoro-6-[5-fluoro-1-(p-tolylsulfonyl)pyrrolo-[2,3-b]pyridin-3-yl]-2-pyridyl]amino]bicyclo[2.2.2]octane-2-carboxylate, 13, (2.30 g, 3.93 mmol) in THF (20 mL) treated with NaOMe (5.16 mL of 4 M, 20.66 mmol) in MeOH (10 mL) at room temperature for 1 hour. NaOH (10.33 mL of 2 M, 20.66 mmol) was then added and the resulting mixture was stirred overnight at room temperature. The crude product solution was evaporated and a yellow precipitate was observed during the evaporation. The mixture was then filtered and the yellow solid was dried and then was washed with Et₂O to afford the desired product: ¹H NMR (300 MHz, MeOD-d4) δ 8.74 (dd, J=9.7, 2.6 Hz, 1H), 8.12 (s, 1H), 7.91 (d, J=2.2 Hz, 1H), 7.19 (dd, J=19.2, 8.8 Hz, 1H), 4.80 (d, J=6.8 Hz, 1H), 3.50-3.19 (m, 2H), 2.69 (d, J=6.8 Hz, 1H), 2.01 (t, J=22.7 Hz, 3H), 1.88-1.19 (m, 7H); LCMS Gradient 10-90%, 0.1% formic acid, 5 min, C18/ACN, RT=3.46 minutes (M+H) 433.54.

### Preparation of Compounds I-33 and I-34

The following compounds were prepared in a similar fashion as described above:

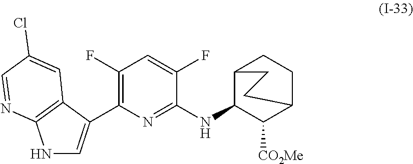 [SMILES: COC(=O)[C@H]1C2CCC(CC2)[C@@H]1Nc1nc(-c2c[nH]c3ncc(Cl)cc23)c(F)cc1F]

### (2S,3S)-methyl 3-((6-(5-chloro-1H-pyrrolo[2,3-b]pyridin-3-yl)-3,5-difluoropyridin-2-yl)amino)bicyclo[2.2.2]octane-2-carboxylate

LCMS RT=3.91 minutes (M+H) 447.56.

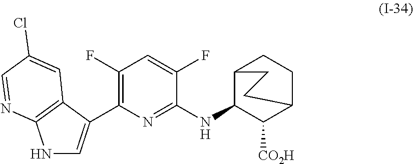 [SMILES: O=C(O)[C@H]1C2CCC(CC2)[C@@H]1Nc1nc(-c2c[nH]c3ncc(Cl)cc23)c(F)cc1F]

### (2S,3S)-3-((6-(5-chloro-1H-pyrrolo[2,3-b]pyridin-3-yl)-3,5-difluoropyridin-2-yl)amino)-bicyclo[2.2.2]octane-2-carboxylic acid

LCMS RT=3.46 minutes (M+H) 433.54.

### Preparation of Compound I-37

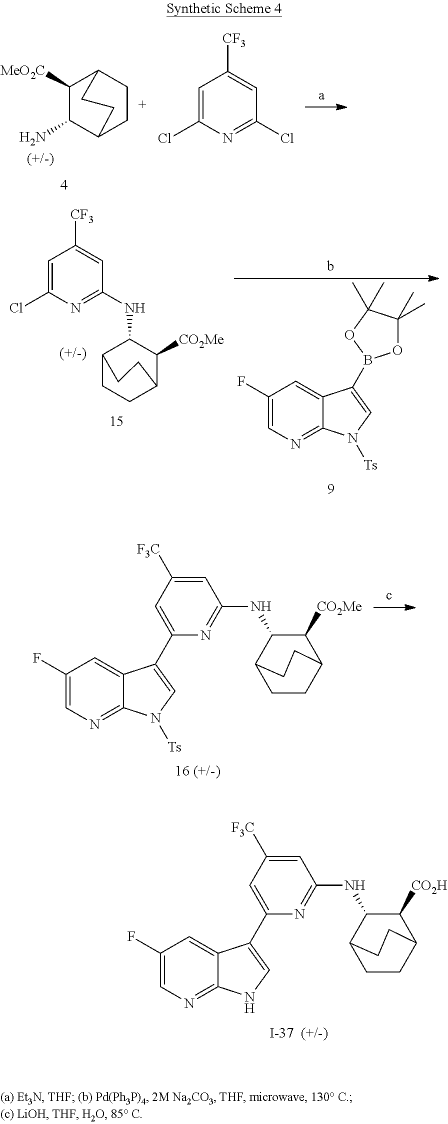

### Formation of (+/−)-trans-methyl 3-((6-chloro-4-(trifluoromethyl)pyridin-2-yl)amino) bicyclo[2.2.2]octane-2-carboxylate (15)

A solution of (+/−)-trans-methyl-3-aminobicyclo[2.2.2]octane-2-carboxylate, 4, (0.25 g, 1.36 mmol), 2,6-dichloro-4-(trifluoromethyl)pyridine (0.29 g, 1.36 mmol), triethylamine (0.45 mL, 3.27 mmol) in THF (1 mL) was heated at 85° C. for 3 days. The solvent was evaporated and the crude product was purified by silica gel chromatography to provide 210 mg of the desired product: LCMS Gradient 10-90%, 0.1% formic acid, 5 min, C18/ACN, RT=3.95 minutes (M+H) 362.42.

### Formation of (+/−)-trans-methyl 3-((6-(5-fluoro-1-tosyl-1H-pyrrolo[2,3-b]pyridin-3-yl)-4-(trifluoromethyl)pyridin-2-yl)amino)bicyclo[2.2.2]octane-2-carboxylate (16)

To a solution of (+/−)-trans-methyl 3-((6-chloro-4-(trifluoromethyl)pyridin-2-yl)amino) bicyclo[2.2.2]octane-2-carboxylate, 15, (0.21 g, 0.58 mmol) and 5-fluoro-1-(p-tolylsulfonyl)-3-(4,4,5,5-tetramethyl-1,3,2-dioxaborolan-2-yl)pyrrolo[2,3-b]pyridine, 9, (0.28 g, 0.68 mmol) in THF (5 mL) was added Na₂CO₃ (1.02 mL of 2 M solution, 2.05 mmol). The reaction was degassed with N₂ for 15 mins. Pd(PPh₃)₄ (0.16 g, 0.14 mmol) was added and the reaction was heated at 130° C. in microwave for 30 mins. The THF layer was separated and the aqueous layer was extracted with EtOAc and the combined organic phases were evaporated to give the crude product, which was purified by silica gel chromatography to provide 320 mg of the desired product: ¹H NMR (300 MHz, CDCl₃) δ 8.36 (dd, J=8.9, 2.8 Hz, 1H), 8.21 (dd, J=7.2, 5.4 Hz, 2H), 8.00 (d, J=8.4 Hz, 2H), 7.19 (d, J=8.1 Hz, 2H), 7.00 (s, 1H), 6.46 (s, 1H), 5.07 (d, J^{=7.6} Hz, 1H), 4.40 (t, J=6.6 Hz, 1H), 3.54 (s, 3H), 2.30 (s, 1H), 2.27 (s, 3H), 1.90-1.22 (m, 10H).

### Formation of (+/−)-trans-3-((6-(5-fluoro-1H-pyrrolo[2,3-b]pyridin-3-yl)-4-(trifluoro-methyl)pyridin-2-yl)amino)bicyclo[2.2.2]octane-2-carboxylic acid (I-37)

A solution of (+/−)-trans-methyl 3-((6-(5-fluoro-1-tosyl-1H-pyrrolo[2,3-b]pyridin-3-yl)-4-(trifluoromethyl)pyridin-2-yl)amino)bicyclo[2.2.2]octane-2-carboxylate, 16, (0.032 g, 0.052 mmol) and LiOH (0.5 mL of 2 M solution, 1.000 mmol) in THF (3 mL) was heated at 85° C. overnight. The reaction was acidified with TFA, extracted with EtOAc and purified by silica gel chromatography (0-10% MeOH/CH₂Cl₂ gradient) to provide 20 mg of the desired product: ¹H NMR (300 MHz, MeOD-d4) δ 8.55 (dd, J=9.5, 2.7 Hz, 1H), 8.15 (s, 2H), 7.70-7.41 (m, 1H), 7.08 (s, 1H), 6.65 (s, 1H), 4.61 (d, J=5.9 Hz, 1H), 2.53 (d, J=6.3 Hz, 1H), 2.23-1.05 (m, 10 H); LCMS Gradient 10-90%, 0.1% formic acid, 5 min, C18/ACN, RT=3.41 minutes (M+H) 449.5.

SFC chiral separation of the racemic mixture, I-37, provided the single enantiomers, I-40 (S,S) enantiomer, and I-41 (R, R) enantiomer.

### Preparation of Compound I-29

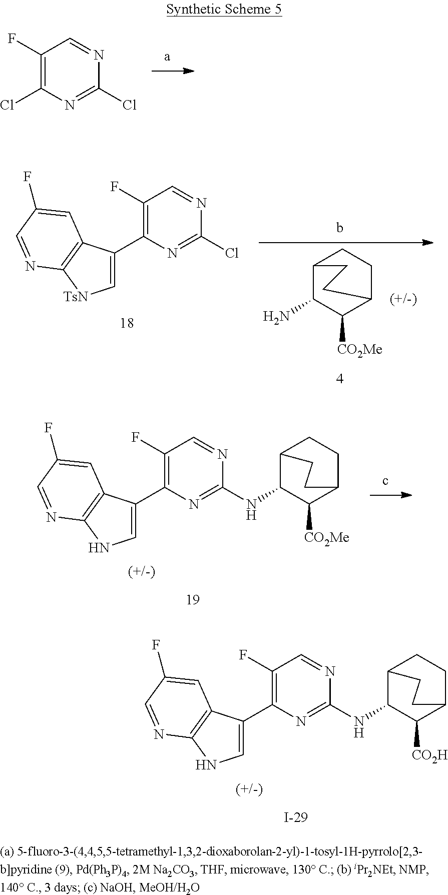

### Formation of 3-(2-chloro-5-fluoropyrimidin-4-yl)-5-fluoro-1-tosyl-1H-pyrrolo[2,3-b]pyridine (18)

To a solution of 2,4-dichloro-5-fluoro-pyrimidine (1.0 g, 6.0 mmol) and 5-fluoro-1-(p-tolylsulfonyl)-3-(4,4,5,5-tetramethyl-1,3,2-dioxaborolan-2-yl)pyrrolo[2,3-b]pyridine, 9, (2.2 g, 5.4 mmol) in THF (47.62 mL) was added Na₂CO₃ (6.0 mL of 2 M solution, 12.0 mmol). The mixture was degassed with N₂. To the mixture was added Pd(PPh₃)₄ (1.4 g, 1.2 mmol) and the mixture was heated to 90° C. for 16 hours. The reaction was cooled to room temperature. A mixture of acetonitrile/water (5/1) was added to the reaction mixture and let it stir for 30 mins. The resulting precipitate was filtered and washed with acetonitrile to generate a white solid: ¹H NMR (300 MHz, DMSO-d₆) δ 9.01 (d, J=3.0 Hz, 1H), 8.67 (d, J=1.9 Hz, 1H), 8.58 (dd, J=2.7, 1.2 Hz, 1H), 8.50 (dd, J=9.0, 2.8 Hz, 1H), 8.13 (d, J=8.4 Hz, 2H), 7.48 (d, J=8.1 Hz, 2H), 2.37 (s, 3H).

### Formation of (+/−) trans-methyl 3-((5-fluoro-4-(5-fluoro-1H-pyrrolo[2,3-b]pyridin-3-yl)pyrimidin-2-yl)amino)bicyclo[2.2.2]octane-2-carboxylate (19)

Charged racemic trans-methyl-3-aminobicyclo[2.2.2]octane-2-carboxylate, 4, (0.44 g, 2.37 mmol), 3-(2-chloro-5-fluoro-pyrimidin-4-yl)-5-fluoro-1-(p-tolylsulfonyl)-pyrrolo[2,3-b]pyridine, 18, (0.50 g, 1.19 mmol) and N,N-diisopropylethylamine (0.62 mL, 3.56 mmol) in NMP (2 mL). The reaction mixture was heated to 140° C. for 3 days. The solvent was evaporated and the crude product was used without further purification.

### Formation of (+/−) trans-3-((5-fluoro-4-(5-fluoro-1H-pyrrolo[2,3-b]pyridin-3-yl)pyrimidin-2-yl)amino)bicyclo[2.2.2]octane-2-carboxylic acid (I-29)

The crude racemic trans-3-[[5-fluoro-4-(5-fluoro-1H-pyrrolo[2,3-b]pyridin-3-yl)pyrimidin-2-yl]amino]bicyclo[2.2.2]octane-2-carboxylate NMP containing residue was dissolved in NaOH (5.94 mL of 2 M solution, 11.88 mmol) and stirred at room temperature overnight. The solvent was diluted with 1 N NaOH, extracted with EtOAc, and separated. The aqeuous phase was acidified to pH=7, and then extracted with EtOAc. The organic phase was dried (MgSO₄), filtered and evaporated to give a yellow oil, which was then purified by preparatory HPLC to provide 10 mg of the desired product: LCMS Gradient 10-90%, 0.1% formic acid, 5 min, C18/ACN, RT=2.81 minutes (M+H) 400.53.

### Preparation of Compound I-36

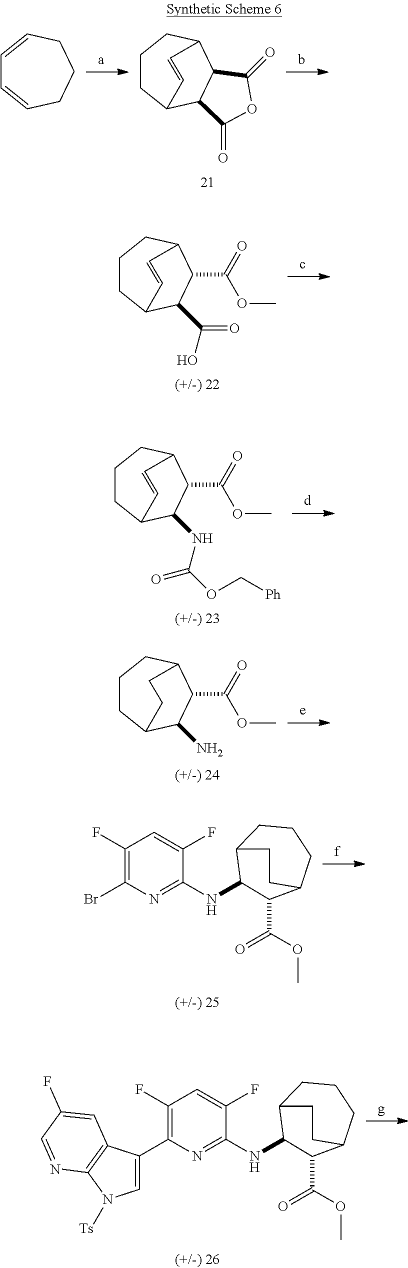

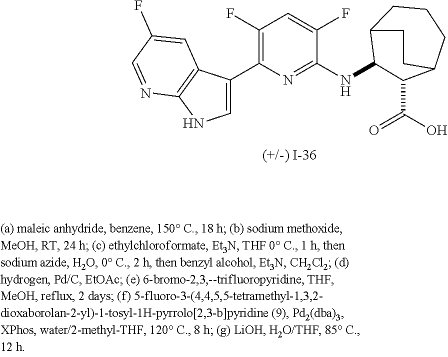

### Formation of (di-exo)-4,5,6,7,8,8a-hexahydro-1H-4,8-ethenocyclohepta[c]furan-1,3(3aH)-dione (21)

Solid maleic anhydride (4.73 g, 48.23 mmol) was added to a stirred solution of cyclohepta-1,3-diene (5.00 g, 53.10 mmol) in benzene (10 mL) in a sealed tube (Q-tube). The suspension was heated at 150° C. for 18 hr to give a clear yellow solution. The reaction mixture was cooled to room temperature and concentrated in vacuo to give 9.3 g of the desired product as an off white solid: ¹H NMR (400 MHz, d6-DMSO) δ 6.16 (dt, J=9.1, 4.5 Hz, 2H), 3.50 (s, 2H), 2.82 (s, 2H), 1.77-1.55 (m, 4H), 1.52-1.38 (m, 2H).

### Formation of (+/−)-(exo)-7-(methoxycarbonyl)bicyclo[3.2.2]non-8-ene-6-carboxylic acid (22)

A solution of sodium methoxide (40.5 mL, 176.9 mmol, 25% W/W) in methanol was added to finely powdered (di-exo)-4,5,6,7,8,8a-hexahydro-1H-4,8-ethenocyclohepta[c]furan-1,3(3aH)-dione, 21, (8.5 g, 44.2 mmol) and the suspension was diluted with methanol (10 mL). The resulting suspension was stirred at room temperature vigorously for 24 hr to give a thick white suspension. The suspension was cooled to 0° C. The cold suspension was added dropwise to a cold solution (0° C.) of concentrated HCl (22.0 mL, 265.3 mmol) in water (22 mL) with cooling on ice. The dropping funnel was washed with methanol (25 mL) and the solution was added dropwise to the HCl solution. The suspension was diluted with water (500 mL) and the aqueous phase was extracted with EtOAc (3×100 mL). The organic layers were dried over Na₂SO₄, filtered and concentrated under reduced pressure. The crude residue was purified by silica gel chromatography (0-50% EtOAC/hexanes) to give 7.5 g of the desired product as a white solid: ¹H NMR (400 MHz, CDCl₃) δ 6.23 (t, J=8.2 Hz, 1H), 6.15-6.03 (m, 1H), 3.76 (s, 3H), 3.52 (d, J=6.9 Hz, 1H), 3.20 (dd, J=6.7, 4.7 Hz, 1H), 3.06-2.85 (m, 2H), 1.79-1.37 (m, 6H).

### Formation of (+/−)-(exo)-methyl 9-(((benzyloxy)carbonyl)amino)bicyclo[3.2.2]-non-6-ene-8-carboxylate (23)

Ethyl chloroformate (3.36 mL, 35.11 mmol) was added dropwise to a stirred solution of racemic-(exo)-7-(methoxycarbonyl)bicyclo[3.2.2]non-8-ene-6-carboxylic acid, 22, (7.50 g, 33.44 mmol) and Et₃N (6.39 mL, 45.81 mmol) in THF (100 mL) at 0° C. with vigorous stirring. A white precipitate was formed and THF (50 mL) was added. The suspension was stirred at 0° C. for 1 hr. A solution of sodium azide (7.39 g, 113.70 mmol) in water (30 mL) was added dropwise at 0° C. The reaction mixture was stirred at room temperature for 2 hr. The mixture was concentrated in vacuo and water (200 mL) was added. The aqueous phase was extracted with EtOAc (3×100 mL). The combined organic phases were dried (MgSO₄), filtered and concentrated in vacuo to give 7.7 g of azide as a clear oil. The crude azide was dissolved in benzene (80 mL) and refluxed for 2 hr. The solution was cooled to room temperature and concentrated in vacuo to give an intermediate isocyanate as a thick oil. The oil was dissolved in dichloromethane (25 mL) and a solution of benzyl alcohol (3.90 mL, 37.69 mmol) and Et₃N (18.65 mL, 133.80 mmol) was added. The clear solution was stirred at room temperature for 18 hr and concentrated in vacuo. The crude product was purified by silica gel chromatography (0-30% EtOAc/hexanes) to give 10.8 g of desired product as a clear oil: ¹H NMR (400 MHz, CDCl₃) δ 7.24 (m, 5H), 6.16 (t, J=8.1 Hz, 1H), 5.98 (t, J=7.8 Hz, 1H), 5.00 (s, 2H), 4.58 (m, 1H), 3.67 (s, 3H), 2.75 (brs, 1H), 2.36-2.44 (m, 2H), 1.66-1.29 (m, 6H); LCMS Gradient 10-90%, 0.1% formic acid, 5 min, C18/ACN, RT=3.4 minutes (M+H) 330.17.

### Formation of (+/−)-(exo)-methyl 7-aminobicyclo[3.2.2]nonane-6-carboxylate (24)

Pd/C (1.65 g, 1.55 mmol, 10% Degussa type, wet) was added to a nitrogen purged solution of racemic-(exo)-methyl 9-(((benzyloxy)carbonyl)amino)bicyclo[3.2.2]-non-6-ene-8-carboxylate, 23 (10.0 g) in EtOAc (50 mL). The solution was hydrogenated (1 atm) at room temperature for 18 hr. The resulting solid suspension was diluted with dichloromethane (100 mL) and stirred at RT for 1 hr. The solution was filtered through a celite pad and the pad was washed thoroughly with DCM (3×50 mL). The filtrate was concentrated in vacuo to afford 5.7 g of desired product: ¹H NMR (400 MHz, CDCl₃) δ 3.77-3.59 (m, 3H), 3.47 (d, J=7.4 Hz, 1H), 2.27 (m, 1H), 2.09 (dd, J=7.4, 3.3 Hz, 1H), 1.85-1.33 (m, 11H).

### Formation of (+/−)-(exo)-methyl 7-((6-bromo-3,5-difluoropyridin-2-yl)amino)bicyclo[3.2.2]nonane-6-carboxylate (25)

To a solution of racemic-(exo)-methyl 7-aminobicyclo[3.2.2]nonane-6-carboxylate, 24, (1.25 g, 6.33 mmol) and 2-bromo-3,5,6-trifluoro-pyridine (1.07 g, 5.07 mmol) in a mixture of THF (20 mL) and MeOH (5 mL) was added diisopropyl-ethylamine (2.21 mL, 12.67 mmol). The reaction mixture was heated at 135° C. for 2 days in a sealed tube (Q-tube). The solvent was evaporated. The crude residue was purified by silica gel chromatography (0%-30% EtOAc/hexanes) to afford 1.14 g of the desired product as an oil: ¹H NMR (400 MHz, CDCl₃) δ 7.06 (dd, J=9.6, 6.8 Hz, 1H), 4.56 (d, J=6.8 Hz, 1H), 3.75 (s, 3H), 2.41 (m, 2H), 2.06-1.60 (brm, 11H); LCMS Gradient 10-90%, 0.1% formic acid, 5 min, C18/ACN, RT=3.95 minutes (M+H) 389.14.

### Formation of (+/−)-(exo)-methyl 7-((3,5-difluoro-6-(5-fluoro-1-tosyl-1H-pyrrolo[2,3-b]pyridin-3-yl)pyridin-2-yl)amino)bicyclo[3.2.2]nonane-6-carboxylate (26)

A solution of 5-fluoro-1-(p-tolylsulfonyl)-3-(4,4,5,5-tetramethyl-1,3,2-dioxaborolan-2-yl)pyrrolo[2,3-b]pyridine, 9, (0.267 g, 0.642 mmol), racemic-(exo)-methyl 7-((6-bromo-3,5-difluoropyridin-2-yl)amino)bicyclo[3.2.2]nonane-6-carboxylate, 25, (0.250 g, 0.642 mmol) and solid K₃PO₄ (0.519 g, 2.447 mmol) in 2-methyl THF (6 mL) and water (1 mL) was purged with nitrogen for 30 min. X-Phos (0.035 g, 0.073 mmol) and Pd₂(dba)₃ (0.014 g, 0.015 mmol) were added to the mixture which was then heated at 120° C. in a Q-tube for 8 hr. The reaction mixture was cooled to room temperature and concentrated in vacuo. The crude product was purified by silica gel chromatography (0-30% EtOAc/hex) to afford 278 mg of the desired product as a white foamy solid: ¹H NMR (400 MHz, CDCl₃) δ 8.65 (dd, J=9.0, 2.7 Hz, 1H), 8.25 (s, 2H), 8.01 (d, J=8.3 Hz, 2H), 7.29-7.18 (m, 2H), 7.08 (t, J=9.8 Hz, 1H), 4.93 (t, J=7.8 Hz, 1H), 4.47 (d, J=7.3 Hz, 1H), 3.58 (s, 3H), 2.43-2.20 (m, 5H), 1.88-1.41 (m, 16H).

### Formation of (+/−)-(exo)-7-((3,5-difluoro-6-(5-fluoro-1H-pyrrolo[2,3-b]pyridin-3-yl)pyridin-2-yl)amino)bicyclo[3.2.2]nonane-6-carboxylic acid (I-36)

Lithium hydroxide hydrate (0.19 g, 4.59 mmol) was added to a stirred solution of racemic-(exo)-methyl 7-((3,5-difluoro-6-(5-fluoro-1-tosyl-1H-pyrrolo[2,3-b]pyridin-3-yl)pyridin-2-yl)amino)bicyclo[3.2.2]nonane-6-carboxylate, 26, (0.28 g, 0.46 mmol) in THF (7 mL) and water (3 mL). The solution was heated at 90° C. for 12 hr and cooled to room temperature. The solution was concentrated in vacuo, water (5 mL) was then added and the solution was neutralized with 2N HCl. The precipitate was extracted with EtOAc (3×10 mL). The organic extracts were dried (MgSO₄) and concentrated in vacuo. The solid was placed in a small buchner funnel and washed with DCM (5 mL). The off-white solid was dried under high vacuum to give 62 mg of the desired product as an off-white solid: ¹H NMR (400 MHz, CD₃OD) δ 8.90 (dd, J=9.6, 2.7 Hz, 1H), 8.16 (s, 1H), 7.98 (d, J=2.5 Hz, 1H), 7.28 (t, J=10.3 Hz, 1H), 5.12 (d, J=7.7 Hz, 1H), 2.71 (dd, J=7.8, 3.6 Hz, 1H), 2.48 (m, 1H), 2.08 (m, 1H), 2.02-1.49 (m, 10H); LCMS Gradient 10-90%, 0.1% formic acid, 5 min, C18/ACN, RT=3.41 minutes (M+H) 431.53.

### Preparation of Compound I-38

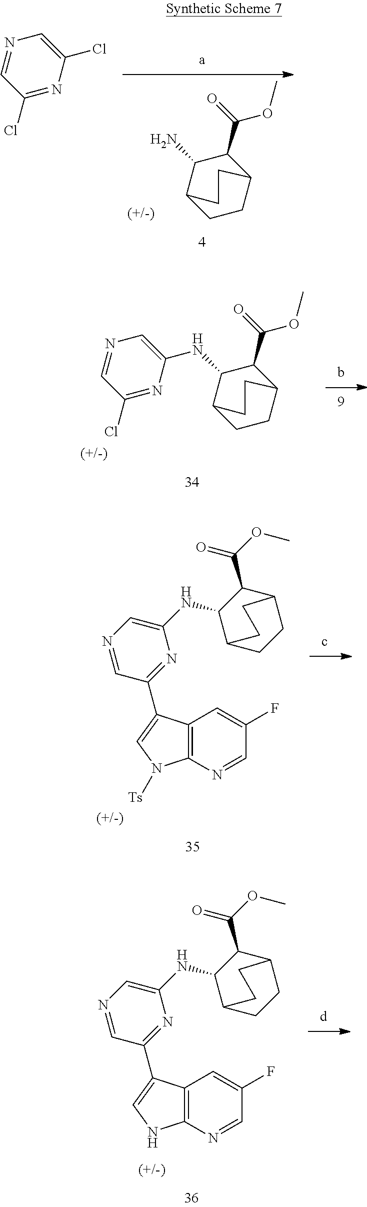 [SMILES: Clc1cncc(Cl)n1 ; COC(=O)[C@H]1C2CCC(CC2)[C@@H]1N ; COC(=O)[C@H]1C2CCC(CC2)[C@@H]1Nc1cncc(Cl)n1 ; COC(=O)[C@H]1C2CCC(CC2)[C@@H]1Nc1cncc(-c2cn(S(=O)(=O)c3ccc(C)cc3)c3ncc(F)cc23)n1 ; COC(=O)[C@H]1C2CCC(CC2)[C@@H]1Nc1cncc(-c2c[nH]c3ncc(F)cc23)n1]

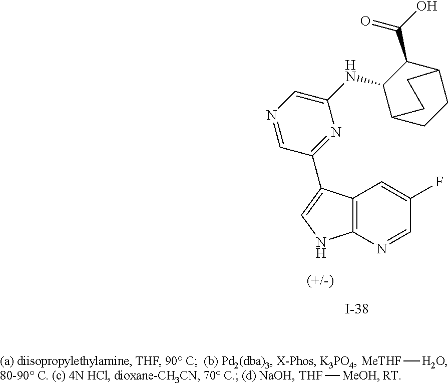

### Formation of (+/−)-trans methyl 3-((6-chloropyrazin-2-yl)amino)bicyclo[2.2.2]octane-2-carboxylate (34)

A solution of 2,6-dichloropyrazine (0.34 g, 2.27 mmol) and methyl (+/−)-trans-7-aminobicyclo[2.2.2]octane-8-carboxylate (0.50 g, 2.73 mmol) and diisopropylethylamine (0.79 mL, 4.55 mmol) in acetonitrile was heated to 70° C. overnight. The reaction was still incomplete and the reaction mixture was heated to 110° C. overnight. The reaction was diluted with EtOAc, washed with brine (2×), dried over Na₂SO₄, filtered and concentrated in vacuo. Flash chromatography (SiO₂, 0-100% EtOAc-hexanes, gradient elution) provided the desired product with sufficient purity for the next reaction: ¹H NMR (400 MHz, CDCl₃) δ 8.02 (s, 1H), 7.74 (s, 1H), 5.71 (s, 1H), 4.32 (s, 1H), 3.80-3.68 (m, 3H), 2.40 (d, J=5.6 Hz, 1H), 2.03 (d, J=2.5 Hz, 1H), 1.87 (d, J=2.7 Hz, 1H), 1.76 (d, J=10.1 Hz, 2H), 1.71-1.40 (m, 6H).

### Formation of (+/−)-trans methyl 3-((6-(5-fluoro-1-tosyl-1H-pyrrolo[2,3-b]pyridin-3-yl)pyrazin-2-yl)amino)bicyclo[2.2.2]octane-2-carboxylate (35)

To a degassed solution of (+/−)-trans methyl 3-((6-chloropyrazin-2-yl)amino)bicyclo-[2.2.2]octane-2-carboxylate, 34, (0.11 g, 0.36 mmol) in MeTHF (2.20 mL) and K₃PO₄ (0.23 g, 1.09 mmol) in water (0.54 mL) was added 5-fluoro-3-(4,4,5,5-tetramethyl-1,3,2-dioxaborolan-2-yl)-1-tosyl-1H-pyrrolo[2,3-b]pyridine, 9, (0.18 g, 0.43 mmol). Degassing was continued for an additional 5 minutes. Then, X-Phos (0.02 g, 0.04 mmol) and Pd₂(dba)₃ (9.9 mg, 0.011 mmol) was added to the mixture. The vessel was sealed and heated to 100° C. After 1 hr., the mixture was cooled, diluted with EtOAc, washed with water, dried over Na₂SO₄, filtered and concentrated in vacuo. Flash chromatography (SiO₂, 0-35% EtOAc/CH₂Cl₂) provided 194 mg of the desired product: ¹H NMR (400 MHz, CDCl₃) δ 8.27 (t, J=7.7 Hz, 3H), 8.08 (s, 1H), 8.03 (d, J=8.3 Hz, 2H), 7.91 (s, 1H), 7.23 (d, J=8.1 Hz, 2H), 4.49 (s, 1H), 3.57 (s, 3H), 2.38 (d, J=6.0 Hz, 1H), 2.32 (s, 3H), 2.02 (s, 1H), 1.90 (s, 1H), 1.85-1.33 (m, 8H); LCMS Gradient 10-90%, 0.1% formic acid, 5 min, C18/ACN, RT=3.88 min (M+H) 550.14.

### Formation of (+/−)-trans methyl 3-((5-fluoro-2-(5-fluoro-1H-pyrrolo[2,3-b]pyridin-3-yl)pyrimidin-4-yl)amino)bicyclo[2.2.2]octane-2-carboxylate (36)

A solution of (+/−)-trans methyl 3-((6-(5-fluoro-1-tosyl-1H-pyrrolo[2,3-b]pyridin-3-yl)pyrazin-2-yl)amino)bicyclo[2.2.2]octane-2-carboxylate, 35, (0.19 g, 0.35 mmol) in acetonitrile (3.88 mL) was treated with HCl (1.77 mL of 4 M solution, 7.06 mmol) in dioxane and heated to 70° C. for 6 hr. The reaction was cooled to 45° C. and kept at this temperature for 3 days. Then, additional HCl (2.0 mL, 8.0 mmol) was added and the mixture was reheated to 70° C. until the reaction was complete (˜20 hr more). The mixture was concentrated in vacuo and diluted with acetonitrile. The resulting solid was sonicated and filtered to provide 81 mg of the desired product as the HCl salt. This material was sufficiently pure to take forward into the hydrolysis step: LCMS Gradient 10-90%, 0.1% formic acid, 5 min, C18/ACN, RT=2.61 min (M+H) 396.14.

### Formation of (+/−)-trans 3-((5-fluoro-2-(5-fluoro-1H-pyrrolo[2,3-b]pyridin-3-yl)pyrimidin-4-yl)amino)bicyclo[2.2.2]octane-2-carboxylic acid (I-38)

A mixture of (+/−)-trans methyl 3-((5-fluoro-2-(5-fluoro-1H-pyrrolo[2,3-b]pyridin-3-yl)pyrimidin-4-yl)amino)bicyclo[2.2.2]octane-2-carboxylate HCl salt, 36, (0.08 mg, 0.18 mmol) in THF (1.5 mL) and MeOH (0.5 mL) was treated with NaOH (0.50 mL of 2 M solution, 1 mmol). The mixture was warmed to 60° C. After 1.75 hr. the reaction was complete as judged by LC-MS. The mixture was concentrated in vacuo to remove the organic solvent, diluted with water (3 mL) and acidified with 2N HCl. The resulting suspension was sonicated, filtered, re-suspended and filtered once more. The wet solid was dried in the a vacuum oven to provide the desired product as the HCl salt: ¹H NMR (300 MHz, MeOD) δ 8.63 (dd, J=9.4, 2.6 Hz, 1H), 8.25 (s, 1H), 8.20 (s, 1H), 8.14 (s, 1H), 7.62 (s, 1H), 4.70 (d, J=6.4 Hz, 1H), 2.56 (d, J=6.4 Hz, 1H), 2.10 (s, 1H), 2.01 (s, 1H), 1.97-1.62 (m, 6H), 1.60-1.43 (m, 2H); LCMS Gradient 10-90%, 0.1% formic acid, 5 min, C18/ACN, RT=2.31 min, (M+H) 382.12.

### Preparation of Compound I-30

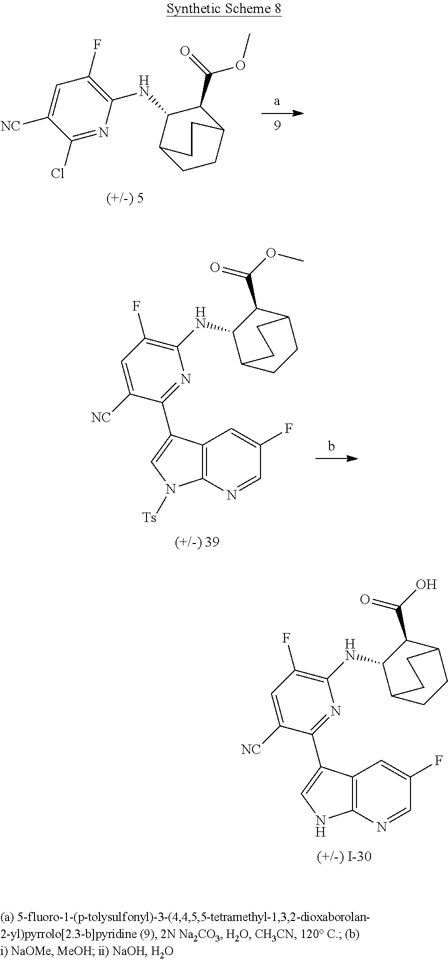 [SMILES: COC(=O)[C@H]1C2CCC(CC2)[C@@H]1Nc1nc(Cl)c(C#N)cc1F ; COC(=O)[C@H]1C2CCC(CC2)[C@@H]1Nc1nc(-c2cn(S(=O)(=O)c3ccc(C)cc3)c3ncc(F)cc23)c(C#N)cc1F ; N#Cc1cc(F)c(N[C@H]2C3CCC(CC3)[C@@H]2C(=O)O)nc1-c1c[nH]c2ncc(F)cc12]

### Formation of (+/−)-trans-methyl 3-((5-cyano-3-fluoro-6-(5-fluoro-1-tosyl-1H-pyrrolo[2,3-b]pyridin-3-yl)pyridin-2-yl)amino)bicyclo[2.2.2]octane-2-carboxylate (39)

To a solution of (+/−)-trans-methyl 3-((6-chloro-5-cyano-3-fluoropyridin-2-yl)amino)-bicycle[2.2.2]-octane-2-carboxylate, 5, (0.11 g, 0.31 mmol) and 5-fluoro-1-(p-tolylsulfonyl)-3-(4,4,5,5-tetramethyl-1,3,2-dioxaborolan-2-yl)pyrrolo[2,3-b]pyridine, 9, (0.16 g, 0.38 mmol) in CH₃CN was added Na₂CO₃ (0.50 mL of 2 M solution, 1.00 mmol). The mixture was degassed for 15 minutes followed by the addition of Pd(PPh₃)₄ (0.05 g, 0.04 mmol). The reaction mixture was heated in a microwave at 120° C. for 30 minutes. Ethyl acetate (10 mL) was added and the mixture was filtered through a bed of celite and the resulting filtrate was concentrated in vacuo. The crude material was purified via silica gel chromatography (EtOAc/hex 0-50%) to afford 125 mg of the desired product as a yellow solid: ¹H NMR (400 MHz, CDCl₃) δ 8.66 (s, 1H), 8.46-8.25 (m, 1H), 8.14 (d, J=8.4 Hz, 1H), 7.78 (s, 1H), 7.69 (d, J=6.4 Hz, 1H), 7.35 (dd, J=21.9, 12.3 Hz, 2H), 5.23 (s, 1H), 4.81 (s, 1H), 3.53 (s, 3H), 2.40 (s, 3H), 2.09 (s, 1H), 1.94 (d, J=14.9 Hz, 1H), 1.72 (d, J=10.5 Hz, 3H), 1.57 (s, 6H), 1.47 (s, 2H); LCMS Gradient 10-90%, 0.1% formic acid, 5 min, C18/ACN, (M+H) 592.42

### Formation of (+/−)-trans-methyl 3-((5-cyano-3-fluoro-6-(5-fluoro-1H-pyrrolo[2,3-b]pyridin-3-yl)pyridin-2-yl)amino)bicyclo[2.2.2]octane-2-carboxylate (I-30)

To a solution of (+/−)-trans-methyl 3-((5-cyano-3-fluoro-6-(5-fluoro-1-tosyl-1H-pyrrolo[2,3-b]pyridin-3-yl)pyridin-2-yl)amino)bicyclo[2.2.2]octane-2-carboxylate, 39, (0.12 g, 0.18 mmol) in methanol/tetrahydrofuran (2:1, 3 ml/1.5 mL) was added sodium methoxide in methanol (0.10 mL 35% w/w) was added. After stirring for 10 minutes at room temperature, a solution of 1N NaOH (0.30 mL of 1M solution) was added and the reaction mixture was stirred overnight at room temperature. The mixture was concentrated in vacuo. The crude product was purified by silica gel chromatography (0-10% methanol/dichloromethane gradient) to give the title compound as a light yellow solid. The product was re-acidified with hydrochloric acid (1N, 400 uL) to afford 30 mg of the desired product as a hydrochloride salt: ¹H NMR (400 MHz, DMSO-d6) δ 12.43 (s, 1H), 8.47 (d, J=9.7 Hz, 1H), 8.36 (d, J=15.5 Hz, 2H), 7.86 (d, J=11.3 Hz, 1H), 7.77 (d, J=7.2 Hz, 1H), 4.77 (d, J=6.6 Hz, 1H), 4.11 (s, 1H), 2.89 (d, J^{=6.7} Hz, 1H), 2.01 (s, 1H), 1.86 (s, 1H), 1.75 (d, J=18.0 Hz, 3H), 1.49 (td, J=35.0, 9.6 Hz, 6H); LCMS Gradient 10-90%, 0.1% formic acid, 5 min, C18/ACN, (M+H) 424.33.

### Preparation of Compound I-39

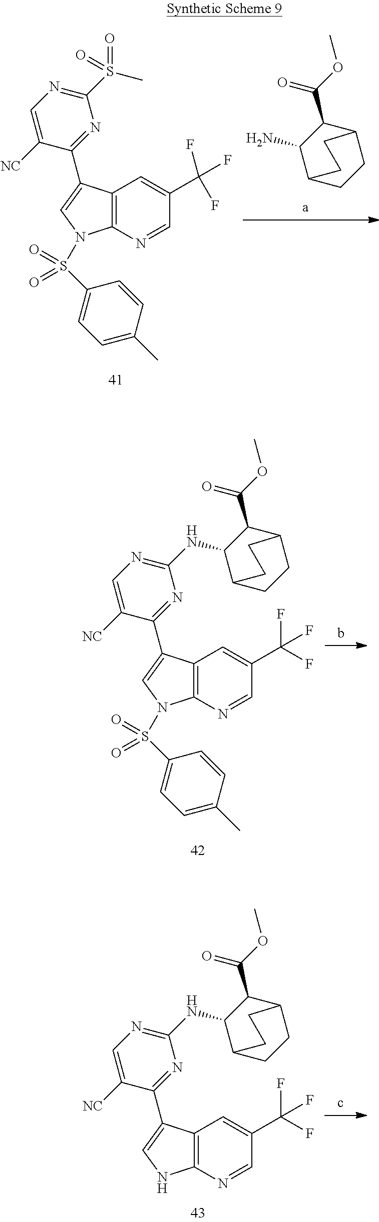

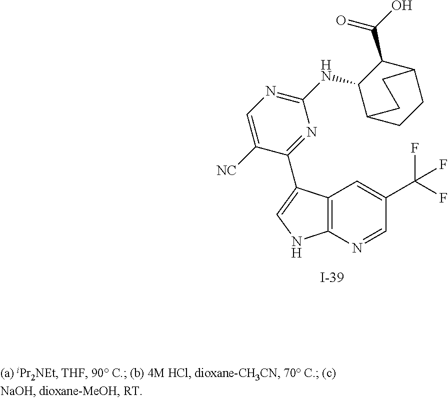

### Formation of (+/−)-trans methyl 3-((5-cyano-4-(1-tosyl-5-(trifluoromethyl)-1H-pyrrolo[2,3-b]pyridin-3-yl)pyrimidin-2-yl)amino)bicyclo[2.2.2]octane-2-carboxylate (42)

The starting material, sulfone 41, was prepared accordingly to the procedure described in WO-2008079346.

A mixture of 2-(methylsulfonyl)-4-(1-tosyl-5-(trifluoromethyl)-1H-pyrrolo[2,3-b]pyridin-3-yl)pyrimidine-5-carbonitrile, 41, (0.250 g, 0.479 mmol) and (+/−)-trans methyl 3-aminobicyclo[2.2.2]octane-2-carboxylate, 4, (0.123 g, 0.671 mmol) and diisopropylethylamine (0.192 mL, 1.100 mmol) in dry THF (10.0 mL) was heated to 90° C. for 2 hr. The solution was concentrated in vacuo. Flash chromatography (SiO₂, 0-100% EtOAc/hexanes gradient) provided 99 mg of the desired product. This material was taken forward into the detosylation step without further purification: LCMS Gradient 10-90%, 0.1% formic acid, 5 min, C18/ACN, RT=2.79 min (M+H) 629.13.

### Formation of (+/−)-trans methyl 3-((5-cyano-4-(5-(trifluoromethyl)-1H-pyrrolo[2,3-b]pyridin-3-yl)-pyrimidin-2-yl)amino)bicyclo[2.2.2]octane-2-carboxylate (43)

A mixture of (+/−)-trans methyl 3-((5-cyano-4-(1-tosyl-5-(trifluoromethyl)-1H-pyrrolo[2,3-b]pyridin-3-yl)pyrimidin-2-yl)amino)bicyclo[2.2.2]octane-2-carboxylate, 42, (0.098 g, 0.157 mmol) in dry acetonitrile (3 mL) was treated with HCl (0.785 mL of 4 M solution, 3.140 mmol) in dioxane and heated to 70° C. until the reaction appeared complete as judged by LC-MS. The cooled reaction mixture was triturated 3 times with acetonitrile and dried in vacuo to provide 35 mg of the desired product as the HCl salt. This material was sufficiently pure to be taken forward into the next step: LCMS Gradient 10-90%, 0.1% formic acid, 5 min, C18/ACN, RT=3.72 min (M+H) 471.36.

### Formation of (+/−)-trans 3-((5-cyano-4-(5-(trifluoromethyl)-1H-pyrrolo[2,3-b]pyridin-3-yl)pyrimidin-2-yl)amino)bicyclo[2.2.2]octane-2-carboxylic acid (I-39)

To a suspension of (+/−)-trans methyl 3-((5-cyano-4-(5-(trifluoromethyl)-1H-pyrrolo[2,3-b]pyridin-3-yl)-pyrimidin-2-yl)amino)bicyclo[2.2.2]octane-2-carboxylate hydrochloric acid salt, 43, (0.035 g, 0.069 mmol) in THF (1.5 mL) and MeOH (0.70 mL) was added NaOH (0.20 mL of 2 M, 0.40 mmol) and the mixture was stirred at room temperature overnight. After 36 hr., additional NaOH (0.10 mL) was added and the mixture was stirred overnight again. Once the reaction was complete, the mixture was concentrated in vacuo to remove the organic solvents. The solution was acidified with HCl forming a thick suspension. The solid was isolated by filtration and dried in vacuo to provide desired product as an off white powder: ¹H NMR (300 MHz, MeOD) (NMR indicated the presence of rotamers major rotamer): δ 9.33 (s, 1H), 8.77 (s, 1H), 8.64 (s, 1H), 8.55 (d, J=16.8 Hz, 1H), 4.58 (d, 1H), 2.65 (d, J=7.2 Hz, 1H), 2.17-2.06 (m, 1H), 2.06-1.95 (m, 1H), 1.94-1.73 (m, 3H), 1.73-1.55 (m, 2H), 1.55-1.42 (m, 2H); LCMS Gradient 10-90%, 0.1% formic acid, 5 min, C18/ACN, RT=3.24 min (M+H) 457.12.

### Preparation of Compound I-22

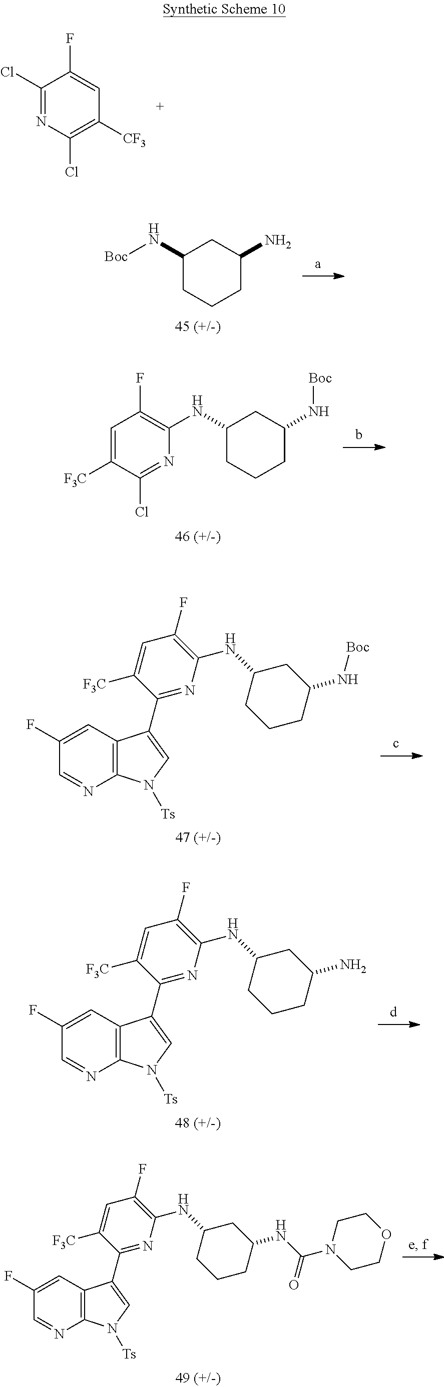

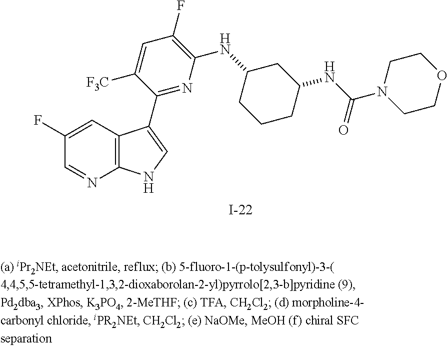

### Formation of (+/−) cis-tert-butyl (3-((6-chloro-3-fluoro-5-(trifluoromethyl)pyridin-2-yl)amino)cyclohexyl)carbamate (46)

A solution of cis-tert-butyl N-cis-3-aminocyclohexyl]carbamate, 45, (0.20 g, 0.93 mmol), 2,6-dichloro-3-fluoro-5-(trifluoromethyl)pyridine (0.22 g, 0.93 mmol) and N,N-diisopropylethylamine (0.25 mL, 1.40 mmol) in acetonitrile (15 mL) was heated to reflux overnight. The solvent was removed under reduced pressure, and the residue was partitioned between EtOAc (20 mL) and saturated aqueous NaHCO₃ (20 mL). The layers were separated, the aqueous layer was extracted with EtOAc (2×20 mL) and the combined organic layers were dried on Na₂SO₄, filtered and concentrated in vacuo to provide 330 mg of the crude product which was used in the next step without further purification: LCMS Gradient 10-90%, 0.1% formic acid, 5 min, C18/ACN, RT=3.86 min (M+H) 412.18.

### Formation of (+/−) cis-tert-butyl (3-((3-fluoro-6-(5-fluoro-1-tosyl-1H-pyrrolo[2,3-b]pyridin-3-yl)-5-(trifluoromethyl)pyridin-2-yl)amino)cyclohexyl)carbamate (47)

K₃PO₄ (0.39 g, 1.82 mmol) was dissolved in a mixture of water (1.5 mL) and 2-Me-THF (5.0 mL). Racemic cis-tert-butyl (3-((6-chloro-3-fluoro-5-(trifluoromethyl)pyridin-2-yl)amino)cyclohexyl)carbamate, 46, (0.25 g, 0.61 mmol) was added and the mixture was purged with N₂ for 30 minutes. 5-Fluoro-1-(p-tolylsulfonyl)-3-(4,4,5,5-tetramethyl-1,3,2-dioxaborolan-2-yl)pyrrolo[2,3-b]pyridine, 9, (0.28 g, 0.66 mmol) was added and the N₂ purging was continued for an additional 15 min. XPhos (0.02 g, 0.04 mmol) and Pd₂(dba)₃ (0.01 g, 0.01 mmol) were added under N₂, and the vial was sealed and heated to 80° C. overnight. After cooling to room temperature, the reaction mixture was diluted with EtOAc (20 mL) and water (20 mL) and the layers were separated. The organic layer was dried on Na₂SO₄, filtered and concentrated in vacuo. The resulting residue was purified by flash chromatography (0-50% EtOAc/CH₂Cl₂) to afford 230 mg of the desired product: ¹H NMR (300 MHz, CDCl₃) δ 8.34 (dd, J=2.7, 1.1 Hz, 1H), 8.12-8.05 (m, 3H), 7.84 (dd, J=8.7, 2.8 Hz, 1H), 7.51 (d, J=11.1 Hz, 1H), 7.32 (d, J^{=8.1} Hz, 2H), 4.88 (d, J=6.0 Hz, 1H), 4.43 (s, 1H), 4.09-3.97 (m, 1H), 3.48 (s, 1H), 2.41 (s, 3H), 2.46-2.34 (m, 1H), 2.14 (d, J=11.9 Hz, 1H), 1.97 (d, J=16.1 Hz, 1H), 1.91-1.77 (m, 1H), 1.52-1.33 (m, 9H), 1.22-0.98 (m, 4H); LCMS Gradient 10-90%, 0.1% formic acid, 5 min, C18/ACN, RT=4.12 minutes (M+H) 665.93.

### Formation of (+/−) cis-N1-(3-fluoro-6-(5-fluoro-1-tosyl-1H-pyrrolo[2,3-b]pyridin-3-yl)-5-(trifluoromethyl)pyridin-2-yl)cyclohexane-1,3-diamine (48)

Racemic cis-tert-butyl (3-((3-fluoro-6-(5-fluoro-1-tosyl-1H-pyrrolo[2,3-b]pyridin-3-yl)-5-(trifluoromethyl)pyridin-2-yl)amino)cyclohexyl)carbamate, 47, (0.22 g, 0.33 mmol) was dissolved in dichloromethane (10 mL) and treated with TFA (5 mL). After stirring for 20 minutes, the solvent was evaporated under reduced pressure and the residue was suspended in water (10 mL) and treated with 1N NaOH (5 mL). The resulting suspension was sonicated for 5 minutes, and the precipitate was collected by filtration to provide 140 mg of the desired product as an off-white solid, which was used for the next step without further purification: ¹H NMR (300 MHz, CDCl₃) δ 8.24 (d, J=1.6 Hz, 1H), 7.99 (d, J=8.2 Hz, 3H), 7.78 (dd, J=8.9, 2.7 Hz, 1H), 7.41 (d, J=11.1 Hz, 1H), 7.31-7.20 (m, 2H), 5.34 (s, 1H), 4.14-3.87 (m, 1H), 2.87 (t, J^{=9.8} Hz, 1H), 2.50 (s, 2H), 2.32 (s, 3H), 2.14 (d, J^{=11.9} Hz, 1H), 1.93 (d, J=10.9 Hz, 1H), 1.79 (d, J=10.8 Hz, 2H), 1.42-0.99 (m, 4H); LCMS Gradient 10-90%, 0.1% formic acid, 5 min, C18/ACN, RT=2.08 minutes (M+H) 566.33.

### Formation of (+/−)-cis-N-(3-((3-fluoro-6-(5-fluoro-1-tosyl-1H-pyrrolo[2,3-b]pyridin-3-yl)-5-(trifluoromethyl)pyridin-2-yl)amino)cyclohexyl)morpholine-4-carboxamide (49)

Racemic-cis-N1-(3-fluoro-6-(5-fluoro-1-tosyl-1H-pyrrolo[2,3-b]pyridin-3-yl)-5-(trifluoro-ethyl)pyridin-2-yl)cyclohexane-1,3-diamine, 48, (0.07 g, 0.11 mmol) was dissolved in CH₂Cl₂ (10 mL) and treated with N,N-diisopropylethylamine (0.20 mL, 1.14 mmol), followed by morpholine-4-carbonyl chloride (0.09 g, 0.57 mmol). After stirring at room temperature overnight, the reaction mixture was washed with saturated aqueous NaHCO₃, the organic layer was collected, dried on Na₂SO₄, filtered and concentrated in vacuo to provide 61 mg of the crude product which was taken onto the next step without further purification: LCMS Gradient 10-90%, 0.1% formic acid, 5 min, C18/ACN, RT=3.68 minutes (M+H) 678.86. Formation of N-((1R,3S)-3-((3-fluoro-6-(5-fluoro-1H-pyrrolo[2,3-b]pyridin-3-yl)-5-(trifluoromethyl)pyridin-2-yl)amino)cyclohexyl)morpholine-4-carboxamide (I-22)

Racemic N-(3-((3-fluoro-6-(5-fluoro-1-tosyl-1H-pyrrolo[2,3-b]pyridin-3-yl)-5-(trifluoromethyl)pyridin-2-yl)amino)cyclohexyl)morpholine-4-carboxamide, 49, (0.06 g, 0.08 mmol) was dissolved in methanol (4 mL) and treated with sodium methoxide (0.17 mL of 25% w/v, 0.81 mmol). After stirring at room temperature for 1 hr, the solvent was evaporated, and the residue was suspended in water and stirred for 1 hr. The resulting suspension was extracted with EtOAc (2×4 mL) and the organic extract was concentrated in vacuo to provide the crude product as a racemic mixture.

Separation of the racemic mixture using chiral SFC chromatographic resolution: 20% MeOH, 80% CO₂ (10 mL/min) provided the individual enantiomers.

### N-[(1R,3S)-3-[[3-fluoro-6-(5-fluoro-1H-pyrrolo[2,3-b]pyridin-3-yl)-5-(trifluoro-methyl)-2-pyridyl]amino]cyclohexyl]morpholine-4-carboxamide (I-22)

¹H NMR (300 MHz, CDCl₃) δ 10.41 (s, 1H), 8.37 (t, J=5.4 Hz, 1H), 8.28 (dd, J=9.8, 2.7 Hz, 1H), 8.25-8.18 (m, 1H), 7.80-7.66 (m, 1H), 4.62 (dd, J=7.7, 1.9 Hz, 1H), 4.38 (t, J=11.5 Hz, 1H), 4.27-4.08 (m, 1H), 3.88-3.60 (m, 5H), 3.33 (dd, J=10.3, 5.3 Hz, 4H), 2.49 (d, J=11.5 Hz, 1H), 2.29 (d, J=10.8 Hz, 1H), 2.09 (d, J^{=10.3} Hz, 1H), 1.87 (dd, J^{=10.8, 3.1} Hz, 1H), 1.68-1.43 (m, 1H), 1.38-1.03 (m, 3H); LCMS Gradient 10-90%, 0.1% formic acid, 5 min, C18/ACN, RT=2.82 minutes (M+H) 525.03.

### Preparation of Compounds I-22

The following compounds can be prepared in the same manner as described above for Compound I-22:

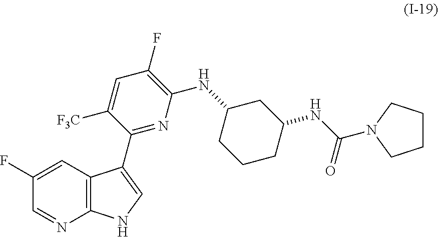

### N-((1R,3S)-3-((3-fluoro-6-(5-fluoro-1H-pyrrolo[2,3-b]pyridin-3-yl)-5-(trifluoromethyl)-pyridin-2-yl)amino)cyclohexyl)pyrrolidine-1-carboxamide

¹H NMR (300 MHz, CDCl₃) δ 10.09 (s, 1H), 8.37 (d, J=2.8 Hz, 1H), 8.28 (dt, J=6.5, 3.2 Hz, 1H), 8.24-8.18 (m, 1H), 7.73 (d, J=12.7 Hz, 1H), 4.61 (dd, J=7.8, 2.0 Hz, 1H), 4.26-4.13 (m, 2H), 3.79 (ddd, J=19.8, 13.5, 7.9 Hz, 1H), 3.38-3.23 (m, 4H), 2.49 (d, J=11.4 Hz, 1H), 2.31 (d, J=10.9 Hz, 1H), 2.09 (d, J=14.5 Hz, 1H), 1.98-1.79 (m, 5H), 1.57 (dd, J^{=26.3, 13.0} Hz, 1H), 1.35-1.04 (m, 3H); LCMS Gradient 10-90%, 0.1% formic acid, 5 min, C18/ACN, RT=2.96 minutes (M+H) 509.54.

### Preparation of Compounds I-42, I-45, and I-46

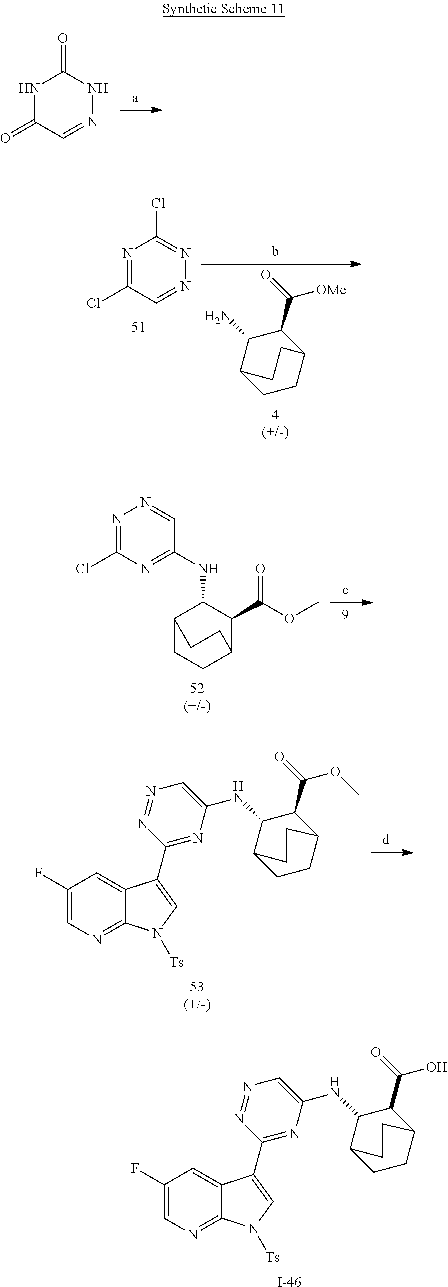

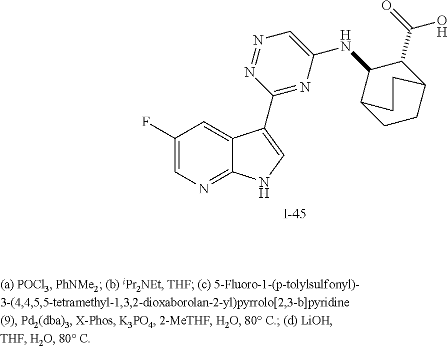

### Formation of 3,5-dichloro-1,2,4-triazine (51)

To 6-azauracil (1.00 g 8.9 mmol) was added phosphorus oxychloride (10.0 mL, 108.0 mmol) and N,N-dimethylaniline (2.0 mL, 16.0 mmol). The reaction mixture was heated in a microwave reactor at 90° C. for 20 minutes. The reaction was extracted with hexane (200 mL) twice and filtered through Celite and sodium sulfate. The organic solvent was evaporated in vacuo to give 530 mg of the desired compound which was used without further purification.

### Preparation of (+/−)-trans-methyl 3-((3-chloro-1,2,4-triazin-5-yl)amino)bicyclo[2.2.2]octane-2-carboxylate (52)

To a solution of 3,5-dichloro[1,2,4]triazine, 51, (0.75 g, 5.00 mmol) in anhydrous dioxane (50 mL) was added N,N-diisopropylethylamine (1.74 mL 10.00 mmol) and racemic-trans-methyl 3-aminobicyclo[2.2.2]octane-2-carboxylate, 4, (0.92 g 5.00 mmol). The mixture was stirred at room temperature for 4 hours. Ethyl Acetate (200 mL) was added. The organic solution was washed with aqueous saturated ammonium chloride solution, water and brine. The organic layer was dried over sodium sulfate, filtered and concentrated in vacuo. The crude product was purified by silica chromatography (25-75% Ethyl Acetate/hexanes gradient) to give 500 mg of the title compound.

### Preparation of (+/−)-trans-methyl 3-((3-(5-fluoro-1-tosyl-1H-pyrrolo[2,3-b]pyridin-3-yl)-1,2,4-triazin-5-yl)amino)bicyclo[2.2.2]octane-2-carboxylate (53)

To a solution of 5-fluoro-3-(4,4,5,5-tetramethyl-1,3,2-dioxaborolan-2-yl)-1-tosyl-1H-pyrrolo[2,3-b]pyridine, 9, (0.31 g 0.74 mmol) and racemic-trans-methyl 3-((3-chloro-1,2,4-triazin-5-yl)amino)bicyclo[2.2.2]octane-2-carboxylate, 52, (0.20 g, 0.67 mmol) in 2-Me-THF (6 mL) and water (1 mL) was added Pd₂(dba)₃ (0.04 g 0.15 mmol) and X-Phos (0.05 g 0.10 mmol). The mixture was degassed under flow of nitrogen for 5 minutes. K₃PO₄ (0.50 g 2.36 mmol) was then added and the reaction mixture was sealed in vial and heated to 80° C. for 2 hours. The mixture was diluted with ethyl Acetate (20 mL) and washed with brine and water. The organic phase was dried over sodium sulfate, filtered and concentrated in vacuo. The resulting crude residue was purified by silica gel chromatography (0-7% MeOH [2N]NH₃ in EtOAc) to give 50 mg of the title compound.

### Preparation of (+/−)-3-[[3-(5-fluoro-1H-pyrrolo[2,3-b]pyridin-3-yl)-1,2,4-triazin-5-yl]amino]bicyclo[2.2.2]octane-2-carboxylic acid (I-42)

To a solution of racemic-trans-methyl 3-((3-(5-fluoro-1-tosyl-1H-pyrrolo[2,3-b]pyridin-3-yl)-1,2,4-triazin-5-yl)amino)bicyclo[2.2.2]octane-2-carboxylate, 53, (0.050 g 0.091 mmol) in THF (10 mL) was added [2N]LiOH (1.0 mL of 2N solution, 2.000 mmol). The reaction was heated to 80° C. for 2 hours. Ethyl Acetate (25 mL) was added and solution was washed with brine and water. The organic layer was dried over sodium sulfate, filtered and concentrated in vacuo. The resulting crude residue was purified by reverse phase-HPLC (0.1% TFA-CH₃CN/H₂O) to give 5 mg of the TFA salt of the title compound, I-42, as a racemic mixture of trans-isomers: ¹H NMR (300 MHz, DMSO-d6) δ 12.91 (s, 1H), 12.52 (s, 1H), 8.62 (s, 1H), 8.46 (d, J=3.0 Hz, 1H), 8.42 (d, J=5.7 Hz, 1H), 8.30 (s, 1H), 4.72 (s, 1H), 2.60 (d, J=6.5 Hz, 1H), 2.06 (s, 1H), 1.99 (s, 1H), 1.82-1.40 (m, 9H).

Separation of the racemic mixture using chiral SFC chromatographic resolution (20% EtOH (0.2% DEA), 80% CO₂) provided the individual enantiomers, I-45 and I-46.

### Preparation of Compound 56

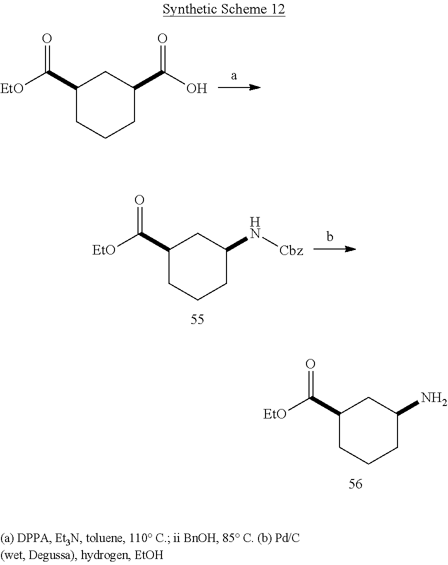 [SMILES: CCOC(=O)[C@@H]1CCC[C@H](C(=O)O)C1 ; CCOC(=O)[C@@H]1CCC[C@H](NC(=O)OCc2ccccc2)C1 ; CCOC(=O)[C@@H]1CCC[C@H](N)C1]

### Formation of (1S,3R)-3-(ethoxycarbonyl)cyclohexanecarboxylic acid

(1S,3R)-3-(ethoxycarbonyl)cyclohexanecarboxylic acid starting material can be prepared following the literature procedures described in: Barnett, C. J., Gu, R. L., Kobierski, M. E., WO-2002024705, Stereoselective process for preparing cyclohexyl amine derivatives.

### Formation of ethyl (1R,3S)-3-benzyloxycarbonylaminocyclohexanecarboxylate (55)

(1S,3R)-3-(Ethoxycarbonyl)cyclohexanecarboxylic acid (10.0 g, 49.9 mmol) was dissolved in toluene (100 mL) and treated with triethylamine (7.6 mL, 54.9 mmol) and DPPA (12.2 mL, 54.9 mmol). The resulting solution was heated to 110° C. and stirred for 1 hour. After cooling to 70° C., benzyl alcohol (7.7 mL, 74.9 mmol) was added, and the mixture was heated to 85° C. overnight. The resulting solution was cooled to room temperature, poured into EtOAc (150 mL) and water (150 mL) and the layers were separated. The aqueous layer was extracted with EtOAc (2×75 mL) and the combined organic extracts were washed with water (100 mL) and brine (100 mL), dried over Na₂SO₄ and concentrated in vacuo. The crude material was purified by silica gel chromatography (0%-50% EtOAc/hexanes) to provide 17 (15.3 g, containing ˜25% benzyl alcohol), which was used for the next step without further purification.

### Formation (1R,3S)-ethyl 3-aminocyclohexanecarboxylate (56)

To a solution of (1R,3S)-ethyl 3-(benzyloxycarbonylamino)cyclohexane-carboxylate, 55, (14.0 g, 45.9 mmol) in ethanol (3 mL) was added Pd/C (wet, Degussa (2.4 g, 2.3 mmol). The mixture was evacuated and then stirred under atmosphere of hydrogen at room temperature overnight. The reaction mixture was filtered through a pad of celite and the resulting filtrate concentrated in vacuo to provide an oil that was used without further purification.

### Preparation of Compound I-6 and I-10

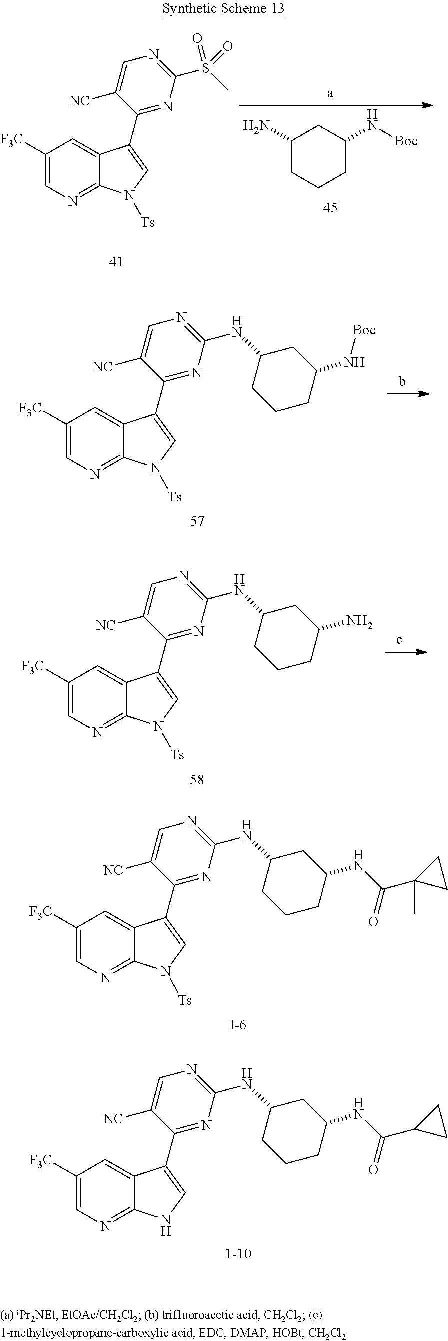

### Formation of tert-butyl ((1R,3S)-3-((5-cyano-4-(5-(trifluoromethyl)-1-tosyl-1H-pyrrolo[2,3-b]pyridin-3-yl)pyrimidin-2-yl)amino)cyclohexyl)carbamate (57)

A suspension of 2-methylsulfonyl-4-[1-(p-tolylsulfonyl)-5-(trifluoromethyl)-pyrrolo[5,4-b]pyridin-3-yl]pyrimidine-5-carbonitrile, 41, (0.101 g, 0.194 mmol) and tert-butyl N-[(1S,3R)-3-aminocyclohexyl]carbamate (0.046 g, 0.213 mmol) in EtOAc: CH₂Cl₂ (10 mL, 1:1 mixture) was treated with N,N-diisopropylethylamine (0.100 mL, 0.574 mmol) and allowed to stir at room temperature for 4 hours. The reaction was diluted with CH₂Cl₂ and washed with 1N HCl. The organic layer was concentrated in vacuo and the residue absorbed onto silica gel and then purified via silica gel chromatography (0-100% EtOAc: hexanes gradient) to afford 65 mg of desired product as a light yellow solid.

### Formation of 2-(((1S,3R)-3-aminocyclohexyl)amino)-4-(5-(trifluoromethyl)-1-tosyl-1H-pyrrolo[2,3-b]pyridin-3-yl)pyrimidine-5-carbonitrile (58)

A solution of tert-butyl ((1S,3R)-3-((5-cyano-4-(5-(trifluoromethyl)-1-tosyl-1H-pyrrolo[2,3-b]pyridin-3-yl)pyrimidin-2-yl)amino)cyclohexyl)carbamate, 57, (65 mg, 0.1 mmol) in TFA: CH₂Cl₂ (10 mL, 1:1 mixture) was stirred at room temperature for 1 hour then concentrated to dryness. The crude product was converted to the free base using PL-HCO3 MP resin with methanol eluent. The filtrate was concentrated in vacuo to give 50 mg of the crude product that was used in the next step without additional purification.

### Formation of N-((1R,3S)-3-((5-cyano-4-(5-(trifluoromethyl)-1H-pyrrolo[2,3-b]pyridin-3-yl)pyrimidin-2-yl)amino)cyclohexyl)-1-methylcyclopropanecarboxamide (amide-Me-cyclopropyl) (I-6) and N-((1S,3R)-3-((5-cyano-4-(5-(trifluoromethyl)-1H-pyrrolo[2,3-b]pyridin-3-yl)pyrimidin-2-yl)amino)cyclohexyl)cyclopropanecarboxamide (cyclopropyl amide) (I-10)

A mixture of 2-(((1R,3S)-3-aminocyclohexyl)amino)-4-(5-(trifluoromethyl)-1-tosyl-1H-pyrrolo[2,3-b]pyridin-3-yl)pyrimidine-5-carbonitrile, 58, (0.070 g, 0.126 mmol), EDC (0.120 g, 0.630 mmol) and HOBT (0.020 g, 0.145 mmol) in CH₂Cl₂ (5 mL) was treated with a solution of 1-methylcyclopropanecarboxylic acid (0.060 g, 0.599 mmol) in CH₂Cl₂ (2 mL). After 70 min at room temperature, LCMS showed incomplete reaction. At 2.25 h, added DMAP (catalytic) to the reaction mixture and stirred at room temperature overnight. The reaction mixture was diluted into EtOAc and washed with water, 0.5N HCl then aqueous saturated NaHCO₃ solution and then brine. The organic phase was concentrated in vacuo to give 186 mg of a yellow-orange residue which was absorbed on silica gel and purified via silica gel chromatography (0-100% EtOAc:hexanes gradient). Two products were obtained from the purification. The less polar (faster moving) compound provided 51 mg of a white solid that was identified as the 1-methyl-cyclopropylamide by LCMS. The second product (more polar, slower moving) showed a M-14 mass by LCMS and was identified as a cyclopropyl amide, presumably obtained due to a cyclopropane carboxylic acid impurity contained in the starting reagent. Both compounds were separately taken forward into the detosylation step.

The major product was dissolved in methanol and treated with Na (62 mg, excess) at room temperature. LCMS at 5 min showed complete conversion to desired product. The reaction mixture was concentrated to dryness then diluted with EtOAc and water. The organic phase was washed with brine and concentrated in vacuo to give 32 mg of a solid after vacuum drying: LCMS RT=3.93 minutes (M+H) 484.58

The minor product was suspended in MeOH and treated with Na metal. After dissolving, LCMS showed product. Concentrated to dryness then diluted with EtOAc and water. Washed organic with brine and concentrated to give 6 mg of a solid after vacuum drying: LCMS Gradient 10-90%, 0.1% formic acid, 5 min, C18/ACN, RT=3.85 minutes (M+H) 470.46

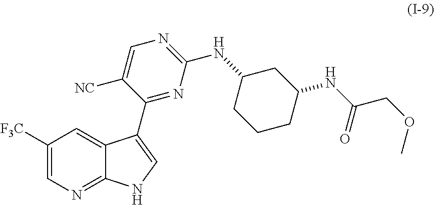

### Formation of N-((1R,3S)-3-((5-cyano-4-(5-(trifluoromethyl)-1H-pyrrolo[2,3-b]pyridin-3-yl)pyrimidin-2-yl)amino)cyclohexyl)-2-methoxyacetamide (I-9)

A sample of 2-[[(1S,3R)-3-aminocyclohexyl]amino]-4-[1-(p-tolylsulfonyl)-5-(trifluoromethyl)pyrrolo[2,3-b]pyridin-3-yl]pyrimidine-5-carbonitrile, 58, (0.085 g, 0.153 mmol) was suspended in CH₂Cl₂ and treated with N,N-diisopropylethylamine (0.200 mL, 1.148 mmol) and three drops of 2-methoxyacetyl chloride (0.016 g, 0.153 mmol). After 15 min, the solvent was evaporated under a stream of nitrogen and the resulting residue was dissolved in MeOH and treated with a small piece of Na metal. After the metal dissolved, the mixture was concentrated to dryness and then partitioned between water and EtOAc. The organic layer was washed with brine, dried over Na₂SO₄, filtered through celite and concentrated in vacuo to give a sticky solid. The crude residue was dissolved in DMSO and purified by preparatory HPLC (0.1% TFA-CH₃CN/H₂O) to yield 17 mg of desired product.

### Preparation of Compound I-4

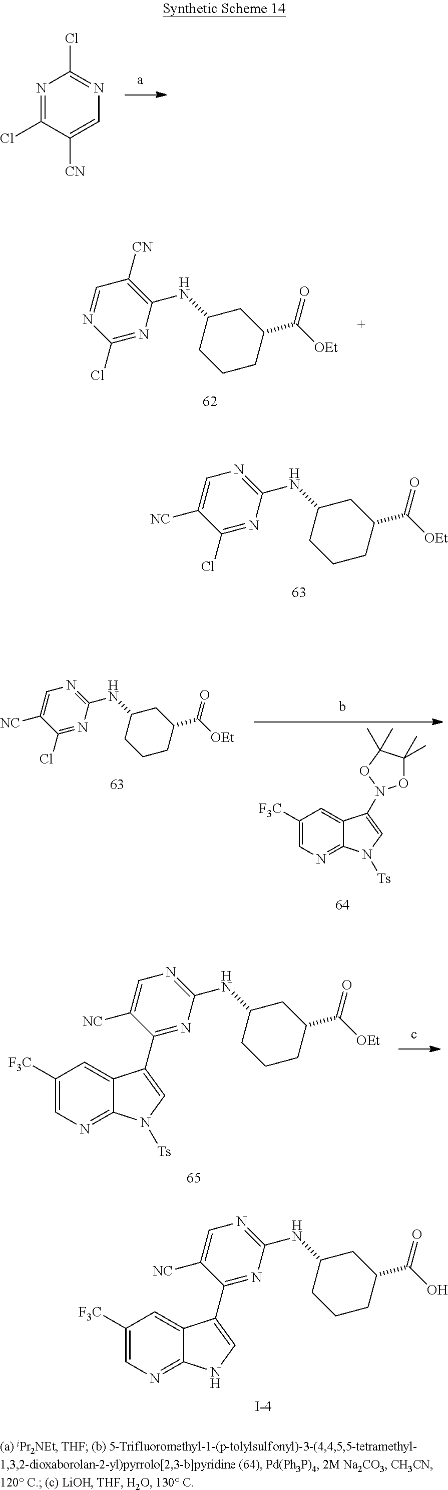

### Formation of (1R,3S)-ethyl 3-((4-chloro-5-cyanopyrimidin-2-yl)amino)cyclohexanecarboxylate (63)

To a solution of 2,4-dichloropyrimidine-5-carbonitrile (0.56 g, 3.23 mmol) and (1R,3S)-ethyl 3-aminocyclohexanecarboxylate, 56, (0.60 g) in THF (50 mL) was added N,N-diisopropylethylamine (1.40 mL, 8.07 mmol) in THF (10 mL). The reaction mixture was stirred at room temperature for 1 hour. The mixture was concentrated to dryness then dissolved in dichloromethane and washed with 1N HCl. The product was absorbed onto silica-gel and purified via silica gel chromatography (0-20% EtOAc/hexanes gradient) to afford 195 mg of the less polar (faster moving) product along with 542 mg of the more polar (slower moving) product, which was determined to be the desired product, 63.

### Formation of (1R,3S)-ethyl 3-((5-cyano-4-(1-tosyl-5-(trifluoromethyl)-1H-pyrrolo[2,3-b]pyridin-3-yl)pyrimidin-2-yl)amino)cyclohexanecarboxylate (65)

(1R,3S)-ethyl-3-[(4-chloro-5-cyano-pyrimidin-2-yl)amino]cyclohexanecarboxylate, 63, (0.165 g, 0.534 mmol), 1-(p-tolylsulfonyl)-3-(4,4,5,5-tetramethyl-1,3,2-dioxaborolan-2-yl)-5-(trifluoromethyl)pyrrolo[2,3-b]pyridine, 64, (0.295 g, 0.633 mmol), and Pd(PPh₃)₄ (0.098 g, 0.085 mmol) were dissolved in MeCN (12 mL) and treated with 2M Na₂CO₃. The mixture was irradiated in a microwave at 120° C. for 21 min. The mixture was poured into water and extracted with EtOAc. The organic phase was washed with brine and dried over Na₂SO₄, filtered and concentrated in vacuo. The crude residue was purified via silica gel chromatography (0-100% EtOAc/hexanes gradient) to afford 236 mg of the desired product.

### Formation of (1R,3S)-3-((5-cyano-4-(5-(trifluoromethyl)-1H-pyrrolo[2,3-b]pyridin-3-yl)pyrimidin-2-yl)amino)cyclohexanecarboxylic acid (I-4)

Dissolved (1S,3R)-ethyl 3-((5-cyano-4-(1-tosyl-5-(trifluoromethyl)-1H-pyrrolo[2,3-b]pyridin-3-yl)pyrimidin-2-yl)amino)cyclohexanecarboxylate, 65, (0.228 g, 0.372 mmol) in THF (10 mL) then treated with LiOH (2 mL of 1N solution, 2.000 mmol). The reaction mixture was irradiated in a microwave at 130° C. for 10 minutes. After cooling to room temperature the mixture was concentrated to reduced volume and then 3 mL 1N HCl was added to give a precipitate that was filtered and washed with water. The resulting solid was dissolved in EtOAc and filtered then concentrated to dryness to give 129 mg of an off-white solid: LCMS Gradient 10-90%, 0.1% formic acid, 5 min, C18/ACN, RT=4.08 (M+H) 431.47

### Preparation of Compound I-1, I-3, I-7, and I-8

The following compounds can be prepared in a similar fashion as the procedure described above for Compound I-4:

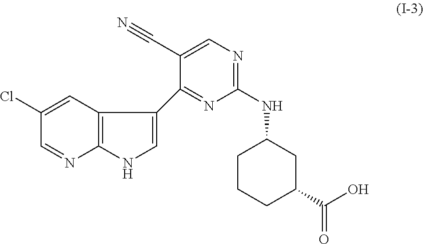 [SMILES: N#Cc1cnc(N[C@H]2CCC[C@@H](C(=O)O)C2)nc1-c1c[nH]c2ncc(Cl)cc12]

### (1R,3S)-3-((4-(5-chloro-1H-pyrrolo[2,3-b]pyridin-3-yl)-5-cyanopyrimidine-2-yl)amino)-cyclohexanecarboxylic acid

LCMS Gradient 10-90%, 0.1% formic acid, 5 min, C18/ACN, RT=4.09 minutes (M+H) 397.43

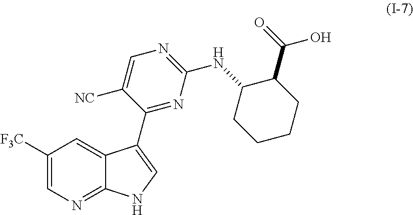

### (1S,2S)-2-((5-cyano-4-(5-(trifluoromethyl)-1H-pyrrolo[2,3-b]pyridin-3-yl)pyrimidin-2-yl)amino)cyclohexanecarboxylic acid

LCMS Gradient 10-90%, 0.1% formic acid, 5 min, C18/ACN, RT=4.08 minutes (M+H) 431.42

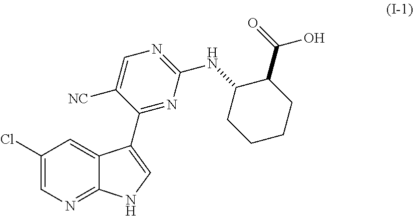

### (1S,2S)-2-((4-(5-chloro-1H-pyrrolo[2,3-b]pyridin-3-yl)-5-cyanopyrimidine-2-yl)amino)-cyclohexanecarboxylic acid

LCMS Gradient 10-90%, 0.1% formic acid, 5 min, C18/ACN, RT=4.04 minutes (M+H) 397.09

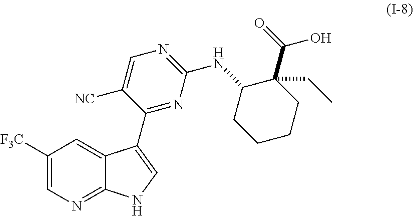

### (1S,2S)-2-((5-cyano-4-(5-(trifluoromethyl)-1H-pyrrolo[2,3-b]pyridin-3-yl)pyrimidin-2-yl)amino)-1-ethylcyclohexanecarboxylic acid

LCMS Gradient 10-90%, 0.1% formic acid, 5 min, C18/ACN, RT=4.13 minutes (M+H) 459.5

### Preparation of Compound I-15

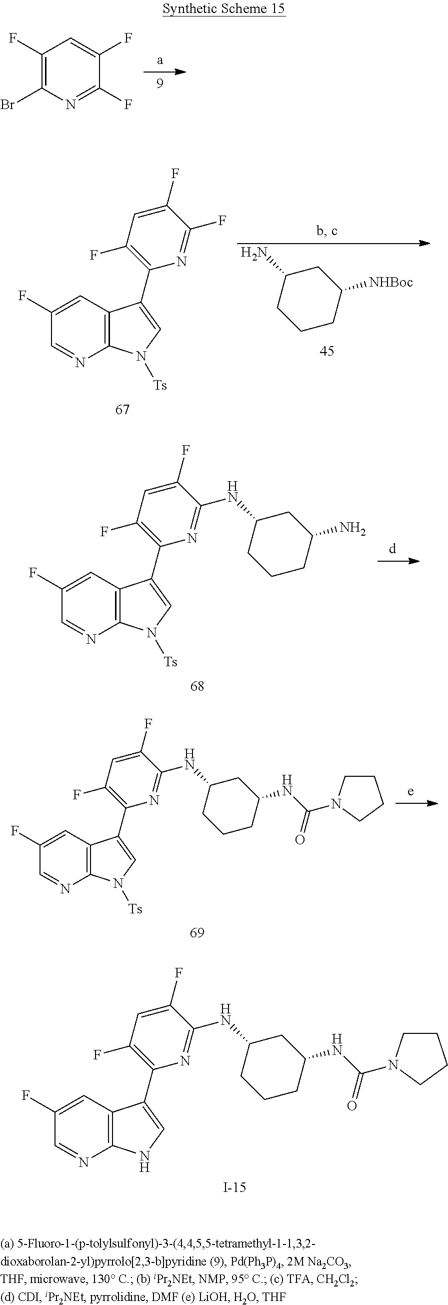

### Formation of 5-fluoro-1-tosyl-3-(3,5,6-trifluoropyridin-2-yl)-1H-pyrrolo[2,3-b]pyridine (67)

To a solution of 2-bromo-3,5,6-trifluoropyridine (0.79 g, 3.75 mmol) and 5-fluoro-1-(p-tolylsulfonyl)-3-(4,4,5,5-tetramethyl-1,3,2-dioxaborolan-2-yl)pyrrolo[2,3-b]pyridine (9) (1.30 g, 3.12 mmol) in THF (10 mL) was added Na₂CO₃ (4.68 mL of 2 M solution, 9.37 mmol). The solution was degassed with N₂ for 15 minutes. Pd(PPh₃)₄ (0.63 mmol) was added and the reaction was irradiated in a microwave for 30 minutes at 130° C. The organic phase was separated and upon shaking, a white precipitate formed quickly. The solution was diluted with acetonitrile, stirred for 30 minutes, filtered and the cake was washed again with acetonitrile to provide the product as a white solid: ¹H NMR (300 MHz, CDCl₃) δ 8.61 (dd, J=9.0, 2.8 Hz, 1H), 8.47 (d, J=2.3 Hz, 1H), 8.37 (dd, J=2.8, 0.8 Hz, 1H), 8.13 (d, J=8.4 Hz, 2H), 7.65-7.48 (m, 1H), 7.33 (d, J=8.1 Hz, 2H), 2.41 (s, 3H), 1.55 (s, 1H); LCMS Gradient 10-90%, 0.1% formic acid, 5 min, C18/ACN, RT=3.96 minutes (M+H) 422.22.

### Formation of (1S,3R)—N1-(3,5-difluoro-6-(5-fluoro-1-tosyl-1H-pyrrolo[2,3-b]pyridin-3-yl)pyridin-2-yl)cyclohexane-1,3-diamine (68)

To a solution of tert-butyl N-[(1R,3S)-3-aminocyclohexyl]carbamate-hydrochloric acid, 45, (0.89 g, 3.56 mmol) and 5-fluoro-1-tosyl-3-(3,5,6-trifluoropyridin-2-yl)-1H-pyrrolo[2,3-b]pyridine, 67, (1.00 g, 2.37 mmol) in NMP (5 mL) was added N,N-diisopropylethylamine (0.83 mL, 4.75 mmol). The reaction was stirred at 95° C. for two days in a sealed flask. The mixture was diluted with EtOAc and washed with H₂O. The aqueous phase was extracted with EtOAc, which was then washed again with H₂O. The combined organic phases were dried with Na₂SO₄, filtered and concentrated in vacuo. The crude product, which contains 5-fluoro-3-(3,5,6-trifluoro-2-pyridyl)-1H-pyrrolo[2,3-b]pyridine as an impurity, was utilized in the next step without further purification: LCMS RT=4.12 minutes (M+H) 616.41.

The crude product (0.40 g, 0.65 mmol) was treated with trifluoroacetic acid (0.18 mL, 2.37 mmol) in dichloromethane for 1 hour. The solvent was then evaporated and the crude product was re-dissolved in small amount of dichloromethane and added to 1 M HCl/Et₂O solution with stirring. After 30 mins the white solid was filtered and washed with dry Et₂O to provide the desired product, 68, which still contains 5-fluoro-3-(3,5,6-trifluoro-2-pyridyl)-1H-pyrrolo[2,3-b]pyridine impurity. The mixture was utilized in the next step without further purification. LCMS Gradient 10-90%, 0.1% formic acid, 5 min, C18/ACN, RT=2.19 minutes (M+H) 516.40.

### Formation of N-((1R,3S)-3-((3,5-difluoro-6-(5-fluoro-1-tosyl-1H-pyrrolo[2,3-b]pyridin-3-yl)pyridin-2-yl)amino)cyclohexyl)pyrrolidine-1-carboxamide (69)

To a solution of (1S,3R)—N1-[3,5-difluoro-6-[5-fluoro-1-(p-tolylsulfonyl)pyrrolo[2,3-b]pyridin-3-yl]-2-pyridyl]cyclohexane-1,3-diamine, 68, (0.050 g, 0.097 mmol) in DMF (1 mL) was added N,N-diisopropylethylamine (0.051 mL, 0.291 mmol). To the stirred solution was added carbonyl diimidazole (0.016 g, 0.097 mmol). The reaction was allowed to stir for 1 hour at room temperature. Another portion of same equivalence of carbonyl diimidazole was added and the reaction was stirred for another 2 hours. Pyrrolidine (0.008 mL, 0.097 mmol) was added and the reaction was stirred overnight at room temperature. DMF was evaporated and the crude product was utilized in the next step without further purification: LCMS Gradient 10-90%, 0.1% formic acid, 5 min, C18/ACN, RT=3.82 minutes (M+H) 613.53.

### Formation of N-((1R,3S)-3-((3,5-difluoro-6-(5-fluoro-1H-pyrrolo[2,3-b]pyridin-3-yl)pyridin-2-yl)amino)cyclohexyl)pyrrolidine-1-carboxamide (I-15)

Crude N-((1R,3S)-3-((3,5-difluoro-6-(5-fluoro-1-tosyl-1H-pyrrolo[2,3-b]pyridin-3-yl)pyridin-2-yl)amino)cyclohexyl)pyrrolidine-1-carboxamide, 69, was dissolved in THF (1 mL) and LiOH (0.10 mL of 2 M solution, 2.00 mmol) was added. The reaction was heated to 90° C. for 6 hours. The crude product was purified by preparatory HPLC (0.1% TFA-CH₃CN/H₂O) to provide 4 mg of the desired product as a trifluoroacetic acid salt: LCMS Gradient 10-90%, 0.1% formic acid, 5 min, C18/ACN, RT=2.97 minutes (M+H) 459.00.

### Preparation of Compounds I-16, I-17, I-18, I-23, I-24, and I-25

The following compounds can be prepared in a similar fashion as the procedure described above for Compound I-15:

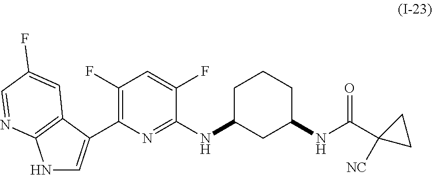

### N-((1R,3S)-3-((6-(5-chloro-1H-pyrazolo[3,4-b]pyridin-3-yl)-3,5-difluoropyridin-2-yl)-amino)cyclohexyl)-1-cyanocyclopropanecarboxamide

LCMS Gradient 10-90%, 0.1% formic acid, 5 min, C18/ACN, RT=3.10 minutes (M+H) 455.49; RT=3.10 minutes.

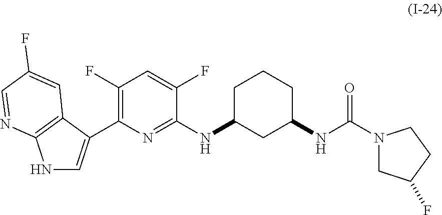

### (S)—N-((1R,3S)-3-((3,5-difluoro-6-(5-fluoro-1H-pyrrolo[2,3-b]pyridin-3-yl)pyridin-2-yl)-amino)cyclohexyl)-3-fluoropyrrolidine-1-carboxamide

LCMS Gradient 10-90%, 0.1% formic acid, 5 min, C18/ACN RT=2.87 minutes (M+H) 477.57.

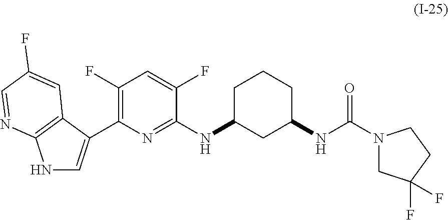

### N-((1R,3S)-3-((3,5-difluoro-6-(5-fluoro-1H-pyrrolo[2,3-b]pyridin-3-yl)pyridin-2-yl)-amino)cyclohexyl)-3,3-difluoropyrrolidine-1-carboxamide

LCMS Gradient 10-90%, 0.1% formic acid, 5 min, C18/ACN RT=3.05 minutes (M+H) 495.61.

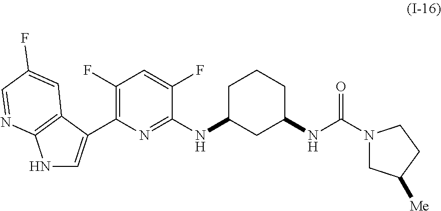

### (R)—N-((1R,3S)-3-((3,5-difluoro-6-(5-fluoro-1H-pyrrolo[2,3-b]pyridin-3-yl)-pyridin-2-yl)amino)cyclohexyl)-3-methylpyrrolidine-1-carboxamide

LCMS Gradient 10-90%, 0.1% formic acid, 5 min, C18/ACN, RT=3.12 minutes (M+H) 473.47.

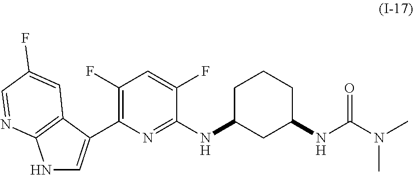

### 3-((1R,3S)-3-((3,5-difluoro-6-(5-fluoro-1H-pyrrolo[2,3-b]pyridin-3-yl)pyridin-2-yl)amino)cyclohexyl)-1,1-dimethylurea

LCMS Gradient 10-90%, 0.1% formic acid, 5 min, C18/ACN, RT=2.84 minutes (M+H) 433.42.

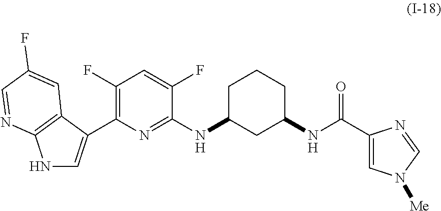

### N-((1R,3S)-3-((3,5-difluoro-6-(5-fluoro-1H-pyrrolo[2,3-b]pyridin-3-yl)pyridin-2-yl)-amino)cyclohexyl)-1-methyl-1H-imidazole-4-carboxamide

LCMS Gradient 10-90%, 0.1% formic acid, 5 min, C18/ACN RT 2.37 minutes (M+H) 470.46.

### Preparation of Compounds I-12 and I-13

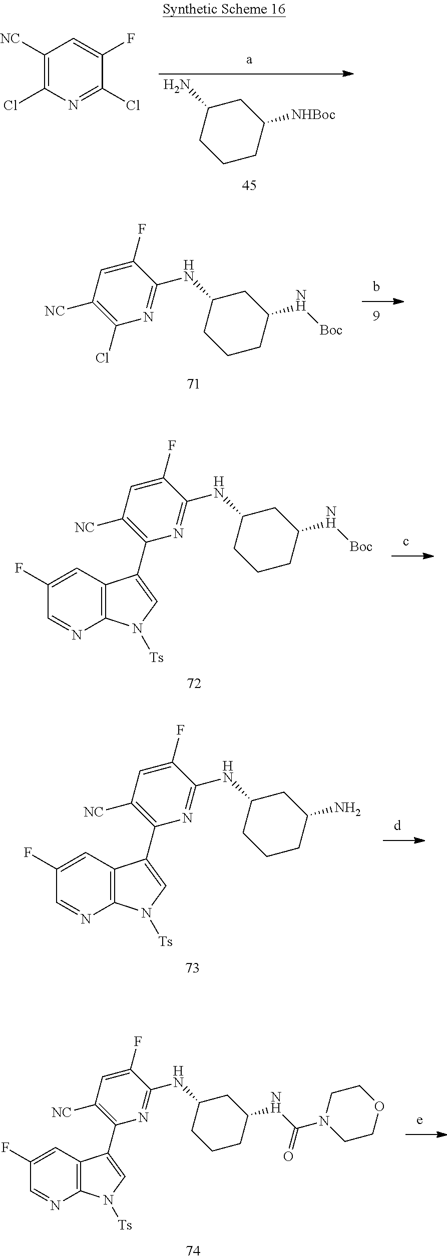 [SMILES: N#Cc1cc(F)c(Cl)nc1Cl ; CC(C)(C)OC(=O)N[C@@H]1CCC[C@H](N)C1 ; CC(C)(C)OC(=O)N[C@@H]1CCC[C@H](Nc2nc(Cl)c(C#N)cc2F)C1 ; Cc1ccc(S(=O)(=O)n2cc(-c3nc(N[C@H]4CCC[C@@H](NC(=O)OC(C)(C)C)C4)c(F)cc3C#N)c3cc(F)cnc32)cc1 ; Cc1ccc(S(=O)(=O)n2cc(-c3nc(N[C@H]4CCC[C@@H](N)C4)c(F)cc3C#N)c3cc(F)cnc32)cc1 ; Cc1ccc(S(=O)(=O)n2cc(-c3nc(N[C@H]4CCC[C@@H](NC(=O)N5CCOCC5)C4)c(F)cc3C#N)c3cc(F)cnc32)cc1]

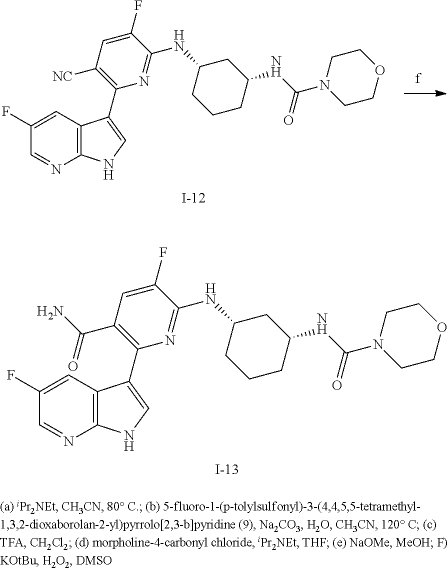

### Formation of tert-butyl ((1R,3S)-3-((6-chloro-5-cyano-3-fluoropyridin-2-yl)amino)cyclohexyl)carbamate (71)

A solution of tert-butyl N-[(1R,3S)-3-aminocyclohexyl]carbamate, 45, (0.064 g, 0.300 mmol), 2,6-dichloro-5-fluoro-pyridine-3-carbonitrile (0.057 g, 0.300 mmol) and N,N-diisopropylethylamine (0.078 mL, 0.450 mmol) in CH₃CN (3 mL) were refluxed for 24 hours. The solvent was removed in vacuo and the residue was dissolved with ether (10 mL) and water (10 mL). The aqueous layer was washed with ether (10 mL) and the combined organic phases were washed with brine, dried (MgSO₄), filtered and concentrated in vacuo to give crude product, which was used without further purification: LCMS Gradient 10-90%, 0.1% formic acid, 5 min, C18/ACN, (M+H) 369.24.

### Formation of tert-butyl ((1R,3S)-3-((5-cyano-3-fluoro-6-(5-fluoro-1-tosyl-1H-pyrrolo[2,3-b]pyridin-3-yl)pyridin-2-yl)amino)cyclohexyl)carbamate (72)

To a solution of tert-butyl N-[(1R,3S)-3-[(6-chloro-5-cyano-3-fluoro-2-pyridyl)amino]cyclohexyl]carbamate, 71, (0.37 g, 1.00 mmol), 5-fluoro-1-(p-tolylsulfonyl)-3-(4,4,5,5-tetramethyl-1,3,2-dioxaborolan-2-yl)pyrrolo[2,3-b]pyridine, 9, (0.42 g, 1.00 mmol) Pd(Ph₃P)₄ (0.12 g, 0.10 mmol) in CH₃CN was added disodium carbonate (0.40 mL of 2 M solution, 0.79 mmol). The reaction was heated to reflux for 3 hours. The reaction was diluted into ethyl acetate and water. The organic phase was separated, dried (MgSO₄), filtered and concentrated in vacuo to give crude product. Purification via silica gel chromatography (0-100% ethyl acetate/hexane gradient) afforded 366 mg of the desired product as a yellow solid: ¹H NMR (300 MHz, CDCl₃) δ 8.66 (s, 1H), 8.28 (d, J=1.8 Hz, 1H), 8.15 (dd, J=8.9, 2.8 Hz, 1H), 8.05 (d, J=8.4 Hz, 2H), 7.39-7.28 (m, 2H), 7.24 (d, J^{=8.1} Hz, 2H), 5.03 (dd, J=7.7, 2.3 Hz, 1H), 4.37 (s, 1H), 3.48 (s, 1H), 2.45-2.22 (m, 1H), 2.13 (d, J=12.3 Hz, 1H), 1.99 (m, J=7.2 Hz, 1H), 1.88-1.70 (m, 1H), 1.48-1.25 (m, 9H), 1.20-0.90 (m, 4H); ¹⁹F NMR (282 MHz, CDCl3) δ°−133.04, 142.37; LCMS Gradient 10-90%, 0.1% formic acid, 5 min, C18/ACN, (M+H) 623.32.

### Formation of 6-(((1S,3R)-3-aminocyclohexyl)amino)-5-fluoro-2-(5-fluoro-1-tosyl-1H-pyrrolo[2,3-b]pyridin-3-yl)nicotinonitrile (73)

To a solution of tert-butyl N-[(1R,3S)-3-[[6-[5-chloro-1-(p-tolylsulfonyl)pyrrolo[2,3-b]pyridin-3-yl]-5-cyano-3-fluoro-2-pyridyl]amino]cyclohexyl]carbamate (0.367 g, 0.573 mmol) in dichloromethane (10 mL) was added trifluoroacetic acid (1.5 mL). After stirring at room temperature for one hour, the mixture was concentrated to dryness to give 385 mg of a light yellow solid that was used without further purification: LCMS Gradient 10-90%, 0.1% formic acid, 5 min, C18/ACN, (M+H) 523.25.

### Formation of N-((1R,3S)-3-((5-cyano-3-fluoro-6-(5-fluoro-1-tosyl-1H-pyrrolo[2,3-b]pyridin-3-yl)pyridin-2-yl)amino)cyclohexyl)morpholine-4-carboxamide (74)

To a solution of 6-(((1S,3R)-3-aminocyclohexyl)amino)-5-fluoro-2-(5-fluoro-1-tosyl-1H-pyrrolo[2,3-b]pyridin-3-yl)nicotinonitrile, 73, (0.060 g, 0.080 mmol) and N,N-diisopropylethylamine (0.400 mmol) in THF (2 ml) was added morpholine-4-carbonyl chloride (0.012 mL, 0.100 mmol). The reaction was stirred for 3 h at room temperature and concentrated in vacuo. The crude product was purified by silica gel chromatography (0-20% MeOH/CH₂Cl₂ gradient) to afford 58 mg of desired product as a yellow solid: LCMS Gradient 10-90%, 0.1% formic acid, 5 min, C18/ACN, (M+H) 636.31.

### Formation of N-((1R,3S)-3-((5-cyano-3-fluoro-6-(5-fluoro-1H-pyrrolo[2,3-b]pyridin-3-yl)pyridin-2-yl)amino)cyclohexyl)morpholine-4-carboxamide (I-12)

N-((1R,3S)-3-((5-cyano-3-fluoro-6-(5-fluoro-1-tosyl-1H-pyrrolo[2,3-b]pyridin-3-yl)-pyridin-2-yl)amino)cyclohexyl)morpholine-4-carboxamide, 74, (0.058 g, 0.091 mmol) was dissolved in methanol (5 ml). Sodium methoxide (0.021 mL of a 34.35 M solution in methanol, 0.091 mmol) was added to the mixture. After 10 minutes of stirring at room temperature, the reaction was neutralized with HCl (0.07 mL, 1.25M solution in MeOH) and concentrated to dryness. The crude product was purified by silica gel chromatography (0-10% MeOH/CH₂Cl₂ gradient) to give the product as a light yellow solid. Re-acidification with HCl provides the HCl salt as yellow solid (33 mg): ¹H NMR (300 MHz, MeOD) δ 8.73 (dd, J=9.2, 2.6 Hz, 1H), 8.50 (s, 1H), 8.43 (s, 1H), 7.52 (d, J=10.9 Hz, 1H), 4.22-4.02 (m, 1H), 3.83-3.67 (m, 1H), 3.67-3.57 (m, 4H), 3.41-3.32 (m, 4H), 2.31 (d, J^{=11.8} Hz, 1H), 2.15 (d, J=12.4 Hz, 1H), 1.92 (dd, J=26.4, 10.0 Hz, 2H), 1.70-1.40 (m, 2H), 1.33 (dt, J=24.6, 8.2 Hz, 2H); ¹⁹F NMR (282 MHz, MeOD) δ−138.34, −145.45; LCMS Gradient 10-90%, 0.1% formic acid, 5 min, C18/ACN, (M+H) 482.30.

### Formation of N-((1R,3S)-3-((5-carbamoyl-3-fluoro-6-(5-fluoro-1H-pyrrolo[2,3-b]pyridin-3-yl)pyridin-2-yl)amino)cyclohexyl)morpholine-4-carboxamide (I-13)

To a solution of N-((1R,3S)-3-((5-cyano-3-fluoro-6-(5-fluoro-1H-pyrrolo[2,3-b]pyridin-3-yl)pyridin-2-yl)amino)cyclohexyl)morpholine-4-carboxamide, I-12, (0.020 g, 0.040 mmol) in DMSO (1 mL) was added H₂O₂(0.400 mL of 30% w/w, 0.395 mmol) followed by K₂CO₃ (0.050 g, 0.362 mmol). The reaction was heated overnight at 45° C. The DMSO solution was used directly in the purification via preparatory HPLC (0.1% TFA-CH₃CN/H₂O) to give 10 mg of the product as a TFA salt. After neutralization by passing a methanol solution of the salt through a bicarbonate cartridge, the solution was re-acidified with hydrochloric acid in methanol and dried under a stream of nitrogen to furnish 10 mg of the desired product as a hydrochloride salt: ¹H NMR (300 MHz, MeOD) δ 8.17 (d, J=9.4 Hz, 1H), 7.87 (s, 1H), 7.46 (d, J=11.2 Hz, 1H), 4.26-4.02 (m, 1H), 3.79-3.65 (m, 1H), 3.67-3.54 (m, 4H), 3.39-3.32 (m, 4H), 2.29 (d, J=11.6 Hz, 1H), 2.14 (d, J=10.9 Hz, 1H), 1.92 (dd, J=18.4, 11.6 Hz, 2H), 1.64-1.43 (m, 1H), 1.44 (d, J=6.0 Hz, 4H); ¹⁹F NMR (282 MHz, MeOD) δ −139.14, −139.39; LCMS Gradient 10-90%, 0.1% formic acid, 5 min, C18/ACN, (M+H) 500.34.

### Preparation of Compounds I-11, I-14, and I-43

The following compounds can be prepared in a similar fashion using the procedure described above for Compounds I-12 and I-13:

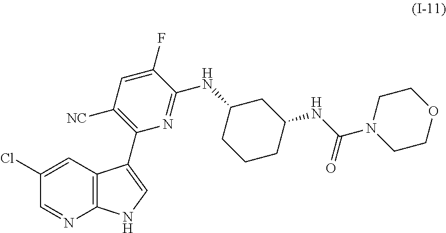 [SMILES: N#Cc1cc(F)c(N[C@H]2CCC[C@@H](NC(=O)N3CCOCC3)C2)nc1-c1c[nH]c2ncc(Cl)cc12]

### N-((1R,3S)-3-((6-(5-chloro-1H-pyrrolo[2,3-b]pyridin-3-yl)-5-cyano-3-fluoro-pyridin-2-yl)amino)cyclohexyl)morpholine-4-carboxamide

LCMS Gradient 10-90%, 0.1% formic acid, 5 min, C18/ACN, (M+H) 498.23

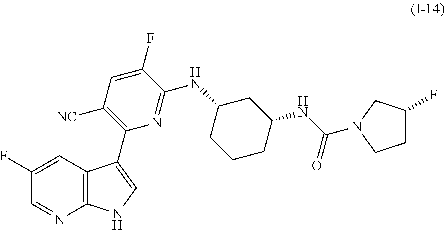 [SMILES: N#Cc1cc(F)c(N[C@H]2CCC[C@@H](NC(=O)N3CC[C@@H](F)C3)C2)nc1-c1c[nH]c2ncc(F)cc12]

### (R)—N-((1R,3S)-3-((5-cyano-3-fluoro-6-(5-fluoro-1H-pyrrolo[2,3-b]pyridin-3-yl)pyridin-2-yl)amino)cyclohexyl)-3-fluoropyrrolidine-1-carboxamide

LCMS Gradient 10-90%, 0.1% formic acid, 5 min, C18/ACN, (M+H) 484.28

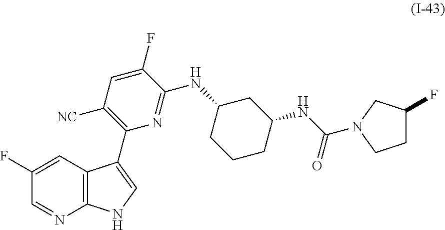 [SMILES: N#Cc1cc(F)c(N[C@H]2CCC[C@@H](NC(=O)N3CC[C@H](F)C3)C2)nc1-c1c[nH]c2ncc(F)cc12]

### (S)—N-((1R,3S)-3-((5-cyano-3-fluoro-6-(5-fluoro-1H-pyrrolo[2,3-b]pyridin-3-yl)pyridin-2-yl)amino)cyclohexyl)-3-fluoropyrrolidine-1-carboxamide

LCMS Gradient 10-90%, 0.1% formic acid, 5 min, C18/ACN, (M+H) 484.29

### Preparation of Compounds I-44

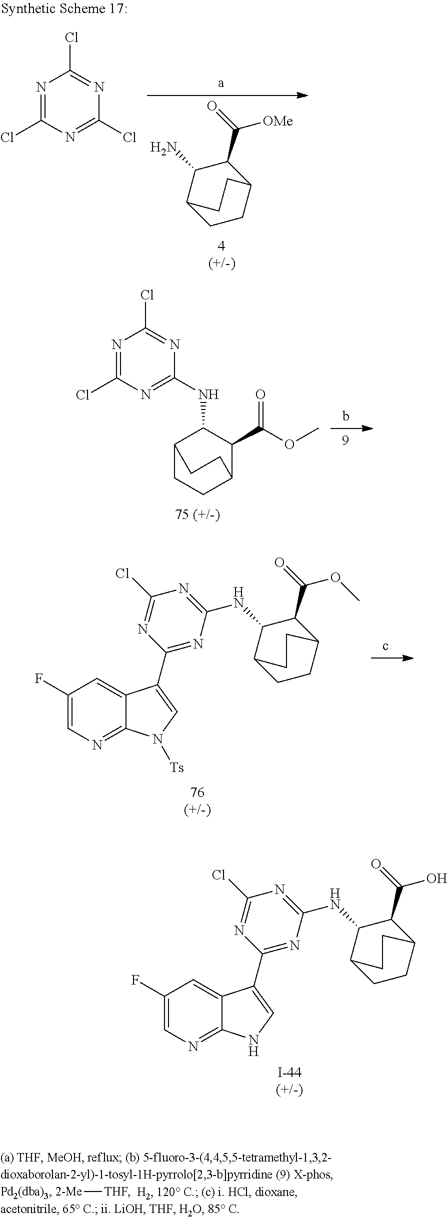

### Formation of (+/−)-trans-methyl 3-(4,6-dichloro-1,3,5-triazin-2-ylamino)bicyclo-[2.2.2]octane-6-carboxylate (75)

A solution of racemic trans-methyl 3-aminobicyclo[2.2.2]octane-2-carboxylate, 4, (0.50 g, 2.95 mmol), 2,4,6-trichloro-1,3,5-triazine (0.55 g, 2.96 mmol) and N,N-diisopropylethylamine (0.56 mL, 2.95 mmol) in THF (8 mL) and MeOH (2 mL) was heated at 85° C. for 3 hr. The solvent was evaporated under reduced pressure. The crude product was purified by silica gel chromatography (0%-30% EtOAc/hexanes gradient) to afford 830 mg of the title compound as an oil: ¹H NMR (400 MHz, CDCl₃) δ 8.55 (s, 1H), 8.31 (s, 1H), 5.20 (m, 1H), 3.74 (s, 3H), 2.62 (m, 1H), 2.53 (m, 1H), 2.24 (s, 1H), 2.04 (m, 1H), 1.98-1.63 (m, 7H); LCMS Gradient 10-90%, 0.1% formic acid, 5 min, C18/ACN, RT=3.45 minutes (M+H) 331.25.

### Formation of (+/−)-trans-methyl 3-(4-chloro-6-(5-fluoro-1-tosyl-1H-pyrrolo[2,3-b]pyridin-3-yl)-1,3,5-triazin-2-ylamino)bicyclo[2.2.2]octane-2-carboxylate (76)

A solution of racemic trans-methyl 3-(4,6-dichloro-1,3,5-triazin-2-ylamino)bicyclo-[2.2.2]octane-6-carboxylate, 75, (0.38 g, 1.14 mmol), 5-fluoro-1-(p-tolylsulfonyl)-3-(4,4,5,5-tetramethyl-1,3,2-dioxaborolan-2-yl)pyrrolo[2,3-b]pyridine, 9, (0.24 g, 0.57 mmol) and K₃PO₄ (0.72 g, 3.41 mmol) in 2-methyl THF (4.0 mL) and H₂O (0.5 mL) was degassed with a stream of nitrogen for 20 min and Pd(PPh₃)₄ (0.07 g, 0.06 mmol) was added. The reaction mixture was heated at 120° C. in a sealed tube for 3 hr. The reaction mixture was cooled to room temperature and concentrated in vacuo. The crude product was purified by silica gel chromatography (0-50% EtOAc/hexanes gradient) to afford 180 mg of the title compound as a white foamy solid: ¹H NMR (300 MHz, CDCl₃) δ 8.85 (s, 1H), 8.50 (m, 1H), 8.35 (s, 1H), 8.14 (d, J=8.8 Hz, 2H), 7.33 (d, J=8.3 Hz, 2H), 5.78 (m, 1H), 4.74 (m, 1H), 4.50 (s, 1H), 3.72 (s, 3H), 2.41 (s, 3H), 2.18-1.41 (m, 10H); LCMS (60-98% ACN/water 7 min with 0.9% FA, C4) m/z 585.05 (M+1) retention time 2.72 min.

### Formation of (+/−)-trans-3-(4-chloro-6-(5-fluoro-1H-pyrrolo[2,3-b]pyridin-3-yl)-1,3,5-triazin-2-ylamino)bicyclo[2.2.2]octane-2-carboxylic acid (I-44)

HCl (0.77 mL of 4M solution in dioxane, 3.07 mmol) was added to a stirred solution of racemic-trans-methyl 3-(4-chloro-6-(5-fluoro-1-tosyl-1H-pyrrolo[2,3-b]pyridin-3-yl)-1,3,5-triazin-2-ylamino)bicyclo[2.2.2]octane-2-carboxylate, 76, in CH₃CN (5 mL). The mixture was heated at 65° C. for 3 hr and cooled to room temperature. The solvent was evaporated and residue was washed with ether (10 mL). The residue was dissolved in THF (5 mL) and a solution of lithium hydroxide (1.5 mL of 2N solution, 3.07 mmol) was added. The solution was heated at 85° C. for 8 hr and cooled to room temperature. The reaction mixture was concentrated in vacuo. The crude product was purified by preparative HPLC (10-80% water/CH₃CN and water, 0.1% TFA, 15 min) to afford 5 mg of the title compound as a white foam: ¹H NMR (300 MHz, MeOD) δ 8.56 (dd, J=9.3, 2.6 Hz, 1H), 8.45 (s, 1H), 8.21 (s, 1H), 4.76 (m, 1H), 2.62 (m, 2H), 2.07-1.52 (m, 9H); LCMS (10-90% ACN/water 5 min with 0.9% FA) m/z 417.09 (M+1) retention time 3.03 min.

### Preparation of Compound 45

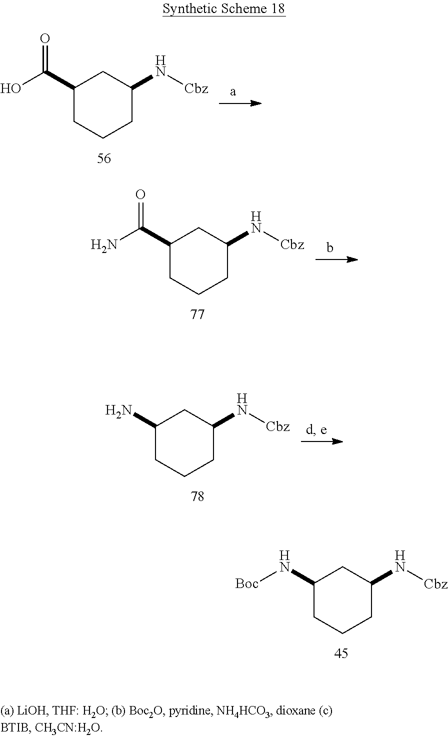 [SMILES: O=C(N[C@H]1CCC[C@@H](C(=O)O)C1)OCc1ccccc1 ; NC(=O)[C@@H]1CCC[C@H](NC(=O)OCc2ccccc2)C1 ; N[C@@H]1CCC[C@H](NC(=O)OCc2ccccc2)C1 ; CC(C)(C)OC(=O)N[C@@H]1CCC[C@H](NC(=O)OCc2ccccc2)C1]

### Formation of (1R,3S)-3-benzyloxycarbonylaminocyclohexanecarboxylic acid (56)

Ethyl (1R,3S)-3-benzyloxycarbonylaminocyclohexanecarboxylate (36.0 g, 117.9 mmol) was dissolved in THF (144.0 mL) and treated with a solution of LiOH (5.7 g, 235.8 mmol) in water (216.0 mL). After stirring overnight, the reaction mixture was diluted with water (100 mL), washed with methyl tert-butyl ether (150 mL) and brought to pH 3 by addition of 3N HCl. The acidic solution was extracted with EtOAc (3×100 mL), and the combined organic layers were washed with water and brine, dried on Na₂SO₄ and concentrated in vacuo.

The crude product was triturated with methyl tert-butyl ether (30 mL) and filtered to provide a first crop of crystals. The filtrate was treated with heptane (20 mL), concentrated to 30 mL and allowed to stand at room temperature for 3 hours to provide a second crop of crystals that were collected by filtration for a total of 14.4 g of desired product: ¹H NMR (300 MHz, CDCl₃) δ 7.38-7.33 (m, 5H), 5.11 (s, 2H), 4.68 (s, 1H), 3.55 (s, 1H), 2.44 (d, J=11.0 Hz, 1H), 2.32 (d, J=11.7 Hz, 1H), 2.03-1.86 (m, 3H) and 1.48-0.88 (m, 4H) ppm.

### Formation of benzyl N-[(1S,3R)-3-carbamoylcyclohexyl]carbamate (77)

To a solution of (1R,3S)-3-Benzyloxycarbonylaminocyclohexanecarboxylic acid, 56, (10.0 g, 36.1 mmol) in 1,4-dioxane (300 mL) was added pyridine (2.9 mL, 36.1 mmol), followed by di-tert-butyl dicarbonate (10.7 mL, 46.9 mmol) and ammonium bicarbonate (10.1 g, 126.2 mmol). After 3 hours, another portion of di-tert-butyl dicarbonate (1.5 g, 6.8 mmol) and ammonium bicarbonate (1.5 g, 6.8 mmol) was added and stirring was continued overnight. The reaction was quenched by addition of 2N HCl (400 mL) and stirred for 1 hour. The resulting suspension was filtered under reduced pressure, washed with 2N HCl (50 mL), water (8×50 mL) and hexanes (3×50 mL) and vacuum dried to provide benzyl N-[(1S,3R)-3-carbamoylcyclohexyl]carbamate (9.1 g, 91%) as a white solid: ¹H NMR (300 MHz, CDCl₃) δ 7.40-7.24 (m, 5H), 5.08 (s, 2H), 3.58-3.44 (m, 1H), 2.38-2.21 (m, 1H), 2.17 (d, J=12.7, 1H), 2.05-1.78 (m, 8H), 1.54-0.97 (m, 5H).

### Formation of benzyl N-[(1S,3R)-3-aminocyclohexyl]carbamate (78)

Benzyl N-[(1S,3R)-3-carbamoylcyclohexyl]carbamate, 77, (9.1 g, 32.9 mmol) was suspended in a mixture of acetonitrile (100 mL) and water (100 mL) and treated with. bis(trifluoroacetoxy)iodobenzene (15.5 g, 36.1 mmol). The suspension was allowed to stir at room temperature overnight and was then quenched with 1N HCl (100 mL). After evaporation of the acetonitrile, the acidic aqueous solution was washed with EtOAc (2×150 mL). The pH was adjusted to basic by addition of solid KOH and the resulting emulsion was extracted with EtOAc (3×200 mL). The combined organic layers were dried over Na₂SO₄ and concentrated in vacuo to provide 6.2 g of the desired product: ¹H NMR (300 MHz, CDCl₃) δ 7.31-7.45 (m, 5H), 5.11 (s, 2H), 4.90 (br. s., 1H), 3.58 (br. s., 1H), 2.72-2.97 (m, 1H), 2.14 (d, J=11.90 Hz, 1H), 1.87-2.02 (m, 1H), 1.73-1.87 (m, 2H), 1.21-1.46 (m, 1H), 0.89-1.18 (m, 3H).

### Formation of benzyl tert-butyl (1R,3S)-cyclohexane-1,3-diyldicarbamate

To a solution of benzyl N-[(1S,3R)-3-aminocyclohexyl]carbamate, 78, (2.04 g, 8.22 mmol) in THF (20 mL) was added potassium carbonate (3.41 g, 24.64 mmol) followed by di-tert-butyldicarbonate (1.97 g, 9.04 mmol). The reaction mixture was stirred overnight at room temperature. The solids were filtered and the filtrate was concentrated in vacuo. The crude residue was purified by silica gel chromatography (10%-25% EtOAc/hexanes) to give the desired Boc-protected intermediate.

### Formation of tert-butyl ((1R,3S)-3-aminocyclohexyl)carbamate (45)

To a solution benzyl tert-butyl (1R,3S)-cyclohexane-1,3-diyldicarbamate (168.0 g, 0.5 mol) in MeOH (2 L) was added Pd/C 10% (24 g). After flushing with nitrogen. The mixture was stirred under 1 bar hydrogen pressure. Conversion had reached 80% overnight according to NMR. After an additional 48 h the conversion was complete. The mixture was filtered through Celite and the filter cake was washed with MeOH. Concentration of the filtrate gave the final product (103 g) that was used without further purification.

### Preparation of Compounds I-47 and I-48

Compounds I-47 and I-48 were prepared in a similar fashion as described above for Compound I-18.

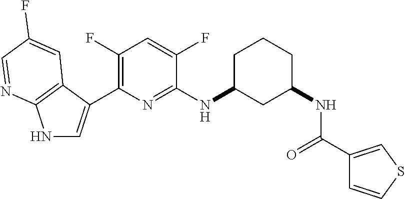

### Formation of N-((1R,3S)-3-((3,5-difluoro-6-(5-fluoro-1H-pyrrolo[2,3-b]pyridin-3-yl)pyridin-2-yl)amino)cyclohexyl)thiophene-3-carboxamide (I-47)

LCMS Gradient 10-90%, 0.1% formic acid, 5 min, C18/ACN, RT=3.17 min, (M+H) 472.25.

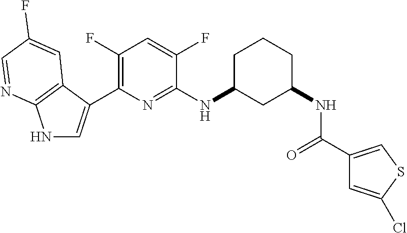

### Formation of 5-chloro-N-((1R,3S)-3-((3,5-difluoro-6-(5-fluoro-1H-pyrrolo[2,3-b]pyridin-3-yl)pyridin-2-yl)amino)cyclohexyl)thiophene-3-carboxamide (I-48)

LCMS Gradient 10-90%, 0.1% formic acid, 5 min, C18/ACN, RT=3.50 min, (M+H) 506.17.

### Preparation of Compound I-49

The compound was prepared in a similar fashion as described above, for example, for Compound I-28.

### Formation of 3-(2-(5-fluoro-1H-pyrrolo[2,3-b]pyridin-3-yl)pyridin-4-ylamino)bicyclo[2.2.2]octane-2-carboxylic acid (I-49)

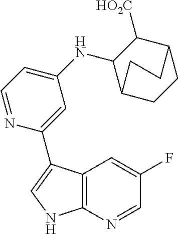 [SMILES: O=C(O)C1C2CCC(CC2)C1Nc1ccnc(-c2c[nH]c3ncc(F)cc23)c1]

¹H NMR (300 MHz, DMSO) δ 8.63 (d, J=8.0 Hz, 1H), 8.34-8.16 (m, 2H), 7.44 (s, 1H), 7.04 (d, J=7.4 Hz, 1H), 6.36 (s, 1H), 4.45 (s, 1H), 1.96 (d, J=20.7 Hz, 2H), 1.86-1.62 (m, 3H), 1.61-1.26 (m, 4H).

### Preparation of Compound I-50

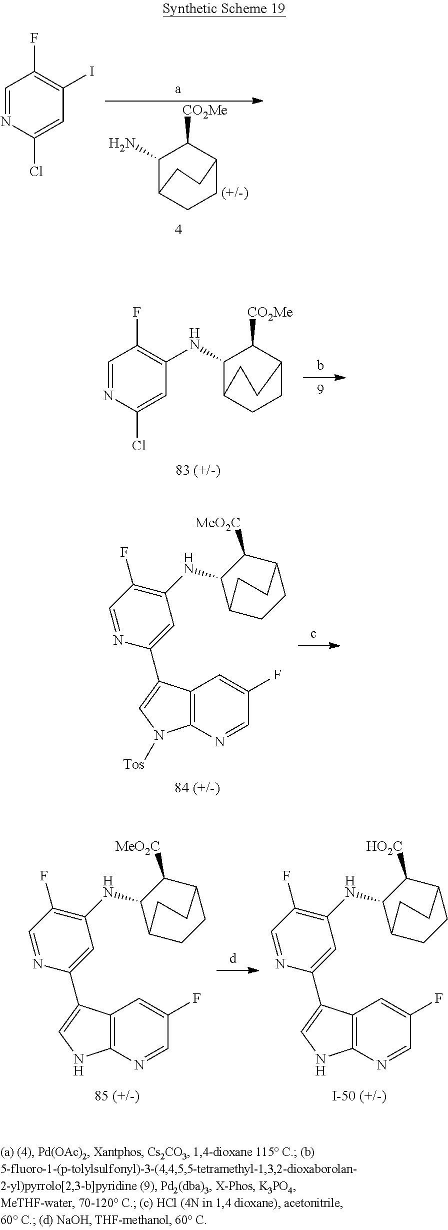

### Formation of (+/−)-(2,3)-trans-methyl 3-((2-chloro-5-fluoropyridin-4-yl)amino)-bicyclo[2.2.2]octane-2-carboxylate (83)

A solution of 2-chloro-5-fluoro-4-iodo-pyridine (0.200 g, 0.777 mmol), racemic-(2,3)-trans-methyl 3-aminobicyclo[2.2.2]octane-2-carboxylate, 4, (0.157 g, 0.855 mmol), Cs₂CO₃ (0.506 g, 1.550 mmol) and Xantphos (0.027 g, 0.047 mmol) in 1,4-dioxane (4.75 mL) was degassed under a stream of N₂. To this mixture was added Pd(OAc)₂ (0.009 g, 0.039 mmol). The vial was capped, heated to 115° C. and the yellow suspension was stirred overnight. After 18 hours, the reaction was cooled to room temperature. The mixture was poured into water (10 mL) and extracted with CH₂Cl₂ (3×20 mL). The combined organic phases were dried over MgSO₄, filtered and concentrated in vacuo. Purification by flash chromatography (SiO₂, 0-100% EtOAc-hexanes, gradient elution) provided the desired product (165 mg, 65% yield): ¹H NMR (300 MHz, CDCl₃) δ 7.89 (d, J=2.6 Hz, 1H), 6.63 (d, J=6.1 Hz, 1H), 4.71-4.39 (m, 1H), 4.11-3.97 (m, 1H), 3.73 (s, 3H), 2.34 (d, J=5.8 Hz, 1H), 2.19-2.05 (m, 1H), 1.86 (s, 1H), 1.80-1.38 (m, 10H) ppm; LCMS Gradient: 10-90%, TFA, 5 min, C18/AcN, Retention Time=3.14 min, (M+H) 313.02.

### Formation of (+/−)-(2,3)-trans-methyl 3-((5-fluoro-2-(5-fluoro-1-tosyl-1H-pyrrolo[2,3-b]pyridin-3-yl)pyridin-4-yl)amino)bicyclo[2.2.2]octane-2-carboxylate (84)

A mixture of racemic-(2,3)-trans-methyl 3-((2-chloro-5-fluoropyridin-4-yl)amino)bicyclo[2.2.2]octane-2-carboxylate, 83, (0.050 g, 0.160 mmol) and 5-fluoro-1-(p-tolylsulfonyl)-3-(4,4,5,5-tetramethyl-1,3,2-dioxaborolan-2-yl)pyrrolo[2,3-b]pyridine, 9, (0.077 g, 0.184 mmol) and K₃PO₄ (0.112 g, 0.528 mmol) in water (0.250 mL) and 2-Me-THF (1.416 mL) was degassed under a stream of N₂ for 5 minutes. Then, X-Phos (0.008 g, 0.016 mmol) and Pd₂(dba)₃ (0.004 g, 0.004 mmol) was added and the mixture was degassed for an additional 3 minutes. The vessel was sealed and heated to 70° C. (thermal). After 1 hour, LC-MS indicated significant amounts of starting material, 83, was still present. Additional racemic-(2,3)-trans-methyl 3-((2-chloro-5-fluoropyridin-4-yl)amino)bicyclo[2.2.2]octane-2-carboxylate (0.050 g, 0.160 mmol) was added and the mixture was reheated to 120° C. (in microwave reactor) for 30 minutes. The cooled biphasic mixture was separated and the aqueous layer was extracted with MeTHF (3 mL). The combined organic phases were concentrated in vacuo. Purification by flash chromatography (SiO₂, 0-100% EtOAc in hexanes, gradient elution) provided 91 mg of the desired product: LCMS Gradient: 10-90%, TFA, 5 min, C18/AcN, Retention Time=2.95 min, (M+H) 567.00.

### Formation of (+/−)-(2,3)-trans-methyl 3-((5-fluoro-2-(5-fluoro-1H-pyrrolo[2,3-b]pyridin-3-yl)pyridin-4-yl)amino)bicyclo[2.2.2]octane-2-carboxylate (85)

A stirred solution of (+/−)-(2,3)-trans-methyl 3-((5-fluoro-2-(5-fluoro-1-tosyl-1H-pyrrolo[2,3-b]pyridin-3-yl)pyridin-4-yl)amino)bicyclo[2.2.2]octane-2-carboxylate, 84, (0.09 g, 0.16 mmol) in acetonitrile (1.5 mL) was treated with HCl (0.40 mL of 4 M solution in 1,4-dioxane, 1.6 mmol). The solution was heated 60° C. for 6 hours. The solution was cooled to room temperature and stirred overnight. The solution was reheated to 70° C. until LC-MS indicated complete conversion 24 hours. The mixture was then cooled, diluted with MTBE and the desired product was isolated as the HCl salt by filtration and rinsing with additional MTBE to afford 35 mg (47% yield) of the desired product as the HCl salt: ¹H NMR (300 MHz, CDCl₃) δ 8.33 (s, 1H), 8.30 (d, J^{=6.0} Hz, 1H), 8.19 (s, 1H), 8.17-8.10 (m, 1H), 7.27 (d, J=7.4 Hz, 1H), 4.48 (d, J=6.0 Hz, 1H), 3.72 (d, J=1.0 Hz, 3H), 2.90 (d, J=5.9 Hz, 1H), 2.14 (s, 1H), 1.92 (s, 1H), 1.84 (d, J=7.2 Hz, 3H), 1.77-1.46 (m, 5H) ppm.

### Formation of (+/−)-(2,3)-trans-3-((5-fluoro-2-(5-fluoro-1H-pyrrolo[2,3-b]pyridin-3-yl)pyridin-4-yl)amino)bicyclo[2.2.2]octane-2-carboxylic acid (I-50)

A mixture of (+/−)-(2,3)-trans-methyl 3-((5-fluoro-2-(5-fluoro-1H-pyrrolo[2,3-b]pyridin-3-yl)pyridin-4-yl)amino)bicyclo[2.2.2]octane-2-carboxylate, 85, (0.028 g, 0.061 mmol) in THF (0.75 mL) and methanol (0.25 mL) was treated with NaOH (0.243 mL of 2 M solution, 0.485 mmol). The resulting clear solution was heated to 60° C. for 3 hours. The reaction mixture was cooled to room temperature and neutralized with aqueous HCl (pH 6) and concentrated in vacuo to remove volatile organics. The suspension was diluted with a small amount of water and filtered. The resulting solid was rinsed with several small portions of water and dried in vacuo overnight to provide 17 mg of the desired product as a white powder (67% yield): ¹H NMR (400 MHz, MeOD) δ 8.24 (d, J=7.1 Hz, 1H), 8.19 (s, 1H), 8.08 (d, J=4.2 Hz, 1H), 7.96 (s, 1H), 7.08 (d, J=7.4 Hz, 1H), 4.27 (d, J=7.2 Hz, 1H), 2.64 (d, J=5.9 Hz, 1H), 2.10 (s, 1H), 1.93-1.74 (m, 4H), 1.74-1.58 (m, 3H), 1.58-1.43 (m, 2H); LCMS Gradient: 10-90%, TFA, 5 min, C18/AcN, Retention Time=2.18 min, (M+H) 399.05.

### Preparation of Compound I-51

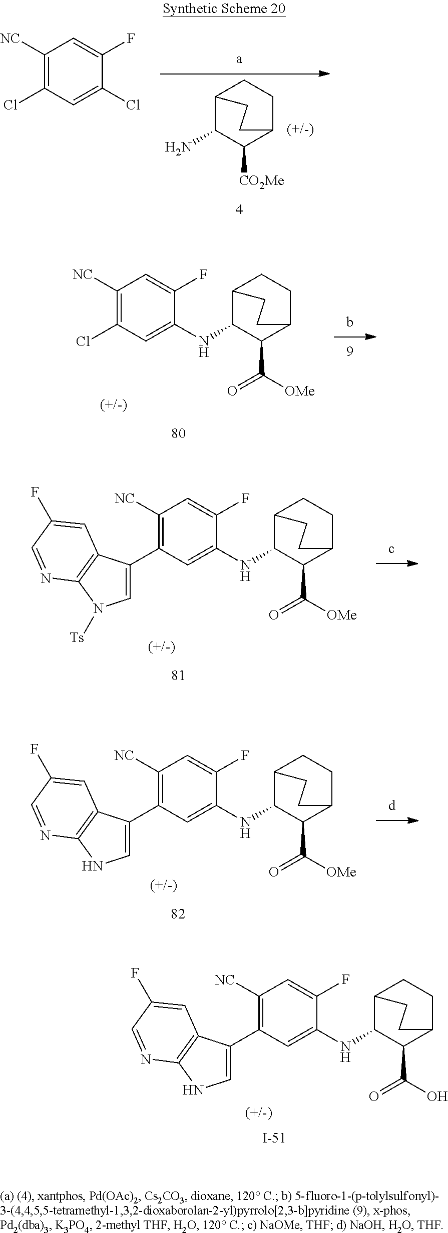

### Formation of (+/−)-methyl 3-((5-chloro-4-cyano-2-fluorophenyl)amino)-bicyclo[2.2.2]octane-2-carboxylate (80)

To a solution of racemic methyl (2S,3S)-3-aminobicyclo[2.2.2]octane-2-carboxylate, 4, (0.557 g, 3.000 mmol) and 2,4-dichloro-5-fluoro-benzonitrile (0.570 g, 3.000 mmol) in 1,4-dioxane (12.0 mL) was added (5-diphenylphosphanyl-9,9-dimethyl-xanthen-4-yl)-diphenyl-phosphane (0.087 g, 0.150 mmol), diacetoxypalladium (0.040 g, 0.180 mmol) and Cs₂CO₃ (1.955 g, 6.000 mmol). The mixture was heated at 120° C. in a pressure tube for 1.5 hours. The reaction mixture was filtered through a pad of celite and the filtrate was concentrated under reduced pressure. The resulting residue was purified by silica gel chromatography (30% EtOAc/Hexanes) to afford 860 mg of the desired product: ¹H NMR (400 MHz, CDCl₃) δ 7.19 (d, J=11.0 Hz, 1H), 6.77 (d, J=7.5 Hz, 1H), 4.58 (d, J=4.0 Hz, 1H), 4.09 (t, J=6.6 Hz, 1H), 3.75 (d, J=1.9 Hz, 3H), 2.34 (d, J=5.8 Hz, 1H), 2.11 (d, J=2.4 Hz, 1H), 1.85 (d, J^{=2.2} Hz, 1H), 1.78-1.62 (m, 5H), 1.60-1.41 (m, 4H); LC/MS Gradient: 10-90%, formic 5 min, C18/AcN, Retention Time=3.76 min, (M+H) 337.02.

### Formation of (+/−)-methyl 3-((4-cyano-2-fluoro-5-(5-fluoro-1-tosyl-1H-pyrrolo[2,3-b]pyridin-3-yl)phenyl)amino)bicyclo[2.2.2]octane-2-carboxylate (81)

A solution of racemic methyl 3-(5-chloro-4-cyano-2-fluoro-anilino)bicyclo[2.2.2]octane-2-carboxylate, 80, (0.430 g, 1.277 mmol), 5-fluoro-1-(p-tolylsulfonyl)-3-(4,4,5,5-tetramethyl-1,3,2-dioxaborolan-2-yl)pyrrolo[2,3-b]pyridine, 9, (0.638 g, 1.532 mmol) and K₃PO₄ (0.813 g, 3.831 mmol) in 2-methyl THF (15 mL) and H₂O (2 mL) was degassed under a stream of nitrogen for 40 minutes. X-phos (0.073 g, 0.153 mmol) and Pd₂(dba)₃ (0.029 g, 0.032 mmol) were added to the reaction mixture, which was then heated at 120° C. in a pressure tube for 45 minutes. The aqueous phase was removed and the remaining organic phase was filtered through a pad of celite and concentrated under reduced pressure. The resulting residue was purified by silica gel chromatography (30% EtOAc/Hexanes) to afford 700 mg of the desired product: ¹H NMR (400 MHz, CDCl₃) δ 8.34 (t, J=8.6 Hz, 1H), 8.12 (d, J=8.3 Hz, 2H), 8.07 (s, 1H), 7.61 (dd, J=8.4, 2.6 Hz, 1H), 7.41-7.29 (m, 3H), 6.86 (d, J=8.1 Hz, 1H), 4.68 (dd, J=7.7, 3.4 Hz, 1H), 4.14 (dd, J=14.2, 7.1 Hz, 1H), 3.70 (s, 3H), 2.40 (s, 3H), 2.13 (d, J=2.1 Hz, 1H), 1.86 (s, 1H), 1.69 (m, 6H), 1.54 (m, 3H). LC/MS Gradient: 10-90%, formic 5 min, C18/AcN, Retention Time=4.03 min, (M+H) 591.00.

### Formation of (+/−)-methyl 3-((4-cyano-2-fluoro-5-(5-fluoro-1H-pyrrolo[2,3-b]pyridin-3-yl)phenyl)amino)bicyclo[2.2.2]octane-2-carboxylate (82)

To a solution of methyl 3-((4-cyano-2-fluoro-5-(5-fluoro-1-tosyl-1H-pyrrolo[2,3-b]pyridin-3-yl)phenyl)amino)bicyclo[2.2.2]octane-2-carboxylate, 81, (0.850 g, 1.439 mmol) in THF (20 mL) was added sodium methoxide (0.311 g, 1.439 mmol). After stirring the reaction mixture at room temperature for 5 minutes, the mixture was diluted into EtOAc and aqueous saturated NaHCO₃ solution. The organic phase was dried (MgSO₄), filtered and concentracted in vacuo. The resulting residue was purified by silica gel chromatography (50% EtOAc/Hexanes) to 580 mg of the desired product: ¹H NMR (400 MHz, CDCl₃) δ 10.16 (s, 1H), 8.12 (s, 1H), 7.63 (s, 1H), 7.59 (d, J=8.9 Hz, 1H), 7.15 (dd, J=11.3, 1.1 Hz, 1H), 6.74 (d, J=8.1 Hz, 1H), 4.45 (d, J=4.7 Hz, 1H), 3.99 (dd, J=9.9, 4.1 Hz, 1H), 3.50 (d, J=1.0 Hz, 3H), 2.25 (d, J=4.9 Hz, 1H), 1.92 (d, J=13.6 Hz, 1H), 1.73 (s, 1H), 1.63-1.43 (m, 6H), 1.42-1.17 (m, 3H). LC/MS Gradient: 10-90%, formic 5 min, C18/AcN, Retention Time=3.54 min, (M+H) 436.89.

### Formation of (+/−)-3-((4-cyano-2-fluoro-5-(5-fluoro-1H-pyrrolo[2,3-b]pyridin-3-yl)phenyl)amino)bicyclo[2.2.2]octane-2-carboxylic acid (I-51)

To a solution of racemic methyl 3-[4-cyano-2-fluoro-5-(5-fluoro-1H-pyrrolo[2,3-b]pyridin-3-yl)anilino]bicyclo[2.2.2]octane-2-carboxylate, 82, (0.220 g, 0.504 mmol) in THF (10 mL) was added NaOH (10.08 mL of 1 M solution, 10.08 mmol). The reaction mixture was heated and stirred at 120° C. for 2 hours. The aqueous phase was isolated and the pH was adjusted to pH 6 and extracted with EtOAc. The organic phase was dried (MgSO4), filtered and concentrated in vacuo. The resulting residue was purified by silica gel chromatography (MeOH/CH₂Cl₂) to afford 150 mg of the desired product: ¹H NMR (400 MHz, d6-DMSO) δ 12.34 (s, 1H), 12.28 (s, 1H), 8.31 (s, 1H), 7.91 (d, J^{=2.5} Hz, 1H), 7.75 (dd, J^{=9.6, 2.6} Hz, 1H), 7.62 (d, J=12.0 Hz, 1H), 6.79 (d, J=8.3 Hz, 1H), 6.61 (d, J=6.6 Hz, 1H), 4.00 (d, J=7.4 Hz, 1H), 2.79 (d, J=6.7 Hz, 1H), 1.97 (s, 1H), 1.84-1.65 (m, 3H), 1.51 (dd, J=43.2, 19.0 Hz, 3H), 1.39 (s, 3H); LC/MS Gradient: 10-90%, formic 5 min, C18/AcN, Retention Time=3.16 min, (M+H) 423.19.

### Influenza Antiviral Assay

Antiviral assays were performed using two cell-based methods:

A 384-well microtiter plate modification of the standard cytopathic effect (CPE) assay method was developed, similar to that of Noah, et al. (Antiviral Res. 73:50-60, 2006). Briefly, MDCK cells were incubated with test compounds and influenza A virus (A/PR/8/34), at a low multiplicity of infection (approximate MOI=0.005), for 72 hours at 37° C., and cell viability was measured using ATP detection (CellTiter Glo, Promega Inc.). Control wells containing cells and virus show cell death while wells containing cells, virus, and active antiviral compounds show cell survival (cell protection). Different concentrations of test compounds were evaluated, in quadruplicate, for example, over a range from approximately 20 μM to 1 nM. Dose-response curves were prepared using standard 4-parameter curve fitting methods, and the concentration of test compound resulting in 50% cell protection, or cell survival equivalent to 50% of the uninfected wells, was reported as the IC₅₀.

A second cell-based antiviral assay was developed that depends on the multiplication of virus-specific RNA molecules in the infected cells, with RNA levels being directly measured using the branched-chain DNA (bDNA), hybridization method (Wagaman et al, J. Virol Meth, 105:105-114, 2002). In this assay, cells are initially infected in wells of a 96-well microtiter plate, the virus is allowed to replicate in the infected cells and spread to additional rounds of cells, then the cells are lysed and viral RNA content is measured. This assay is stopped earlier that the CPE assay, usually after 18-36 hours, while all the target cells are still viable. Viral RNA is quantitated by hybridization of well lysates to specific oligonucleotide probes fixed to wells of an assay plate, then amplification of the signal by hybridization with additional probes linked to a reporter enzyme, according to the kit manufacturer's instructions (Quantigene 1.0, Panomics, Inc.). Minus-strand viral RNA is measured using probes designed for the consensus type A hemagglutination gene. Control wells containing cells and virus were used to define the 100% viral replication level, and dose-response curves for antiviral test compounds were analyzed using 4-parameter curve fitting methods. The concentration of test compound resulting in viral RNA levels equal to that of 50% of the control wells were reported as EC₅₀.

Virus and Cell culture methods: Madin-Darby Canine Kidney cells (CCL-34 American Type Culture Collection) were maintained in Dulbecco's Modfied Eagle Medium (DMEM) supplemented with 2 mM L-glutamine, 1,000 U/ml penicillin, 1,000 ug/ml streptomycin, 10 mM HEPES, and 10% fetal bovine medium. For the CPE assay, the day before the assay, cells were suspended by trypsinization and 10,000 cells per well were distributed to wells of a 384 well plate in 50 μl. On the day of the assay, adherent cells were washed with three changes of DMEM containing 1 ug/ml TPCK-treated trypsin, without fetal bovine serum. Assays were initiated with the addition of 30 TCID₅₀ of virus and test compound, in medium containing 1 μg/ml TPCK-treated trypsin, in a final volume of 501. Plates were incubated for 72 hours at 37° C. in a humidified, 5% CO₂ atmosphere. Alternatively, cells were grown in DMEM+fetal bovine serum as above, but on the day of the assay they were trypsinized, washed 2 times and suspended in serum-free EX-Cell MDCK cell medium (SAFC Biosciences, Lenexa, Kans.) and plated into wells at 20,000 cells per well. These wells were then used for assay after 5 hours of incubation, without the need for washing.

Influenza virus, strain A/PR/8/34 (tissue culture adapted) was obtained from ATCC (VR-1469). Low-passage virus stocks were prepared in MDCK cells using standard methods (WHO Manual on Animal Influenza Diagnosis and Surveillance, 2002), and TCID₅₀ measurements were performed by testing serial dilutions on MDCK cells in the 384-well CPE assay format, above, and calculating results using the Karber method.

Mean IC₅₀ values (mean all) for certain specific compounds are summarized in Tables 1 and 2:

A: IC₅₀ <3.3 μM; and

B IC₅₀ ≧3.3 μM.

Mean EC₅₀ values (mean all) for certain compounds are also summarized in Tables 1 and 2:

A: EC₅₀ <3.3 μM; and

B EC₅₀ 3.3 μM.

For example, IC₅₀ and EC₅₀ values of Compound I-28 are 0.005 μM and 0.005 μM.

TABLE 1
IC₅₀, EC₅₀, NMR and LCMS Data of Compounds of Invention.

| | | Flu, | | | | |
|---|---|---|---|---|---|---|
| | | MDCK | bDNA | | | |
| | | IC50 | EC50 | RT | | |
| Nos. | Molecule | (uM) | (uM) | (min) | M + 1 | NMR |
| I-1 | 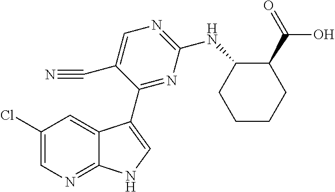 [SMILES: N#Cc1cnc(N[C@H]2CCCC[C@@H]2C(=O)O)nc1-c1c[nH]c2ncc(Cl)cc12] | A | A | 4.04 | 397 | |
| I-3 | 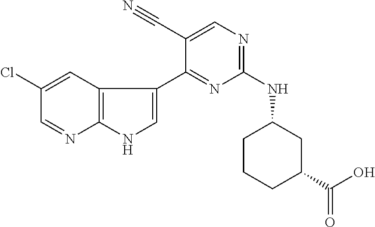 [SMILES: N#Cc1cnc(N[C@H]2CCC[C@@H](C(=O)O)C2)nc1-c1c[nH]c2ncc(Cl)cc12] | A | A | 4.09 | 397.43 | |
| I-4 | 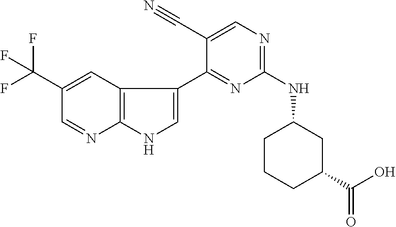 [SMILES: N#Cc1cnc(N[C@H]2CCC[C@@H](C(=O)O)C2)nc1-c1c[nH]c2ncc(C(F)(F)F)cc12] | A | A | 4.08 | 431.47 | |
| I-5 | 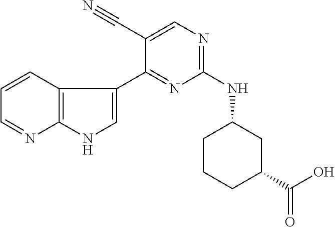 [SMILES: N#Cc1cnc(N[C@H]2CCC[C@@H](C(=O)O)C2)nc1-c1c[nH]c2ncccc12] | B | | 3.83 | 363.43 | |
| I-6 | 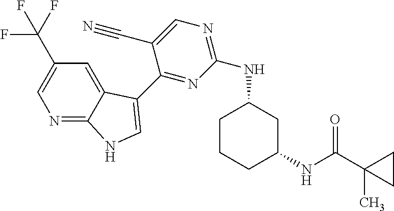 [SMILES: CC1(C(=O)N[C@@H]2CCC[C@H](Nc3ncc(C#N)c(-c4c[nH]c5ncc(C(F)(F)F)cc45)n3)C2)CC1] | A | A | 4.07 | 485 | |
| I-7 | 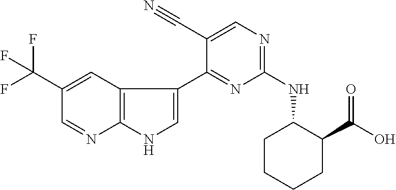 [SMILES: N#Cc1cnc(N[C@H]2CCCC[C@@H]2C(=O)O)nc1-c1c[nH]c2ncc(C(F)(F)F)cc12] | A | A | 4.08 | 431 | |
| I-8 | 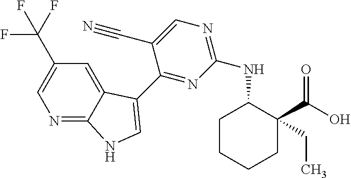 [SMILES: CC[C@]1(C(=O)O)CCCC[C@@H]1Nc1ncc(C#N)c(-c2c[nH]c3ncc(C(F)(F)F)cc23)n1] | A | A | 4.13 | 459.5 | |
| I-9 | 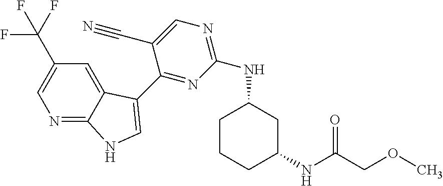 [SMILES: COCC(=O)N[C@@H]1CCC[C@H](Nc2ncc(C#N)c(-c3c[nH]c4ncc(C(F)(F)F)cc34)n2)C1] | A | A | 3.95 | 474 | |
| I-10 | 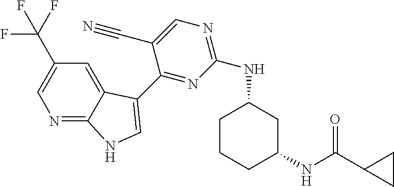 [SMILES: N#Cc1cnc(N[C@H]2CCC[C@@H](NC(=O)C3CC3)C2)nc1-c1c[nH]c2ncc(C(F)(F)F)cc12] | A | A | 3.85 | 470 | |
| I-11 | 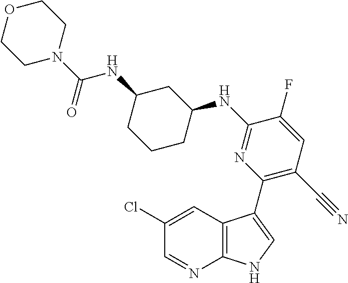 [SMILES: N#Cc1cc(F)c(N[C@H]2CCC[C@@H](NC(=O)N3CCOCC3)C2)nc1-c1c[nH]c2ncc(Cl)cc12] | A | A | 2.76 | 498.23 | |
| I-12 | 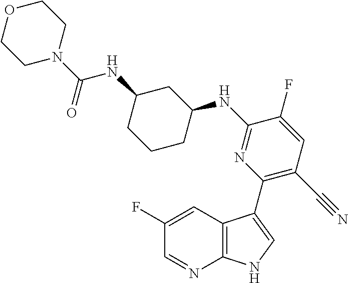 [SMILES: N#Cc1cc(F)c(N[C@H]2CCC[C@@H](NC(=O)N3CCOCC3)C2)nc1-c1c[nH]c2ncc(F)cc12] | A | A | 2.62 | 482 | |
| I-13 | 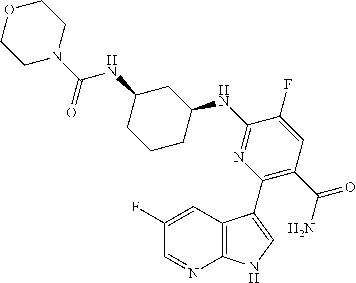 [SMILES: NC(=O)c1cc(F)c(N[C@H]2CCC[C@@H](NC(=O)N3CCOCC3)C2)nc1-c1c[nH]c2ncc(F)cc12] | A | A | 2.03 | 500 | |
| I-14 | 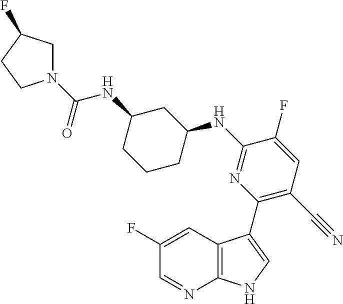 [SMILES: N#Cc1cc(F)c(N[C@H]2CCC[C@@H](NC(=O)N3CC[C@@H](F)C3)C2)nc1-c1c[nH]c2ncc(F)cc12] | A | A | 2.74 | 484.28 | |
| I-15 | 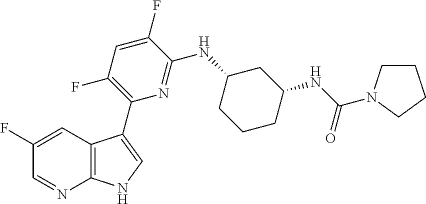 [SMILES: O=C(N[C@@H]1CCC[C@H](Nc2nc(-c3c[nH]c4ncc(F)cc34)c(F)cc2F)C1)N1CCCC1] | A | A | 2.97 | 459 | |
| I-16 | 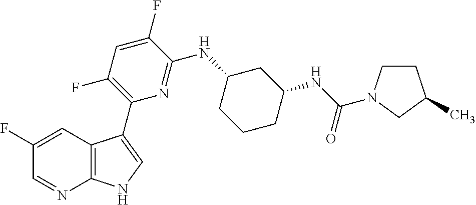 [SMILES: C[C@@H]1CCN(C(=O)N[C@@H]2CCC[C@H](Nc3nc(-c4c[nH]c5ncc(F)cc45)c(F)cc3F)C2)C1] | A | A | 3.12 | 473 | |
| I-17 | 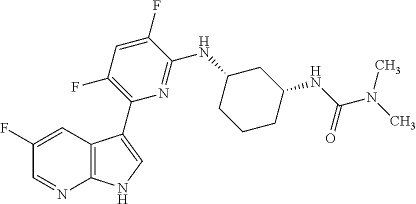 [SMILES: CN(C)C(=O)N[C@@H]1CCC[C@H](Nc2nc(-c3c[nH]c4ncc(F)cc34)c(F)cc2F)C1] | A | A | 2.84 | 433.42 | |
| I-18 | 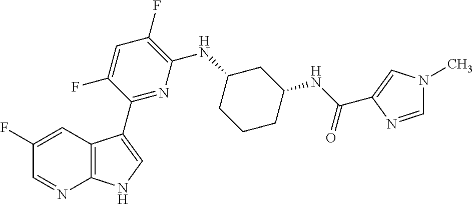 [SMILES: Cn1cnc(C(=O)N[C@@H]2CCC[C@H](Nc3nc(-c4c[nH]c5ncc(F)cc45)c(F)cc3F)C2)c1] | A | A | 2.37 | 470 | |
| I-19 | 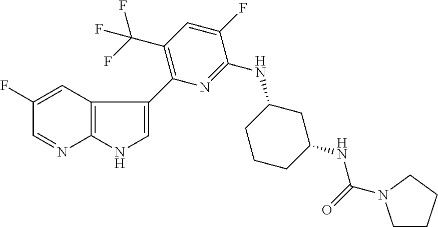 [SMILES: O=C(N[C@@H]1CCC[C@H](Nc2nc(-c3c[nH]c4ncc(F)cc34)c(C(F)(F)F)cc2F)C1)N1CCCC1] | A | A | 2.96 | 510 | |
| I-20 | 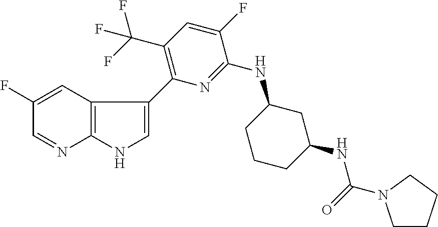 [SMILES: O=C(N[C@H]1CCC[C@@H](Nc2nc(-c3c[nH]c4ncc(F)cc34)c(C(F)(F)F)cc2F)C1)N1CCCC1] | A | A | 2.96 | 509.54 | |
| I-21 | 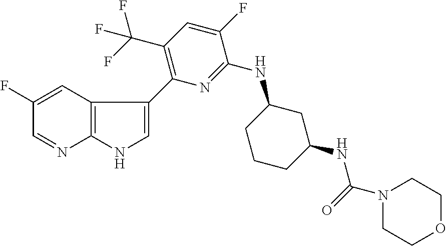 [SMILES: O=C(N[C@H]1CCC[C@@H](Nc2nc(-c3c[nH]c4ncc(F)cc34)c(C(F)(F)F)cc2F)C1)N1CCOCC1] | A | A | 2.82 | 525 | |
| I-22 | 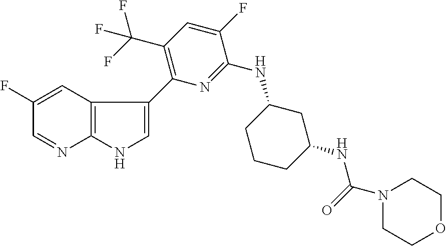 [SMILES: O=C(N[C@@H]1CCC[C@H](Nc2nc(-c3c[nH]c4ncc(F)cc34)c(C(F)(F)F)cc2F)C1)N1CCOCC1] | A | A | 2.82 | 525 | |
| I-23 | 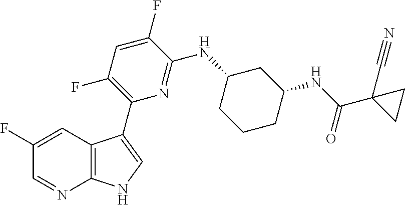 [SMILES: N#CC1(C(=O)N[C@@H]2CCC[C@H](Nc3nc(-c4c[nH]c5ncc(F)cc45)c(F)cc3F)C2)CC1] | A | A | 3.1 | 455.49 | |
| I-24 | 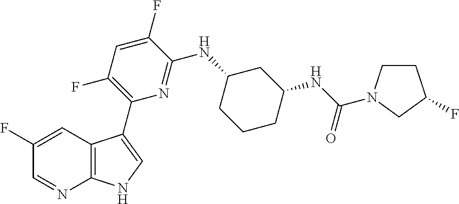 [SMILES: O=C(N[C@@H]1CCC[C@H](Nc2nc(-c3c[nH]c4ncc(F)cc34)c(F)cc2F)C1)N1CC[C@H](F)C1] | A | A | 2.87 | 478 | |
| I-25 | 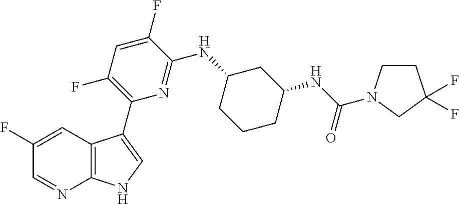 [SMILES: O=C(N[C@@H]1CCC[C@H](Nc2nc(-c3c[nH]c4ncc(F)cc34)c(F)cc2F)C1)N1CCC(F)(F)C1] | A | A | 3.05 | 496 | |
| I-26 | 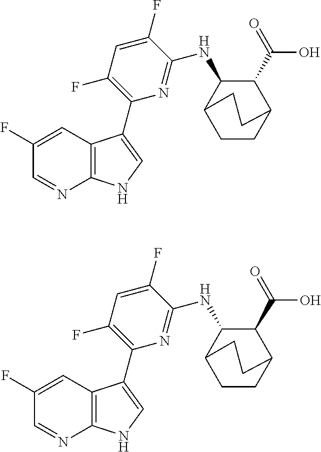 [SMILES: O=C(O)[C@@H]1C2CCC(CC2)[C@H]1Nc1nc(-c2c[nH]c3ncc(F)cc23)c(F)cc1F ; O=C(O)[C@H]1C2CCC(CC2)[C@@H]1Nc1nc(-c2c[nH]c3ncc(F)cc23)c(F)cc1F] | A | A | 3.27 | 417.48 | |
| I-27 | 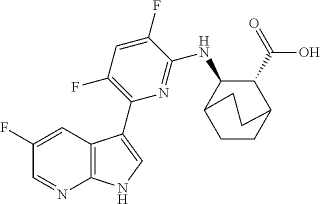 [SMILES: O=C(O)[C@@H]1C2CCC(CC2)[C@H]1Nc1nc(-c2c[nH]c3ncc(F)cc23)c(F)cc1F] | B | A | 3.28 | 418 | 1H NMR (300 MHz, MeOD) δ 8.81 (dd, J = 9.7, 2.7 Hz, 1H), 8.15 (s, 1H), 7.98 (d, J = 2.5 Hz, 1H), 7.28 (t, J = 10.3 Hz, 1H), 4.82 (d, J = 7.1 Hz, 1H), 3.33 (s, 1H), 2.65 (d, J = 6.9 Hz, 1H), 2.12-1.91 (m, 3H), 1.89-1.30 (m, 7H). |
| I-28 | 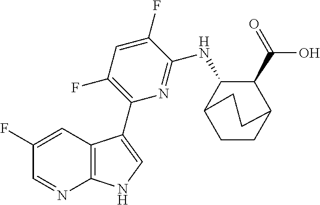 [SMILES: O=C(O)[C@H]1C2CCC(CC2)[C@@H]1Nc1nc(-c2c[nH]c3ncc(F)cc23)c(F)cc1F] | A | A | 3.28 | 418 | 1H NMR (300 MHz, MeOD) δ 8.74 (dd, J = 9.7, 2.6 Hz, 1H), 8.12 (s, 1H), 7.91 (d, J = 2.2 Hz, 1H), 7.19 (dd, J = 19.2, 8.8 Hz, 1H), 4.80 (d, J = 6.8 Hz, 1H), 3.50-3.19 (m, 2H), 2.69 (d, J = 6.8 Hz, 1H), 2.01 (t, J = 22.7 Hz, 3H), 1.88-1.19 (m, 7H). |
| I-29 | 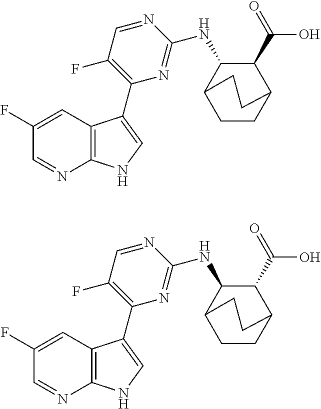 [SMILES: O=C(O)[C@H]1C2CCC(CC2)[C@@H]1Nc1ncc(F)c(-c2c[nH]c3ncc(F)cc23)n1 ; O=C(O)[C@@H]1C2CCC(CC2)[C@H]1Nc1ncc(F)c(-c2c[nH]c3ncc(F)cc23)n1] | A | A | 2.81 | 400.53 | |
| I-30 | 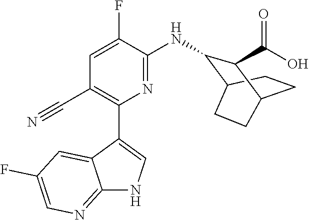 [SMILES: N#Cc1cc(F)c(N[C@H]2C3CCC(CC3)[C@@H]2C(=O)O)nc1-c1c[nH]c2ncc(F)cc12] | A | A | 3.13 | 424.33 | |
| I-31 | 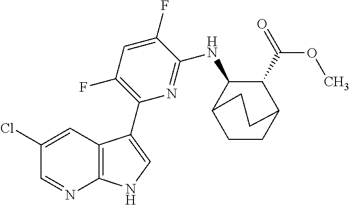 [SMILES: COC(=O)[C@@H]1C2CCC(CC2)[C@H]1Nc1nc(-c2c[nH]c3ncc(Cl)cc23)c(F)cc1F] | B | B | 3.9 | 447.56 | |
| I-32 | 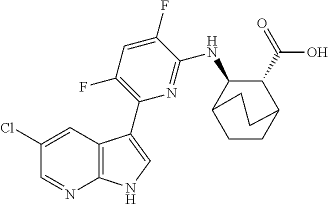 [SMILES: O=C(O)[C@@H]1C2CCC(CC2)[C@H]1Nc1nc(-c2c[nH]c3ncc(Cl)cc23)c(F)cc1F] | A | A | 3.47 | 433.23 | |
| I-33 | 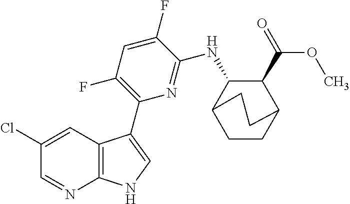 [SMILES: COC(=O)[C@H]1C2CCC(CC2)[C@@H]1Nc1nc(-c2c[nH]c3ncc(Cl)cc23)c(F)cc1F] | A | A | 3.91 | 448 | |
| I-34 | 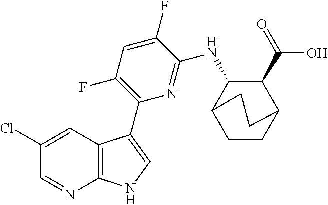 [SMILES: O=C(O)[C@H]1C2CCC(CC2)[C@@H]1Nc1nc(-c2c[nH]c3ncc(Cl)cc23)c(F)cc1F] | A | A | 3.46 | 434 | |
| I-35 | 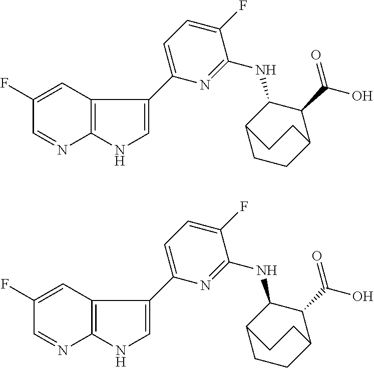 [SMILES: O=C(O)[C@H]1C2CCC(CC2)[C@@H]1Nc1nc(-c2c[nH]c3ncc(F)cc23)ccc1F ; O=C(O)[C@@H]1C2CCC(CC2)[C@H]1Nc1nc(-c2c[nH]c3ncc(F)cc23)ccc1F] | A | A | 3.14 | 399 | 1H NMR (400 MHz, CDCl3) δ 10.75 (s, 1H), 8.12 (dd, J = 9.2, 2.4 Hz, 1H), 7.76 (d, J = 11.5 Hz, 2H), 7.21 (dd, J = 10.7, 8.1 Hz, 1H), 6.74 (dd, J = 8.0, 2.8 Hz, 1H), 5.06 (d, J = 6.8 Hz, 1H), 4.70 (s, 1H), 2.44 (d, J = 4.2 Hz, 1H), 2.02 (d, J = 17.8 Hz, 2H), 1.96- 1.82 (m, 3H), 1.80- 1.61 (m, 4H), 1.48 (t, J = 11.4 Hz, 1H). [1] |
| I-36 | 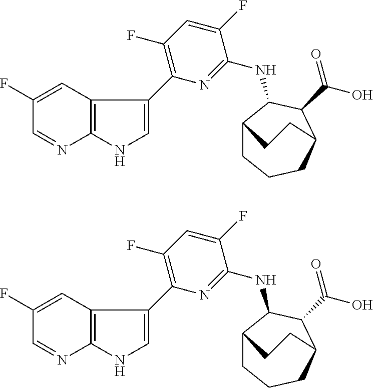 | | A | 3.41 | 432 | 1H NMR (400 MHz, MeOD) δ 8.90 (dd, J = 9.6, 2.7 Hz, 1H), 8.16 (s, 1H), 7.98 (d, J = 2.5 Hz, 1H), 7.28 (t, J = 10.3 Hz, 1H), 5.12 (d, J = 7.7 Hz, 1H), 2.71 (dd, J = 7.8, 3.6 Hz, 1H), 2.48 (m, 1H), 2.08 (m, 1H), 2.02-1.49 (m, 10H). [1] |
| I-37 | 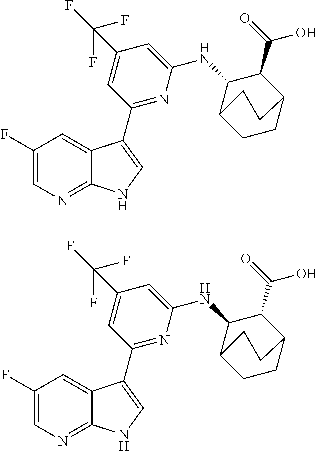 | A | A | 3.41 | 450 | 1H NMR (300 MHz, MeOD) δ 8.55 (dd, J = 9.5, 2.7 Hz, 1H), 8.15 (s, 2H), 7.70-7.41 (m, 1H), 7.08 (s, 1H), 6.65 (s, 1H), 4.61 (d, J = 5.9 Hz, 1H), 2.53 (d, J = 6.3 Hz, 1H), 2.23- 1.05 (m, 11H). |
| I-38 | 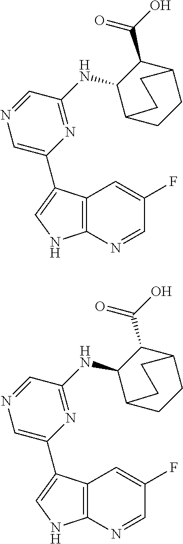 [SMILES: O=C(O)[C@H]1C2CCC(CC2)[C@@H]1Nc1cncc(-c2c[nH]c3ncc(F)cc23)n1 ; O=C(O)[C@@H]1C2CCC(CC2)[C@H]1Nc1cncc(-c2c[nH]c3ncc(F)cc23)n1] | A | A | 2.31 | 382 | 1H NMR (300 MHz, MeOD) δ 8.63 (dd, J = 9.4, 2.6 Hz, 1H), 8.25 (s, 1H), 8.20 (s, 1H), 8.14 (s, 1H), 7.62 (s, 1H), 4.70 (d, J = 6.4 Hz, 1H), 2.56 (d, J = 6.4 Hz, 1H), 2.10 (s, 1H), 2.01 (s, 1H), 1.97- 1.62 (m, 6H), 1.60- 1.43 (m, 2H). |
| I-39 | 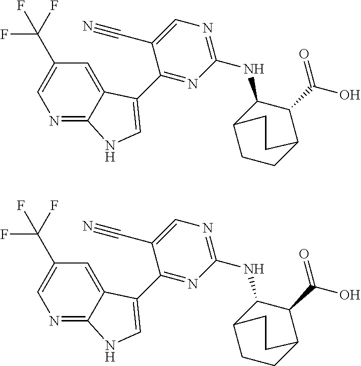 [SMILES: N#Cc1cnc(N[C@@H]2C3CCC(CC3)[C@H]2C(=O)O)nc1-c1c[nH]c2ncc(C(F)(F)F)cc12 ; N#Cc1cnc(N[C@H]2C3CCC(CC3)[C@@H]2C(=O)O)nc1-c1c[nH]c2ncc(C(F)(F)F)cc12] | A | A | 3.24 | 457 | 1H NMR (300 MHz, MeOD) major rotamer: δ 9.33 (s, 1H), 8.77 (s, 1H), 8.64 (s, 1H), 8.55 (d, J = 16.8 Hz, 1H), 4.58 (d, 1H), 2.65 (d, J = 7.2 Hz, 1H), 2.17- 2.06 (m, 1H), 2.06- 1.95 (m, 1H), 1.94- 1.73 (m, 3H), 1.73- 1.55 (m, 2H), 1.55- 1.42 (m, 2H). |
| I-40 | 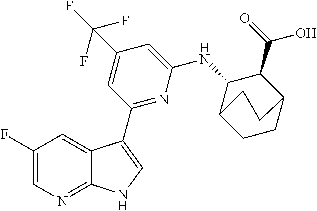 [SMILES: O=C(O)[C@H]1C2CCC(CC2)[C@@H]1Nc1cc(C(F)(F)F)cc(-c2c[nH]c3ncc(F)cc23)n1] | A | A | 3.42 | 449 | |
| I-41 | 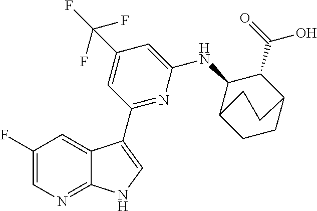 [SMILES: O=C(O)[C@@H]1C2CCC(CC2)[C@H]1Nc1cc(C(F)(F)F)cc(-c2c[nH]c3ncc(F)cc23)n1] | A | A | 3.43 | 449 | |
| I-42 | 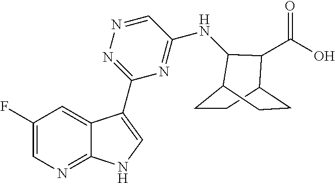 [SMILES: O=C(O)C1C2CCC(CC2)C1Nc1cnnc(-c2c[nH]c3ncc(F)cc23)n1] | A | A | 1.78 | 383.32 | 1H NMR (300 MHz, DMSO-d6) δ 12.91 (s, 1H), 12.52 (s, 1H), 8.62 (s, 1H), 8.46 (d, J = 3.0 Hz, 1H), 8.42 (d, J = 5.7 Hz, 1H), 8.30 (s, 1H), 4.72 (s, 1H), 2.60 (d, J = 6.5 Hz, 1H), 2.06 (s, 1H), 1.99 (s, 1H), 1.82-1.40 (m, 9H). |
| I-43 | 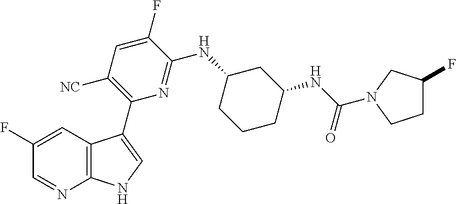 [SMILES: N#Cc1cc(F)c(N[C@H]2CCC[C@@H](NC(=O)N3CC[C@H](F)C3)C2)nc1-c1c[nH]c2ncc(F)cc12] | A | A | | 484.29 | |
| I-44 | 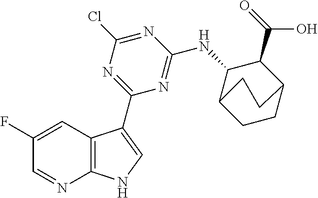 [SMILES: O=C(O)[C@H]1C2CCC(CC2)[C@@H]1Nc1nc(Cl)nc(-c2c[nH]c3ncc(F)cc23)n1] | A | A | 3.03 | 417.09 | |

TABLE 2
IC₅₀, EC₅₀, NMR and LCMS Data of Compounds of Invention.

| | | Flu, | | | | |
|---|---|---|---|---|---|---|
| | | MDCK | bDNA | | | |
| | | IC50 | EC50 | LCMS | | |
| | Molecule | (uM) | (uM) | RT | M + 1 | NMR |
| I-45 | 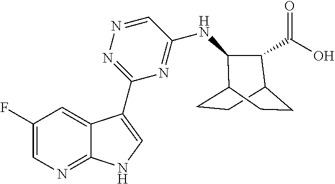 [SMILES: O=C(O)[C@@H]1C2CCC(CC2)[C@H]1Nc1cnnc(-c2c[nH]c3ncc(F)cc23)n1] | B | B | 1.78 | 383.32 | |
| I-46 | 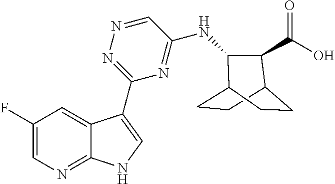 [SMILES: O=C(O)[C@H]1C2CCC(CC2)[C@@H]1Nc1cnnc(-c2c[nH]c3ncc(F)cc23)n1] | A | A | 1.78 | 383.32 | |
| I-47 | 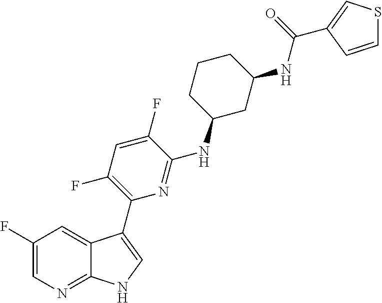 [SMILES: O=C(N[C@@H]1CCC[C@H](Nc2nc(-c3c[nH]c4ncc(F)cc34)c(F)cc2F)C1)c1ccsc1] | A | A | 3.17 | 472.25 | |
| I-48 | 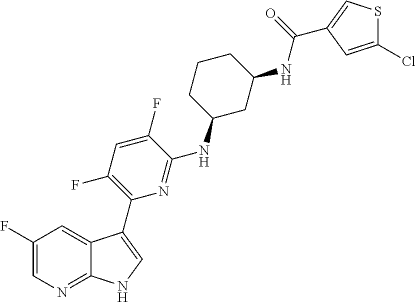 [SMILES: O=C(N[C@@H]1CCC[C@H](Nc2nc(-c3c[nH]c4ncc(F)cc34)c(F)cc2F)C1)c1csc(Cl)c1] | A | A | 3.5 | 506.17 | |
| I-49 | 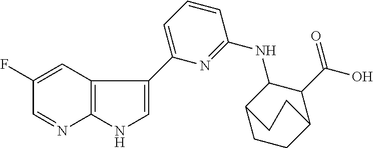 [SMILES: O=C(O)C1C2CCC(CC2)C1Nc1cccc(-c2c[nH]c3ncc(F)cc23)n1] | A | A | 1.91 | 381.27 | 1H NMR (300 MHz, DMSO) δ 8.63 (d, J = 8.0 Hz, 1H), 8.34-8.16 (m, 2H), 7.44 (s, 1H), 7.04 (d, J = 7.4 Hz, 1H), 6.36 (s, 1H), 4.45 (s, 1H), 1.96 (d, J = 20.7 Hz, 2H), 1.86-1.62 (m, 3H), 1.61- 1.26 (m, 4H). |
| I-50 | 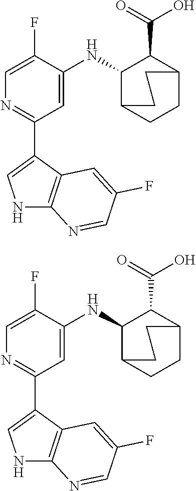 | A | A | 2.18 | 399.05 | 1H NMR (400 MHz, MeOD) δ 8.24 (d, J = 7.1 Hz, 1H), 8.19 (s, 1H), 8.08 (d, J = 4.2 Hz, 1H), 7.96 (s, 1H), 7.08 (d, J = 7.4 Hz, 1H), 4.27 (d, J = 7.2 Hz, 1H), 2.64 (d, J = 5.9 Hz, 1H), 2.10 (s, 1H), 1.93-1.74 (m, 4H), 1.74- 1.58 (m, 3H), 1.58- 1.43 (m, 2H). |
| I-51 | 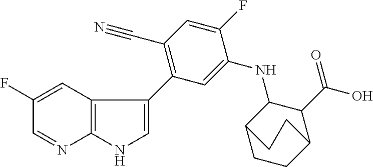 [SMILES: N#Cc1cc(F)c(NC2C3CCC(CC3)C2C(=O)O)cc1-c1c[nH]c2ncc(F)cc12] | A | A | 3.16 | 422.79 | 1 H NMR (400 MHz, DMSO) δ 12.34 (s, 1H), 12.28 (s, 1H), 8.31 (s, 1H), 7.91 (d, J = 2.5 Hz, 1H), 7.75 (dd, J = 9.6, 2.6 Hz, 1H), 7.62 (d, J = 12.0 Hz, 1H), 6.79 (d, J = 8.3 Hz, 1H), 6.61 (d, J = 6.6 Hz, 1H), 4.00 (d, J = 7.4 Hz, 1H), 2.79 (d, J = 6.7 Hz, 1H), 1.97 (s, 1H), 1.84-1.65 (m, 3H), 1.51 (dd, J = 43.2, 19.0 Hz, 3H), 1.39 (s, 3H). |

### In Vivo Assay

For efficacy studies, Balb/c mice (4-5 weeks of age) were challenged with 5×10³ TCID₅₀ in a total volume of 50 μl by intranasal by intranasal instillation (25 μl/nostril) under general anesthesia (Ketamine/Xylazine). Uninfected controls were challenged with tissue culture media (DMEM, 50 μl total volume). 48 hours post infection mice began treatment with Compound I-28 at 30 mg/kg bid for 10 days. Body weights and survival is scored daily for 21 days. In addition, Whole Body Plethysmography is conducted approximately every third day following challenge (Penh). Total Survival, Percent Body Weight Loss on post challenge day 8 and Penh on study day 6/7 are reported.

TABLE 3
Influneza Therapeutic Mouse Model (Dosing @ 48
hours post infection with 30 mg/kg BID X 10 days)

| | Percent | Percent Weight | WBP (Penh; |
|---|---|---|---|
| Compounds | Survival | Loss (Day 8)¹ | Day 6)² |
| I-28 | 100 | 24.8 | 1.84 |

¹Average weight loss for untreated controls on day 8 is 30-32%.
²Average Penh scores for untreated controls on study day 6 or 7 are 2.2-2.5, and for uninfected mice is ~0.35-0.45.

All references provided herein are incorporated herein in its entirety by reference. As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound represented by Structural Formula (I): 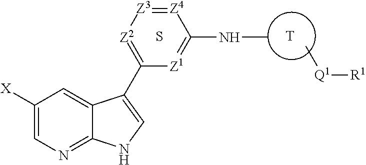
or a pharmaceutically acceptable salt thereof, wherein:
X is —Cl, —Br, —F, —CN, —O(C₁₋₄ alkyl), or C₁-C₆ aliphatic optionally substituted with one or more instances of J¹;
Z¹, Z², Z³, and Z⁴ are each and independently CR² or N, provided that up to three N are selected for Z¹, Z², Z³, and Z⁴, and provided that when Z³ and Z⁴ are both CR², then Z¹ and Z² are not both N at the same time;
Ring S is a 6-membered aromatic ring;
Ring T is a C₃-C₁₀ carbocycle optionally further substituted with one or more instances of J^{T};
Q¹ is —C(O)—, —CO₂—, —OC(O)—, —O(CR^{t}R^{s})ₖ—C(O)O—, —C(O)NR′—, —C(O)N(R′)—O—, —C(O)NRC(O)O—, —NRC(O)—, —NRC(O)NR′—, —NRCO₂—, —OC(O)NR′—, —OSO₂NR′—, —S(O)—, —SO₂—, —SO₂NR′—, —NRSO₂—, —NRSO₂NR′—, —P(O)(OR)O—, —OP(O)(OR^{a})O—, —P(O)₂O—, —CO₂SO₂—, —B(O)₂—, or —(CR^{t}R^{s})ₚ—Y¹—;
Y¹ is —C(O)—, —CO₂—, —OC(O)—, —O(CR^{t}R^{s})ₖ—C(O)O—, —C(O)NR′—, —C(O)N(R′)—O—, —C(O)NRC(O)O—, —NRC(O)—, —NRC(O)NR′—, —NRCO₂—, —OC(O)NR′—, —OSO₂NR′—, —S(O)—, —SO₂—, —SO₂NR′—, —NRSO₂—, —NRSO₂NR′—, —P(O)(OR)O—, —OP(O)(OR^{a})O—, —P(O)₂O—, —B(O)₂—, or —CO₂SO₂—;
R¹ is: i) —H; ii) a C₁-C₆ aliphatic group optionally substituted with one or more instances of J^{A}; iii) a C₃-C₁₀ carbocyclic group or 4-10 membered heterocyclic group, each optionally and independently substituted with one or more instances of J^{B}; or iv) a 6-10 membered aryl group or 5-10 membered heteroaryl group, each optionally and independently substituted with one or more instances of J^{C}; or
optionally R¹, together with R′ and the nitrogen to which they are attached, form a 4-8 membered heterocyclic group optionally substituted with one or more instances of J²; or
optionally -Q¹-R¹ forms, together with Ring T, a 4-10 membered, non-aromatic, spiro ring optionally substituted with one or more instances of J⁴; and
R² is —H, halogen, —CN, —NO₂, —C(O)NH₂, —C(O)NH(CH₃), —C(O)N(CH₃)₂, or C₁-C₆ aliphatic optionally substituted with one or more instances of J¹;
J^{A}, J^{B}, and J^{T} are each and independently oxo or J^{C};
J^{C} are each and independently selected from the group consisting of halogen, cyano, M, R^{a}, or R^{a}-M;
M is independently selected from the group consisting of —OR^{b}, —SR^{b}, —S(O)R^{a}, —SO₂R^{a}, —NR^{b}R^{c}, —C(O)R^{a}, —C(═NR)R^{c}, —C(═NR)NR^{b}R^{c}, —NRC(═NR)NR^{b}R^{c}, —C(O)OR^{b}, —OC(O)R^{b}, —NRC(O)R^{b}, —C(O)NR^{b}R^{c}, —NRC(O)NR^{b}R^{c}, —NRC(O)OR^{b}, —OCONR^{b}R^{c}, —C(O)NRCO₂R^{b}, —NRC(O)NRC(O)OR^{b}, —C(O)NR(OR^{b}), —OSO₂NR^{b}R^{c}, —SO₂NR^{c}R^{b}, —NRSO₂R^{b}, —NRSO₂NR^{c}R^{b}, —P(O)(OR^{b})₂, —OP(O)(OR^{b})₂, —P(O)₂OR^{b} and —CO₂SO₂R^{b}; or
optionally, two J^{T}, two J^{A}, two J^{B}, and two J^{C}, respectively, together with the atom(s) to which they are attached, independently form a 4-10-membered ring that is optionally substituted with one or more instances of J⁴; and
R^{a} is independently:
i) a C₁-C₆ aliphatic group optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), C₃-C₈ carbocyclic group optionally substituted with one or more instances of J², 4-8 membered heterocyclic group optionally substituted with one or more instances of J², 5-10 membered heteroaryl group optionally substituted with one or more instances of J³, and 6-10 membered aryl group optionally substituted with one or more instances of J³;
ii) a C₃-C₈ carbocyclic group, or 4-8 membered heterocyclic group, each of which is optionally and independently substituted with one or more instances of J²; or
iii) a 5-10 membered heteroaryl group, or 6-10 membered aryl group, each of which is optionally and independently substituted with one or more instances of J³; and
R^{b} and R^{c} are each independently R^{a} or —H; or optionally, R^{b} and R^{c}, together with the nitrogen atom(s) to which they are attached, each independently form a 4-8 membered heterocyclic group optionally substituted with one or more instances of J²;
R^{t} and R^{s} are each independently —H, halogen, or C₁-C₆ alkyl optionally substituted with one or more instances of J¹, or optionally, R^{t} and R^{s}, together with the carbon atom to which they are attached, form a cyclopropane ring optionally substituted with one or more instances of methyl;
R and R′ are each independently —H or C₁-C₆ alkyl optionally and independently substituted with one or more instances of J¹, or optionally R and R′, together with the nitrogen to which they are attached, form a 4-8 membered heterocyclic group optionally substituted with one or more instances of J²;
each J¹ is independently selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), and phenyl;
each J² is independently selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl);
each of J³ and J⁴ is independently selected from the group consisting of halogen, cyano, hydroxy, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl);
p is independently 1, 2, 3 or 4; and
k is 1, 2, 3 or 4; and
provided that Q¹-R¹ is not at the same carbon atom to which —NH group that is attached to Ring S is attached.

2. The compound of claim 1, wherein at least one of Z¹-Z⁴ is N;
X is —Cl, —Br, —F, —CN, —O(C₁₋₄ alkyl), C₁₋₆ alkyl, or C₁₋₆haloalkyl;
Ring S is selected from: 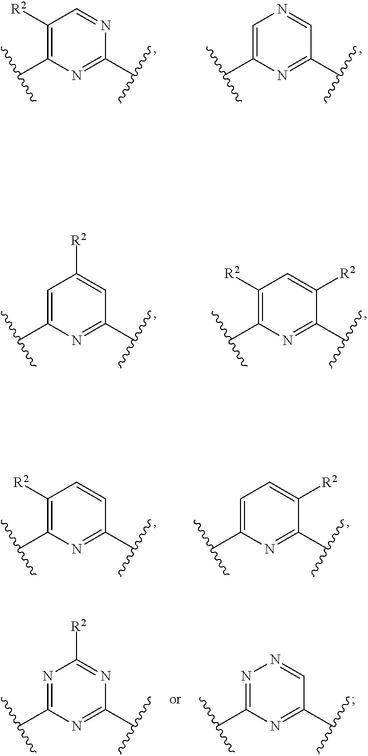
Q¹ is —C(O)—, —CO₂—, —OC(O)—, —O(CR^{t}R^{s})ₖ—C(O)O—, —C(O)NR′—, —C(O)N(R′)—O—, —C(O)NRC(O)O—, —NRC(O)—, —NRC(O)NR′—, —NRCO₂—, —OC(O)NR′—, —OSO₂NR′—, —S(O)—, —SO₂—, —SO₂NR′—, —NRSO₂—, —NRSO₂NR′—, —B(O)₂—, or —(CR^{t}R^{s})ₚ—Y—;
Y¹ is —C(O)—, —CO₂—, —OC(O)—, —O(CR^{t}R^{s})ₖ—C(O)O—, —C(O)NR′—, —C(O)N(R′)—O—, —C(O)NRC(O)O—, —NRC(O)—, —NRC(O)NR′—, —NRCO₂—, —OC(O)NR′—, —OSO₂NR′—, —S(O)—, —SO₂—, —SO₂NR′—, —NRSO₂—, —B(O)₂—, or —NRSO₂NR′—.
R¹ is independently i) —H; ii) a C₁-C₆-aliphatic group optionally substituted with one or more instances of J^{A}; iii) a C₃-C₈ carbocyclic group or 4-8 membered heterocyclic group, each of which is optionally and independently substituted with one or more instances of J^{B}; iv) a phenyl group or 5-6 membered heteroaryl group, each of which is optionally and independently substituted with one or more instances of J^{C}; or
optionally R¹, together with R′ and the nitrogen to which they are attached, form an optionally substituted, 4-8 membered heterocyclic group; or optionally -Q¹-R¹ forms, together with Ring T, an optionally substituted, 4-10 membered, non-aromatic, spiro ring; and
R² is —F, —Cl, —CN, C₁-C₄ aliphatic, or C₁-C₄ haloalkyl;
J^{A}, J^{B}, and J^{T} are each independently oxo or J^{C};
J^{C} is selected from the group consisting of halogen, cyano, R^{a}, —OR^{b}, —SR^{b}, —S(O)R^{a}, —SO₂R^{a}, —NHR^{c}, —C(O)R^{b}, —C(O)OR^{b}, —OC(O)R^{b}, —NHC(O)R^{b}, —C(O)NHR^{c}, —NHC(O)NHR^{c}, —NHC(O)OR^{b}, —OCONHR^{c}, —NHC(O)NHC(O)OR^{b}, —N(CH₃)R^{c}, —N(CH₃)C(O)R^{b}, —C(O)N(CH₃)R^{c}, —N(CH₃)C(O)NHR^{c}, —N(CH₃)C(O)OR^{b}, —OCON(CH₃)R^{c}, —C(O)NHCO₂R^{b}, —C(O)N(CH₃)CO₂R^{b}, —N(CH₃)C(O)NHC(O)OR^{b}, —NHSO₂R^{b}, —SO₂NHR^{b}, —SO₂N(CH₃)R^{b}, and —N(CH₃)SO₂R^{b},
optionally, two J^{T}, two J^{A}, two J^{B}, and two J^{C}, respectively, together with the atom(s) to which they are attached, independently form an optionally substituted, 4-10-membered, non-aromatic ring; and
R^{a} is independently: i) a C₁-C₆ alkyl group optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), optionally substituted C₃-C₈ carbocyclic group, optionally substituted 4-8 membered heterocyclic group, optionally substituted 5-6 membered heteroaryl, and optionally substituted phenyl group; ii) an optionally substituted C₃-C₈ carbocyclic group; iii) optionally substituted 4-8 membered heterocyclic group; iv) an optionally substituted 5-6 membered heteroaryl group; v) or optionally substituted phenyl group;
R^{b} and R^{c} are each independently R^{a} or —H; or optionally, R^{b} and R^{c}, together with the nitrogen atom(s) to which they are attached, each independently form an optionally substituted, 4-8 membered heterocyclic group; and
R and R′ are each and independently —H or C₁₋₄ alkyl, or optionally R and R′, together with the nitrogen to which they are attached, form an optionally substituted 4-8 membered heterocyclic group, or optionally R′, together with R¹ and the nitrogen to which they are attached, form an optionally substituted 4-8 membered heterocyclic group.

3. (canceled)

4. (canceled)

5. (canceled)

6. The compound of claim 2, wherein
X is —Cl, —Br, —F, —CN, —CH₃, or CF₃;
Q¹ is —CO₂—, —O(CR^{t}R^{s})ₖ—C(O)O—, —P(O)(OR)O—, —OP(O)(OR^{a})O—, —P(O)₂O—, —CO₂SO₂— or —(CR^{t}R^{s})ₚ—Y¹—;
Y¹ is —CO₂—, —O(CR^{t}R^{s})ₖ—C(O)O—, —P(O)(OR)O—, —OP(O)(OR^{a})O—, —P(O)₂O—, or —CO₂SO₂—; and
Ring S is

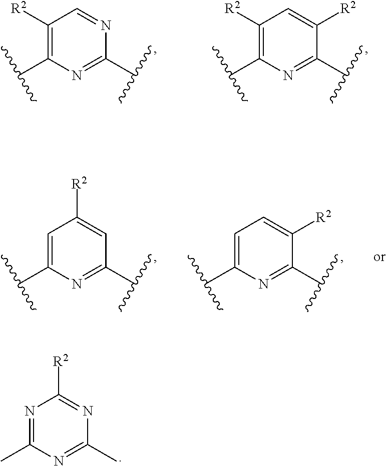

7. (canceled)

8. The compound of claim 6, wherein Ring T is an optionally substituted, bridged, C₅-C₁₀ carbocyclic group, or an optionally substituted, monocyclic, C₅-C₈ carbocyclic group.

9. (canceled)

10. (canceled)

11. (canceled)

12. (canceled)

13. (canceled)

14. (canceled)

15. The compound of claim, wherein Ring S is selected from:

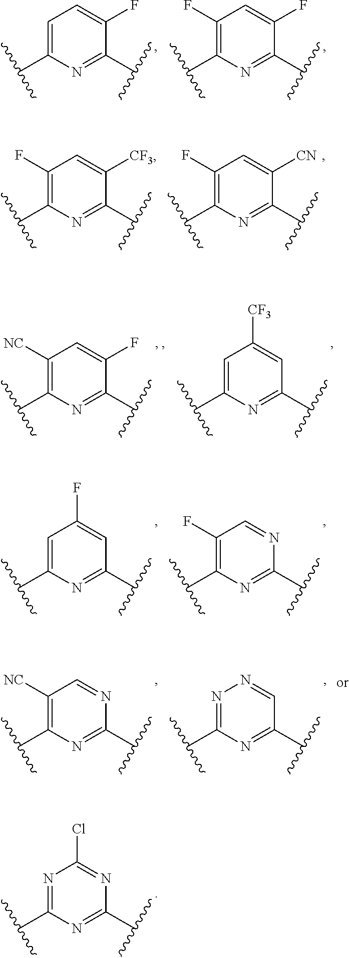

16. The compound of claim 15, wherein Ring T is: 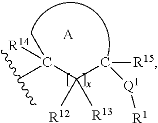
and wherein:
Ring A is a 5-10 membered carbocyclic group optionally further substituted with one or more substituents selected from the group consisting of halogen, cyano, hydroxy, oxo, —NH₂, —NH(C₁-C₄ alkyl), —N(C₁-C₄ alkyl), —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl); or Ring A and R¹⁵, Ring A and R¹⁴, or Ring A and R¹³ independently and optionally form a bridged carbocyclic group optionally and independently substituted with one or more substituents selected from the group consisting of halogen, cyano, hydroxy, oxo, —NH, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl);
X is —F or —Cl;
Q¹ is —C(O)O—, —NRC(O)—, —C(O)NR—, —NRC(O)NR′—, or —(CR^{t}R^{s})_{1,2}—Y¹—;
Y¹ is —C(O)O—, —NRC(O)—, —C(O)NR—, or —NRC(O)NR′—;
R¹ is independently: i) —H; ii) a C₁-C₆ aliphatic group optionally substituted with one or more substituents independently selected from the group consisting of halogen, cyano, hydroxy, oxo, —O(C₁-C₄ alkyl), —NH₂, —NH(C₁-C₄ alkyl), —N(C₁-C₄ alkyl)₂, —C(O)(C₁-C₄ alkyl), —OC(O)(C₁-C₄ alkyl), —C(O)O(C₁-C₄ alkyl), —CO₂H, C₃-C₈ carbocyclic group, 4-8 membered heterocyclic group, phenyl, and 5-6 membered heteroaryl; iii) a C₃-C₇ carbocyclic group; iv) a 4-7 membered heterocyclic group; v) a phenyl group; or vi) a 5-6 membered heteroaryl group;
optionally R¹, together with R′ and the nitrogen to which they are attached, form an optionally substituted, 4-8 membered heterocyclic group;
each of said carbocyclic, phenyl, heterocyclic, and heteroaryl groups represented by R¹ and for the substituents of the C₁-C₆-aliphatic group represented by R¹, and said heterocyclic group formed with R¹ and R′ is independently and optionally substituted with one or more substituents independently selected from the group consisting of halogen, cyano, hydroxy, oxo, —NH, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl);
each of R¹², R¹³, and R¹⁴ is independently —H, halogen, cyano, hydroxy, C₁-C₆ alkyl, —O(C₁-C₆ alkyl), —NH₂, —NH(C₁-C₆ alkyl), —N(C₁-C₆ alkyl)₂, —OCO(C₁-C₆ alkyl), —CO(C₁-C₆ alkyl), —CO₂H, or —CO₂(C₁-C₆ alkyl), wherein each said C₁-C₆ alkyl is optionally and independently substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), and —O(C₁-C₄ alkyl);
each R¹⁵ is independently —H, halogen, cyano, hydroxy, or C₁-C₆ alkyl optionally and independently substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), and —O(C₁-C₄ alkyl);
x is 0, 1 or 2; and
J^{A}, J^{B}, J^{C}, and J^{T} are each independently selected from the group consisting of halogen, cyano, R^{a}, —OR^{b}, —NHR^{c}, —C(O)R^{b}, —C(O)OR^{b}, —OC(O)R^{b}, —NHC(O)R^{b}, —C(O)NHR^{c}, —NHC(O)NHR^{c}, —NHC(O)OR^{b}, —OCONHR^{c}, —N(CH₃)R^{c}, —N(CH₃)C(O)R^{b}, —C(O)N(CH₃)R^{c}, —N(CH₃)C(O)NHR^{c}, —N(CH₃)C(O)OR^{b}, —NHSO₂R^{b}, —SO₂NHR^{b}, —SO₂N(CH₃)R^{b}, and —N(CH₃)SOR^{b}; or
optionally, two J^{T}, two J^{A}, two J^{B}, and two J^{C}, respectively, together with the atom(s) to which they are attached, independently form a 4-10-membered ring that is optionally substituted with one or more substituents selected from the group consisting of halogen, cyano, hydroxy, oxo, —NH, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), and —O(C₁-C₄ alkyl); and
R^{a} is independently: i) a C₁-C₆ alkyl group optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), C₃-C₈ carbocycle, 4-8 membered heterocycle, 5-6 membered heteroaryl, and phenyl; ii) a C₃-C₈ carbocyclic group or 4-8 membered heterocyclic group, each of which is independently and optionally substituted with one or more substituents selected from the group consisting of halogen, cyano, hydroxy, oxo, —NH, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl); or iii) a 5-6 membered heteroaryl group or phenyl group, each of which is independently and optionally substituted with one or more substituents selected from the group consisting of halogen, cyano, hydroxy, —NH, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl); and
R^{b} and R^{c} are each independently R^{a} or —H; or optionally, R^{b} and R^{c}, together with the nitrogen atom(s) to which they are attached, each independently form a 4-8 membered heterocyclic group optionally substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl).

17. (canceled)

18. (canceled)

19. (canceled)

20. The compound of claim 16, wherein Ring S is selected from:

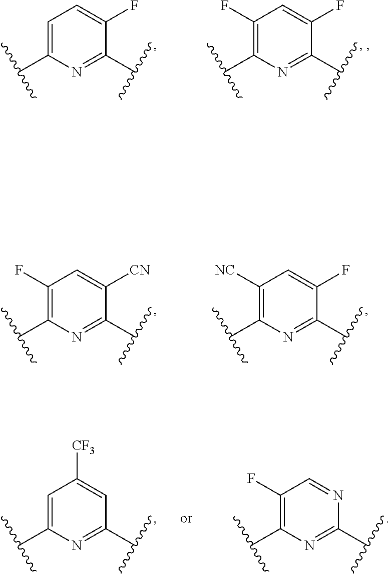 [SMILES: *c1ccc(F)c(*)n1 ; *c1nc(*)c(F)cc1F ; *c1nc(*)c(C#N)cc1F ; *c1nc(*)c(C#N)cc1F ; *c1cc(C(F)(F)F)cc(*)n1 ; *c1ncc(F)c(*)n1]

21. The compound of claim 20, wherein:
(a) R¹², R¹³, and R¹⁴ are each and independently —H, halogen, cyano, hydroxy, —O(C₁-C₆ alkyl), or optionally substituted C₁-C₆ alkyl;
R¹⁵ is —H or optionally substituted C₁-C₆ alkyl; and
R^{t} and R^{s} are each independently —H, halogen, C₁-C₆ alkyl, or C₁-C₆ haloalkyl; or
(b) R¹² and R¹³ are each independently —H, halogen, hydroxy, C₁-C₆ alkyl, C₁-C₆ haloalkyl, or —O(C₁-C₆ alkyl);
R¹⁴ and R¹⁵ are each independently —H, C₁-C₆ alkyl, or C₁-C₆ haloalkyl; and
R^{t} and R^{s} are each independently —H or C₁-C₆ alkyl.

22. (canceled)

23. (canceled)

24. (canceled)

25. The compound of claim 21, wherein Ring A and R¹⁵, Ring A and R¹⁴, or Ring A and R¹³ independently form an optionally substituted, bridged carbocyclic group.

26. The compound of claim 25, wherein Ring T is: 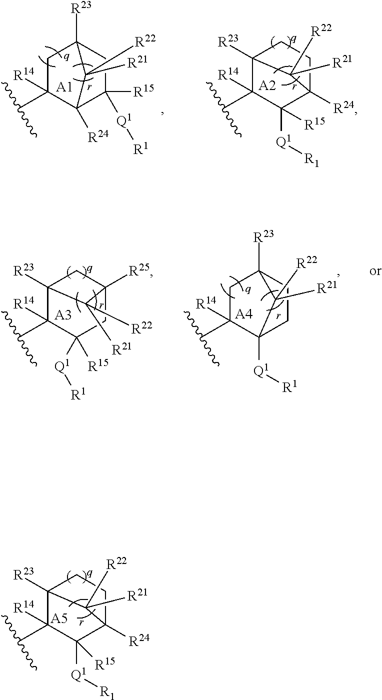
wherein:
each of Rings A1-A5 is independently a 5-10 membered, bridged carbocycle optionally further substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl);
Q¹ is independently —C(O)O—, —NRC(O)—, —C(O)NR—, —NRC(O)NR′—, or —(CH₂)_{1,2}—Y¹—;
Y¹ is independently —C(O)O—, —NRC(O)—, —C(O)NR—, or —NRC(O)NR′—;
each R¹⁴ is independently —H, halogen, cyano, hydroxy, C₁-C₆ alkyl, —O(C₁-C₆ alkyl), —NH₂, —NH(C₁-C₆ alkyl), —N(C₁-C₆ alkyl)₂, —OCO(C₁-C₆ alkyl), —CO(C₁-C₆ alkyl), —CO₂H, or —CO₂(C₁-C₆ alkyl), wherein each said C₁-C₆ alkyl is optionally and independently substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), and —O(C₁-C₄ alkyl);
each R¹⁵ is independently —H, halogen, cyano, hydroxy, or C₁-C₆ alkyl optionally and independently substituted with one or more substituents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), and —O(C₁-C₄ alkyl); and
R²¹, R²², R²³, R²⁴, and R²⁵ are each independently —H, halogen, —OH, C₁-C₆ alkoxy, or C₁-C₆ alkyl optionally substituted with one or more substituents independently selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl);
q is 0, 1 or 2; and
r is 1 or 2.

27. The compound of claim 26, wherein:
R¹⁴ and each R¹⁵ are each independently —H, C₁-C₆ alkyl, or C₁-C₆ haloalkyl; and
R²¹, R²², R²³, R²⁴, and R²⁵ are each independently —H, halogen, hydroxy, C₁-C₆ alkoxy, C₁-C₆ alkyl, or C₁-C₆ haloalkyl.

28. (canceled)

29. The compound of claim 27, wherein
Q¹ is independently —C(O)O—, —NRC(O)—, or —C(O)NR—
R¹ is independently —H or an optionally substituted C₁-C₆ aliphatic group; and
R and R′ are each and independently —H or —CH₃; or optionally R¹, together with R′ and the nitrogen to which they are attached, form an optionally substituted, 4-8 membered heterocyclic group.

30. (canceled)

31. (canceled)

32. The compound of claim 29, wherein
Ring T is: 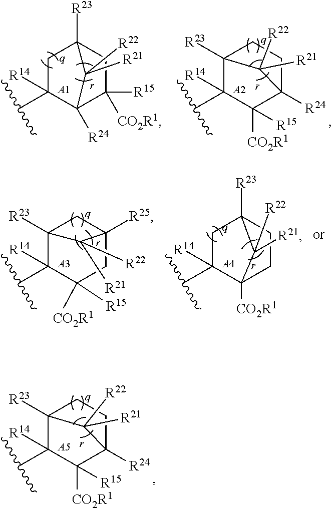
wherein each of Rings A1-A5 is independently and optionally further substituted with one or more substituents selected from the group consisting of halogen, cyano, hydroxy, C₁-C₄ alkyl, C₁-C₄ haloalkyl, and —O(C₁-C₄ alkyl);
R¹⁴ and each R¹⁵ are each independently —H or C₁₋₆ alkyl; and
R²¹, R²², R²³, R²⁴, and R²⁵ are each independently —H or C₁₋₆ alkyl.

33. (canceled)

34. The compound of claim 32 any one of claims 26-32, wherein:
R¹ is H or optionally substituted C₁₋₆ alkyl;
R¹⁴, R¹⁵, R²¹, R²², R²³, R²⁴, and R²⁵ are each independently —H; and
q is 1.

35. (canceled)

36. The compound of claim 21, wherein
Ring T is selected from: 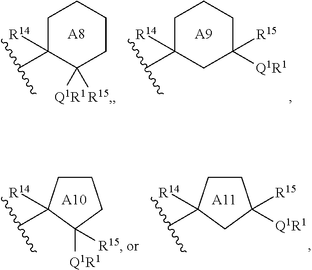
wherein:
Q¹ is independently —C(O)—, —C(O)O—, —NRC(O)—, —C(O)NR—, —NRC(O)NR′—, or —(CH₂)_{1,2}—Y—;
Y¹ is independently —C(O)—, —C(O)O—, —NRC(O)—, —C(O)NR—, or —NRC(O)NR′—;
R and R′ are each and independently —H or —CH₃; and
R¹ is independently a 4-7 membered heterocyclic group, a phenyl group, or a 5-6 membered heteroaryl group, wherein each of said heterocyclic, phenyl and heteroaryl groups is independently and optionally substituted with one or more substituents independently selected from the group consisting of halogen, cyano, hydroxy, oxo, —NH, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl); or
optionally R¹ and R′, together with the nitrogen atom to which they are attached, form an optionally substituted, 4-8 membered heterocyclic group; and
R¹⁴ and each R¹⁵ are each independently —H, C₁-C₆ alkyl, or C₁-C₆ haloalkyl; and
each of Rings A8-A11 is independently and optionally substituted with one or more substitutents selected from the group consisting of halogen, cyano, hydroxy, oxo, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), C₁-C₄ alkyl, C₁-C₄ haloalkyl, and —O(C₁-C₄ alkyl).

37. (canceled)

38. The compound of claim 36, wherein:
Q¹ is independently —NRC(O)—, —C(O)NR—, or —NRC(O)NR′—;
R¹⁴ and each R¹⁵ are each independently —H or C₁₋₆ alkyl;
each of Rings A8-A11 is independently and optionally substituted with one or more substitutents selected from the group consisting of halogen, cyano, hydroxy, C₁-C₄ alkyl, C₁-C₄ haloalkyl, and —O(C₁-C₄ alkyl); and
R and R′ are each and independently —H or —CH₃.

39. (canceled)

40. (canceled)

41. (canceled)

42. (canceled)

43. (canceled)

44. A compound selected from any of one of the structures depicted below or a pharmaceutically acceptable salt thereof: 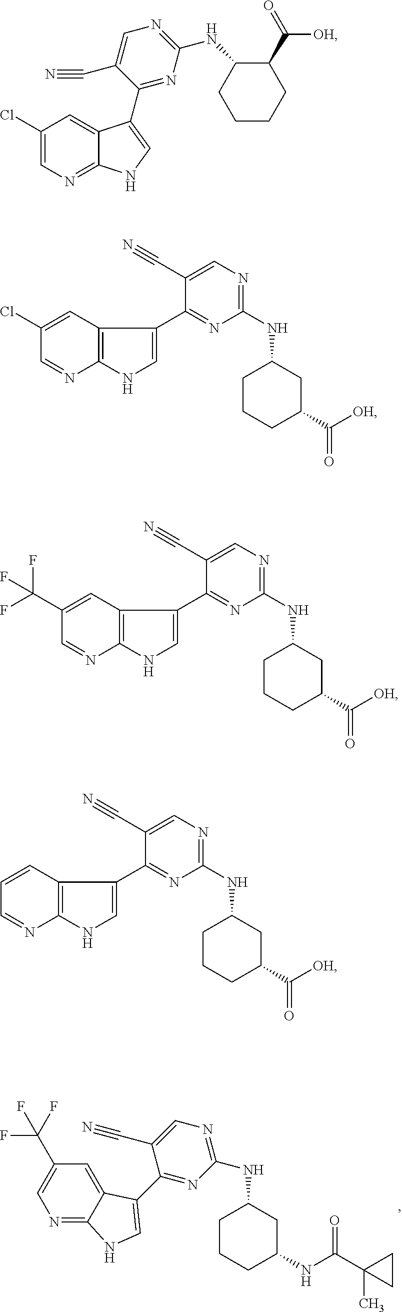 [SMILES: N#Cc1cnc(N[C@H]2CCCC[C@@H]2C(=O)O)nc1-c1c[nH]c2ncc(Cl)cc12 ; N#Cc1cnc(N[C@H]2CCC[C@@H](C(=O)O)C2)nc1-c1c[nH]c2ncc(Cl)cc12 ; N#Cc1cnc(N[C@H]2CCC[C@@H](C(=O)O)C2)nc1-c1c[nH]c2ncc(C(F)(F)F)cc12 ; N#Cc1cnc(N[C@H]2CCC[C@@H](C(=O)O)C2)nc1-c1c[nH]c2ncccc12 ; CC1(C(=O)N[C@@H]2CCC[C@H](Nc3ncc(C#N)c(-c4c[nH]c5ncc(C(F)(F)F)cc45)n3)C2)CC1] 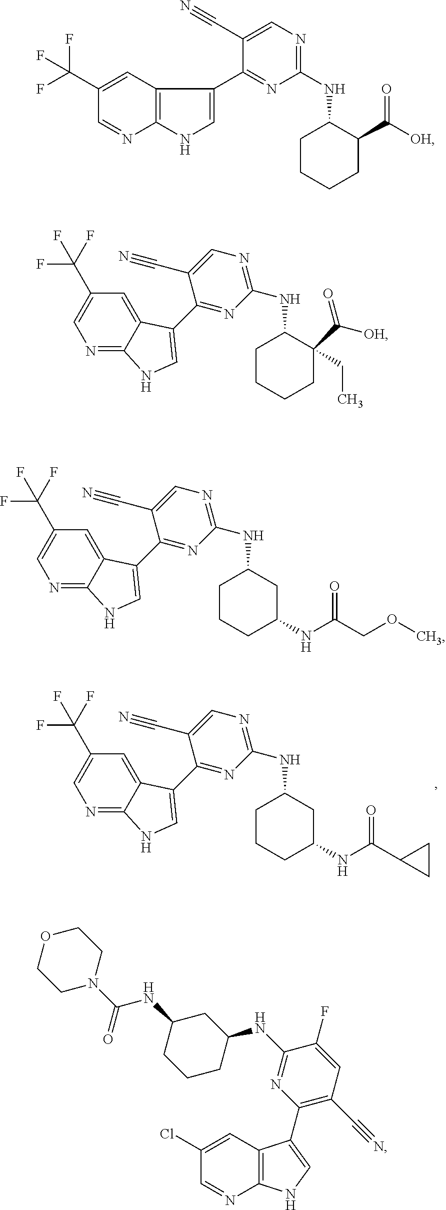 [SMILES: N#Cc1cnc(N[C@H]2CCCC[C@@H]2C(=O)O)nc1-c1c[nH]c2ncc(C(F)(F)F)cc12 ; CC[C@]1(C(=O)O)CCCC[C@@H]1Nc1ncc(C#N)c(-c2c[nH]c3ncc(C(F)(F)F)cc23)n1 ; COCC(=O)N[C@@H]1CCC[C@H](Nc2ncc(C#N)c(-c3c[nH]c4ncc(C(F)(F)F)cc34)n2)C1 ; N#Cc1cnc(N[C@H]2CCC[C@@H](NC(=O)C3CC3)C2)nc1-c1c[nH]c2ncc(C(F)(F)F)cc12 ; N#Cc1cc(F)c(N[C@H]2CCC[C@@H](NC(=O)N3CCOCC3)C2)nc1-c1c[nH]c2ncc(Cl)cc12] 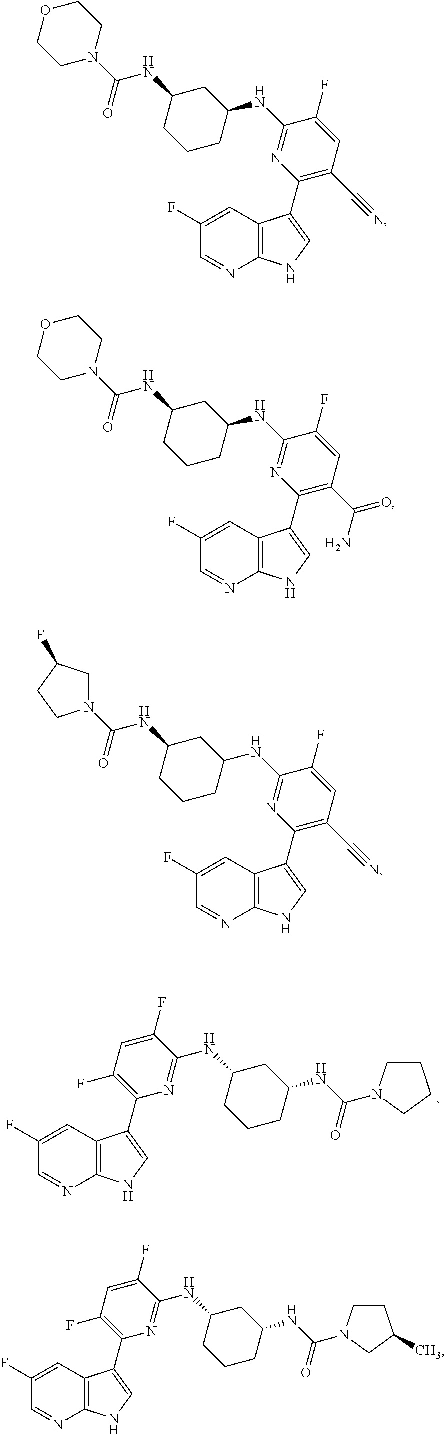 [SMILES: N#Cc1cc(F)c(N[C@H]2CCC[C@@H](NC(=O)N3CCOCC3)C2)nc1-c1c[nH]c2ncc(F)cc12 ; NC(=O)c1cc(F)c(N[C@H]2CCC[C@@H](NC(=O)N3CCOCC3)C2)nc1-c1c[nH]c2ncc(F)cc12 ; N#Cc1cc(F)c(NC2CCC[C@@H](NC(=O)N3CC[C@@H](F)C3)C2)nc1-c1c[nH]c2ncc(F)cc12 ; O=C(N[C@@H]1CCC[C@H](Nc2nc(-c3c[nH]c4ncc(F)cc34)c(F)cc2F)C1)N1CCCC1 ; C[C@@H]1CCN(C(=O)N[C@@H]2CCC[C@H](Nc3nc(-c4c[nH]c5ncc(F)cc45)c(F)cc3F)C2)C1] 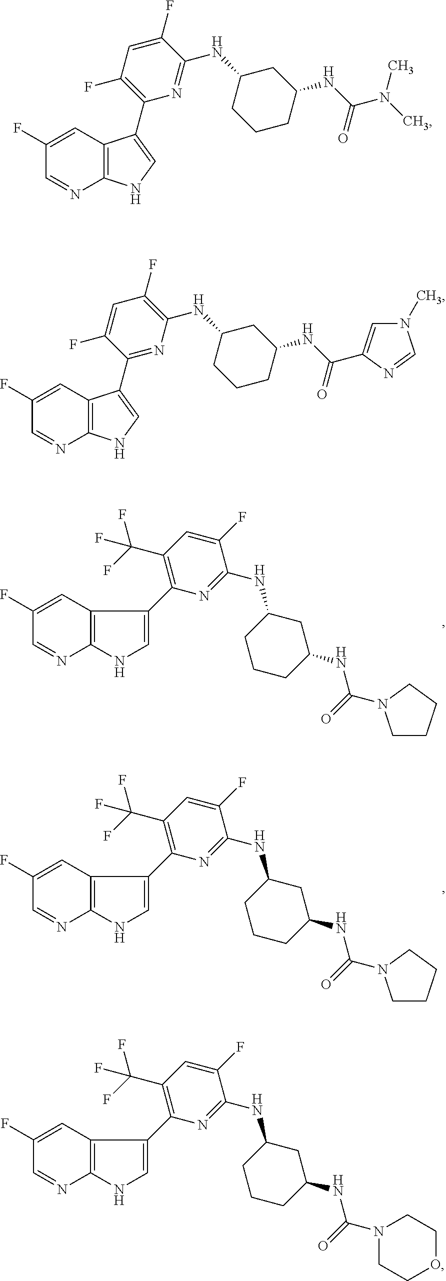 [SMILES: CN(C)C(=O)N[C@@H]1CCC[C@H](Nc2nc(-c3c[nH]c4ncc(F)cc34)c(F)cc2F)C1 ; Cn1cnc(C(=O)N[C@@H]2CCC[C@H](Nc3nc(-c4c[nH]c5ncc(F)cc45)c(F)cc3F)C2)c1 ; O=C(N[C@@H]1CCC[C@H](Nc2nc(-c3c[nH]c4ncc(F)cc34)c(C(F)(F)F)cc2F)C1)N1CCCC1 ; O=C(N[C@H]1CCC[C@@H](Nc2nc(-c3c[nH]c4ncc(F)cc34)c(C(F)(F)F)cc2F)C1)N1CCCC1 ; O=C(N[C@H]1CCC[C@@H](Nc2nc(-c3c[nH]c4ncc(F)cc34)c(C(F)(F)F)cc2F)C1)N1CCOCC1] 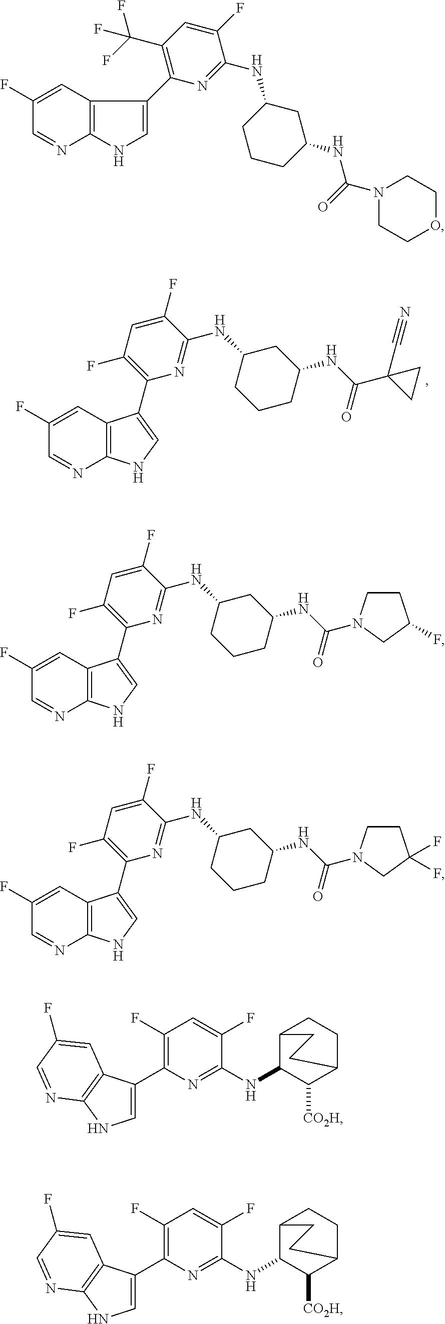 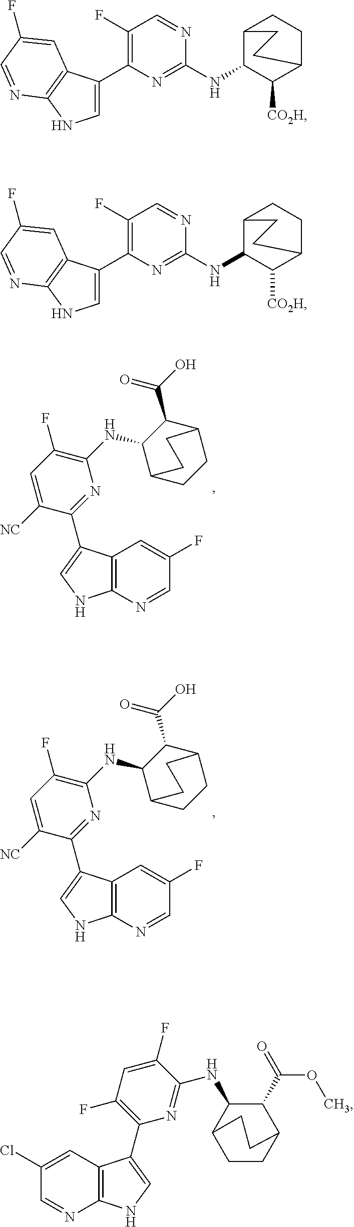 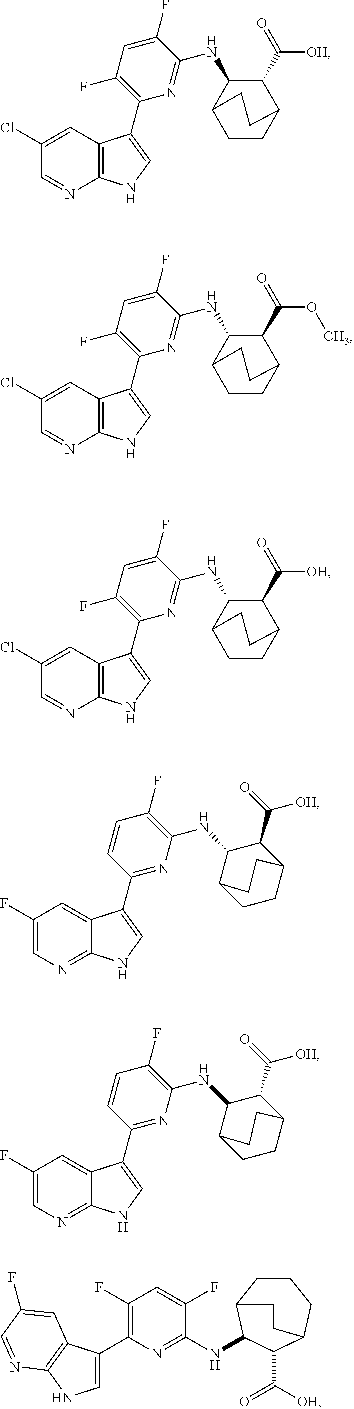 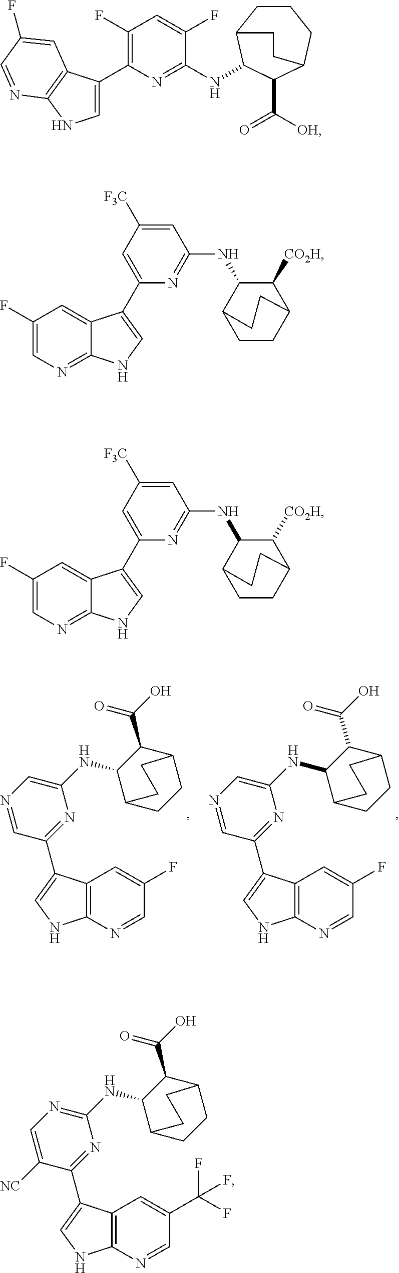 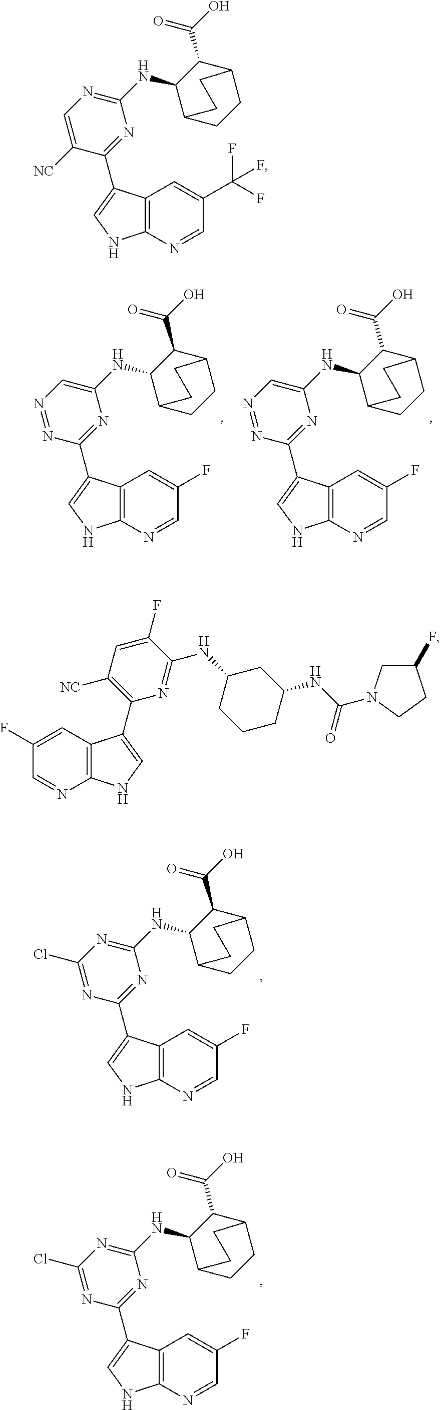 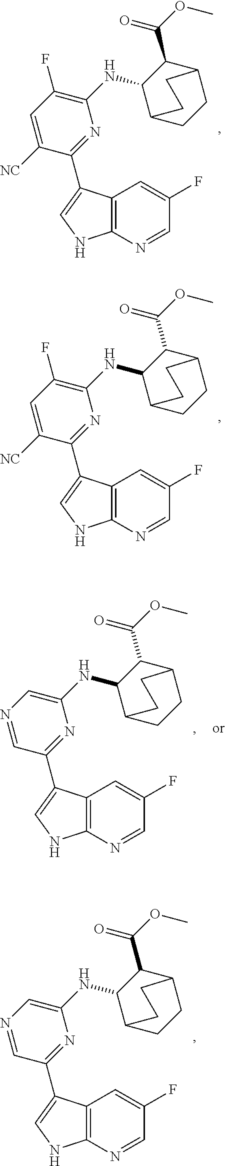 [SMILES: COC(=O)[C@H]1C2CCC(CC2)[C@@H]1Nc1nc(-c2c[nH]c3ncc(F)cc23)c(C#N)cc1F ; COC(=O)[C@@H]1C2CCC(CC2)[C@H]1Nc1nc(-c2c[nH]c3ncc(F)cc23)c(C#N)cc1F ; COC(=O)[C@@H]1C2CCC(CC2)[C@H]1Nc1cncc(-c2c[nH]c3ncc(F)cc23)n1 ; COC(=O)[C@H]1C2CCC(CC2)[C@@H]1Nc1cncc(-c2c[nH]c3ncc(F)cc23)n1]
or a pharmaceutically acceptable salt thereof.

45. A compound selected from any of one of the structures depicted below or a pharmaceutically acceptable salt thereof: 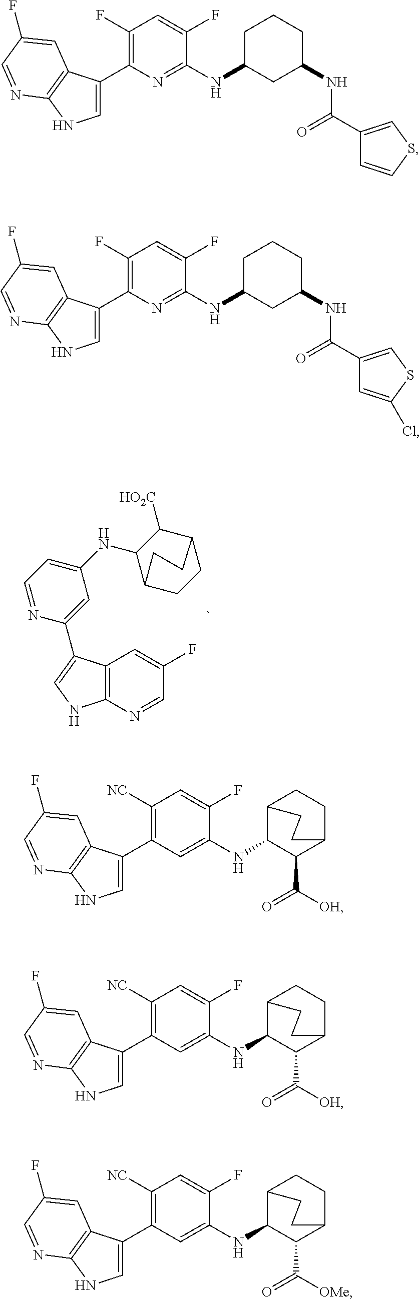 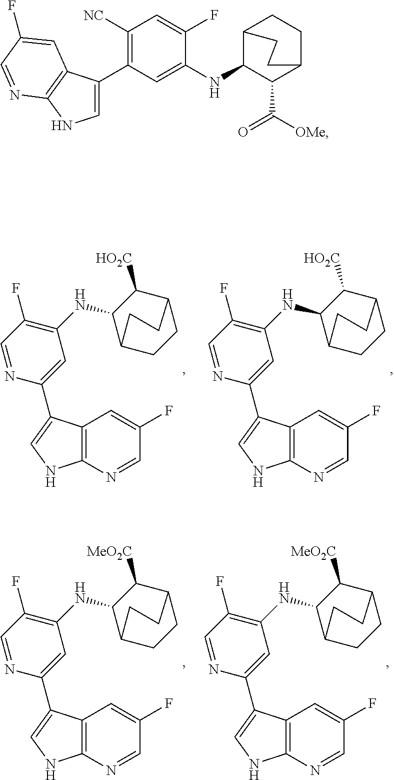
or a pharmaceutically acceptable salt thereof.

46. A pharmaceutical composition, comprising a compound according to any one of claims 1, 2, 6, 8, 15, 16, 20, 21, 25, 26, 27, 29, 32, 34, 36, 38, 44, and 45, and a pharmaceutically acceptable carrier, adjuvant or vehicle.

47. A method of inhibiting the replication of influenza viruses in a biological sample or patient, comprising the step of administering to said biological sample or patient an effective amount of a compound as described in any one of claims 1, 2, 6, 8, 15, 16, 20, 21, 25, 26, 27, 29, 32, 34, 36, 38, 44, and 45.

48. (canceled)

49. (canceled)

50. A method of reducing the amount of influenza viruses in a biological sample or in a patient, comprising administering to said biological sample or patient an effective amount of a compound as described in any one of claims 1, 2, 6, 8, 15, 16, 20, 21, 25, 26, 27, 29, 32, 34, 36, 38, 44, and 45.

51. A method of treating influenza in a patient, comprising administering to said patient an effective amount of a compound as described in any one of claims 1, 2, 6, 8, 15, 16, 20, 21, 25, 26, 27, 29, 32, 34, 36, 38, 44, and 45.

52. A method preparing a compound represented by Structural Formula (I): 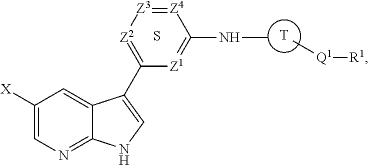
or a pharmaceutically acceptable salt thereof, comprising the steps of:
i) reacting compound A: 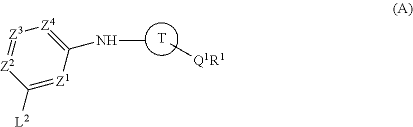
with compound (B): 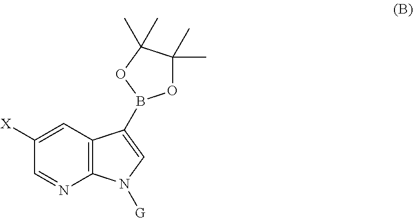
to form a compound represented by Structural Formula (XX): 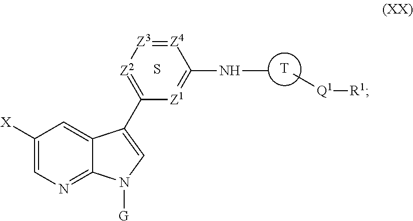
and
ii) deprotecting the G group of the compound of Structural Formula (XX) under suitable conditions to form the compound of Structural Formula (I), wherein:
the variables of Structural Formulae (I) and (XX), and compounds (A) and (B) are each independently as defined in any one of claims 1, 2, 6, 8, 15, 16, 20, 21, 25, 26, 27, 29, 32, 34, 36, 38, 44, and 45;
L² is a halogen; and
G is tosyl or trityl.

53. (canceled)

54. A method preparing a compound represented by Structural Formula (I): 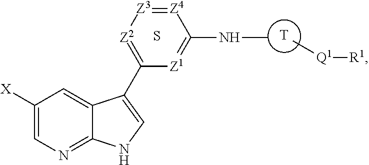
or a pharmaceutically acceptable salt thereof, comprising the steps of:
i) reacting compound (K) or (L): 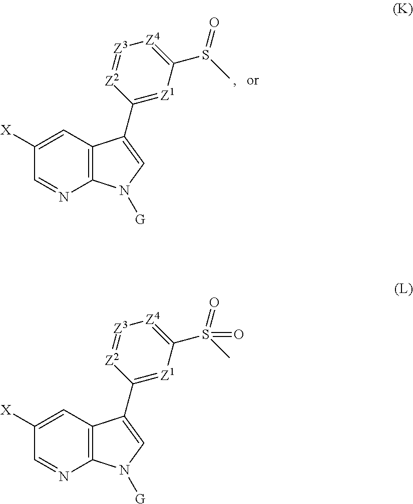
with compound (D): 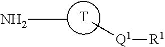
under suitable conditions to form a compound represented by Structural Formula (XX): 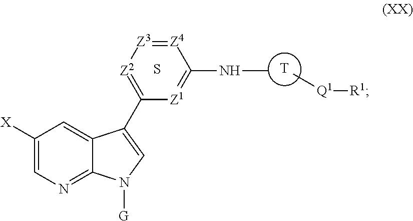
and
ii) deprotecting the G group of the compound of Structural Formula (XX) under suitable conditions to form the compound of Structural Formula (I), wherein:
the variables of Structural Formulae (I) and (XX), and compounds (K), (L), and (D) are each and independently as defined in any one of claims 1, 2, 6, 8, 15, 16, 20, 21, 25, 26, 27, 29, 32, 34, 36, 38, 44, and 45;
G is tosyl or trityl.

55. A method preparing a compound represented by Structural Formula (I): 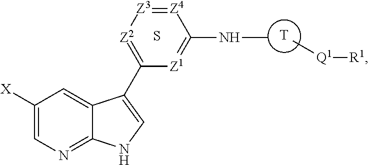
or a pharmaceutically acceptable salt thereof, comprising the steps of:
i) reacting Compound (G) with Compound (D): 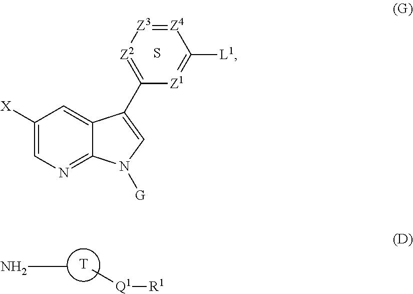
under suitable conditions to form a compound represented by Structural Formula (XX): 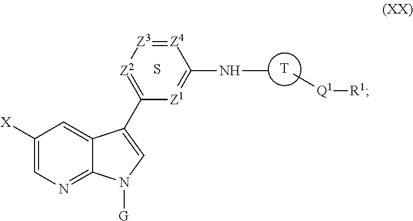
and
ii) deprotecting the G group of the compound of Structural Formula (XX) under suitable conditions to form the compound of Structural Formula (I), wherein:
the variables of Structural Formulae (I) and (XX), and Compounds (A) and (B) are each and independently as defined in any one of claims 1, 2, 6, 8, 15, 16, 20, 21, 25, 26, 27, 29, 32, 34, 36, 38, 44, and 45;
L¹ is a halogen; and
G is tosyl or trityl.

56. (canceled)

57. A compound represented by Structural Formula (XX): 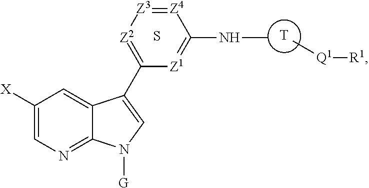
wherein the variables of Structural Formula (XX) are each and independently as defined in any one of claims 1, 2, 6, 8, 15, 16, 20, 21, 25, 26, 27, 29, 32, 34, 36, 38, 44, and 45; and
G is tosyl or trityl.

58. (canceled)

59. The compound of claim 57, represented by any one of the following structural formulae: 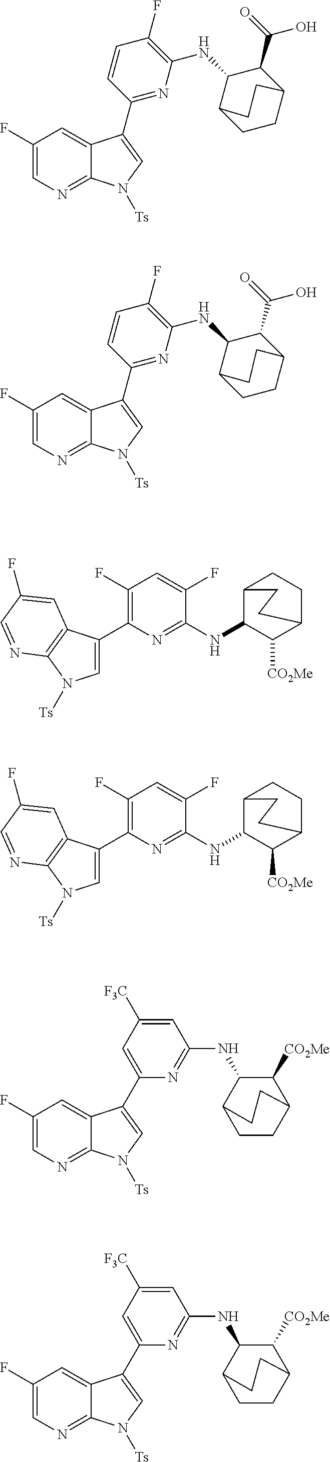 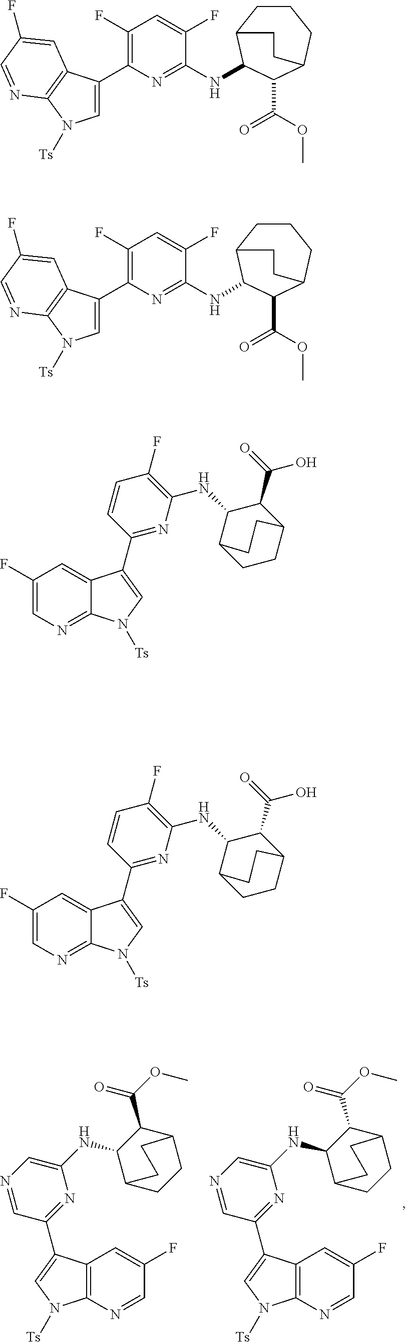 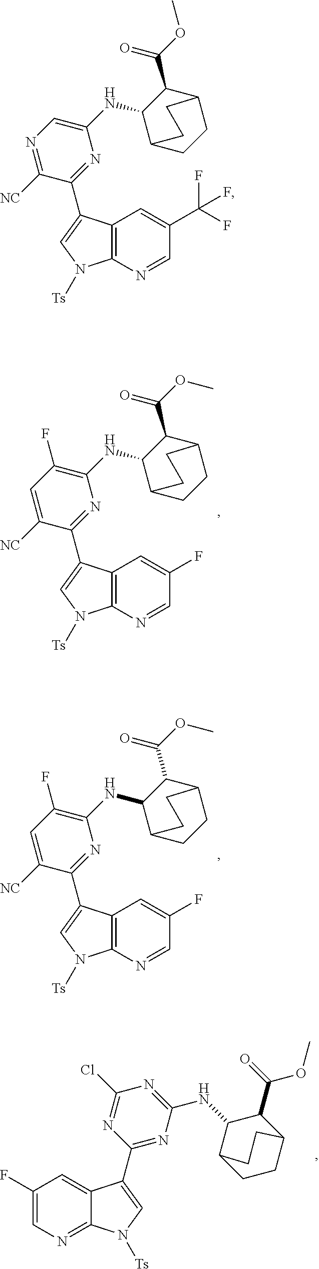 [SMILES: COC(=O)[C@H]1C2CCC(CC2)[C@@H]1Nc1cnc(C#N)c(-c2cn(S(=O)(=O)c3ccc(C)cc3)c3ncc(C(F)(F)F)cc23)n1 ; COC(=O)[C@H]1C2CCC(CC2)[C@@H]1Nc1nc(-c2cn(S(=O)(=O)c3ccc(C)cc3)c3ncc(F)cc23)c(C#N)cc1F ; COC(=O)[C@@H]1C2CCC(CC2)[C@H]1Nc1nc(-c2cn(S(=O)(=O)c3ccc(C)cc3)c3ncc(F)cc23)c(C#N)cc1F ; COC(=O)[C@H]1C2CCC(CC2)[C@@H]1Nc1nc(Cl)nc(-c2cn(S(=O)(=O)c3ccc(C)cc3)c3ncc(F)cc23)n1] 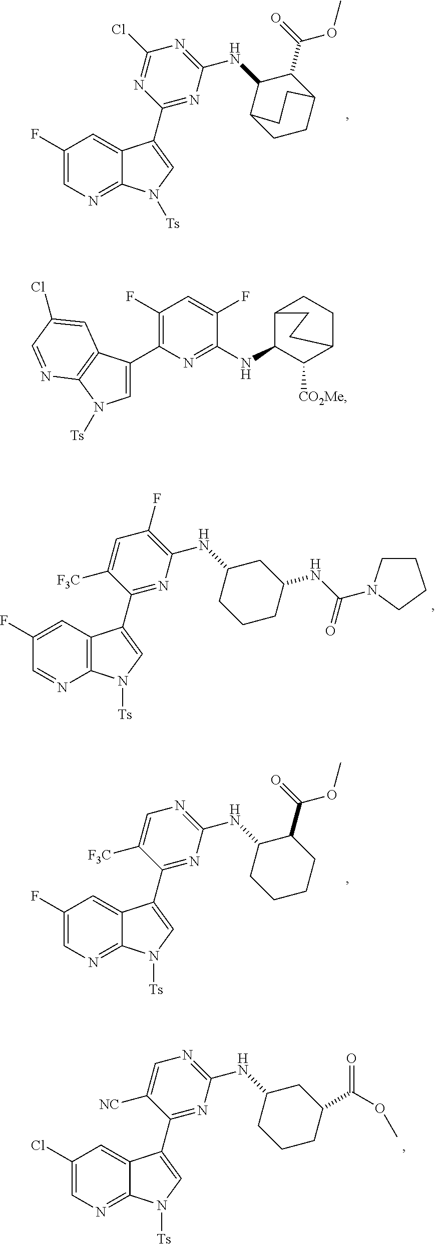 [SMILES: COC(=O)[C@@H]1C2CCC(CC2)[C@H]1Nc1nc(Cl)nc(-c2cn(S(=O)(=O)c3ccc(C)cc3)c3ncc(F)cc23)n1 ; COC(=O)[C@H]1C2CCC(CC2)[C@@H]1Nc1nc(-c2cn(S(=O)(=O)c3ccc(C)cc3)c3ncc(Cl)cc23)c(F)cc1F ; Cc1ccc(S(=O)(=O)n2cc(-c3nc(N[C@H]4CCC[C@@H](NC(=O)N5CCCC5)C4)c(F)cc3C(F)(F)F)c3cc(F)cnc32)cc1 ; COC(=O)[C@H]1CCCC[C@@H]1Nc1ncc(C(F)(F)F)c(-c2cn(S(=O)(=O)c3ccc(C)cc3)c3ncc(F)cc23)n1 ; COC(=O)[C@@H]1CCC[C@H](Nc2ncc(C#N)c(-c3cn(S(=O)(=O)c4ccc(C)cc4)c4ncc(Cl)cc34)n2)C1] 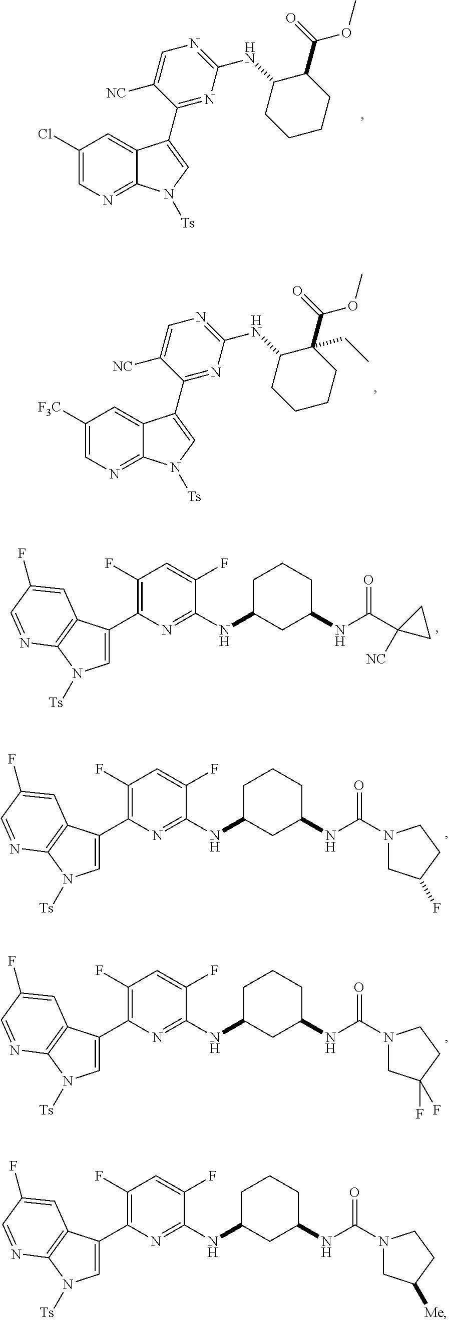 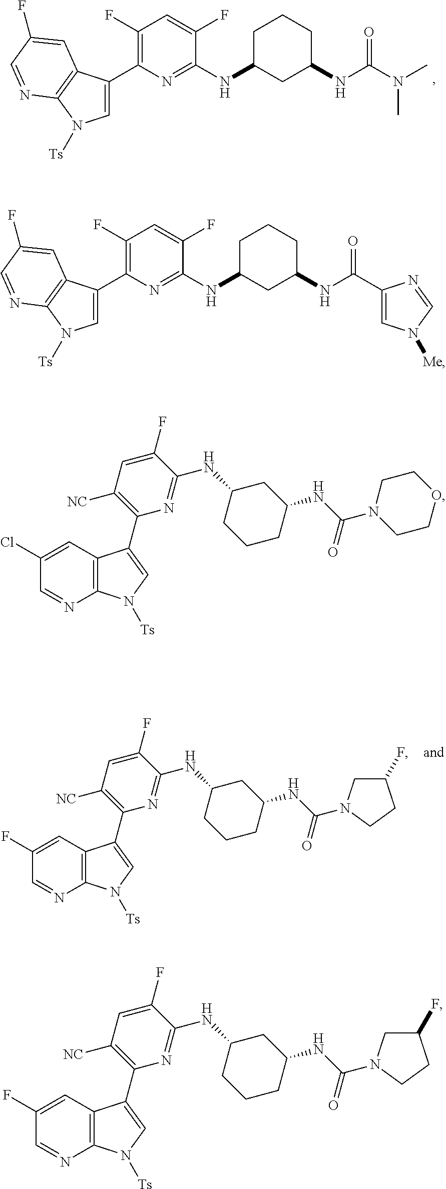
or a pharmaceutically acceptable salt thereof, wherein Ts is tosyl.

60. The compound of claim 57, represented by any one of the following structural formulae: 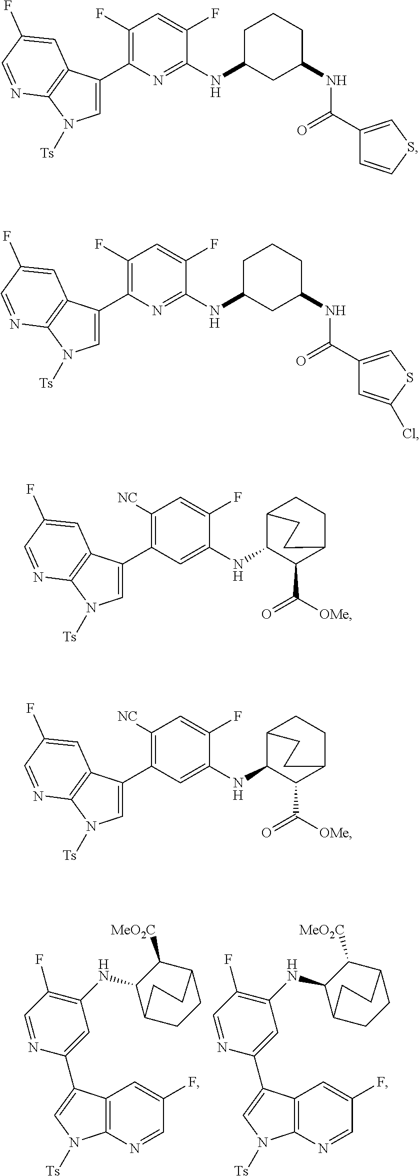
or a pharmaceutically acceptable salt thereof, wherein Ts is tosyl.
